SEQUENCE LISTING (152 sequences)

SEQ ID NO: 1 - protein, 431 aa, Homo sapiens
MSSIRFLCQRCHQALKLSGSSESRSLPAAPAPTSGQAEPGDTREPGVTTREVTDAEEQQDGASSRSPPGDGSVSKGHANIFTLLGELGAMHMLSSIQKAAGDIFDIVSGQAVVDHPLCEECTDSLLEQLDIQLALTEADSQNYQRCLETGELATSEDEAAALRAELRDLELEEARLVQELEDVDRNNARAAADLQAAQAEAAELDQQERQHYRDYSALKRQQLELLDQLGNVENQLQYARVQRDRLKEINCFTATFEIWVEGPLGVINNFRLGRLPTVRVGWNEINTAWGQAALLLLTLANTIGLQFQRYRLIPCGNHSYLKSLTDDRTELPLFCYGGQDVFLNNKYDRAMVAFLDCMQQFKEEAEKGELGLSLPYGIQVETGLMEDVGGRGECYSIRTHLNTQELWTKALKFMLINFKWSLIWVASRYQK

SEQ ID NO: 2 - DNA, 1296 bp, Homo sapiens
atgtcttccatccgcttcctgtgccagcgctgccaccaggccctgaagctgagcggctcctcggagtctaggagcctccctgcagccccggcgcccacctctgggcaggctgagcccggagacacccgggagcccggcgtcaccaccagggaggtgacagacgctgaggagcaacaggacggtgcctctagcagatcccctccaggcgatggcagtgtgtccaagggccatgccaacatcttcaccctgctgggggagcttggcgccatgcacatgctcagtagcatccagaaggcagctggtgacatttttgacatagtctctggccaagcagttgtggaccatcccctgtgtgaagaatgcaccgacagtcttttagagcagctggacatccagctcgctctcacagaagctgacagtcagaactaccaacgctgcctggagaccggggagctggcgaccagcgaggacgaggcggcggcgctgcgggcggagctgcgggacctggagctggaggaggccaggctggtgcaggagctggaggatgtggacaggaacaatgcaagagcagcggcggatctccaggcagcccaggcagaggctgcggagctggaccagcaggagaggcagcactacagggactacagtgccttgaagcggcagcagctggaactgcttgatcagctggggaacgtggagaaccagctgcagtatgccagggtccagagggaccggctgaaggaaatcaactgtttcaccgccacgtttgagatctgggtggagggccccttgggcgtcatcaataacttcaggttgggccgcctccccactgtccgtgtgggctggaatgagattaacactgcctggggacaggcggccttgctgctccttaccctggccaatacaattggactgcagtttcagaggtatcgactcatcccctgcggaaaccattcgtatctgaagtctttaacagatgaccgcactgagctgccgttgttctgttatggggggcaggatgttttcctcaataacaagtatgaccgcgcgatggtggccttcctggactgcatgcagcagttcaaggaagaggctgagaagggtgagctgggcctctctctgccctacgggatccaggtggagacaggcctgatggaggacgttggcggccgaggggaatgctattccatcagaacccatctgaacacgcaggagctgtggacaaaggcactcaagttcatgcttataaatttcaagtggagtctcatctgggttgcctcaaggtatcaaaagtag

SEQ ID NO: 3 - protein, 323 aa, Homo sapiens
MQAVVDHPLCEECTDSLLEQLDIQLALTEADSQNYQRCLETGELATSEDEAAALRAELRDLELEEARLVQELEDVDRNNARAAADLQAAQAEAAELDQQERQHYRDYSALKRQQLELLDQLGNVENQLQYARVQRDRLKEINCFTATFEIWVEGPLGVINNFRLGRLPTVRVGWNEINTAWGQAALLLLTLANTIGLQFQRYRLIPCGNHSYLKSLTDDRTELPLFCYGGQDVFLNNKYDRAMVAFLDCMQQFKEEAEKGELGLSLPYGIQVETGLMEDVGGRGECYSIRTHLNTQELWTKALKFMLINFKWSLIWVASRYQK

SEQ ID NO: 4 - DNA, 972 bp, Homo sapiens
atgcaagcagttgtggaccatcccctgtgtgaagaatgcaccgacagtcttttagagcagctggacatccagctcgctctcacagaagctgacagtcagaactaccaacgctgcctggagaccggggagctggcgaccagcgaggacgaggcggcggcgctgcgggcggagctgcgggacctggagctggaggaggccaggctggtgcaggagctggaggatgtggacaggaacaatgcaagagcagcggcggatctccaggcagcccaggcagaggctgcggagctggaccagcaggagaggcagcactacagggactacagtgccttgaagcggcagcagctggaactgcttgatcagctggggaacgtggagaaccagctgcagtatgccagggtccagagggaccggctgaaggaaatcaactgtttcaccgccacgtttgagatctgggtggagggccccttgggcgtcatcaataacttcaggttgggccgcctccccactgtccgtgtgggctggaatgagattaacactgcctggggacaggcggccttgctgctccttaccctggccaatacaattggactgcagtttcagaggtatcgactcatcccctgcggaaaccattcgtatctgaagtctttaacagatgaccgcactgagctgccgttgttctgttatggggggcaggatgttttcctcaataacaagtatgaccgcgcgatggtggccttcctggactgcatgcagcagttcaaggaagaggctgagaagggtgagctgggcctctctctgccctacgggatccaggtggagacaggcctgatggaggacgttggcggccgaggggaatgctattccatcagaacccatctgaacacgcaggagctgtggacaaaggcactcaagttcatgcttataaatttcaagtggagtctcatctgggttgcctcaaggtatcaaaagtag

SEQ ID NO: 5 - protein, 750 aa, Artificial Sequence
  Synthetic construct
MKKTAIAIAVALAGFATVAQAAELDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHSPLTFGAGTKLELKSSGGGGSGGGGGGSSRSSLEVQLQQSGPELVKPGASVKISCKTSEYTFTEYTKHWVKQSHGKSLEWIGSINPNNGDTYYNQKFTDKATLTVDKSSTTASMELRSLTFEDSAVYYCAMGDSAWFAYWGQGTLVTVSAAKTTPPSVTSGQAGQGGGGSGGGGSGGGGSSSIRFLCQRCHQALKLSGSSESRSLPAAPAPTSGQAEPGDTREPGVTTREVTDAEEQQDGASSRSPPGDGSVSKGHANIFTLLGELGAMHMLSSIQKAAGDIFDIVSGQAVVDHPLCEECTDSLLEQLDIQLALTEADSQNYQRCLETGELATSEDEAAALRAELRDLELEEARLVQELEDVDRNNARAAADLQAAQAEAAELDQQERQHYRDYSALKRQQLELLDQLGNVENQLQYARVQRDRLKEINCFTATFEIWVEGPLGVINNFRLGRLPTVRVGWNEINTAWGQAALLLLTLANTIGLQFQRYRLIPCGNHSYLKSLTDDRTELPLFCYGGQDVFLNNKYDRAMVAFLDCMQQFKEEAEKGELGLSLPYGIQVETGLMEDVGGRGECYSIRTHLNTQELWTKALKFMLINFKWSLIWVASRYQKHHHHHHGAYPYDVPDYAS

SEQ ID NO: 6 - DNA, 2253 bp, Artificial Sequence
  Synthetic construct
atgaagaaaaccgccattgccattgccgtggctctggccggatttgccacagttgctcaggccgccgaactggacgttgtgatgacacagacccctctgacactgagcgtgaccattggacagcctgccagcatcagctgcaagagcagtcagagcctgctgtactccaacggcaagacctacctgaactggctgctgcagaggcctggccagagtcctaagagactgatctacctggtgtccaagctggacagcggcgtgcccgatagattcacaggctctggcagcggcaccgacttcaccctgaagatctctagagtggaagccgaggacctgggcgtgtactattgtgtgcagggcacacacagccctctgacctttggagccggcacaaagctggaactgaagtctagcggaggcggcggatctggtggtggtggcggaggatctagcagatccagtctggaagtccagctgcagcagagcggacctgagcttgtgaaacctggcgcctccgtgaagatttcctgcaagaccagcgagtacaccttcaccgagtacacaaagcactgggtcaagcagtcccacggcaagagcctggaatggatcggcagcatcaaccccaacaacggcgacacctactacaaccagaagttcaccgacaaggccacactgaccgtggacaagagcagcaccacagccagcatggaactgcggagcctgaccttcgaagatagcgccgtgtactactgcgccatgggcgactctgcttggtttgcctattggggccagggcaccctggttacagtgtctgccgccaagacaacccctcctagcgttacatctggacaggccggacaaggtggcggtggttctggcggcggaggtagtggcggaggcggatcttcttccatccgcttcctgtgccagcgctgccaccaggccctgaagctgagcggctcctcggagtctaggagcctccctgcagccccggcgcccacctctgggcaggctgagcccggagacacccgggagcccggcgtcaccaccagggaggtgacagacgctgaggagcaacaggacggtgcctctagcagatcccctccaggcgatggcagtgtgtccaagggccatgccaacatcttcaccctgctgggggagcttggcgccatgcacatgctcagtagcatccagaaggcagctggtgacatttttgacatagtctctggccaagcagttgtggaccatcccctgtgtgaagaatgcaccgacagtcttttagagcagctggacatccagctcgctctcacagaagctgacagtcagaactaccaacgctgcctggagaccggggagctggcgaccagcgaggacgaggcggcggcgctgcgggcggagctgcgggacctggagctggaggaggccaggctggtgcaggagctggaggatgtggacaggaacaatgcaagagcagcggcggatctccaggcagcccaggcagaggctgcggagctggaccagcaggagaggcagcactacagggactacagtgccttgaagcggcagcagctggaactgcttgatcagctggggaacgtggagaaccagctgcagtatgccagggtccagagggaccggctgaaggaaatcaactgtttcaccgccacgtttgagatctgggtggagggccccttgggcgtcatcaataacttcaggttgggccgcctccccactgtccgtgtgggctggaatgagattaacactgcctggggacaggcggccttgctgctccttaccctggccaatacaattggactgcagtttcagaggtatcgactcatcccctgcggaaaccattcgtatctgaagtctttaacagatgaccgcactgagctgccgttgttctgttatggggggcaggatgttttcctcaataacaagtatgaccgcgcgatggtggccttcctggactgcatgcagcagttcaaggaagaggctgagaagggtgagctgggcctctctctgccctacgggatccaggtggagacaggcctgatggaggacgttggcggccgaggggaatgctattccatcagaacccatctgaacacgcaggagctgtggacaaaggcactcaagttcatgcttataaatttcaagtggagtctcatctgggttgcctcaaggtatcaaaagcatcatcatcatcatcatggcgcgtatccgtatgatgtgccggattatgcgagctag

SEQ ID NO: 7 - protein, 114 aa, Artificial Sequence
  Synthetic construct
ELDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHSPLTFGAGTKLELK

SEQ ID NO: 8 - protein, 110 aa, Artificial Sequence
  Synthetic construct
LEVQLQQSGPELVKPGASVKISCKTSEYTFTEYTKHWVKQSHGKSLEWIGSINPNNGDTYYNQKFTDKATLTVDKSSTTASMELRSLTFEDSAVYYCAMGDSAWFAYWGQ

SEQ ID NO: 9 - protein, 242 aa, Artificial Sequence
  Synthetic construct
ELDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHSPLTFGAGTKLELKSSGGGGSGGGGGGSSRSSLEVQLQQSGPELVKPGASVKISCKTSEYTFTEYTKHWVKQSHGKSLEWIGSINPNNGDTYYNQKFTDKATLTVDKSSTTASMELRSLTFEDSAVYYCAMGDSAWFAYWGQ

SEQ ID NO: 10 - DNA, 20 bp, Artificial Sequence
  Synthetic construct (Primer)
actacagtgccttgaagcgg

SEQ ID NO: 11 - DNA, 20 bp, Artificial Sequence
  Synthetic construct (Primer)
ctcaaacgtggcggtgaaac

SEQ ID NO: 12 - DNA, 21 bp, Artificial Sequence
  Synthetic construct (Primer)
ataaggacacaggtcacccaa

SEQ ID NO: 13 - DNA, 22 bp, Artificial Sequence
  Synthetic construct (Primer)
tgctccacatcttcgtatctca

SEQ ID NO: 14 - DNA, 20 bp, Artificial Sequence
  Synthetic construct (Primer)
aggttcggatcaagccttcc

SEQ ID NO: 15 - DNA, 21 bp, Artificial Sequence
  Synthetic construct (Primer)
tgtcgctcaggtacatatccc

SEQ ID NO: 16 - DNA, 21 bp, Artificial Sequence
  Synthetic construct (Primer)
cggatgagccgttacagtatc

SEQ ID NO: 17 - DNA, 23 bp, Artificial Sequence
  Synthetic construct (Primer)
actccaagcgtttaatagtgtcg

SEQ ID NO: 18 - DNA, 21 bp, Artificial Sequence
  Synthetic construct (Primer)
tgccttactacactgctgctc

SEQ ID NO: 19 - DNA, 21 bp, Artificial Sequence
  Synthetic construct (Primer)
aagctgtaccaaaatcgcaga

SEQ ID NO: 20 - DNA, 21 bp, Artificial Sequence
  Synthetic construct (Primer)
ggcgaattatagcgttcagcc

SEQ ID NO: 21 - DNA, 23 bp, Artificial Sequence
  Synthetic construct (Primer)
gggacaacagcaatatcctaagg

SEQ ID NO: 22 - DNA, 19 bp, Artificial Sequence
  Synthetic construct (Primer)
caggtgcggatcaaggctt

SEQ ID NO: 23 - DNA, 20 bp, Artificial Sequence
  Synthetic construct (Primer)
ccgctcaggaacatagccag

SEQ ID NO: 24 - DNA, 20 bp, Artificial Sequence
  Synthetic construct (Primer)
ccggtgagatgatcgaagcc

SEQ ID NO: 25 - DNA, 22 bp, Artificial Sequence
  Synthetic construct (Primer)
gccgaagctctacaataaacgc

SEQ ID NO: 26 - DNA, 22 bp, Artificial Sequence
  Synthetic construct (Primer)
aaaccaccaaacttactgctgg

SEQ ID NO: 27 - DNA, 22 bp, Artificial Sequence
  Synthetic construct (Primer)
cgcgttatcacttcccaaagaa

SEQ ID NO: 28 - DNA, 28 bp, Artificial Sequence
  Synthetic construct (Primer)
gtgagtcgtattaatttcgataagccag

SEQ ID NO: 29 - DNA, 27 bp, Artificial Sequence
  Synthetic construct (Primer)
cccggcttagacttttttctaaagatg

SEQ ID NO: 30 - DNA, 26 bp, Artificial Sequence
  Synthetic construct (Primer)
gaggtaagcagagtaaaagtgcagag

SEQ ID NO: 31 - DNA, 20 bp, Artificial Sequence
  Synthetic construct (sgRNA)
aagagcagcggcggatctcc

SEQ ID NO: 32 - DNA, 20 bp, Artificial Sequence
  Synthetic construct (sgRNA)
ccacgtgcgcaagaccagag

SEQ ID NO: 33 - DNA, 20 bp, Artificial Sequence
  Synthetic construct (sgRNA)
ggtgcagaacgttagctgaa

SEQ ID NO: 34 - DNA, 20 bp, Artificial Sequence
  Synthetic construct (sgRNA)
caatttagtcccggacatga

SEQ ID NO: 35 - DNA, 20 bp, Artificial Sequence
  Synthetic construct (sgRNA)
atttattgaaactcctcgcc

SEQ ID NO: 36 - DNA, 20 bp, Artificial Sequence
  Synthetic construct (sgRNA)
ttcaagcagcgccgcacctt

SEQ ID NO: 37 - DNA, 20 bp, Artificial Sequence
  Synthetic construct (sgRNA)
tctggaaacggaggatgcgg

SEQ ID NO: 38 - DNA, 20 bp, Artificial Sequence
  Synthetic construct (sgRNA)
tctgcggtttcaggtcgcgg

SEQ ID NO: 39 - DNA, 20 bp, Artificial Sequence
  Synthetic construct (sgRNA)
atgatggcggccacactctg

SEQ ID NO: 40 - DNA, 20 bp, Artificial Sequence
  Synthetic construct (sgRNA)
ttagacccgtagcgtttccg

SEQ ID NO: 41 - DNA, 20 bp, Artificial Sequence
  Synthetic construct (sgRNA)
gcttggcaaatgtgttgcta

SEQ ID NO: 42 - DNA, 20 bp, Artificial Sequence
  Synthetic construct (sgRNA)
ttgccgagtacttgtaatga

SEQ ID NO: 43 - DNA, 20 bp, Artificial Sequence
  Synthetic construct (sgRNA)
caagtactcggcaagttgtc

SEQ ID NO: 44 - DNA, 20 bp, Artificial Sequence
  Synthetic construct (sgRNA)
ttatgacccagaatgtggag

SEQ ID NO: 45 - DNA, 20 bp, Artificial Sequence
  Synthetic construct (sgRNA)
acggttccagtctctcggtg

SEQ ID NO: 46 - DNA, 20 bp, Artificial Sequence
  Synthetic construct (sgRNA)
gttgtcgaaggacttctcgt

SEQ ID NO: 47 - DNA, 20 bp, Artificial Sequence
  Synthetic construct (sgRNA)
gtcccagttgaggaccttga

SEQ ID NO: 48 - DNA, 20 bp, Artificial Sequence
  Synthetic construct (sgRNA)
ggatgttagaagagatcctc

SEQ ID NO: 49 - DNA, 20 bp, Artificial Sequence
  Synthetic construct (sgRNA)
gagttacaagatcatccaag

SEQ ID NO: 50 - DNA, 48 bp, Artificial Sequence
  Synthetic construct (Primer)
ggggacaagtttgtacaaaaaagcaggcttcatgtcttccatccgctt

SEQ ID NO: 51 - DNA, 47 bp, Artificial Sequence
  Synthetic construct (Primer)
ggggaccactttgtacaagaaagctgggtcctacttttgataccttg

SEQ ID NO: 52 - DNA, 49 bp, Artificial Sequence
  Synthetic construct (Primer)
ggggacaagtttgtacaaaaaagcaggcttcatggagagtaaatacaag

SEQ ID NO: 53 - DNA, 48 bp, Artificial Sequence
  Synthetic construct (Primer)
ggggaccactttgtacaagaaagctgggtcctatgttttttttttggc

SEQ ID NO: 54 - DNA, 48 bp, Artificial Sequence
  Synthetic construct (Primer)
ggggacaagtttgtacaaaaaagcaggcttcatgaagatggcaagcac

SEQ ID NO: 55 - DNA, 47 bp, Artificial Sequence
  Synthetic construct (Primer)
ggggaccactttgtacaagaaagctgggtcctaccaagaaggctcaa

SEQ ID NO: 56 - DNA, 48 bp, Artificial Sequence
  Synthetic construct (Primer)
ggggacaagtttgtacaaaaaagcaggcttcatggctggcggagcctg

SEQ ID NO: 57 - DNA, 48 bp, Artificial Sequence
  Synthetic construct (Primer)
ggggaccactttgtacaagaaagctgggtcctagtatctcctggcgtc

SEQ ID NO: 58 - DNA, 47 bp, Artificial Sequence
  Synthetic construct (Primer)
ggggacaagtttgtacaaaaaagcaggcttcatgaatttcataaagg

SEQ ID NO: 59 - DNA, 50 bp, Artificial Sequence
  Synthetic construct (Primer)
ggggaccactttgtacaagaaagctgggtcctattaagcagttactagtt

SEQ ID NO: 60 - DNA, 46 bp, Artificial Sequence
  Synthetic construct (Primer)
ggggacaagtttgtacaaaaaagcaggcttcatggcgcagtttgac

SEQ ID NO: 61 - DNA, 46 bp, Artificial Sequence
  Synthetic construct (Primer)
ggggaccactttgtacaagaaagctgggtcctataccttgtgcacc

SEQ ID NO: 62 - DNA, 45 bp, Artificial Sequence
  Synthetic construct (Primer)
ggggacaagtttgtacaaaaaagcaggcttcatggagcccggccg

SEQ ID NO: 63 - DNA, 50 bp, Artificial Sequence
  Synthetic construct (Primer)
ggggaccactttgtacaagaaagctgggtcctatcaggcacagatgccag

SEQ ID NO: 64 - DNA, 47 bp, Artificial Sequence
  Synthetic construct (Primer)
ggggacaagtttgtacaaaaaagcaggcttcggcgccatgcacatgc

SEQ ID NO: 65 - DNA, 51 bp, Artificial Sequence
  Synthetic construct (Primer)
ggggaccactttgtacaagaaagctgggtcctacttttgataccttgaggc

SEQ ID NO: 66 - DNA, 47 bp, Artificial Sequence
  Synthetic construct (Primer)
ggggacaagtttgtacaaaaaagcaggcttccaagcagttgtggacc

SEQ ID NO: 67 - DNA, 51 bp, Artificial Sequence; identical to SEQ ID NO: 65
  Synthetic construct (Primer)

SEQ ID NO: 68 - DNA, 45 bp, Artificial Sequence
  Synthetic construct (Primer)
ggggacaagtttgtacaaaaaagcaggcttcatcaactgtttcac

SEQ ID NO: 69 - DNA, 51 bp, Artificial Sequence; identical to SEQ ID NO: 65
  Synthetic construct (Primer)

SEQ ID NO: 70 - DNA, 51 bp, Artificial Sequence
  Synthetic construct (Primer)
ggggacaagtttgtacaaaaaagcaggcttcatgtcttccatccgcttcct

SEQ ID NO: 71 - DNA, 50 bp, Artificial Sequence
  Synthetic construct (Primer)
ggggaccactttgtacaagaaagctgggtcctattccttcagccggtccc

SEQ ID NO: 72 - DNA, 50 bp, Artificial Sequence
  Synthetic construct (Primer)
ggggacaagtttgtacaaaaaagcaggcttcatggagagtaaatacaagg

SEQ ID NO: 73 - DNA, 50 bp, Artificial Sequence
  Synthetic construct (Primer)
ggggaccactttgtacaagaaagctgggtcctaatttctgatggctgcag

SEQ ID NO: 74 - DNA, 48 bp, Artificial Sequence
  Synthetic construct (Primer)
ggggacaagtttgtacaaaaaagcaggcttcgatgtcgcaaagcaacg

SEQ ID NO: 75 - DNA, 48 bp, Artificial Sequence; identical to SEQ ID NO: 53
  Synthetic construct (Primer)

SEQ ID NO: 76 - DNA, 32 bp, Artificial Sequence
  Synthetic construct (Primer)
attgctagccaccatgtcttccatccgcttcc

SEQ ID NO: 77 - DNA, 30 bp, Artificial Sequence
  Synthetic construct (Primer)
tatctcgagcttttgataccttgaggcaac

SEQ ID NO: 78 - DNA, 42 bp, Artificial Sequence
  Synthetic construct (Primer)
gggagacccaagctggctagccaccatggcgcagtttgacac

SEQ ID NO: 79 - DNA, 42 bp, Artificial Sequence
  Synthetic construct (Primer)
cccttgctcaccatctcgagtaccttgtgcacctgagggggt

SEQ ID NO: 80 - DNA, 41 bp, Artificial Sequence
  Synthetic construct (Primer)
agctgtacaagtagggctccatgtcctgccgggatcggacc

SEQ ID NO: 81 - DNA, 41 bp, Artificial Sequence
  Synthetic construct (Primer)
cactgtgctggatatctgcatcaagcaaggaagaccacaaa

SEQ ID NO: 82 - DNA, 41 bp, Artificial Sequence
  Synthetic construct (Primer)
agctgtacaagtagggctccatgtccatggaggaccccttc

SEQ ID NO: 83 - DNA, 41 bp, Artificial Sequence
  Synthetic construct (Primer)
cactgtgctggatatctgcatcacagcactaagaagaggat

SEQ ID NO: 84 - DNA, 41 bp, Artificial Sequence
  Synthetic construct (Primer)
agctgtacaagtagggctccatgtctatgattttatctgcc

SEQ ID NO: 85 - DNA, 41 bp, Artificial Sequence
  Synthetic construct (Primer)
cactgtgctggatatctgcatcagacatcataatcgggagc

SEQ ID NO: 86 - DNA, 40 bp, Artificial Sequence
  Synthetic construct (Primer)
agctgtacaagtagggctccatggagagtaaatacaagga

SEQ ID NO: 87 - DNA, 40 bp, Artificial Sequence
  Synthetic construct (Primer)
cactgtgctggatatctgcactatgttttttttttggcct

SEQ ID NO: 88 - DNA, 20 bp, Artificial Sequence
  Synthetic construct (sgRNA)
aagagcagcggcggatctcc

SEQ ID NO: 89 - DNA, 20 bp, Artificial Sequence
  Synthetic construct (sgRNA)
taacaagtatgaccgcgcga

SEQ ID NO: 90 - DNA, 20 bp, Artificial Sequence
  Synthetic construct (sgRNA)
ttcaagcagcgccgcacctt

SEQ ID NO: 91 - DNA, 20 bp, Artificial Sequence
  Synthetic construct (sgRNA)
caatttagtcccggacatga

SEQ ID NO: 92 - DNA, 20 bp, Artificial Sequence
  Synthetic construct (sgRNA)
atttattgaaactcctcgcc

SEQ ID NO: 93 - DNA, 20 bp, Artificial Sequence
  Synthetic construct (sgRNA)
tctggaaacggaggatgcgg

SEQ ID NO: 94 - DNA, 20 bp, Artificial Sequence
  Synthetic construct (sgRNA)
cctcggcgacgtgcaccaac

SEQ ID NO: 95 - DNA, 20 bp, Artificial Sequence
  Synthetic construct (sgRNA)
tctgcggtttcaggtcgcgg

SEQ ID NO: 96 - DNA, 20 bp, Artificial Sequence
  Synthetic construct (sgRNA)
atgatggcggccacactctg

SEQ ID NO: 97 - DNA, 20 bp, Artificial Sequence
  Synthetic construct (sgRNA)
cacaaatctggagttcgtca

SEQ ID NO: 98 - DNA, 20 bp, Artificial Sequence
  Synthetic construct (sgRNA)
gaagacctgcacctctccga

SEQ ID NO: 99 - DNA, 20 bp, Artificial Sequence
  Synthetic construct (sgRNA)
tccgtgaccgtaccatgcag

SEQ ID NO: 100 - DNA, 20 bp, Artificial Sequence
  Synthetic construct (sgRNA)
tgtgcaacactacccgccgg

SEQ ID NO: 101 - DNA, 20 bp, Artificial Sequence
  Synthetic construct (sgRNA)
ggtggactccgtggacgatg

SEQ ID NO: 102 - DNA, 20 bp, Artificial Sequence
  Synthetic construct (sgRNA)
atcgttagactagcctatgc

SEQ ID NO: 103 - DNA, 20 bp, Artificial Sequence
  Synthetic construct (sgRNA)
ttagacccgtagcgtttccg

SEQ ID NO: 104 - DNA, 20 bp, Artificial Sequence
  Synthetic construct (sgRNA)
ttgccgagtacttgtaatga

SEQ ID NO: 105 - DNA, 20 bp, Artificial Sequence
  Synthetic construct (sgRNA)
gatagatgtacactgaaaac

SEQ ID NO: 106 - DNA, 20 bp, Artificial Sequence
  Synthetic construct (sgRNA)
aactcgctattcatggcata

SEQ ID NO: 107 - DNA, 20 bp, Artificial Sequence
  Synthetic construct (sgRNA)
ttatgacccagaatgtggag

SEQ ID NO: 108 - DNA, 20 bp, Artificial Sequence
  Synthetic construct (sgRNA)
acggttccagtctctcggtg

SEQ ID NO: 109 - DNA, 20 bp, Artificial Sequence
  Synthetic construct (sgRNA)
gttgtcgaaggacttctcgt

SEQ ID NO: 110 - DNA, 20 bp, Artificial Sequence
  Synthetic construct (sgRNA)
gtcccagttgaggaccttga

SEQ ID NO: 111 - DNA, 20 bp, Artificial Sequence
  Synthetic construct (sgRNA)
ggatgttagaagagatcctc

SEQ ID NO: 112 - DNA, 20 bp, Artificial Sequence
  Synthetic construct (sgRNA)
gagttacaagatcatccaag

SEQ ID NO: 113 - DNA, 20 bp, Artificial Sequence
  Synthetic construct (sgRNA)
caatttctcggatattggat

SEQ ID NO: 114 - DNA, 20 bp, Artificial Sequence
  Synthetic construct (sgRNA)
ccttgctaaactccatcggg

SEQ ID NO: 115 - DNA, 21 bp, Artificial Sequence
  Synthetic construct (Primer)
tcaagctgaaatgagtcctgc

SEQ ID NO: 116 - DNA, 21 bp, Artificial Sequence
  Synthetic construct (Primer)
cttgggtctcaaacgtgaagg

SEQ ID NO: 117 - DNA, 21 bp, Artificial Sequence
  Synthetic construct (Primer)
gtcaccagttcctcagttgtg

SEQ ID NO: 118 - DNA, 23 bp, Artificial Sequence
  Synthetic construct (Primer)
cacctccattgtccctgttttat

SEQ ID NO: 119 - DNA, 23 bp, Artificial Sequence
  Synthetic construct (Primer)
cgtgagtcccattaagatggagt

SEQ ID NO: 120 - DNA, 23 bp, Artificial Sequence
  Synthetic construct (Primer)
cccgacagtggatatagaacaga

SEQ ID NO: 121 - DNA, 20 bp, Artificial Sequence
  Synthetic construct (Primer)
atcaacaggcgagacctctg

SEQ ID NO: 122 - DNA, 21 bp, Artificial Sequence
  Synthetic construct (Primer)
gtcctcctggcataccataga

SEQ ID NO: 123 - protein, 6 aa, Artificial Sequence
  Synthetic construct (Linker)
LEGGGS

SEQ ID NO: 124 - protein, 4 aa, Artificial Sequence
  Synthetic construct (Linker)
TGSG

SEQ ID NO: 125 - protein, 8 aa, Artificial Sequence
  Synthetic construct (Linker)
GGSGGGSG

SEQ ID NO: 126 - protein, 5 aa, Artificial Sequence
  Synthetic construct (Linker)
GGGGS

SEQ ID NO: 127 - protein, 10 aa, Artificial Sequence
  Synthetic construct (Linker)
GGGGSGGGGS

SEQ ID NO: 128 - protein, 15 aa, Artificial Sequence
  Synthetic construct (Linker)
GGGGSGGGGSGGGGS

SEQ ID NO: 129 - protein, 20 aa, Artificial Sequence
  Synthetic construct (Linker)
GGGGSGGGGSGGGGSGGGGS

SEQ ID NO: 130 - protein, 4 aa, Artificial Sequence
  Synthetic construct (Linker)
GGGS

SEQ ID NO: 131 - protein, 8 aa, Artificial Sequence
  Synthetic construct (Linker)
GGGSGGGS

SEQ ID NO: 132 - protein, 12 aa, Artificial Sequence
  Synthetic construct (Linker)
GGGSGGGSGGGS

SEQ ID NO: 133 - protein, 16 aa, Artificial Sequence
  Synthetic construct (Linker)
GGGSGGGSGGGSGGGS

SEQ ID NO: 134 - protein, 6 aa, Artificial Sequence
  Synthetic construct (Linker)
GSGGGG

SEQ ID NO: 135 - protein, 12 aa, Artificial Sequence
  Synthetic construct (Linker)
GSGGGGGSGGGG

SEQ ID NO: 136 - protein, 18 aa, Artificial Sequence
  Synthetic construct (Linker)
GSGGGGGSGGGGGSGGGG

SEQ ID NO: 137 - protein, 24 aa, Artificial Sequence
  Synthetic construct (Linker)
GSGGGGGSGGGGGSGGGGGSGGGG

SEQ ID NO: 138 - protein, 6 aa, Artificial Sequence
  Synthetic construct (Linker)
GGGGGG

SEQ ID NO: 139 - protein, 7 aa, Artificial Sequence
  Synthetic construct (Linker)
GGGGGGG

SEQ ID NO: 140 - protein, 8 aa, Artificial Sequence
  Synthetic construct (Linker)
GGGGGGGG

SEQ ID NO: 141 - protein, 5 aa, Artificial Sequence
  Synthetic construct (Linker)
EAAAK

SEQ ID NO: 142 - protein, 10 aa, Artificial Sequence
  Synthetic construct (Linker)
EAAAKEAAAK

SEQ ID NO: 143 - protein, 15 aa, Artificial Sequence
  Synthetic construct (Linker)
EAAAKEAAAKEAAAK

SEQ ID NO: 144 - protein, 20 aa, Artificial Sequence
  Synthetic construct (Linker)
EAAAKEAAAKEAAAKEAAAK

SEQ ID NO: 145 - protein, 12 aa, Artificial Sequence
  Synthetic construct (Linker)
AEAAAKEAAAKA

SEQ ID NO: 146 - protein, 17 aa, Artificial Sequence
  Synthetic construct (Linker)
AEAAAKEAAAKEAAAKA

SEQ ID NO: 147 - protein, 22 aa, Artificial Sequence
  Synthetic construct (Linker)
AEAAAKEAAAKEAAAKEAAAKA

SEQ ID NO: 148 - protein, 27 aa, Artificial Sequence
  Synthetic construct (Linker)
AEAAAKEAAAKEAAAKEAAAKEAAAKA

SEQ ID NO: 149 - protein, 5 aa, Artificial Sequence
  Synthetic construct (Linker)
PAPAP

SEQ ID NO: 150 - protein, 12 aa, Artificial Sequence
  Synthetic construct (Linker)
APAPAPAPAPAP

SEQ ID NO: 151 - protein, 14 aa, Artificial Sequence
  Synthetic construct (Linker)
APAPAPAPAPAPAP

SEQ ID NO: 152 - protein, 16 aa, Artificial Sequence
  Synthetic construct (Linker)
APAPAPAPAPAPAPAP

(19) United States
(12) Patent Application Publication
(10) Pub. No.: US 2024/0025959 A1
(43) Pub. Date: Jan. 25, 2024

(54) BECLIN 2 AND USES THEREOF FOR TREATING CANCER AND NEURODEGENERATIVE DISEASES

(71) Applicants: UNIVERSITY OF SOUTHERN CALIFORNIA, Los Angeles, CA (US); THE METHODIST HOSPITAL SYSTEM, Houston, TX (US)

(72) Inventors: Rongfu Wang, Temple City, CA (US); Yicheng Wang, Temple City, CA (US); Motao Zhu, Beijing (CN)

(73) Assignees: UNIVERSITY OF SOUTHERN CALIFORNIA, Los Angeles, CA (US); THE METHODIST HOSPITAL SYSTEM, Houston, TX (US)

(21) Appl. No.: 18/020,502

(22) PCT Filed: Aug. 24, 2021
(86) PCT No.: PCT/US2021/047388

§ 371 Date: Feb. 9, 2023

Related U.S. Application Data
(60) Provisional application No. 63/069,413, filed on Aug. 24, 2020.

(51) Int. Cl. C07K 14/705; C07K 16/28; C07K 16/18; A61P 25/28

(52) U.S. Cl. CPC C07K 14/705; C07K 16/28; C07K 16/18; A61P 25/28; C07K 2319/00; C07K 2317/569; C07K 2317/55; C07K 2317/622

(57) ABSTRACT

Disclosed herein are recombinant proteins and/or polypeptides comprising a Beclin 2 polypeptide and/or a targeting moiety and methods relating to treating, preventing, reducing, and/or inhibiting a cancer, metastasis, and/or a neurodegenerative disease comprising administering said recombinant protein or polypeptides.

### CROSS-REFERENCE TO RELATED APPLICATIONS

This application includes a claim of priority under 35 U.S.C. § 119(e) to U.S. provisional patent application No. 63/069,413, filed Aug. 24, 2020, the entirety of which is hereby incorporated by reference.

### REFERENCE TO SEQUENCE LISTING

The Sequence Listing submitted Aug. 24, 2021 as a text file named “SequenceListing-065715-000133WO00_ST25” created on Aug. 24, 2021 and having a size of 58 kilobytes, is hereby incorporated by reference.

### TECHNICAL FIELD

The present invention relates to recombinant proteins and/or polypeptides comprising a Beclin 2 polypeptide and/or a targeting moiety and methods relating to treating, preventing, reducing, and/or inhibiting a cancer, metastasis, and/or a neurodegenerative disease.

### BACKGROUND

Chemotherapy, radiotherapy, and immunotherapy have been extensively used to treat cancer, however, new targets for the control of tumor development are still urgently demanded. Neurodegenerative diseases affect millions of people worldwide. Alzheimer's disease and Parkinson's disease are the most common neurodegenerative diseases. Clinical trials are ongoing and the search for effective drug(s) against neurodegenerative diseases and cancers being pursued worldwide. Autophagy is an essential cellular process for maintaining cell homeostasis and attenuating cell stresses through a “self-eating” mechanism. Some autophagy-related (ATG) proteins have been identified to function in autophagy to control physiological and pathological processes. What is need are compositions and method for treating cancers and/or neurodegenerative diseases. The compositions and methods disclosed herein address these and other needs.

### SUMMARY

Disclosed herein are methods relates to methods for treating, inhibiting, reducing, decreasing, ameliorating, and/or preventing a cancer, metastasis, and/or a neurodegenerative disease in a subject.

The details of one or more embodiments of the invention are set forth in the accompanying drawings and the description below. Other features, objects, and advantages of the invention will be apparent from the description and drawings, and from the claims.

In some aspects, disclosed herein are engineered Beclin 2 polypeptides and proteins including, but not limited to Beclin 2 polypeptides or proteins. For example, disclosed herein are substituted or truncated Beclin 2 proteins or polypeptides comprising binding domains ATG9A, STX5/6, and/or target proteins. In some aspects, the substituted or truncated Beclin 2 can comprise partial or substituted binding domains for ATG9A, STX5/6, and/or target proteins so long as binding to ATG9A, STX5/6, and/or target proteins is retained. Also disclosed herein are modified Beclin2 proteins and or polypeptides operatively linked (such as a fusion protein or chemically linked protein) to an antibody, antibody fragment, or small molecule. For example, a full-length, substituted, or truncated Beclin 2 operatively linked to an anti-Tau antibody or fragment thereof (such as, for example, an anti-Tau ScFV) resulting in, for example, an anti-Tau ScFV-Beclin 2 fusion or an anti-Tau ScFV-ATG9a fusion.

Also disclosed herein recombinant proteins and polypeptides and proteins comprising i) a modified or unmodified Beclin protein or polypeptide and ii) a targeting moiety (such as, for example, a small molecule, antibody, or antibody fragment). In some aspects, the Beclin-2 protein or polypeptide (of either the engineered Beclin 2 or the recombinant protein or polypeptide) comprises an ATG9A-binding domain, including, but not limited to an ATG9A-binding domain comprising a polypeptide sequence at least 70% identical to SEQ ID NO: 3 or a fragment thereof.

Also disclosed herein are recombinant proteins and polypeptides of any preceding aspect, wherein the targeting moiety specifically binds to a peptide, protein, and/or pathogenic molecule related to a neurodegenerative disease (such as, for example, TAU, β-amyloid, APOE, SUPT5H, TDP43, GAK, PINK1, PARK2, PARK7, or TREM2 protein) or specifically binds to a pathogenic molecule, peptide, or protein related to a cancer and/or metastasis (such as, for example, MEKK3, TAK1, NLRP3, NLRC4, NLRP1, AIM2, as well as tumor antigens or oncogenes (derived from point mutations, amplification and fusion) such as a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LiCAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COLIA1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKARIA, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAa, TRBa, TRDa, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, or ZNFN1A1.

For example, disclosed herein are recombinant proteins and polypeptides of any preceding aspect, wherein the targeting moiety comprises an antibody or antibody fragment that specifically binds TAU (such as, for example, an antibody or antibody fragment comprising a variable region comprising a polypeptide sequence at least 80% identical to SEQ ID NO: 9, including, but not limited to an antibody or antibody fragment comprising a polypeptide sequence at least 80% identical to SEQ ID NO: 9).

Also disclosed herein are recombinant polynucleotides encoding the recombinant protein or polypeptide of any preceding aspect, wherein the recombinant polynucleotide comprises a polynucleotide sequence at least 80% identical to SEQ ID NO: 2.

In some aspects, disclosed herein are methods of treating, inhibiting, reducing, decreasing, ameliorating, and/or preventing a cancer or metastasis (such as, for example, a metastatic lymphoma, lung cancer, T cell lymphoma, or B cell lymphoma) in a subject in need thereof, comprising administering to the subject a therapeutically effective amount of an engineered Beclin 2 protein or polypeptide, a recombinant protein or polypeptide, or a polynucleotide of any preceding aspect. For example, in some aspects, disclosed herein are methods of treating, inhibiting, reducing, decreasing, ameliorating, and/or preventing a cancer or metastasis (such as, for example, a metastatic lymphoma, lung cancer, T cell lymphoma, or B cell lymphoma) in a subject in need thereof, comprising administering to the subject a therapeutically effective amount of a recombinant protein or polypeptide comprising a Beclin 2 polypeptide or a fragment thereof comprising a polypeptide sequence at least 70% identical to SEQ ID NO: 3 or a fragment thereof (including, but not limited to a Beclin-2 polypeptide comprising an ATG9A-binding domain as set forth in SEQ ID NO: 3). In one aspect, disclosed herein are methods of treating, inhibiting, reducing, decreasing, ameliorating, and/or preventing a cancer or metastasis of any preceding aspect, wherein the Beclin-2 polypeptide is at least 70% identical to SEQ ID NO: 1 or 3. Also disclosed herein are methods of treating, inhibiting, reducing, decreasing, ameliorating, and/or preventing a cancer or metastasis of any preceding aspect, wherein the recombinant protein or polypeptide further comprises a targeting moiety operatively linked to the Beclin 2 polypeptide or a fragment thereof. In some embodiments, the targeting moiety specifically binds to a pathogenic molecule, peptide, or protein associated with a cancer, wherein the targeting moiety specifically binds to MEKK3, TAK1, NLRP3, NLRC4, NLRP1, AIM2 protein, as well as tumor antigens or oncogenes (derived from point mutations, amplification and fusion) such as a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LiCAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COLIA1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEll0, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYHI 1, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKARIA, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC L1 8, WRN, WT1, XPA, XPC, ZNF145, ZNF198, ZNF278, ZNF384, or ZNFN1A1.

In one example, the targeting moiety comprises an antibody or functional fragment thereof. In one example, the targeting moiety comprises a small molecule.

Also disclosed herein is a method of treating, inhibiting, reducing, decreasing, ameliorating, and/or preventing a cancer or metastasis of any preceding aspect comprising increasing Beclin-2 polypeptide levels in a cancer cell, wherein the method comprises administering to the subject a therapeutically effective amount of the recombinant protein or polypeptide and/or the recombinant polynucleotide of any preceding aspect, wherein the recombinant protein or polypeptide or the recombinant polynucleotide decreases a level of TAK1, and/or MEKK3 in the cancer cell, and wherein the recombinant protein or polypeptide or the recombinant polynucleotide decreases cancer cell proliferation.

Also disclosed herein are methods of treating, inhibiting, reducing, decreasing, ameliorating, and/or preventing a neurodegenerative disease (such as, for example, Alzheimer's disease (AD), Parkinson's disease (PD), Huntington's Disease, Amyotrophic Lateral Sclerosis (ALS), or Multiple Sclerosis (MS)) in a subject in need thereof, comprising administering to the subject a therapeutically effective amount of a recombinant protein or polypeptide comprising a modified or unmodified Beclin 2 protein, polypeptide, or a fragment thereof (including, but not limited to a Beclin-2 polypeptide comprising an ATG9A-binding domain comprising a polypeptide sequence at least 70% identical to SEQ ID NO: 3 or a fragment thereof). In some embodiments, disclosed herein are methods of treating, inhibiting, reducing, decreasing, ameliorating, and/or preventing a neurodegenerative disease (such as, for example, Alzheimer's disease (AD), Parkinson's disease (PD), Huntington's Disease, Amyotrophic Lateral Sclerosis (ALS), or Multiple Sclerosis (MS)) of any preceding aspect, wherein the recombinant protein or polypeptide further comprises a targeting moiety (such as, for example, an antibody, antibody fragment, or small molecule that specifically binds to a pathogenic molecule, peptide, or protein related to a neurodegenerative disease including, but not limited to TAU, β-amyloid, APOE, SUPT5H, TDP43, GAK, PINK1, PARK2, PARK7, or TREM2 protein) operatively linked to the Beclin 2 polypeptide or a fragment thereof. For example, disclosed herein are methods of treating, inhibiting, reducing, decreasing, ameliorating, and/or preventing a neurodegenerative disease (such as, for example, Alzheimer's disease (AD), Parkinson's disease (PD), Huntington's Disease, Amyotrophic Lateral Sclerosis (ALS), or Multiple Sclerosis (MS)) in a subject in need thereof, comprising administering to the subject a therapeutically effective amount of a recombinant protein or polypeptide of any preceding aspect. In one example, disclosed herein are methods of treating, inhibiting, reducing, decreasing, ameliorating, and/or preventing a neurodegenerative disease of any preceding aspect wherein the targeting moiety comprises an antibody or functional fragment thereof, wherein the antibody or antibody fragment comprises a variable region comprising a polypeptide sequence at least 80% identical to SEQ ID NO: 9, and wherein the antibody or antibody fragment comprises a polypeptide sequence at least 80% identical to SEQ ID NO: 9.

Also disclosed herein is a method of treating, inhibiting, reducing, decreasing, ameliorating, and/or preventing a neurodegenerative disease of any preceding aspect comprising increasing Beclin-2 polypeptide levels in a neural cell, comprising administering to the subject a therapeutically effective amount of the recombinant protein or polypeptide or the recombinant polynucleotide of any preceding aspects, wherein the recombinant protein or polypeptide or the recombinant polynucleotide decreases a level of an inflammatory cytokine (e.g., IL-1β or IL-6), wherein the recombinant protein or polypeptide or the recombinant polynucleotide decreases a level of AIM2, NLRP3, NLRP1, and/or NLRC4 in the neural cell.

Also disclosed herein is a method of decreasing a level of TAK1 and/or MEKK3 in a subject, comprising administering to the subject a therapeutically effective amount of the recombinant protein or polypeptide or the recombinant polynucleotide of any preceding aspect, wherein the subject is a cancer patient.

Also disclosed herein is a method of decreasing a level of AIM2, NLRP3, NLRP1, and/or NLRC4 in a subject, comprising administering to the subject a therapeutically amount of the recombinant protein or polypeptide or a recombinant polynucleotide of any preceding aspect, wherein the subject is a neurodegenerative disease patient.

### BRIEF DESCRIPTION OF DRAWINGS

FIGS. 1A, 1B, 1C, 1D, 1E, and 1F show splenomegaly and lymphadenopathy in Becn2-deficient mice. FIG. 1A shows RT-PCR analysis of human Beclin 2 expression in different tissues and cells. The mRNA levels of BECN2 in different tissues or cells were normalized to the mRNA levels of the GAPDH gene. The data are plotted as the mean±s.e.m. and are representative of at least three independent experiments. FIG. 1B shows the sizes of the spleen (upper) and lymph nodes (LNs, lower) from Becn2 KO and WT mice (n=4 per group). FIG. 1C shows H&E staining of spleen and LNs from Becn2 KO and WT mice. Images are representative of 4 mice in each group. FIG. 1D shows the total numbers of splenocytes and lymphocytes from LNs counted from 6- to 8-week-old Becn2 KO and WT mice (n=6 mice per group). FIG. 1E and FIG. 1F show representative flow cytometry plots and quantification of 6- to 8-week-old B220⁺ and CD3⁺ cell populations in spleens (FIG. 1E) and LNs (FIG. 1F) (n=8 mice per group). BM, bone marrow; LN, lymph nodes. Statistical differences between groups were calculated using Student's unpaired t-test. **P<0.01.

FIGS. 2A, 2B, 2C, 2E, and 2F show mouse genotyping and characterization for immune cell populations in Becn2 KO mouse. FIG. 2A shows real-time PCR for analysis of the mRNA levels of the mouse Becn2 in indicated tissues or cell types (3 biological replicates for each group). FIG. 2B shows genotyping for WT, heterozygous Becn2 KO and homozygous Becn2 KO mice. FIG. 2C shows H&E staining of thymus from WT and Becn2 KO mice. FIG. 2D and FIG. 2E show CYTOF analysis of T cell and B cell subsets in WT and Becn2 KO splenocytes. viSNE map of representative WT and Becn2 KO T cell and B cell subpopulations illustrated by color-coded cell populations that were clustered together based on similarity in cell surface marker expression (FIG. 2D). FIG. 2E shows the percentage of lymphocyte subpopulations in total CD45⁺ cells from spleen based on CYTOF analysis. FIG. 2F shows flow cytometry and quantification analysis of CD11b⁺Gr1b⁺ neutrophil and CD11b⁺F4/80⁺ macrophage populations in the spleens from WT and Becn2 KO mice. Statistical analysis is shown for at least three independent experiments and were calculated using Student's unpaired t-test.

FIGS. 3A, 3B, 3C, 3D, 3E, 3F, and 3G show that ablation of Becn2 increases proinflammatory cytokine production and the mouse sensitivity to LPS-induced septic shock. FIGS. 3A, 3B, and 3C show production of TNF-α, IL-6 and IL-1β by BMDCs (FIG. 3A), BMDMs (FIG. 3B), and peritoneal neutrophils (FIG. 3C) treated with LPS (100 ng/ml) alone or followed by ATP (5 mM) treatment for 1 h. FIG. 3D shows heatmap of the DEGs in Becn2 KO BMDMs compared with WT BMDMs after 3 h of LPS (100 ng/ml) stimulation. The most significant DEGs in the TNF signaling, NOD-like receptor signaling, cytokine-cytokine receptor interaction and chemokine signaling pathways between Becn2 KO BMDMs and WT BMDMs are listed on the right side. FIG. 3E shows survival of 6- to 8-week-old WT and Becn2 KO mice after high-dose LPS (i.p. 30 mg/kg) treatment (n=5 mice per group). FIG. 3F shows cytokine levels in serum samples from 6- to 8-week-old WT and Becn2 KO mice after high-dose LPS (i.p. 30 mg/kg) treatment (n=6 mice per group). FIG. 3G shows the IL-6 levels in 8- to 12-week-old mouse sera (n=10 mice per group). Data in FIGS. 3A-3C are plotted as the mean±s.e.m. and are representative of at least three independent experiments (n=2 mice each time/group). Statistical differences between groups were calculated using Student's unpaired t-test (for FIGS. 3A-3C, 3F, and 3G) and Log-rank test (FIG. 3E). *P<0.05; **P<0.01; ***P<0.001.

FIGS. 4A, 4B, 4C, 4D, 4E, and 4F show that loss of Beclin 2 enhances pro-inflammatory cytokine production at mRNA levels. FIG. 4A shows the significantly enriched KEGG pathways in Becn2 KO macrophages compared to WT macrophages after 3 h of LPS (100 ng/mL) stimulation. FIG. 4B shows production of TNF-α and IL-6 from BMDMs transfected with poly I:C (2 μg/ml). FIG. 4C shows production of TNF-α and IL-6 by BMDCs treated with CpG (10 μg/ml). FIG. 4D shows IFN-β production of BMDMs at 24 h post VSV infection or poly(dA:dT) transfection. FIG. 4E shows that 293T cells were co-transfected with increasing amounts of plasmids encoding Beclin 2 along with ISRE-Luc reporter, followed by stimulation with mock, poly(I:C) or poly(dA:dT). The luciferase expression in 293T cells was determined at 24 h post-stimulation. FIG. 4F shows TNF-α, IL-1β, IL-17, IFN-γ and IL-10 levels in 8- to 12-week old mice serum (n=10 per group). Error bars represent mean±s.e.m. Statistical differences between groups were calculated using Student's unpaired t-test (FIGS. 4B, 4C, 4D, and 4F) or 1-way ANOVA with Dunnett's multiple comparison test (FIG. 4E). Data are representative of at least three independent experiments (FIGS. 4B-4E). *P<0.05. NS, no significance; ND, not detectable.

FIGS. 5A, 5B, and 5C show that Becn2-deficiency enhances ERK, NF-κB and STAT3 signaling. (FIGS. 5A-5C) Immunoblot analysis of cell lysates from WT or Becn2 KO BMDCs (FIG. 5A), BMDMs (FIG. 5B), or peritoneal neutrophils (FIG. 5C) treated with LPS (100 ng/ml) for the indicated time points using the indicated antibodies. Samples were run on parallel gels contemporaneously (FIGS. 5A-5C). Bottom: Quantitative comparison of activation of signaling molecules between WT and Becn2 KO cells based on band intensity of three independent experiments. The data are plotted as the mean±s.e.m. Statistical differences between groups were calculated using Student's unpaired t-test (A-C). *P<0.05; **P<0.01.

FIGS. 6A, 6B, 6C, 6D, 6E, and 6F show that Beclin 2 deficiency enhances ERK1/2 and STAT3 signaling in response to poly I:C but not CpG, Pam3CSK4, or TNF-α. FIG. 6A shows immunoblot analysis of cell lysates from WT or Becn2 KO BMDMs transfected with poly I:C (2 μg/ml) for the indicated time points using indicated antibodies. FIG. 6B shows immunoblot analysis of cell lysates from WT or Becn2 KO BMDCs treated with CpG (10 μg/ml) for the indicated time points using the indicated antibodies. FIG. 6C shows immunoblot analysis of cell lysates from WT or Becn2 KO peritoneal macrophages treated with Pam3CSK4 (200 ng/ml) for the indicated time points using the indicated antibodies. FIG. 6D shows immunoblot analysis of cell lysates from WT or Becn2 KO BMDMs treated with TNF-α for the indicated time points using the indicated antibodies. FIG. 6E shows immunoblot analysis of cell lysates from WT or BECN2 KO THP-1 cells treated with LPS (100 ng/ml) for the indicated time points. FIG. 6F shows immunoblot analysis of pro-IL-1β of cell lysates from WT or Becn2 KO BMDMs treated with LPS (100 ng/ml) for 3 h. Data are representative of at least three independent experiments.

FIGS. 7A, 7B, 7C, 7D, 7E, 7F, 7G, and 7H show that Beclin 2 inhibits ERK1/2 signaling by targeting MEKK3 and TAK1 for autophagic degradation. FIG. 7A shows schematic illustration of proinflammatory gene expression controlled by key adaptors, kinases and transcription factors involved in NF-κB and MAPK signaling. FIG. 7B shows IL-6 production from WT, Becn2 KO, and sgRNA-guided Mapk3/1 KO Becn2 KO macrophages treated with LPS (100 ng/ml) for the indicated periods. FIG. 7C shows immunoblot analysis of p-STAT3, STAT3, and ERK1/2 in WT, Becn2 KO, and sgRNA-guided Mapk3/1 KO Becn2 KO macrophages treated with LPS (100 ng/ml). FIG. 7D shows that 293T cells co-transfected with the Flag-Beclin 2 plasmid alone or together with an HA-tagged plasmid were immunoprecipitated using anti-Flag beads, then immunoblotting with the indicated antibodies. FIG. 7E shows that BMDMs were left untreated or treated by LPS (100 ng/ml) for 3 h. Cell lysates of BMDMs were immunoprecipitated using MEKK3 or TAK1 specific antibodies against each protein, then immunoblotted with indicated antibodies. FIG. 7F shows immunoblot analysis of endogenous TAK1 and MEKK3 in the lysates of 293T cells transfected with an increasing amount of pcDNA-Beclin 2 plasmid (0, 500 and 1000 ng/10⁶ cells). FIG. 7G shows immunoblot analysis of TAK1 and MEKK3 in cell lysates of WT and Becn2 KO T cells, B cells, and macrophages, neutrophils and DCs before and after LPS (100 ng/ml, 2 h) treatment. Samples were run on parallel gels contemporaneously. FIG. 7H shows that 293T cells were transfected with HA-TAK1 or HA-MEKK3 alone or together with Flag-Beclin 2 plasmid, followed by treatment for 6-10 h with 3MA (5 mM), Bafilomycin A (BafA, 500 nM), CQ (10 μM), MRT68921 (1 μM) or MG132 (10 μM). Cell lysates were immunoblotted with the indicated antibodies. The data are representative of at least three independent experiments. Statistical differences between groups were calculated using 1-way ANOVA with Tukey's multiple comparison test (B). *P<0.05; **P<0.01.

FIGS. 8A, 8B, 8C, 8D, 8E, 8F, and 8G show that Beclin 2 targets TAK1 and MEKK3 for degradation. FIG. 8A shows that 293T cells were co-transfected with the Flag-Beclin 2 plasmid alone or together with an HA-tagged plasmid were immunoprecipitated using anti-Flag beads, followed by immunoprecipitation with anti-Flag beads, then immunoblotting with the indicated antibodies. FIG. 8B shows immunoblot analysis of 293T cells transfected with HA-MKK1 HA-MKK2 or HA-MKK3 alone, or together with increasing amount of Flag-Beclin 2 plasmid (100, 200 and 400 ng/10⁶ cells). FIG. 8C shows real-time PCR analysis of the mRNA levels of endogenous mouse TAK1 and MEKK3 in WT and Becn2 KO BMDMs, splenic T cells and B cells. FIG. 8D shows immunoblot analysis of p-TAK1 and TAK1 in cell lysates of WT and Becn2 KO T cells, B cells, and macrophages, neutrophils and DCs before and after LPS (100 ng/ml, 30 min) treatment. FIG. 8E shows cell lysates of BMDMs with or without LPS (100 ng/ml, 30 min) treatment were immunoprecipitated using anti-MEKK3 antibody, followed by immunoblotting with anti-phosphoserine antibody. FIG. 8F shows immunoblot analysis of endogenous TAK1 and MEKK3 levels in 293T cells treated with Bafilomycin A (BafA, 500 nM), CQ (10 μM), 3MA (5 mM), MRT68921 (1 μM) or MG132 (10 μM) for 6-10 h. Figure G shows real-time PCR analysis of human TAK1 and MEKK3 mRNA levels in Beclin 2-overexpressed 293T cells or 293T cells treated with Bafilomycin A (BafA, 500 nM), CQ (10 μM), 3MA (5 mM), MRT68921 (1 μM) or MG132 (10 μM) for 6-10 h. Data are representative of three independent experiments. Statistical differences between groups were calculated using Student's unpaired t-test (FIG. 8C) or 1-way ANOVA (FIG. 8G). NS, no significance.

FIGS. 9A, 9B, 9C, and 9D show that Beclin 2 mediates the degradation of TAK1 and MEKK3 through ATG16L/LC3/Beclin 1-independent pathway. FIGS. 9A-9C show WT 293T cells and sgRNA-guided ATG16L KO (FIG. 9A), MAP1LC3B KO (FIG. 9B) or BECN1 KO (FIG. 9C) 293T cell clones were transfected with HA-TAK1 or HA-MEKK3 alone or together with Flag-Beclin 2 plasmid, followed by immunoblotting with the indicated antibodies. Samples were run on parallel gels contemporaneously in panel (FIG. 9B). Bottom: Quantitative analysis of HA-TAK1 and HA-MEKK3 expression and degradation percentage in WT and KO cells after normalization based on band intensity of three independent experiments. The data are plotted as the mean±s.e.m. (FIG. 9D) Cell lysates of 293T cells were immunoprecipitated using specific antibodies against each protein, then immunoblotted using indicated antibodies. Statistical differences between EmpVec-transfected and Flag-Beclin 2-transfected cells were calculated using Student's unpaired t-test (FIGS. 9A-9C). Statistical differences of degradation percentages between WT and KO groups were calculated using 1-way ANOVA with Dunnett's multiple comparison test (FIGS. 9A-9C). *P<0.05; **P<0.01; ***P<0.001. NS, no significance.

FIGS. 10A, 10B, 10C, and 10D show that Beclin 2 mediates the MEKK3 and TAK1 degradation through an ATG16L/LC3/Beclin 1-independent pathway. FIG. 10A shows that 293T cells were co-transfected with HA-Beclin 2 and a Flag-tagged plasmid as indicated, followed by immunoprecipitation with anti-Flag beads, and then immunoblotting with the indicated antibodies. FIG. 10B shows that 293T cells were co-transfected with Flag-Beclin 2 and HA-ULK1, 24 h post-transfection, cells were left in either complete medium or Earle's Balanced Salts medium for 4 h to induce starvation, followed by immunoprecipitation with anti-Flag beads, and then immunoblotting with the indicated antibodies. FIG. 10C shows that WT, WIPI1 and WIPI2-sgRNA knockout 293T cells were co-transfected with HA-MEKK3 together with empty vector or Flag-Beclin 2 plasmid. Cell lysates were then immunoblotted with the indicated antibodies. FIG. 10D shows immunoblot analysis of endogenous TAK1 and MEKK3 protein levels in the lysates of WT and AIG KO 293T cells using indicated antibodies. Data are representative of three independent experiments.

FIGS. 11A, 11B, 11C, 11D, 11E, and 11F show that Beclin 2 mediates the degradation of TAK1 and MEKK3 through an ATG9A- and ULK-dependent autophagic pathway. FIGS. 11A and 11B show that WT 293T cells, sgRNA-guided ATG9A KO 293T cells (FIG. 11A), and sgRNA-guided ULK1 KO 293T cells (FIG. 11B) were transfected with HA-TAK1 or HA-MEKK3 alone or together with Flag-Beclin 2 plasmid, followed by immunoblotting with the indicated antibodies. The blots were run contemporaneously with the same protein samples (FIG. 11A). Bottom: Quantitative analysis of HA-TAK1 and HA-MEKK3 expression and degradation percentage in WT and KO cells after normalization based on band intensity of three independent experiments. FIGS. 11C and 11D show that WT and ATG9A KO (FIG. 11C) or ULK1 KO (FIG. 11D) 293T cells were transfected with the HA-Beclin 2 plasmid alone or together with Flag-MEKK3. Cell lysates were immunoprecipitated using anti-Flag beads, followed by immunoblotting with the indicated antibodies. The data are representative of three independent experiments. FIG. 11E shows confocal images of WT, ATG9A KO, and ULK1 KO 293T cells co-transfected with GFP-MEKK3 and Flag-Beclin 2, then stained with anti-Flag antibody, followed by Alexa Fluor 633-conjugated secondary antibody staining. Hoechst 33342 was applied for nucleus staining. Scale bar, 10 μm. FIG. 11F shows confocal images of WT, ATG9A KO and ULK1 KO 293T cells transfected with GFP-MEKK3, then stained with lysotracker and Hoechst 33342. Scale bar, 10 sm. The data are plotted as the mean±s.e.m. Pearson's correlation coefficient for colocalization in FIG. 11E and FIG. 11F was analyzed using Image J Coloc 2 (at least 30 cells were analyzed per condition). Statistical differences between EmpVec-transfected and Flag-Beclin 2-transfected cells were calculated using Student's unpaired t-test (FIGS. 11A and 11B). Statistical differences between WT and KO groups were calculated using Student's unpaired t-test (FIG. 11A) or 1-way ANOVA with Dunnett's multiple comparison test (FIGS. 11B, 11E, 11F). *P<0.05; **P<0.01; ***P<0.001. NS, no significance.

FIGS. 12A, 12B, 12C, 12D, and 12E show that Beclin 2 mediates the degradation of TAK1 and MEKK3 through an ATG9A- and ULK-dependent autophagic pathway. FIG. 12A shows that WT and ATG13 shRNA knockdown (KD) 293T cells were transfected with HA-MEKK3 alone or together with Flag-Beclin 2 plasmid, followed by immunoblotting with the indicated antibodies. Quantitative analysis of HA-TAK1 and HA-MEKK3 expression and degradation percentage in WT and ATG13 knockdown cells based on band intensity of 3 independent experiments. FIG. 12B shows that 293T cells were transfected with indicated Flag-tagged plasmid, followed by immunoprecipitation with anti-Flag beads, and then immunoblotting with indicated antibodies. FIG. 12C shows confocal images of WT and ULK1 KO 293T cells co-transfected with GFP-Beclin 2 and RFP-ATG9A, then stained with Hoechst 33342 for nucleus imaging. Scale bar, 10 μm. Pearson's correlation coefficient for Beclin 2-ATG9A colocalization was analyzed using Image J Coloc 2. Graph represents mean±s.d. (at least 30 cells were analyzed per condition). FIG. 12D shows that BMDMs cells were left untreated or stimulated with LPS (100 ng/ml) for indicated time points, the cell lysates were then immunoprecipitated using MEKK3 antibodies, followed by immunoblotted with indicated antibodies. FIG. 12E shows immunoblot analysis of endogenous MEKK3 protein levels in the lysates of WT, Becn1 KO and Becn2 KO BMDMs cells before and after LPS (100 ng/ml) stimulation. Data are representative of at least three independent experiments. Statistical differences between groups were calculated using Student's unpaired t-test (Figure A, Figure C). *P<0.05; **P<0.01; ***P<0.001.

FIGS. 13A, 13B, 13C, 13D, and 13E show that Beclin 2 binds to STX5/6 to promote the fusion of ATG9A-vesicles with phagophores for MEKK3 degradation. FIG. 13A shows transmission electron microscopy (TEM) images of WT and Flag-Beclin 2 transfected 293T cells. Black arrows indicate the double-membrane autophagosomal structures. The numbers of autophagosomes were counted. Graph represents mean±s.d. (at least 30 cells were counted per condition). FIG. 13B shows EM image of vesicle fusion with pre-existing double-membrane structure in Flag-Beclin 2 transfected 293T cells. Black arrow indicates the pre-existing double-membrane structure; red arrows indicate the isolated membrane vesicles. FIG. 13C shows that 293T cells were co-transfected with HA-Beclin 2 and a Flag-tagged plasmid as indicated, followed by immunoprecipitation with anti-Flag beads, and then immunoblotting with the indicated antibodies. FIG. 13D shows that WT 293T cells or sgRNA-guided RAB or SNARE KO 293T cell were transfected with HA-MEKK3 alone or together with Flag-Beclin 2 plasmid, followed by immunoblotting with the indicated antibodies. FIG. 13E shows schematic illustration of canonical autophagy pathway and Beclin 2-mediated non-canonical autophagic degradation of MEKK3/TAK1. PAS, phagophore assembly site. Statistical differences between groups were calculated using Student's unpaired t-test (FIG. 13A). ***P<0.001.

FIGS. 14A, 14B, 14C, 14D, and 14E show that Beclin 2 binds to STX5/6 to promote the fusion of ATG9A-vesicles with phagophores for MEKK3 degradation. FIG. 14A shows that WT, sgRNA-guided STX5 KO, STX6 KO, or STX5:STX6 DKO 293T cells were transfected with HA-MEKK3 alone or together with Flag-Beclin 2 plasmid, followed by immunoblotting with the indicated antibodies. The blots were run contemporaneously with the same protein samples. Bottom: Quantitative analysis of HA-MEKK3 expression and degradation percentage. FIG. 14B shows that cell lysates of 293T cells were immunoprecipitated using anti-STX5 or anti-STX6 antibody, respectively, and then immunoblotting with the indicated antibodies. FIG. 14C shows representative EM images of WT and STX5:STX6 DKO 293T cells co-overexpressing Flag-Beclin 2 and MEKK3-APEX2 processed in parallel under identical conditions. Black arrow indicates MEKK3 associated-vesicle; white arrowhead indicates MEKK3-containing autophagosome. FIG. 14D shows that WT, STX5 KO and STX6 KO were co-transfected with Flag-ATG9A and HA-Beclin 2, while BECN2 KO 293T cells were transfected with Flag-ATG9A alone. Half of the cells were left untreated for immuno-isolation of Flag-ATG9A⁺ vesicles, the other half were pre-treated by CQ (10 μM, 4 h) to inhibit the fusion of autophagosomes with lysosomes for the enrichment of autophagosomes. Cells were resuspended in specific fractionation buffers for isolation of Flag-ATG9A+ vesicles or autophagosome enrichment, respectively, followed by immunoblotting with indicated antibodies. FIG. 14E shows confocal images of WT and STX5:STX6 DKO 293T cells transfected with GFP-MEKK3 and Flag-Beclin 2, then stained with lysotracker. Scale bar, 10 μm. Pearson's correlation coefficient for colocalization was analyzed using Image J Coloc 2. Graph represents mean±s.e.m. (at least 30 cells were analyzed per condition). The data are representative of three independent experiments. Statistical differences between EmpVec-transfected and Flag-Beclin 2-transfected cells were calculated using Student's unpaired t-test (FIG. 14A). Statistical differences of degradation percentages between WT and KO groups were calculated using 1-way ANOVA with Dunnett's multiple comparison test (FIG. 14A). Statistical analyses of colocalization between groups were calculated using Student's unpaired t-test (FIG. 14E). *P<0.05; **P<0.01; ***P<0.001. NS, no significance.

FIGS. 15A, 15B, 15C, 15D, 15E, 15F, and 15G show that ablation of Map3k3 rescues the phenotypes observed in Becn2 KO mice. FIG. 15A shows spleens and lymph nodes of 6- to 8-week-old WT, Becn2 KO, Map3k3^{ΔM/ΔM}:Becn2 KO and Map3k7^{ΔM/ΔM}:Becn2 KO mice. FIG. 15B shows H&E staining of spleens from WT, Becn2 KO, Map3k3^{ΔM/ΔM}:Becn2 KO and Map3k7^{ΔM/ΔM}:Becn2 KO mice. Scale bar, 1 mm. Representative images (FIGS. 15A and 15B) are shown for each group (n=5 mice per group). FIG. 15C shows total numbers of splenocytes, and flow cytometry-based quantification of B220⁺ and CD3⁺ cell populations in spleens counted from 6- to 8-week-old Becn2 KO and WT mice (n=5 mice per group). FIG. 15D shows the basal IL-6 level in 8- to 12-week-old mouse sera (n=8 mice per group). FIG. 15E shows cytokine levels in serum samples from 6- to 8-week-old WT, Becn2 KO, and Becn2 KO:Map3k3^{ΔM/ΔM} mice after high-dose LPS (i.p. 30 mg/kg) treatment (n=5 mice per group). FIG. 15F shows IL-6 production by WT, Becn2 KO, Map3k3^{ΔM/ΔM}:Becn2 KO and Map3k7^{ΔM/ΔM}:Becn2 KO macrophages treated with LPS (100 ng/ml) for 0, 2, 4, or 6 h (mean f s.e.m.). FIG. 15G shows immunoblot analysis of macrophages from WT, Becn2 KO, Map3k3^{ΔM/ΔM}:Becn2 KO and Map3k7^{ΔM/ΔM}:Becn2 KO mice treated with LPS (100 ng/ml) for the indicated periods. The data (FIG. 15F and FIG. 15G) are representative of three independent experiments. Statistical differences between groups were calculated using 1-way ANOVA with Dunnett's multiple comparison test (FIGS. 15C-15F). **P<0.01; ***P<0.001.

FIGS. 16A, 16B, 16C, and 16D show that loss of Beclin 2 increases the incidence of spontaneous lymphoma development. FIG. 16A shows the tumor incidence of WT, Becn2 KO, and Becn2 KO Map3k3^{ΔM/ΔM} mice at 6-36 weeks of age (n=45 in WT group, 38 in Becn2 KO group, and 18 in Becn2 KO:Map3k3^{ΔM/ΔM} group). FIG. 16B shows photography and H&E staining of two different tumors from Becn2 KO mice. Red arrows indicate tumors. FIG. 16C shows photographs of tumors, lymph nodes, spleens and livers from WT and Becn2 KO mice. FIG. 16D shows H&E staining of lungs and livers from two Becn2 KO mice with lymphomas presented in (FIG. 16B). Arrows indicate metastatic tumor nodules in the lung and metastatic tumor cells in the liver. Log-rank test was used for calculating statistical differences in panel A. *P<0.05.

FIGS. 17A, 17B, and 17C show that lymphoma-bearing Becn2 KO mice can develop metastatic nodules in the lung and liver. FIGS. 17A and 17B show immunohistochemical staining of CD3, B220, or Ki67 in lymphoma and lung samples from tumor #1 (FIG. 17 A) and #2 (FIG. 17B). FIG. 17C shows representative confocal images of lymphoma and lung tissue from tumor #2 stained with antibodies against CD3, B220 and Ki67 followed by FITC- and Alexa Fluor 555-conjugated secondary antibodies. Scale bar, 250 μm.

FIGS. 18A, 18B, 18C, 18D, 18E, and 18F show that Beclin 2 deficiency enhances ERK, STAT3 signaling in T cells and B cells and alters multiple immune signaling pathways. FIG. 18A and FIG. 18B show immunoblot analysis of cell lysates from WT or Becn2 KO splenic T cells (FIG. 18A) and B cells (FIG. 18B) using the indicated antibodies. Data are representative of three independent experiments. FIGS. 18C and 18D show heatmap of the DEGs in Becn2 KO T cells (FIG. 18C) and B cells (FIG. 18D) compared with WT controls. The most significant DEGs involved in cytokine-cytokine receptor interaction and chemokine signaling pathways are listed on the right side. FIG. 18E and FIG. 18F show the significantly enriched KEGG pathways in Becn2 KO splenic T cells (FIG. 18E) and B cells (FIG. 18F) compared to WT controls.

FIGS. 19A, 19B, 19C, 19D, and 19E show STAT3 activation and cytokine/chemokine expression in the Becn2 KO lymphoma. FIG. 19A shows confocal images of Becn2 KO lymphomas, lymphoma-derived lung metastases and WT lymph nodes stained with antibodies against B220 and p-STAT3 followed by FITC- or Alexa Fluor 555-conjugated secondary antibodies. Scale bar, 250 μm. FIG. 19B shows confocal images of Becn2 KO lymphomas and WT lymph nodes stained with antibodies against B220 and p-ERK1/2, followed by labeling with FITC or Alexa Fluor 555-conjugated secondary antibodies. Scale bar, 250 μm. FIG. 19C shows immunoblot analysis of p-STAT3 and STAT3 expression in lymph nodes or lymphomas from WT and Becn2 KO mice. FIG. 19D shows heatmap of the DEGs in Becn2 KO lymphoma samples compared with WT or Becn2 KO lymph nodes. The most significant DEGs involved in STAT3 signaling, cytokine-cytokine receptor interaction, chemokine signaling pathways and cell-cell adhesion are listed on the right side. FIG. 19E shows the significantly enriched KEGG pathways in Becn2 KO lymphomas compared to WT or Becn2 KO lymph nodes.

FIGS. 20A, 20B, 20C, 20D, and 20E show that spontaneous lymphoma development is associated with persistent activation of STAT3 signaling in Becn2 KO mice. FIG. 20A shows immunoblot analysis of TAK1 and MEKK3 expression in lymph nodes or lymphomas from WT and Becn2 KO mice. FIG. 20B shows real-time PCR for indicated gene mRNA levels in WT lymph nodes, Becn2 KO lymph nodes, and Becn2 KO lymphoma (4 biological replicates of each group) as compared to FPKM value of each gene from RNAseq analysis. FIG. 20C shows real-time PCR analysis of the mRNA levels of Bcl2, Bclxl and Mcl1 in WT lymph nodes, Becn2 KO lymph nodes, and Becn2 KO lymphoma (4 biological replicates for each group). Center line represents median in the whisker boxes, and upper and lower lines represent lowest to highest value. Statistical differences between groups were calculated using 1-way ANOVA with Tukey's multiple comparison test for Q-PCR data (FIG. 20B, FIG. 20C). *P (Q-PCR) or Padj (RNAseq)<0.05, **P (Q-PCR) or Padj (RNAseq)<0.01, ***P (Q-PCR) or Padj (RNAseq)<0.001. NS, no significance. FIG. 20D shows immunoblot analysis of for p-Bcl-2, Bcl-2, Bcl-xl and Mcl-1 protein levels in cell lysates of T cell, B cell, lymph nodes (LN), and splenocytes from WT and Becn2 mice. FIG. 20E shows Kaplan-Meier plots depicting the association between high BECN2 expression and overall prolonged survival of patients with different cancer types. HR, hazard ratio. Log-rank test was used in the analysis.

FIGS. 21A and 21B show pro-tumorigenic cytokine, chemokine, and oncogene expression in Becn2 KO lymphoma. FIG. 21A shows real-time PCR analysis for the mRNA levels of the indicated genes in WT lymph nodes, Becn2 KO lymph nodes, and Becn2 KO lymphomas (4 biological replicates for each group) compared to the FPKM value of each gene from RNA-seq analysis. The whisker boxes represent median (center line), and upper and lower lines represent lowest to highest value. FIG. 21B shows immunohistochemical staining of lymphomas and lung metastases using antibodies against Cxcr4, IL-21, Bcl-7a and Ccl3. Scale bar, 100 μm. Statistical differences between groups were calculated using 1-way ANOVA with Tukey's multiple comparison test (A). *P<0.05; **P<0.01; ***P<0.001.

FIGS. 22A, 22B, 22C, and 22D show that IL-6 neutralizing antibody treatment rescues the phenotypes observed in Becn2 KO mice. FIG. 22A shows real-time PCR analysis for the mRNA levels of the indicated genes in lymph nodes from WT or Becn2 KO mice receiving control or IL-6 neutralizing antibody treatment (n=5, i.p., 200 μg/mouse, every other day for 4 consecutive weeks). FIG. 22B shows spleens of WT and Becn2 KO mice receiving control or IL-6 neutralizing antibody treatment (n=5). FIG. 22C shows immunoblot analysis of p-STAT3 in splenocytes from indicated groups. Representative images of three independent western blotting. FIG. 22D shows total numbers of splenocytes, and flow cytometry-based quantification of B220⁺ and CD3⁺ cell populations in spleens counted from mice of indicated groups (n=5). Statistical differences between groups were calculated using 1-way ANOVA with Tukey's multiple comparison test (FIG. 22A and FIG. 22D). *P<0.05; **P<0.01; ***P<0.001.

FIGS. 23A, 23B, 23C, 23D, 23E, 23F, 23G, 23H, 23I, and 23J show that Beclin 2 negatively regulates inflammasome activation and IL-1β production. FIGS. 23A and 23B show that PMA pre-treated mCherry-EV and mCherry-Beclin 2 overexpressed THP-1 cells were primed with LPS (200 ng/ml) for 3 h, and then stimulated with nigericin (1 uM, 6 h), poly(dA:dT) (1 μg/ml, 6 h), anthrax lethal factor (LF) (1 μg/ml, 4-6 h), or flagellin (20 μg/ml, 6-8 h), respectively. IL-1β production in supernatants (SN) was examined by ELISA (FIG. 23A). Protein levels of pro-caspase 1 in whole cell lysates (WCL) and cleavage caspase 1 in SN were detected using immunoblotting (FIG. 23B). FIG. 23C and FIG. 23D shows that HEK293T-CIA cells (HEK293T cells stably expressing caspase 1, pro-IL-1β, and ASC) were transfected with AIM2-encoding plasmid along with increasing doses of HA-tagged Beclin 2 plasmids, then stimulated with poly(dA:dT) (1 μg/ml, 6 h). Production of IL-1β in SN was examined by ELISA (FIG. 23C). Cleavages of caspase 1 in SN were detected by immunoblotting (FIG. 23D). FIGS. 23E and 23F show that HEK293T-CIA cells were transfected with NLRP3-encoding plasmid along with increasing doses of HA-tagged Beclin 2 plasmids then stimulated with nigericin (1 uM, 6 h). Production of IL-1β in SN was examined by ELISA (FIG. 23E). Cleaved caspase 1 in SN was detected by immunoblotting (FIG. 23F). FIG. 23G and FIG. 23H show that PMA pre-treated WT and BECN2 KO THP-1 cells were primed with LPS (200 ng/ml) for 3 h, and then stimulated with nigericin (1 uM, 6 h), poly(dA:dT) (1 μg/ml, 6 h), anthrax lethal factor (LF) (1 μg/ml, 4-6 h), flagellin (20 μg/ml, 6-8 h). IL-1β production in SN was examined by ELISA (FIG. 23G). Protein levels of pro-caspase 1 in WCL and cleavage of caspase 1 in SN were detected using immunoblotting (FIG. 23H). FIG. 23I and FIG. 23J show WT and Becn2-deficient mouse bone marrow macrophages (BMDMs) were primed with LPS (100 ng/ml, 3 h), and then stimulated with nigericin (1 uM, 6 h), poly(dA:dT) (1 μg/ml, 6 h), anthrax lethal factor (LF) (1 μg/ml, 4-6 h), flagellin (20 μg/ml, 6-8 h). IL-1p production in SN was examined by ELISA (FIG. 23I). Protein levels of pro-caspase 1 in WCL and cleaved caspase 1 in SN were detected using immunoblotting (FIG. 23J). *P<0.05, **P<0.01, ***P<0.001. (Student's t-test). Data are mean±SD of three independent experiments.

FIGS. 24A, 24B, and 24C show characterization of mouse Becn2 mRNA levels and Becn2 KO mouse. FIG. 24A shows RT-PCR analysis of mouse Beclin 2 expression in different tissues and cell types. The mRNA levels of mouse Becn2 were normalized to the mRNA levels of the GAPDH gene. FIG. 24B shows fluorescence microscopic analysis of the transduction efficiency of mCherry-Beclin 2 transduced THP-1 cells. FIG. 24C shows genotyping for WT, Becn2 heterozygous and Becn2 homozygous KO mouse.

FIGS. 25A, 25B, 25C, 25D, 25E, 25F, 25G, 25H, 25I, 25J, 25K, and 25L show that Beclin 2 interacts with inflammasome sensors through its CCDECD domain. FIG. 25A shows that HEK293T cells were co-transfected with HA-tagged Beclin 2 and Flag-tagged EV, AIM2, NLRP1, NLRP3, NLRC4, ASC or caspase 1. Cell lysates were immunoprecipitated with anti-Flag beads and immunoblotted using indicated antibodies. FIG. 25B and FIG. 25C show that mCherry-Beclin 2 overexpressing THP-1 cells were left untreated or primed with LPS, then stimulated with poly(dA:dT) (1 μg/ml, 6 h) (FIG. 25B), or Nigericin (1 uM, 6 h) (FIG. 25C). Cell lysates were immunoprecipitated using anti-mCherry antibody and protein A/G beads and then immunoblotted using indicated antibodies. FIGS. 25D and 25E show Hela cells co-transfected with GFP-Beclin 2 and Flag-AIM2 (FIG. 25D) or Flag-NLRP3 (FIG. 25E) were fixed and stained with anti-Flag antibody and the secondary antibody conjugated to Alexa Fluor 633. Colocalization was observed under confocal microscopy. Scale bar: 5 μm. (FIG. 25F) Schematic diagram of NLRP3 and domain truncation constructs. FIG. 25G shows that HEK293T cells were co-transfected with Flag-tagged Beclin 2 and HA-tagged NLRP3 full-length or NLRP3 domain truncations. Cell lysates were immunoprecipitated with anti-Flag beads and immunoblotted using indicated antibodies. PYD, pyrin domain; LRR, leucine-rich repeat motif, NACHT, NOD or NBD-nucleotide-binding domain. FIG. 25H shows schematic diagram of AIM2 and domain truncation constructs. FIG. 25I shows that HEK293T cells were co-transfected with HA-tagged Beclin 2 and Flag-tagged AIM2 full-length or AIM2 domains, then immunoprecipitated using anti-Flag beads and immunoblotted using indicated antibodies. FIG. 25J shows schematic diagram of Beclin 2 and domain truncation constructs. N, N-terminal domain; BH3 domain, Bcl-2 binding domain; CCD, central coiled-coil domain; and ECD, C-terminal evolutionarily conserved domain. FIG. 25K and FIG. 25L show HA-tagged Beclin 2 full-length or domains were co-transfected with Flag-tagged AIM2 (FIG. 25K) or NLRP3 (FIG. 25L), followed by immunoprecipitation using anti-Flag beads and immunoblotted using indicated antibodies.

FIGS. 26A, 26B, 26C, 26D, 26E, 26F, 26G, and 26H show that Beclin 2 degrades inflammasome sensors through lysosomal pathway. FIG. 26A and FIG. 26B show that HEK293T cells were transfected with Flag-tagged AIM2 (FIG. 26A) or NLRP3 (FIG. 26B) along with HA-tagged ASC, HA-tagged caspase 1 and HA-tagged Beclin 2, the cell lysates were immunoprecipitated with anti-Flag beads and then immunoblotted using indicated antibodies. FIG. 26C shows that HEK293T cells were co-transfected with increasing doses of HA-tagged Beclin 2 plasmids along with Flag-tagged NLRP1, NLRC4, ASC or caspase1, and immunoblotted using indicated antibodies. FIG. 26D shows RT-PCR analysis of Aim2 and Nlrp3 expression in WT and Becn2 KO mouse BMDMs with and without LPS (200 ng/ml, 3 h) priming. The mRNA levels of mouse Aim2 and Nlrp3 were normalized to the mRNA levels of the GAPDH gene. FIG. 26E shows RT-PCR analysis of AIM2 and NLRP3 expression in WT and BECN2 KO THP-1 cells. The mRNA levels of ARM2 and NLRP3 were normalized to the mRNA levels of the GAPDH gene. FIG. 26F shows sequence alignment analysis of the sgRNA-targeted DNA sequence in WT and BECN2 KO HEK293T cells generated using BECN2 sgRNA #2. FIGS. 26G and 26H show that HA-tagged Beclin 2 full-length or domain truncations were co-transfected with Flag-tagged AIM2 (FIG. 26G) or NLRP3 (FIG. 26H) into HEK293T cells, then immunoblotted using indicated antibodies. Quantification analysis based on the band density.

FIGS. 27A, 27B, 27C, 27D, 27E, 27F, and 27G show that Beclin 2 degrades inflammasome sensors through the lysosomal pathway. FIG. 27A shows that HEK293T cells were co-transfected with increasing doses of HA-tagged Beclin 2 plasmids along with Flag-tagged AIM2 or NLRP3, then immunoblotted using indicated antibodies. FIGS. 27B, 27C, and 27D show mCherry-Beclin 2-overexpressing THP-1 cells (FIG. 27B), BECN2 KO THP-1 cells (FIG. 27C), and BMDMs from Becn2-deficient mouse (FIG. 27D) were left untreated or primed with LPS (200 ng/ml) for 3 h, then the protein levels of inflammasome sensors were detected by immunoblot using indicated antibodies. FIG. 27E shows that WT and BECN2 KO HEK293T cells were transfected with Flag-tagged AIM2 or NLRP3 for 20 h, then treated with cycloheximide (CHX) (50 μg/ml) for the indicated time. Protein levels of AIM2 or NLRP3 were detected by immunoblot using indicated antibodies. FIG. 27F and FIG. 27G show that HEK293T cells were co-transfected with HA-tagged Beclin 2 and Flag-tagged AIM2 (FIG. 27F) or NLRP3 (FIG. 27G), then treated with DMSO (vehicle), MG132, chloroquine (CQ) and bafilomycin A1 (Baf A1) for 6 h. Cell lysates were then immunoblotted using indicated antibodies. Quantification analysis for (FIGS. 27E-27G) is presented as mean±SD and is calculated based on the band density of three independent experiments. *P<0.05, **P<0.01, ***P<0.001. (Student's t-test).

FIGS. 28A, 28B, 28C, 28D, 28E, 28F, 28G, 28H, 28I, and 28J show that Beclin 2-mediated degradation of inflammasome sensors is through the ULK1/ATG9A-dependent but Beclin 1/ATG16L/ATG7/LC3B-independent lysosomal pathway. WT and autophagy gene KO HEK293T cells were co-transfected with HA-tagged Beclin 2 and Flag-tagged NLRP3 or AIM2. Protein levels of AIM2 and NLRP3 were immunoblotted using indicated antibodies in BECN1 KO cells (FIG. 28A and FIG. 28B), ATG16L and ATG7 KO cells (FIG. 28C and FIG. 28D), MAP1LC3B KO cells (FIG. 28E and FIG. 28F), ULK1 KO cells (FIG. 28G and FIG. 28H), and ATG9A KO cells (FIG. 28I and FIG. 28J). Quantification analysis is presented as mean±SD and is calculated based on the band density of three independent experiments. *P<0.05, **P<0.01, ***P<0.001, ****P<0.0001 (Student's t-test).

FIGS. 29A, 29B, 29C, 29D, 29E, and 29F show that the interaction between Beclin 2 and inflammasome components requires ULK1 and ATG9A. WT and autophagy gene KO HEK293T cells were co-transfected with HA-tagged Beclin 2 and Flag-tagged NLRP3 or AIM2. Protein levels of AIM2 and NLRP3 were immunoblotted using indicated antibodies in ATG14 KO cells (FIG. 29A, FIG. 29B), WIPI2 KO cells (FIG. 29C, FIG. 29D), and VPS15 KO cells (FIG. 29E, FIG. 29F). Quantification analysis based on the band density. *P<0.05, **P<0.01, ****P<0.0001 (Student's t-test). Data are mean±SD of three independent experiments.

FIGS. 30A, 30B, 30C, 30D, 30E, 30F, 30G, 30H, and 30I show that the interaction between Beclin 2 and inflammasome components requires ULK1 and ATG9A. FIG. 30A and FIG. 30B show that WT, ULK1 KO, and ATG9A KO HEK293T were co-transfected with HA-tagged Beclin 2 and Flag-tagged AIM2 (FIG. 30A) or NLRP3 (FIG. 30B), the cell lysates were immunoprecipitated with anti-Flag beads and then immunoblotted using indicated antibodies. FIG. 30C shows that WT, ULK1 KO and ATG9A KO HEK293T cells co-transfected with GFP-AIM2 and mCherry-Beclin 2 were fixed with 4% (wt/vol) paraformaldehyde. Pearson's correlation coefficient for colocalization was analyzed using Image J Coloc 2. Graph represents mean±s.e.m. (at least 30 cells were analyzed per condition). Representative images showing the colocalization were obtained using confocal microscopy. Scale bar: 5 μm. FIG. 30D and FIG. 30E show that WT, BECN2 KO, and ULK1 KO HEK293T were co-transfected with HA-tagged ATG9A and Flag-tagged AIM2 (FIG. 30D) or NLRP3 (FIG. 30E), the cell lysates were immunoprecipitated with anti-Flag beads and then immunoblotted using indicated antibodies. FIG. 30F shows that WT and Becn2 KO BMDMs cells were left untreated or stimulated with LPS (200 ng/ml) for 3 h, the cell lysates were then immunoprecipitated using ATG9A antibodies, followed by immunoblotted with indicated antibodies. FIG. 30G shows WT and ULK1 KO HEK293T cells co-transfected with GFP-AIM2 and mCherry-ATG9A were fixed with 4% (wt/vol) paraformaldehyde. Pearson's correlation coefficient for colocalization was analyzed using Image J Coloc 2. Graph represents mean±s.e.m. (at least 30 cells were analyzed per condition). Representative images showing the colocalization were obtained using confocal microscopy. Scale bar: 5 μm. FIG. 30H shows that WT and ULK1 KO HEK293T were co-transfected with Flag-tagged Beclin 2 and HA-tagged ATG9A, the cell lysates were immunoprecipitated with anti-Flag beads and then immunoblotted using indicated antibodies. Data are representative of at least three independent experiments. FIG. 30I shows that WT and BECN2 KO 293T cells were transfected with AIM2-APEX2 encoding plasmid alone or along with Flag-Beclin 2. Representative EM images of cells expressing AIM2-APEX2 were processed in parallel under identical conditions. Black arrow indicates the AIM2 location. MVBs, multivesicular bodies. AP, autophagosome or amphisome. ***P<0.001 (Student's t-test).

FIGS. 31A and 31B show that Beclin 2-mediated degradation of inflammasome sensors is independent of ATG14/WIPI2/VPS15. FIG. 31A shows that HEK293T cells were co-transfected with HA-tagged Beclin 2 and Flag-tagged NLRP3, then treated with DMSO (vehicle), SBI-0206965 (10 μM) or MRT 68921 (1 μM) for 6 h. Cell lysates were then immunoblotted using indicated antibodies. Quantification analysis based on the band density. *P<0.05, **P<0.01 (Student's t-test). Data are mean±SD of three independent experiments. FIG. 31B shows that WT, ULK1 KO or ATG9A KO HEK293T cells transfected with GFP-AIM2 were stained with lysotracker. Colocalization was examined by confocal microscopy. Scale bar: 5 μm.

FIGS. 32A, 32B, 32C, and 32D show that SEC22A, STX5, and STX6 are required for Beclin 2-mediated degradation of inflammasome components. FIG. 32A shows that HEK293T were co-transfected with HA-tagged Beclin 2 and Flag-tagged SNARE family or RAB proteins, the cell lysates were immunoprecipitated with anti-Flag beads and then immunoblotted using indicated antibodies. FIG. 32B and FIG. 32C show that WT, SEC22A KO, STX5 KO, STX6 KO, and STX5:STX6 DKO HEK293T cells were co-transfected with HA-tagged Beclin 2 and Flag-tagged AIM2 (FIG. 32B) or NLRP3 (FIG. 32C). Protein levels of AIM2 and NLRP3 were immunoblotted using indicated antibodies. Quantification analysis based on the band density. FIG. 32D shows that WT and BECN2 THP-1 cells were lysed and immunoprecipitated using ATG9A antibodies, followed by immunoblotted with indicated antibodies. *P<0.05, **P<0.01, ***P<0.001, ****P<0.0001 (Student's t-test). Data are mean±SD of three independent experiments.

FIGS. 33A, 33B, 33C, 33D, and 33E show that Beclin 2-mediated degradation of inflammasome components requires SNAREs-mediated membrane fusion. FIG. 33A shows sequence alignment analysis of the sgRNA-targeted DNA sequence in WT and SEC22A KO HEK293T cells. FIG. 33B and FIG. 33C show that WT, RAB or SNARE gene KO HEK293T cells were co-transfected with HA-tagged Beclin 2 and Flag-tagged NLRP3. Protein levels of NLRP3 and Beclin 2 were immunoblotted using indicated antibodies. Quantification analysis based on the band density. *P<0.05, **P<0.01, ***P<0.001 (Student's t-test). Data are mean±SD of three independent experiments. FIG. 33D shows schematic illustration of Beclin 2-mediated ULK1-ATG9A-dependent degradation of inflammasome sensor. FIG. 33E shows genotyping for WT, Casp1 heterozygous and Casp1 homozygous KO mouse. Statistical differences between groups were calculated using Student's unpaired t-test (FIG. 33A). *P<0.05, **P<0.01, ***P<0.001, (Student's t-test).

FIGS. 34A, 34B, 34C, 34D, 34E, 34F, and 34G show loss of Beclin 2 exacerbates alum-induced peritoneal inflammation. FIG. 34A and FIG. 34B show that WT and Becn2 KO mice were i.p. injected with alum (700 μg). The peritoneal exudate cells (PECs) were collected 12 h after injection. FIG. 34A shows that absolute numbers of PECs recruited to the peritoneum were counted (n=4). **P<0.01. (Student's t-test). FIG. 34B shows protein levels of pro-caspase 1 and inflammasome sensors in whole cell lysates (WCL) and cleaved caspase 1 in SN of PECs at 12 h post-ex vivo culture that was detected by immunoblot using indicated antibodies. FIGS. 34C, 34D, 34E, 34F, 34G show that WT, Becn2 KO and Becn2:Casp1 DKO mice were i.p. injected with alum (700 μg). IL-1β production in peritoneal lavage obtained at 12 h post-injection was determined by ELISA (FIG. 34C). Data are mean±SD of three independent experiments. Percentage of CD11b⁺Gr1(Ly6C/Ly6G)⁺ neutrophils (FIG. 34D, FIG. 34E), monocytic (mMDSC, CD11b⁺Ly6C^{high}Ly6G^{low}) and granulocytic (gMDSC, CD11b⁺Ly6C^{int}Ly6G^{high}) subsets of myeloid-derived suppressor cells (FIG. 34F, FIG. 34G) recruited to the peritoneum were analyzed by flow cytometry (n=4). *P<0.05, **P<0.01, ***P<0.001, ****P<0.0001 (Student's t-test).

FIGS. 35A, 35B, 35C, and 35D show that Beclin 2 can interact with key molecules involved in neurodegenerative diseases and mediate their degradation. FIG. 35A shows that 293T cells were co-transfected with Flag-Beclin 2 and a HA-tagged plasmid as indicated, followed by immunoprecipitation with anti-Flag beads, and then immunoblotting with the indicated antibodies. FIG. 35B shows that 293T cells were co-transfected with Flag-Beclin 2 and GFP-TAU, followed by immunoprecipitation with anti-Flag beads, and then immunoblotting with the indicated antibodies. FIG. 35C shows immunoblot analysis of HA-tagged proteins in the lysates of 293T cells transfected with indicated HA-tagged plasmid alone, or together with increasing amount of Flag-Beclin 2 plasmid. FIG. 35D shows immunoblot analysis of endogenous proteins in the lysates of 293T cells transfected with increasing amount of pcDNA-Beclin 2 (no tag) plasmid.

FIG. 36 shows real-time PCR detection of BECN2 gene expression in different cell types, the mRNA levels of BECN2 were normalized with internal GAPDH expression.

FIGS. 37A and 37B show that BECN2 interacts with APP and APOE and targets them for degradation. FIG. 37A shows interaction of BECN2 with APP or APOE4, but not with Tau and TREM2 in 293T cells transfected with BECN2 along with APP, Tau, APOE4 or TREM2. 36 h after transfection, cell lysates were subjected to immunoprecipitation with anti-FLAG beads, followed by western blotting analysis with anti-FLAG and anti-HA antibodies. Rapamycin and Bafilomycin A were used to induce autophagy and inhibit protein degradation. FIG. 37B shows immunoblot analysis of APP and APOE levels in 293T cells transfected with FLAG-APOE or APP plus an increasing amount of pcDNA-HA-BECN2 plasmid (0, 200 and 400 ng/106 cells). SOD1 served as a negative control, which could not be degraded by BECN2.

FIG. 38 shows phosphorylated Tau levels between WT and BECN2 KO mice, tissue lysates of mouse brains were used to detect phosphorylated Tau and total Tau protein with specific antibodies.

FIG. 39 shows strategies for the application of Beclin 2 in the prevention and treatment of inflammatory diseases, cancer, and neurodegenerative disease. Strategy I. Specific manipulation of the levels of Beclin 2 for the treatment of inflammatory diseases, the development of neurodegenerative diseases or cancer. The possible approaches for Beclin 2 manipulation include but not limit to recombinant DNA-, siRNA-, or sgRNA-CRISPR-technology or small molecules induction for specific overexpression/knockdown/knockout of Beclin 2. These approaches can be applied to control the levels of Beclin 2 target protein (MEKK3, TAK1, inflammasome sensors, or a series of critical molecules in neurodegenerative diseases including APOE, SUPT5H TDP43, GAK, PINK1, PARK2, PARK7, TREM2, and TAU) through autophagic degradation. Strategy II. Generation of a Beclin 2 fusion protein with target molecule-binding single-chain fragment variable (scFv) to lead the degradation of targeted molecules through Beclin 2-mediated ATG9A-dependent non-conventional autophagy. Either full-length Beclin 2 or the ATG9A-binding domain of Beclin 2 can be utilized for the generation of Beclin 2-scFv for sorting and degrading the Beclin 2-interactive molecules through Beclin 2-mediated non-conventional autophagy. Strategy III. Generation of a Beclin 2-small molecule complex to induce endogenous protein degradation through Beclin 2-mediated ATG9A-dependent non-conventional autophagy pathway. Beclin 2 protein can be chemically linked to a small molecule drug through a PEG-based linker. Beclin 2 functions to interact with the target protein, while small molecule functions as a ligand for endogenous protein. The connection of target endogenous protein with Beclin 2 can lead the protein degradation through Beclin 2-mediated ATG9A-dependent non-conventional autophagy pathway.

FIG. 40 shows annotated SEQ ID NO: 5.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The “vector” described herein comprises a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence (for example, the ribosome binding site).

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides. (Used together with “polynucleotide” and “polypeptide”.)

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids. The term “operatively linked” can also refer to at least two chemical structures joined together in such a way as to remain linked through the various manipulations described herein.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operatively linked to the promoter/reglatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Recombinant Beclin 2 Polypeptide and Polynucleotide

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Beclin-2 is disclosed and discussed and a number of modifications that can be made to a number of molecules including the Beclin-2 are discussed, specifically contemplated is each and every combination and permutation of Beclin-2 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Until 2013, Beclin 1 was considered the only Beclin encoded by the mammalian genome. Beclin 2 was first described as an autophagy gene that shared 57% and 44% homology with Beclin 1 in humans and mice respectively. While Beclin 1 and Beclin 2 do share some functional properties, Beclin 2, unlike Beclin 1, also plays a role in additional lysosomal degradation pathways. Beclin 2 plays a critical role in G protein-coupled receptor degradation related to viral tumorigenesis and cannabinoid receptor degradation related to drug tolerance.

Disclose herein in one aspect are engineered Beclin2 proteins and polypeptides. It is understood and herein contemplated that the engineered Beclin 2 can comprise truncated or substituted Beclin 2. In some aspects, the engineered Beclin 2 comprises a truncation or substitution of the ATG9a, STX5/6 or target protein binding domains, but retains the binding properties (i.e., is a functional mutant). In some aspects, the truncated Beclin 2 comprises only one or a combination of two or more of the ATG9a, STX5/6 or target protein binding domains (for example, a truncated Beclin 2 comprising only all or a functional mutant (either substituted or truncated mutant) of the ATG9a or STX5/6 binding domain. The engineered Beclin 2 can also comprise modifications such as fusions or chemical linkages to other molecules including antibodies, antibody fragments, or small molecules. Thus, in one aspect, disclosed herein are full length, substituted, or truncated Beclin 2 operatively linked to an antibody, antibody fragment, or small molecule, including, but not limited to anti-Tau ScFV-Beclin 2 or anti-Tau ScFV-ATG9a.

It is understood and herein contemplated that unmodified Beclin 2 protein, as well as, any modified Beclin 2 polypeptides and proteins disclosed herein, and any fragments thereof can be used as a component of a recombinant protein or polypeptide that can target Beclin 2 to a specific cell, tissue, organ, or microenvironment. Thus, in one aspect, disclosed herein are recombinant proteins or polypeptides comprising i) a modified or unmodified Beclin 2 polypeptide or protein or a fragment thereof and/or ii) a targeting moiety and the uses there of for reducing inflammation; treating, inhibiting, reducing, decreasing, ameliorating, and/or preventing a cancer, metastasis; or a neurodegenerative disease.

“Beclin 2” refers herein to a protein or polypeptide that synthesizes and hydrolyzes cyclic adenosine 5′-diphosphate-ribose, and in humans, is encoded by the BECN2 gene. In some embodiments, the Beclin 2 protein or polypeptide is that identified in one or more publicly available databases as follows: HGNC: 38606, Entrez Gene: 441925, Ensembl: ENSG00000196289, OMIM: 615687, UniProtKB: A8MW95. In some embodiments, the Beclin 2 protein comprises the sequence of SEQ ID NO: 1, or a polypeptide sequence having at or greater than about 70%, about 75%, about 80%, about 85%, about 90%, about 95%, or about 98% sequence identity with SEQ ID NO: 1. The Beclin 2 protein or polypeptide may represent an immature or pre-processed form of mature Beclin 2, and accordingly, included herein are mature or processed portions of the Beclin 2 protein in SEQ ID NO: 1. In some embodiments, the recombinant protein or polypeptide comprises an ATG9A-binding domain of the Beclin 2 polypeptide or protein, such as, for example, an ATG9A-binding domain polypeptide sequence comprising about 70%, about 75%, about 80%, about 85%, about 90%, about 95%, or about 98%, or about 99% sequence identity to SEQ ID NO: 3. In some aspects, the recombinant protein or polypeptide comprises the ATG9A-binding domain as set forth in SEQ ID NO: 3.

In some embodiments, the recombinant protein or polypeptide described herein comprises a targeting moiety that specifically binds to a target. The term “target” refers to a biomolecule, small molecule, or a cell that can be the focus of a therapeutic drug strategy, diagnostic assay, or a combination thereof. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others. In one example, the targeting moiety specifically binds to a pathogenic molecule, peptide, or protein related to a neurodegenerative disease or a cancer. For example, in some embodiments, the targeting moiety specifically binds to neurodegenerative associated pathogenic molecule, peptide, or protein including, but not limited to TAU, β-amyloid, APOE, SUPT5H, TDP43, GAK, PINK1, PARK2, PARK7, TREM2; or a pathogenic molecule, peptide, or protein related to a cancer, including but not limited to NLRP3, NLRP1, NLRC4, AIM2, TAK1 and/or MEKK3, as well as tumor antigens or oncogenes (derived from point mutations, amplification and fusion) such as a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, L1CAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCLIA, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIPI 1, TRK, TSC1, TSC2, TSHR, VHL, WAS, WHSC1L1 8, WRN, WT1, XPA, XPC, ZNF145, ZNF198, ZNF278, ZNF384, or ZNFN1A1.

The targeting moiety can be any molecule that can specifically binds to a target, for example, a small molecule, ligand, agonist, antagonist, nucleic acid, a protein, a peptide, a lipid, or a sugar. In one example, the targeting moiety comprises a small molecule (e.g., a chemical compound). In one example, the targeting moiety comprises an antibody or a functional fragment thereof.

The term “antibodies” is used herein in a broad sense and includes both polyclonal 5 and monoclonal antibodies. As used herein, the term “antibody” encompasses, but is not limited to, whole immunoglobulin (i.e., an intact antibody) of any class. In addition to intact immunoglobulin molecules, also included in the term “antibodies” are fragments or polymers of those immunoglobulin molecules, and human or humanized versions of immunoglobulin molecules or fragments thereof. It should be understood that the “antibody” can be monoclonal antibodies, polyclonal antibodies, chimeric antibodies, bi-specific antibodies (diabody), or tri-specific antibody (triabody).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of crosslinking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, a VHH antibody and the like, including hybrid fragments. Thus, fragments of the antibodies that retain the ability to bind their specific antigens are provided. For example, fragments of antibodies which maintain Annexin A2 binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

In a complete antibody, typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant (C(H)) domains. Each light chain has a variable domain at one end (V(L)) and a constant(C(L)) domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively. In some embodiments, the antibody is of IgG1 isotype. In some embodiments, the antibody is of IgG2 isotype. In some embodiments, the antibody is of IgG3 isotype. In some embodiments, the antibody is of IgG4 isotype. In some embodiments, the antibody is of IgM isotype. In some embodiments, the antibody is of IgA isotype.

In some embodiments, the recombinant protein or polypeptide comprises i) a modified or unmodified Beclin 2 protein, polypeptide, or fragment thereof and/or ii) a targeting moiety, wherein the targeting moiety comprises an antibody or a functional fragment thereof, wherein the antibody fragment is selected from the group consisting of a Fab antibody, a single-chain variable fragment (scFv) antibody, and a V_{H}H antibody, and wherein the antibody or antibody fragment specifically binds to TAU. In some embodiments, the antibody or antibody fragment comprises a light chain variable region comprising a polypeptide sequence at least about 70%, about 75%, about 80%, about 85%, about 90%, about 95%, or about 98% identical to SEQ ID NO: 7 or a portion thereof. In some embodiments, the antibody or antibody fragment comprises a heavy chain variable region comprising a polypeptide sequence at least about 70%, about 75%, about 80%, about 85%, about 90%, about 95%, or about 98% identical to SEQ ID NO: 8 or a portion thereof. In one example, the antibody or antibody fragment comprises a polypeptide sequence at least 80% (e.g, at least about 80%, about 85%, about 90%, about 95%, or about 98%) identical to SEQ ID NO: 9. In one example, disclosed herein is a recombinant protein or polypeptide comprising i) a modified or unmodified Beclin 2 protein or polypeptide and ii) a targeting moiety, wherein the recombinant protein or polypeptide comprises a sequence at least about 70% (e.g, at least about 70%, about 75%, about 80%, about 85%, about 90%, about 95%, or about 98%) identical to SEQ ID NO: 5.

In some aspects, disclosed herein is a recombinant polynucleotide encoding any of the recombinant proteins or polypeptides disclosed herein. In some embodiments, the recombinant polynucleotide comprises a polynucleotide sequence encoding a Beclin 2 polypeptide or a fragment thereof. In some embodiments, the recombinant polynucleotide comprises a polynucleotide sequence at least about 70% (e.g, at least about 70%, about 75%, about 80%, about 85%, about 90%, about 95%, or about 98%) identical to SEQ ID NO: 2 or a fragment thereof. In some embodiments, the recombinant polynucleotide comprises a polynucleotide sequence encoding a ATG9A-binding domain of Beclin 2 peptide, wherein the recombinant polynucleotide comprises a polynucleotide sequence at least about 70% (e.g, at least about 70%, about 75%, about 80%, about 85%, about 90%, about 95%, or about 98%) identical to SEQ ID NO: 4. In some embodiments, the recombinant polynucleotide comprises a polynucleotide sequence at least about 70% (e.g, at least about 70%, about 75%, about 80%, about 85%, about 90%, about 95%, or about 98%) identical to SEQ ID NO: 6.

It is understood and herein contemplated that the modified or unmodified Beclin 2 polypeptide, protein, or a fragment used in the disclosed recombinant proteins or polypeptides can be operatively linked the targeting moiety. This operative linkage can occur via a chemical bond, or indirectly via a linker. A direct chemical bond is for example a covalent bond (e.g., peptide bond, ester bond, or the like), or alternatively, a non-covalent bond (e.g., ionic, electrostatic, hydrogen, hydrophobic, Van der Waal interactions, or π-effects). An indirect link can be achieved using a linker, i.e., a chemical group that connects one or more other chemical groups via at least one covalent bond. Suitable linkers include amino acids, peptides, nucleotides, nucleic acids, dimeric hinged Fc, organic linker molecules (e.g., maleimide derivatives, N-ethoxybenzylimidazole, biphenyl-3,4′,5-tricarboxylic acid, p-aminobenzyloxycarbonyl, and the like), disulfide linkers, and polymer linkers (e.g., PEG). The linker can include one or more spacing groups including, but not limited to alkylene, alkenylene, alkynylene, alkyl, alkenyl, alkynyl, alkoxy, aryl, heteroaryl, aralkyl, aralkenyl, aralkynyl and the like. The linker can be neutral, or carry a positive or negative charge. Additionally, the linker can be cleavable such that the linker's covalent bond that connects the linker to another chemical group can be broken or cleaved under certain conditions, including pH, temperature, salt concentration, light, a catalyst, or an enzyme. Examples of suitable peptide linkers are well known in the art, and programs to design linkers are readily available (see, e.g., Crasto et al., Protein Eng., 2000, 13(5):309-312). The peptide linker can be a restriction site linker such as the short sequence RS, or a flexible amino acid linker (e.g., comprising small, non-polar or polar amino acids). Non-limiting examples of flexible linkers include LEGGGS (SEQ ID NO: 123), TGSG (SEQ ID NO: 124), GGSGGGSG (SEQ ID NO: 125), GGGGS (SEQ ID NO: 126), GGGGSGGGGS (SEQ ID NO: 127), GGGGSGGGGSGGGGS (SEQ ID NO: 128), GGGGSGGGGSGGGGSGGGGS (SEQ ID NO: 129), GGGS (SEQ ID NO: 130), GGGSGGGS (SEQ ID NO: 131), GGGSGGGSGGGS (SEQ ID NO: 132), GGGSGGGSGGGSGGGS (SEQ ID NO: 133), GSGGGG (SEQ ID NO: 134), GSGGGGGSGGGG (SEQ ID NO: 135), GSGGGGGSGGGGGSGGGG (SEQ ID NO: 136), GSGGGGGSGGGGGSGGGGGSGGGG (SEQ ID NO: 137), GGGGGG (SEQ ID NO: 138), GGGGGGG (SEQ ID NO: 139), and GGGGGGGG (SEQ ID NO: 140). Alternatively, the peptide linker can be a rigid amino acid linker. Such linkers include EAAAK (SEQ ID NO: 141), EAAAKEAAAK (SEQ ID NO: 142), EAAAKEAAAKEAAAK (SEQ ID NO: 143), EAAAKEAAAKEAAAKEAAAK (SEQ ID NO: 144), AEAAAKEAAAKA (SEQ ID NO: 145), AEAAAKEAAAKEAAAKA (SEQ ID NO: 146), AEAAAKEAAAKEAAAKEAAAKA (SEQ ID NO: 147), AEAAAKEAAAKEAAAKEAAAKEAAAKA (SEQ ID NO: 148), PAPAP (SEQ ID NO: 149), APAPAPAPAPAP (SEQ ID NO: 150), APAPAPAPAPAPAP (SEQ ID NO: 151), and APAPAPAPAPAPAPAP (SEQ ID NO: 152). In one aspect, the disclosed modified or unmodified Beclin 2 polypeptide, protein or fragment thereof can be linked to the targeting moiety via a polyethylene glycol (PEG) based linker. It understood and herein contemplated that the linkage of Beclin 2 and the targeting moiety can occur at any location that that allows successful linkage. In one aspect, the targeting moiety can be operatively linked to the N-terminal or the C-terminal of the unmodified or modified Beclin 2 polypeptide or protein.

Also disclosed herein are vectors comprising any of the recombinant polynucleotides disclosed herein. As used herein, “vector” comprises a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

Also disclosed herein are cells comprising any of the recombinant protein or polypeptide disclosed herein, any of the engineered Beclin 2 proteins or polypeptides disclosed herein, any of the recombinant polynucleotides disclosed herein, or any of the vectors disclosed herein.

In some embodiments, the recombinant protein or polypeptide, recombinant polynucleotide, and/or the vector is formulated with a pharmaceutically acceptable compound.

### Methods of Treatment

In one aspect, it is understood and herein contemplated that the disclosed engineered Beclin-2, recombinant proteins or polypeptides, and polynucleotides encoding said recombinant proteins polypeptides can be used to treat, modulate, inhibit, decrease, reduce, ameliorate, and/or prevent diseases or conditions where the upregulation, overexpression, or application of Beclin-2 can have a therapeutic effect on said disease or condition. For example, as shown herein, the disclosed herein the disclosed recombinant Beclin-2 comprising polypeptides and polynucleotides can modulate inflammatory pathways or initiate autophagy the effect of which can treat, modulate, inhibit, decrease, reduce, ameliorate, and/or prevent diseases or condition.

As used herein, the term “treatment” or “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “therapeutically effective” refers to the amount of the composition used is of sufficient quantity to ameliorate one or more causes or symptoms of a disease or disorder. Such amelioration only requires a reduction or alteration, not necessarily elimination.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Method of Treating Cancers

Disclosed herein are methods of treating, inhibiting, reducing, decreasing, ameliorating, and/or preventing a cancer and/or metastasis (such as, for example, a metastatic lymphoma, lung cancer, T cell lymphoma, or B cell lymphoma), comprising administering to the subject a therapeutically effective amount of a) an engineered Beclin 2 or b) a recombinant protein or polypeptide comprising i) a modified or unmodified Beclin 2 protein, polypeptide, or a fragment thereof. In some embodiments the recombinant protein or polypeptide further comprises ii) a targeting moiety. The targeting moiety can be operatively linked to the N-terminal or the C-terminal of the modified or unmodified Beclin 2 protein or polypeptide.

“Beclin 2” refers herein to a polypeptide that synthesizes and hydrolyzes cyclic adenosine 5′-diphosphate-ribose, and in humans, is encoded by the BECN2 gene. In some embodiments, the Beclin 2 polypeptide is that identified in one or more publicly available databases as follows: HGNC: 38606, Entrez Gene: 441925, Ensembl: ENSG00000196289, OMIM: 615687, UniProtKB: A8MW95. In some embodiments, the Beclin 2 polypeptide comprises the sequence of SEQ ID NO: 1, or a polypeptide sequence having at or greater than about 70%, about 75%, about 80%, about 85%, about 90%, about 95%, or about 98% homology with SEQ ID NO: 1, or a polypeptide comprising a portion of SEQ ID NO: 1. The Beclin 2 polypeptide of SEQ ID NO: 1 may represent an immature or pre-processed form of mature Beclin 2, and accordingly, included herein are mature or processed portions of the Beclin 2 polypeptide in SEQ ID NO: 1. In some embodiment, the engineered Beclin 2 or recombinant protein or polypeptide comprises an ATG9A-binding domain of the Beclin 2 protein or polypeptide. In one aspect, the engineered Beclin 2 or the recombinant protein or polypeptide can comprise an ATG9A-binding domain polypeptide sequence at least 70% identical to SEQ ID NO: 3.

In one example, the targeting moiety specifically binds to a peptide, protein, and/or pathogenic molecule related to a cancer. In one example, the peptide, protein, and/or pathogenic molecule comprises a cell surface molecule, an intracellular molecule, or an extracellular molecule. In some embodiments, the targeting moiety specifically binds to NLRP3, NLRP1, NLRC4, AIM2, TAK1 MEKK3, or tumor antigens and/or oncogenes (derived from point mutations, amplification and fusion) such as a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LiCAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIPIL1, FLI1, FLT3, FLT4, FMS, FNBP1, FOXOIA, FOXO3A, FPS, FSTL3, FUS, GAS7, GATA1, GIP, GMPS, GNAS, GOLGA5, GPC3, GPHN, GRAF, HEI10, HER3, HIP1, HIST1H41, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, or ZNFN1A1.

Accordingly, in some aspects, disclosed herein are methods of treating, inhibiting, reducing, decreasing, ameliorating, and/or preventing a cancer and/or metastasis (such as, for example, a metastatic lymphoma, lung cancer, T cell lymphoma, or B cell lymphoma), comprising administering to the subject a therapeutically effective amount of a) an engineered Beclin 2 or b) a recombinant protein or polypeptide comprising i) a modified or unmodified Beclin 2 protein, polypeptide, or a fragment thereof and/or ii) a targeting moiety. In some aspects, the Beclin 2 protein polypeptide can comprises a sequence at least about 70% (e.g., at least about 70%, about 75%, about 80%, about 85%, about 90%, about 95%, or about 98%) identical to SEQ ID NO: 1 or 3. In some aspects, the targeting moiety specifically binds to a peptide, protein, and/or pathogenic molecule related to a cancer (e.g., NLRP3, NLRP1, NLRC4, AIM2, TAK1 MEKK3, or tumor antigens and/or oncogenes (derived from point mutations, amplification and fusion) such as a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LiCAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSFi7, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, or ZNFN1A1).

The disclosed methods of treating, inhibiting, reducing, decreasing, ameliorating, and/or preventing a cancer and/or metastasis can be used to treat, inhibit, reduce, decrease, ameliorate, and/or prevent any uncontrolled cellular proliferation occurs such as cancers. A representative but non-limiting list of cancers that the disclosed compositions can be used to treat is the following: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, leukemia lymphocytic, liver cholangiocarcinoma, liver hepatocellular carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, non-Hodgkin's lymphoma, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epitheloid cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma, Multiple endocrine neoplasia, Hereditary Paraganglioma-Pheochromocytoma Syndromes, paragangliomas 1, PTEN hamartoma tumor syndrome, hereditary cutaneous melanoma, multiple fibrofolliculomas, Familial cancer of breast, DICER1-related pleuropulmonary blastoma cancer predisposition syndrome, lynch syndrome, Neurofibromatosis, Axillary freckling, Focal T2 hyperintense basal ganglia lesion, Hereditary cancer-predisposing syndrome, Multiple cafe-au-lait spots, type 1 Neurofibromatosis, Von Hippel-Lindau syndrome, Civic and clinvar, pediatric adrenocortical carcinoma, Li-Fraumeni syndrome, Neoplasm of the breast, neoplasm of ovary, and any combination thereof. In some embodiments, the cancer comprises metastatic lymphoma, lung cancer, T cell lymphoma, or B cell lymphoma. The methods disclosed herein may also be used for the treatment of precancer conditions such as cervical and anal dysplasias, other dysplasias, severe dysplasias, hyperplasias, atypical hyperplasias, and neoplasias.

Also disclosed herein is a method of treating, inhibiting, reducing, decreasing, ameliorating, and/or preventing a cancer and/or metastasis, comprising administering to the subject a therapeutically effective amount of a recombinant polynucleotide encoding an of the engineered Becline 2 proteins or polypeptides or the recombinant protein or polypeptide disclosed herein. In some embodiments, the recombinant polynucleotide comprises a sequence at least 80% identical to SEQ ID NO: 2.

It is understood and herein contemplated that the timing of a cancer or metastasis onset can often not be predicted. The disclosed methods of treating, preventing, reducing, and/or inhibiting a cancer or metastasis can be used prior to or following the onset of a cancer or metastasis. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2, or 1 hour prior to onset of a cancer or metastasis; or 1, 2, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10, 11, 12, 15, 18, 24, 30, 36, 48, 60 or more years after onset of a cancer or metastasis.

The studies herein describe that upregulation of Beclin 2 induces degradation of key proteins that left unchecked lead to inflammation, cancer, and/or neurodegenerative diseases. The studies indicate that Beclin 2 decreases inflammation and disease progression via targeting multiple signaling pathways: 1) NF-κB and MAPK signaling, 2) NLRP3 and AIM2 inflammasome activation, and 3) β-amyloid and TAU proteins. The studies related to NF-κB and MAPK signaling indicate that Beclin 2 targets the upstream inflammatory signaling proteins, Tak1 and MEKK3, for degradation, which ultimately leads to a decrease in proinflammatory cytokine production, reduced inflammation, and/or impaired tumorigenesis. It should be understood and herein contemplated that a decrease in a level of Tak1 and MEKK upon the administering of the disclosed engineered Beclin 2 proteins or polypeptides or the disclosed recombinant proteins or polypeptides is through an ATG16L/LC3/Beclin 1-indepenent pathway. Accordingly, disclosed herein are methods of treating, inhibiting, reducing, decreasing, ameliorating, and/or preventing a cancer and/or metastasis, comprising administering to the subject a therapeutically effective amount of a) an engineered Beclin 2 protein or polypeptide, b) a recombinant protein or polypeptide comprising i) a modified or unmodified Beclin 2 protein, polypeptide, or a fragment thereof and/or ii) a targeting moiety, or c) a polynucleotide encoding the engineered Beclin 2 protein or polypeptide or the recombinant protein or polypeptide; wherein the engineered Beclin 2 protein or polypeptide, the recombinant protein or polypeptide, or polynucleotide decreases a level of Tak1, MEKK3, or a molecule related to NF-κB and MAPK signaling pathway in a cell. In some embodiments, the targeting moiety specifically binds to Tak1, MEKK3, or a molecule related to NF-κB and MAPK signaling pathway. In some embodiments, the cell is a cancer cell. In some embodiments, the cell comprises a benign or metastatic tumor cell. In some embodiments, the cell is a non-cancer cell (e.g., an immune cell, endothelial cell, or epithelial cell). In some embodiments, the administering of the engineered Beclin 2 protein or polypeptide, the recombinant protein or polypeptide, or polynucleotide decreases cancer cell proliferation and metastasis, and decreases a level of a proinflammatory cytokine (e.g., a local or systemic level of a proinflammatory cytokine), (such as, for example proinflammatory cytokine including, but not limited to, IL-6, IL-1β, IL-1α, TNF-α, IL-17, or IFN-γ or any combination thereof).

The studies related to NLRP3 and AIM2 inflammasome activation reveals that Beclin 2 interacts directly with the inflammasome sensors (e.g., NLRP3, AIM2, Pyrin, NLRP1, NLRP6, NLRP7, NLRC4, NAIP, or IFI16), and initiates their degradation. This Beclin 2-mediated degradation and/or reduced activation of inflammasome sensors yield less proinflammatory cytokine secretion (e.g., IL-1β) and reduces inflammatory pathways that are involved in an inflammation-related disorder (e.g., cancer or neuronal dysfunction). Accordingly, the method disclosed herein is a method of treating, inhibiting, reducing, decreasing, ameliorating, and/or preventing a cancer and/or metastasis, comprising administering to the subject a therapeutically effective amount of a) an engineered Beclin 2 protein or polypeptide, b) a recombinant protein or polypeptide comprising i) a modified or unmodified Beclin 2 protein, polypeptide, or a fragment thereof and/or ii) a targeting moiety, or c) a polynucleotide encoding the engineered Beclin 2 protein or polypeptide or the recombinant protein or polypeptide; wherein the engineered Beclin 2 protein or polypeptide, the recombinant protein or polypeptide, or the polynucleotide encoding decreases a level of an inflammasome sensor and/or decreases the activation of an inflammasome sensor (e.g., improper phosphorylation or improper assembly of an inflammasome signaling complex) in a cell, wherein the inflammasome sensor comprises NLRP3, AIM2, Pyrin, NLRP1, NLRP6, NLRP7, NLRC4, NAIP, or IFI16 or any combination thereof. In some embodiments, the targeting moiety specifically binds to an inflammasome sensors (e.g., NLRP3, AIM2, Pyrin, NLRP1, NLRP6, NLRP7, NLRC4, NAIP, or IFI16). In some embodiments, the Beclin 2 polypeptide directly or indirectly binds to an inflammasome sensors (e.g., NLRP3, AIM2, Pyrin, NLRP1, NLRP6, NLRP7, NLRC4, NAIP, or IFI16). In some embodiments, the cell is a cancer cell. In some embodiments, the cell comprises a benign or metastatic tumor cell. In some embodiments, the cell is a non-cancer cell (e.g., an immune cell, endothelial cell, or epithelial cell). In some embodiments, the administration of the engineered Beclin 2 protein or polypeptide, the recombinant protein or polypeptide, or the polynucleotide encoding the engineered Beclin 2 protein or polypeptide or the recombinant protein or polypeptide decreases a level of a proinflammatory cytokine (e.g., a local or systemic level of a proinflammatory cytokine), wherein the proinflammatory cytokine comprises IL-1β, IL-1α, or IL-18 or any combination thereof.

The targeting moiety can be any molecule that can specifically binds to a target, wherein the targeting moiety can be, for example, a small molecule, ligand, agonist, antagonist, nucleic acid, a lipid, or a sugar. In one example, the targeting moiety comprises a small molecule. In one example, the targeting moiety comprises an antibody or a functional fragment thereof, wherein the antibody fragment is selected from the group consisting of a Fab antibody, a single-chain variable fragment (scFv) antibody, and a V_{H}H antibody.

Also disclosed herein is a method of treating, inhibiting, reducing, decreasing, ameliorating, and/or preventing a cancer or metastasis in a subject comprising administering to the subject a therapeutically effective amount of a recombinant polynucleotide that encodes any of the engineered Beclin 2 proteins or polypeptides or any of the recombinant protein or polypeptide disclosed herein.

In some aspects, disclosed herein is a method of treating, inhibiting, reducing, decreasing, ameliorating, and/or preventing a cancer or metastasis in a subject by increasing a level of Beclin 2 polypeptide in a cell (e.g., a cancer cell or a non-cancer cell), wherein the method comprising administering to the subject a therapeutically effective amount of a) an engineered Beclin 2 protein or polypeptide, b) a recombinant protein or polypeptide comprising i) a modified or unmodified Beclin 2 protein, polypeptide, or a fragment thereof and/or ii) a targeting moiety, and/or c) a polynucleotide encoding the engineered Beclin 2 protein or polypeptide or the recombinant protein or polypeptide; wherein the engineered Beclin 2 protein or polypeptide, the recombinant protein or polypeptide, and/or polynucleotide decreases a level of Tak1, MEKK3, or a molecule related to NF-κB and MAPK signaling pathway in a cell (e.g., a cancer cell or a non-cancer cell). In some aspects, disclosed herein is a method of treating, inhibiting, reducing, decreasing, ameliorating, and/or preventing a cancer or metastasis in a subject by increasing a level of Beclin 2 polypeptide in a cell (e.g., a cancer cell or a non-cancer cell), wherein the method comprising administering to the subject a therapeutically effective amount of the recombinant nucleotide encoding any of the engineered Beclin 2 proteins or polypeptides or the recombinant proteins or polypeptides disclosed herein, wherein the recombinant polynucleotide decreases a level of Tak1, MEKK3, or a molecule related to NF-κB and MAPK signaling pathway in a cell (e.g., a cancer cell or a non-cancer cell).

Also disclosed herein is a method of treating, inhibiting, reducing, decreasing, ameliorating, and/or preventing a cancer or metastasis in a subject by decreasing a level of Tak1, MEKK3, and/or a molecule related to NF-κB and MAPK signaling pathway in a cell (e.g., a cancer cell or a non-cancer cell), wherein the method comprising administering to the subject a therapeutically effective amount of the engineered Beclin 2 proteins or polypeptides, the recombinant proteins or polypeptides, and/or polynucleotides disclosed herein, wherein the recombinant protein or polypeptide comprises i) a modified or unmodified Beclin 2 protein or polypeptide or a fragment thereof and/or ii) a targeting moiety. Also disclosed herein is a method of treating, inhibiting, reducing, decreasing, ameliorating, and/or preventing a cancer or metastasis in a subject by decreasing a level of Tak1, MEKK3, and/or a molecule related to NF-κB and MAPK signaling pathway in a cell (e.g., a cancer cell or a non-cancer cell), wherein the method comprising administering to the subject a therapeutically effective amount of the recombinant polynucleotide encoding any of the engineered Beclin 2 proteins or polypeptides or the recombinant proteins or polypeptides disclosed herein. In some embodiments, the subject is a cancer patient.

Also disclosed herein is a method of decreasing a level of Tak1 and/or MEKK3 in a subject, comprising administering to the subject a therapeutically effective amount of a) an engineered Beclin 2 protein or polypeptide, b) a recombinant protein or polypeptide, wherein the recombinant protein or polypeptide comprises i) a modified or unmodified Beclin 2 protein, polypeptide, or a fragment thereof and/or ii) a targeting moiety. Also disclosed herein is a method of decreasing a level of Tak1 and/or MEKK3 in a subject, comprising administering to the subject a therapeutically effective amount of the recombinant polynucleotide encoding said engineered Beclin 2 protein or polypeptide or said recombinant protein or polypeptide. In some embodiments, the subject is a cancer patient.

In some aspects, disclosed herein is a method of treating, inhibiting, reducing, decreasing, ameliorating, and/or preventing a cancer and/or metastasis, wherein the method further comprises administering to the subject a therapeutically effective amount of an anti-cancer therapeutic agent, including, but not limited to, Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). In some embodiments, the at least one anti-cancer therapeutic agent comprises an antibody targeting immune checkpoint blockade. The blockade inhibitor that can be used in the disclosed methods can be any inhibitor of an immune checkpoint such as for example, a PD-1/PD-L1 blockade inhibitor, a CTLA-4/B7-1/2 blockade inhibitor (such as for example, Ipilimumab), and CD47/Signal Regulator Protein alpha (SIRPa) blockade inhibitor (such as for example, Hu5F9-G4, CV1, B6H12, 2D3, CC-90002, and/or TTI-621). Examples, of PD-1/PD-L1 blockade inhibitors for use in the disclosed bioresponsive hydrogels can include any PD-1/PD-L1 blockade inhibitor known in the art, including, but not limited to nivolumab, pembrolizumab, pidilizumab, atezolizumab, avelumab, durvalumab, and BMS-936559).

### Methods of Treating Neurodegenerative Diseases

Disclosed herein are methods of treating, inhibiting, reducing, decreasing, ameliorating, and/or preventing a neurodegenerative disease, comprising administering to the subject a therapeutically effective amount of a) an engineered Beclin 2 protein or polypeptide, b) a recombinant proteins or polypeptides comprising a modified or unmodified Beclin 2 polypeptide, protein, or a fragment thereof, or c) a recombinant polynucleotide encoding any of the said engineered Beclin 2 proteins or polynucleotides or any of the recombinant proteins or polynucleotides disclosed herein. In some embodiments the recombinant protein or polypeptide further comprises a targeting moiety. The targeting moiety can be operatively linked to the N-terminal or the C-terminal of a Beclin 2 protein or polypeptide as described herein.

As used herein, the term “neurodegenerative disease” refers to a varied assortment of central nervous system disorders characterized by gradual and progressive loss of neural tissue and/or neural tissue function. A neurodegenerative disease is a class of neurological disorder or disease, and where the neurological disease is characterized by a gradual and progressive loss of neural tissue, and/or altered neurological function, typically reduced neurological function as a result of a gradual and progressive loss of neural tissue. Examples of neurodegenerative diseases include for example, but are not limited to, Alzheimer's disease (AD), Parkinson's disease (PD), Huntington's Disease, Amyotrophic Lateral Sclerosis (ALS, also termed Lou Gehrig's disease) and Multiple Sclerosis (MS), polyglutamine expansion disorders (e.g., HD, dentatorubropallidoluysian atrophy, Kennedy's disease (also referred to as spinobulbar muscular atrophy), spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple system atrophy, Pelizaeus-Merzbacher disease, Pick's disease, primary lateral sclerosis, Refsum's disease, Sandhoff disease, Schilder's disease, spinal cord injury, spinal muscular atrophy (SMA), SteeleRichardson-Olszewski disease, Tabes dorsalis, and the like. In some embodiments, the neurodegenerative disease is Alzheimer's disease. In some embodiments, the neurodegenerative disease is Parkinson's disease.

“Alzheimer's Disease” as used herein refers to all form of dementia, identified as a degenerative and terminal cognitive disorder. The disease may be static, the result of a unique global brain injury, or progressive, resulting in long-term decline in cognitive function due to damage or disease in the body beyond what might be expected from normal aging. The beta-amyloid protein, or Ap, involved in Alzheimer's has several different molecular forms that collect between neurons. It is formed from the breakdown of a larger protein, called amyloid precursor protein. One form, beta-amyloid 42, is thought to be especially toxic. An abnormal level of this protein is found in the Alzheimer's brain, wherein the protein clump together to form plaques between neurons, leading to neuron function disruption.

In one example, the targeting moiety specifically binds to a peptide, protein, and/or pathogenic molecule related to a neurodegenerative disease. In one example, the peptide, protein, and/or pathogenic molecule comprises a cell surface molecule, an intracellular molecule, or an extracellular molecule. In some embodiments, the targeting moiety specifically binds to TAU, β-amyloid, APOE, SUPT5H TDP43, GAK, PINK1, PARK2, PARK7, and/or TREM2.

Accordingly, in some aspects, disclosed herein is a method of treating, inhibiting, reducing, decreasing, ameliorating, and/or preventing a neurodegenerative disease, comprising administering to the subject a therapeutically effective amount of a) an engineered Beclin 2 protein or polypeptide, b) a recombinant proteins or polypeptides comprising a modified or unmodified Beclin 2 polypeptide, protein, or a fragment thereof and/or ii) a targeting moiety, or c) a recombinant polynucleotide encoding any of the said engineered Beclin 2 proteins or polynucleotides or any of the recombinant proteins or polynucleotides disclosed herein; wherein the modified or unmodified Beclin 2 polypeptide or protein comprises a sequence at least 70% identical to SEQ ID NO: 1, and wherein the targeting moiety specifically binds to TAU, β-amyloid, APOE, SUPT5H TDP43, GAK, PINK1, PARK2, PARK7, and/or TREM2.

It is understood and herein contemplated that the timing of a neurodegenerative disease onset can often not be predicted. The disclosed methods of treating, preventing, reducing, and/or inhibiting a neurodegenerative disease can be used prior to or following the onset of a neurodegenerative disease. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2, or 1 hour prior to onset of a neurodegenerative disease; or 1, 2, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10, 11, 12, 15, 18, 24, 30, 36, 48, 60 or more years after onset of a neurodegenerative disease.

The studies disclosed herein also describe that upregulation of Beclin 2 induces degradation of key proteins that, left unchecked, lead to inflammation, cancer and neurodegenerative diseases. The studies indicate that Beclin 2 decreases inflammation and diseases progression via targeting multiple signaling pathways: 1) NF-κB and MAPK signaling, 2) NLRP3 and AIM2 inflammasome activation, and 3) β-amyloid and TAU proteins. The studies herein show that a neurodegenerative disease-related protein (e.g., β-amyloid or TAU protein) can interact with Beclin 2 and or by the targeting moiety of the recombinant protein or polypeptide disclosed herein.

Accordingly, disclosed herein are methods of treating, inhibiting, reducing, decreasing, ameliorating, and/or preventing a neurodegenerative disease, comprising administering to the subject a therapeutically effective amount of a) an engineered Beclin 2 protein or polypeptide, b) aa recombinant protein or polypeptide comprising i) a modified or unmodified Beclin 2 protein, polypeptide, or a fragment thereof, or c) a recombinant polynucleotide encoding any of said engineered Beclin 2 proteins or polynucleotides encoding any of said recombinant proteins or polynucleotides disclosed herein. In some aspects, the recombinant protein or polypeptide used in the disclosed methods can further comprise ii) a targeting moiety. It is understood and herein contemplated that the engineered Beclin 2 protein or polypeptide, the recombinant protein polypeptide, or recombinant polynucleotide decreases the level of a peptide, protein, or pathogenic molecule related to a neurodegenerative disease in a cell, wherein the peptide, protein, or pathogenic molecule comprises TAU, β-amyloid, APOE, SUPT5H TDP43, GAK, PINK1, PARK2, PARK7, and/or TREM2. In some embodiments, the targeting moiety specifically binds to TAU, β-amyloid, APOE, SUPT5H TDP43, GAK, PINK1, PARK2, PARK7, and/or TREM2. In some embodiments, the cell is a neural cell. In some embodiments, the administration of the engineered Beclin 2 protein or polypeptide or the recombinant protein or polypeptide decreases the pathogenesis of a neurodegenerative disease.

The studies related to NLRP3 and AIM2 inflammasome activation reveals that Beclin 2 interacts directly with the inflammasome sensors (e.g., NLRP3, AIM2, Pyrin, NLRP1, NLRP6, NLRP7, NLRC4, NAIP, or IFI16), and initiates their degradation. This Beclin 2-mediated degradation and/or reduced activation of inflammasome sensors yield less proinflammatory cytokine secretion (e.g., IL-1p) and reduces inflammatory pathways that are involved in an inflammation-related disorder (e.g., cancer or neuronal dysfunction). Accordingly, the method disclosed herein is a method of treating, inhibiting, reducing, decreasing, ameliorating, and/or preventing a neurodegenerative disease, comprising administering to the subject a therapeutically effective amount of a) an engineered Beclin 2 protein or polypeptide or b) a recombinant protein or polypeptide comprising i) a modified or unmodified Beclin 2 protein, polypeptide, or a fragment thereof and/or ii) a targeting moiety, wherein the engineered Beclin 2 protein or polypeptide or the recombinant protein or polypeptide decreases a level of an inflammasome sensor and/or decreases the activation of an inflammasome sensor (e.g., improper phosphorylation or improper assembly of an inflammasome signaling complex) in a cell, wherein the inflammasome sensor comprises NLRP3, AIM2, Pyrin, NLRP1, NLRP6, NLRP7, NLRC4, NAIP, or IFI16 or any combination thereof. In some embodiments, the targeting moiety specifically binds to an inflammasome sensors (e.g., NLRP3, AIM2, Pyrin, NLRP1, NLRP6, NLRP7, NLRC4, NAIP, or IFI16). In some embodiments, the Beclin 2 polypeptide directly or indirectly binds to an inflammasome sensors (e.g., NLRP3, AIM2, Pyrin, NLRP1, NLRP6, NLRP7, NLRC4, NAIP, or IFI16). In some embodiments, the cell is a neural cell. In some embodiments, the cell is a non-neural cell (e.g., an immune cell, endothelial cell, or epithelial cell). In some embodiments, the administration of any of the engineered Beclin 2 proteins or polypeptides, any of the recombinant proteins or polypeptides, or any of the recombinant polynucleotides decreases a level of a proinflammatory cytokine (e.g., a local or systemic level of a proinflammatory cytokine), wherein the proinflammatory cytokine comprises IL-1β, IL-1α, or IL-18 or any combination thereof.

The targeting moiety can be any molecule that can specifically binds to a target, wherein the targeting moiety can be, for example, a small molecule, ligand, agonist, antagonist, nucleic acid, a lipid, or a sugar. In one example, the targeting moiety comprises a small molecule. In one example, the targeting moiety comprises an antibody or a functional fragment thereof, wherein the antibody fragment is selected from the group consisting of a Fab antibody, a single-chain variable fragment (scFv) antibody, and a V_{H}H antibody. In some embodiments, the antibody or antibody fragment comprises a light chain variable region comprising a polypeptide sequence at least about 70%, about 75%, about 80%, about 85%, about 90%, about 95%, or about 98% identical to SEQ ID NO: 7 or a portion thereof. In some embodiments, the antibody or antibody fragment comprises a heavy chain variable region comprising a polypeptide sequence at least about 70%, about 75%, about 80%, about 85%, about 90%, about 95%, or about 98% identical to SEQ ID NO: 8 or a portion thereof. In some embodiments, the antibody or antibody fragment comprises SEQ ID NO: 7 and SEQ ID NO: 8. In one example, the antibody or antibody fragment comprises a polypeptide sequence at least 80% (e.g., about 80%, about 85%, about 90%, about 95%, or about 98%) identical to SEQ ID NO: 9. In some embodiments, the targeting moiety is a scFv antibody specifically binding to TAU protein.

Accordingly, disclose herein is a method of treating, inhibiting, reducing, decreasing, ameliorating, and/or preventing a neurodegenerative disease, comprising administering to the subject a therapeutically effective amount of a) an engineered Beclin 2 protein or polypeptide, b) a recombinant proteins or polypeptides comprising a modified or unmodified Beclin 2 polypeptide, protein, or a fragment thereof and/or ii) a scFV antibody specifically binds to TAU; wherein the Beclin 2 protein or polypeptide comprises a sequence at least about 70% (e.g, at least about 70%, about 75%, about 80%, about 85%, about 90%, about 95%, or about 98%) identical to SEQ ID NO: 1 or 3 or a fragment thereof, wherein the scFv antibody comprises a light chain variable region comprising a polypeptide sequence at least about 70%, about 75%, about 80%, about 85%, about 90%, about 95%, or about 98% identical to SEQ ID NO: 7 or a portion thereof. In some embodiments, the scFv antibody comprises a heavy chain variable region comprising a polypeptide sequence at least about 70%, about 75%, about 80%, about 85%, about 90%, about 95%, or about 98% identical to SEQ ID NO: 8 or a portion thereof. In one example, the scFv antibody comprises a polypeptide sequence at least 80% (e.g., about 80%, about 85%, about 90%, about 95%, or about 98%) identical to SEQ ID NO: 9. In some embodiments, disclose herein is a method of treating, inhibiting, reducing, decreasing, ameliorating, and/or preventing a neurodegenerative disease, comprising administering to the subject a therapeutically effective amount of an engineered Beclin 2 protein or polypeptide or a recombinant protein or polypeptide comprising a sequence at least about 70% (e.g, at least about 70%, about 75%, about 80%, about 85%, about 90%, about 95%, or about 98%) identical to SEQ ID NO: 5.

Also disclosed herein is a method of treating, inhibiting, reducing, decreasing, ameliorating, and/or preventing a neurodegenerative disease, comprising administering to the subject a therapeutically effective amount of a recombinant polynucleotide encoding any of the engineered Beclin 2 proteins or polypeptides or any of the recombinant proteins or polypeptides disclosed herein. In some embodiments, the recombinant polynucleotide comprises a polynucleotide sequence encoding a Beclin 2 polypeptide or a fragment thereof. In some embodiments, the recombinant polynucleotide comprises a polynucleotide sequence at least about 70% (e.g, at least about 70%, about 75%, about 80%, about 85%, about 90%, about 95%, or about 98%) identical to SEQ ID NO: 2 or a fragment thereof. In some embodiments, the recombinant polynucleotide comprises a polynucleotide sequence encoding a ATG9A-binding domain of Beclin 2 peptide, wherein the recombinant polynucleotide comprises a polynucleotide sequence at least about 70% (e.g, at least about 70%, about 75%, about 80%, about 85%, about 90%, about 95%, or about 98%) identical to SEQ ID NO: 4. In some embodiments, the recombinant polynucleotide comprises a polynucleotide sequence at least about 70% (e.g, at least about 70%, about 75%, about 80%, about 85%, about 90%, about 95%, or about 98%) identical to SEQ ID NO: 6.

In some aspects, disclosed herein is a method of treating, inhibiting, reducing, decreasing, ameliorating, and/or preventing a neurodegenerative disease in a subject in need thereof by increasing a level of Beclin-2 polypeptide in a cell, comprising administering to the subject a therapeutically effective amount of any of the engineered Beclin 2 proteins or polypeptides or recombinant proteins or polypeptides, or a recombinant polynucleotide encoding the recombinant proteins or polypeptide, wherein the recombinant proteins or polypeptides comprise i) a modified or unmodified Beclin 2 protein, polypeptide, or a fragment thereof and/or ii) a targeting moiety; and wherein the engineered Beclin 2 protein or polypeptide, the recombinant protein or polypeptide, or the recombinant polynucleotide decreases a level of an inflammasome sensor and/or decreases the activation of an inflammasome sensor (e.g., decreased phosphorylation or improper assembly of an inflammasome signaling complex) in a cell (e.g., a neural cell or a non-neural cell), wherein the inflammasome sensor comprises NLRP3, AIM2, Pyrin, NLRP1, NLRP6, NLRP7, NLRC4, NAIP, or IFI16 or any combination thereof. In some embodiments, the engineered Beclin 2 protein or polypeptide, the recombinant protein or polypeptide, or the recombinant polynucleotide decreases a level of a neurogenerative disease-related molecule in the neural cell, wherein the molecule comprises TAU, β-amyloid, APOE, SUPT5H TDP43, GAK, PINK1, PARK2, PARK7, or TREM2 protein.

Also disclosed herein is a method of treating, inhibiting, reducing, decreasing, ameliorating, and/or preventing a neurodegenerative disease in a subject by decreasing a level of an inflammasome sensor and/or decreasing the activation of an inflammasome sensor (e.g., decreased phosphorylation or improper assembly of an inflammasome signaling complex) in a cell (e.g., a neural cell or a non-neural cell), wherein the inflammasome sensor comprises NLRP3, AIM2, Pyrin, NLRP1, NLRP6, NLRP7, NLRC4, NAIP, or IFI16 or any combination thereof, wherein the method comprising administering to the subject a therapeutically effective amount of any of the engineered Beclin 2 protein or polypeptide disclosed herein, any of the recombinant proteins or polypeptides disclosed herein, or the recombinant polynucleotide encoding said engineered Beclin 2 proteins or polypeptides or encoding said recombinant proteins or polypeptide, wherein the recombinant protein or polypeptide comprises i) a modified or unmodified Beclin 2 protein or polypeptide or a fragment thereof and/or ii) a targeting moiety. In some embodiments, the subject is a neurodegenerative disease patient.

Also disclosed herein is a method of decreasing a level of an inflammasome sensor and/or decreasing the activation of an inflammasome sensor (e.g., decreased phosphorylation or improper assembly of an inflammasome signaling complex) in a cell (e.g., a neural cell or a non-neural cell) in a subject, wherein the inflammasome sensor comprises NLRP3, AIM2, Pyrin, NLRP1, NLRP6, NLRP7, NLRC4, NAIP, or IFI16 or any combination thereof, wherein the method comprises administering to the subject a therapeutically effective amount of any of the engineered Beclin 2 proteins or polypeptides disclosed herein, any of the recombinant proteins or polypeptides disclosed herein, or any of the recombinant polynucleotides disclosed herein, wherein the recombinant protein or polypeptide comprises i) a modified or unmodified Beclin 2 protein or polypeptide or a fragment thereof and in some aspects further comprising ii) a targeting moiety. In some embodiments, the subject is a neurodegenerative disease patient.

### EXAMPLES

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. While the invention has been described with reference to particular embodiments and implementations, it will be understood that various changes and additional variations may be made and equivalents may be substituted for elements thereof without departing from the scope of the invention or the inventive concept thereof. In addition, many modifications may be made to adapt a particular situation or device to the teachings of the invention without departing from the essential scope thereof. Such equivalents are intended to be encompassed by the following claims. It is intended that the invention not be limited to the particular implementations disclosed herein, but that the invention will include all implementations falling within the scope of the appended claims.

### Example 1: Beclin 2 Negatively Regulates Innate Immune Signaling and Tumor Development

Autophagy is an essential cellular process for maintaining cell homeostasis and attenuating cell stresses through a “self-eating” mechanism. Autophagy-related (ATG) proteins play an important role in infectious, autoimmune, and inflammatory diseases by regulating the innate immune signaling such as inflammasome and type I interferon. Autophagy protein has been reported to regulate the inflammasome activation through autophagic mechanisms, including the removal of intracellular inflammasome-activating damage-associated molecular patterns (DAMPs), the sequestration and degradation of inflammasome components, and the control of biogenesis and secretion of interleukin (IL)-1β protein. Emerging evidence also shows that autophagy-related proteins can function in autophagy-independent pathways such as vesicular trafficking, innate immunity, cell death and proliferation.

Many autophagy-related (ATG) proteins have been identified to function in autophagy to control physiological and pathological processes by breaking down misfolded or dysfunctional components for recycling. Notably, growing evidence shows that ATG proteins can regulate immune responses through canonical macroautophagy, non-canonical macroautophagy, or autophagy-independent pathways. Coiled-coil, myosin-like BCL2-interacting protein 2 (Becn2) has recently been identified as a homolog of Becn1 with both autophagy-dependent and -independent functions, and targets G protein-coupled receptors (GPCRs) for degradation through endosomal-lysosomal pathway. Heterozygous Becn2 knockout (KO) mice develop obesity and insulin resistance, due to excessive cannabinoid 1 receptor (CB1R) signaling. Furthermore, monoallelic deletion of Becn2 significantly increases Kaposi's sarcoma-associated herpesvirus (KSHV)-induced oncogenesis through elevation of KSHV GPCR signaling in ikGPCR+Becn2+/− mice. Obesity-associated chronic inflammation plays a leading role in the pathogenesis of type II diabetes and cancer. Despite the importance of autophagy and its related proteins in immunity and cancer development, the function and mechanisms of Beclin 2 in the regulation of innate immune signaling, inflammasome regulation, autophagy, and cancer remain largely unknown.

Innate immune signaling pathways, including the NF-κB, type I interferon (IFN), and inflammasome pathways, are activated through innate immune receptors, such as Toll-like receptors, RIG-I-like receptors, DNA sensors, and NOD-like receptors. Ligation of these immune receptors with pathogen-associated molecular patterns (PAMPs) and damage-associated molecular patterns (DAMPs) recruits key adaptor molecules to trigger downstream signaling pathways for the production of inflammatory cytokines. The regulatory functions of ATG proteins in innate immune signaling are crucial for the control of systemic inflammation. For instance, the ULK1/2 complex and Beclin 1 protect the host against viral infection, while ATG16L1 suppresses endotoxin-induced inflammasome activation. Autophagy can also function as a tumor suppressor to inhibit cancer development by regulating innate immune signaling.

The present application describes the role of Beclin 2 as a negative regulator in the control of the ERK1/2 and NF-κB signaling pathways to suppress inflammation and tumor development. Mice with homozygous ablation of Becn2 developed splenomegaly and lymphadenopathy, and enhanced phosphorylation of ERK1/2 and NF-κB signaling in innate immune cells for proinflammatory cytokine productions. Mechanistically, Beclin 2 targeted mitogen-activated protein kinase kinase kinase 3 (MEKK3) and mitogen-activated protein kinase kinase kinase 7 (TAK1) for autophagic degradation through an ATG9A-dependent but ATG16L/LC3/Beclin 1-independent pathway. Beclin 2 promoted the membrane fusion of TAK1/MEKK3-associated ATG9A+ vesicles with phagophores through its interaction with STX5/STX6. Importantly, Becn2-deficient mice developed spontaneous lymphoma at a high incidence (˜13.2%). Persistent activation of STAT3 signaling by interleukin (IL)-6 and other cytokines. These findings have identified an important role of Beclin 2 in the regulation of innate immune signaling and tumor development, thus providing a therapeutic target for the prevention and treatment of inflammatory diseases and cancer.

Further, the present application has also identified a previously unrecognized role of Beclin 2 in the negative regulation of inflammasome pathways. Inflammasome pathways play crucial roles in the hosts' defense against invading pathogens, cancer development, metabolic diseases, and neurodegenerative diseases. Inflammasomes are multiprotein complexes that consist of a sensor protein, an adaptor protein ASC (apoptosis-associated speck-like protein containing a caspase-recruitment domain), and the pro-caspase 1 protein. Several established inflammasome complexes are composed of different NOD-like receptors, including NLRP3 (sensors for particulates, ATP and nigericin), AIM2 (sensors for dsDNA from microbes or host origin), NLRC4 (sensor for bacterial flagellin), and NLRP1 (sensor for Bacillus anthracis lethal toxin, Toxoplasma gondii, and muramyl dipeptide). Ligand stimulation of these sensors leads to the recruitment and assembly of the inflammasome complex to activate caspase 1 enzyme activity. The processed and active caspase 1 further catalyzes the proteolytic cleavage of pro-IL-1p and pro-IL-18 to release mature IL-1β and IL-18 proteins. Various regulatory proteins have been identified to control the activation of inflammasomes, including GBP5, NEK7, PKR, BRCC3, and autophagy proteins, but their molecular mechanisms remain to be further defined. This application shows that Beclin 2 regulates inflammasome activation by interacting with NLRP3, AIM2, NLRC4, and NLRP1 and mediating their degradation through a ULK1- and ATG9A-dependent, but ATG7-, ATG16L-, LC3-, WIPI2-, and Beclin 1-independent lysosomal pathway. Importantly, the soluble NSF attachment protein receptors (SNAREs), including STX5, STX6, and SEC22A, are required for the Beclin 2-ATG9A-dependent lysosomal degradation of inflammasome sensor proteins. Therefore, the findings here have identified a previously unrecognized role of Beclin 2 in the negative regulation of inflammasome sensor proteins through a ULK1- and ATG9A-dependent lysosomal pathway, thus providing therapeutic targets for the treatment of inflammatory diseases.

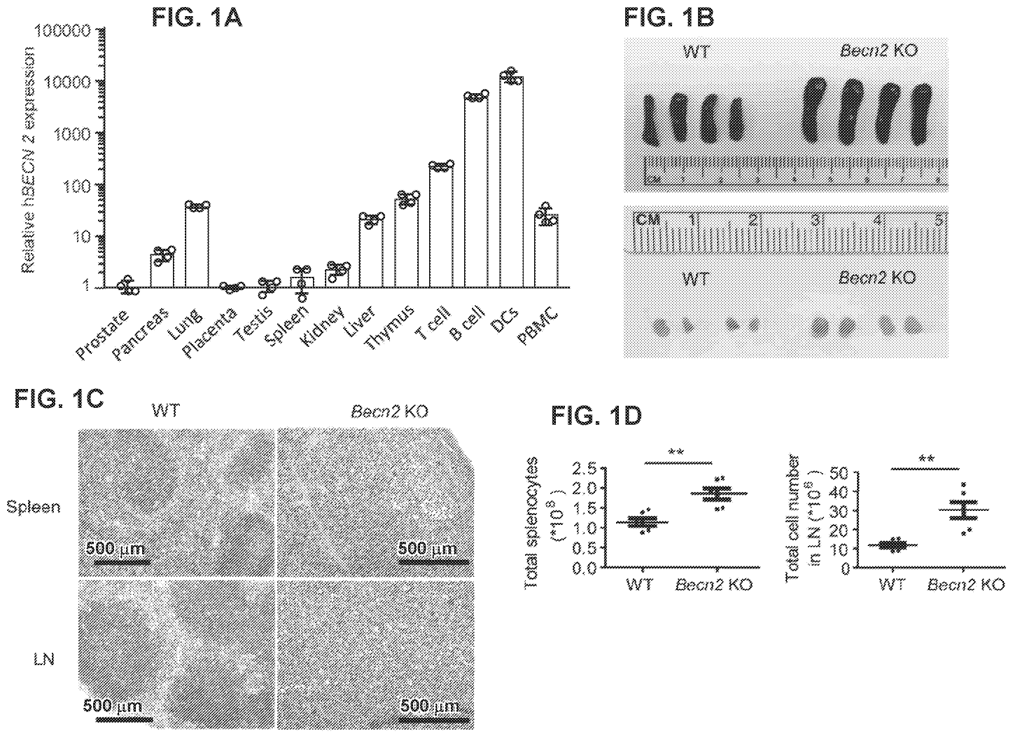

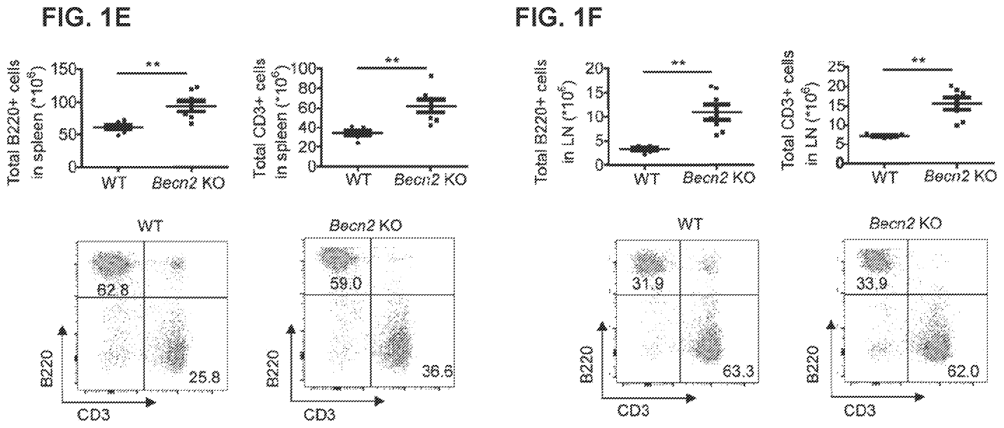

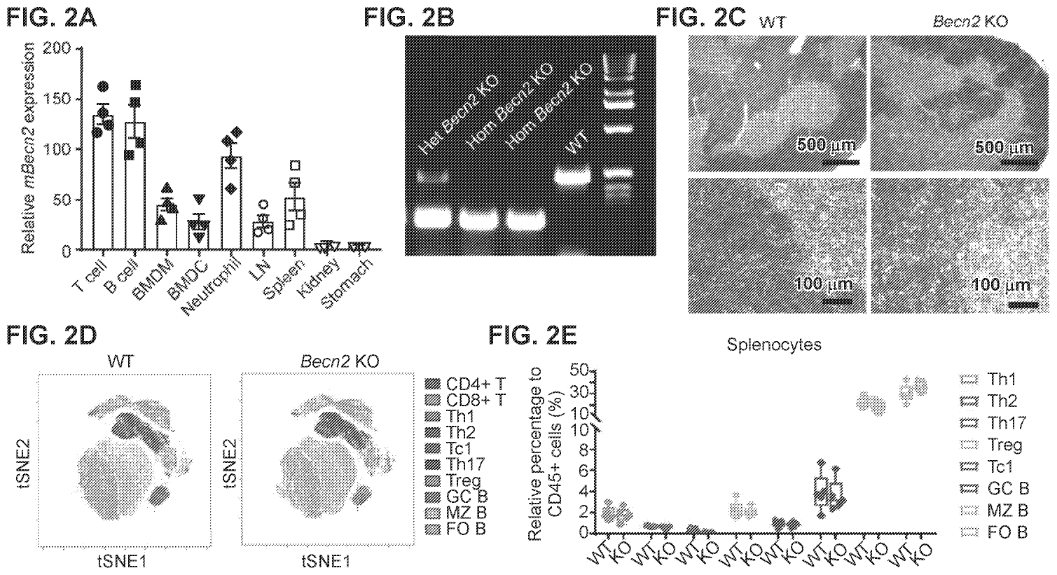

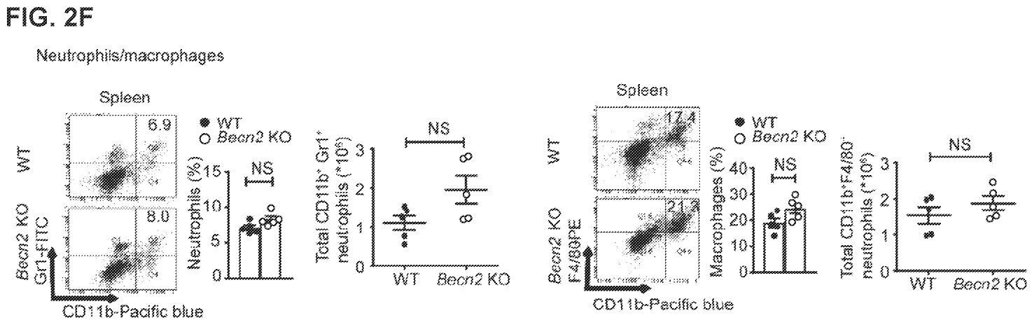

Splenomegaly and lymphadenopathy in Becn2-deficient mice. To investigate the role of Beclin 2 in the immune system, human BECN2 expression was analyzed in multiple organs and different cell types, and found that human BECN2 was highly expressed in the thymus, lung, liver, and pancreas, particularly in peripheral blood mononuclear cells (PBMCs), dendritic cells (DCs), T cells, and B cells (FIG. 1A). Similarly, the mouse Becn2 was also highly expressed in immune cells and lymphoid tissues such as neutrophils, T cells, B cells, and spleen compared to non-immune tissues (FIG. 2A). To assess the function of Beclin 2 in immune cells, homozygous Becn2 KO mice were generated despite the partial lethality of Becn2 ablation. Becn2 deficiency was validated by polymerase chain reaction (PCR) using specific primers (FIG. 2B). Becn2 KO mice developed splenomegaly and lymphadenopathy (FIG. 1B). Histological examination revealed grossly disorganized architectures of spleens and lymph nodes in Becn2 KO mice, with complete loss of the marginal zone barrier (FIG. 1C). Mild architecture difference was also found in the thymus of Becn2 KO mice compared with WT mice (FIG. 2C). Consistent with the splenomegaly and enlarged lymph nodes, marked increases were observed in the total numbers of splenocytes and lymphocytes from inguinal lymph nodes, particularly the B220⁺ B cell and CD3⁺ T cell numbers, in Becn2 KO mice compared with WT mice (FIGS. 1D-1F). However, no significant differences in the subsets of either T cells or B cells were observed between WT and Becn2 KO mice by CYTOF (mass cytometry) analyses (FIGS. 2D and 2E). There was no appreciable difference in macrophage/neutrophil populations in the spleens between WT and Becn2 KO mice (FIG. 2F). These results indicate that loss of Beclin 2 results in splenomegaly and lymph node enlargement with grossly disorganized architectures and increased total lymphocyte populations.

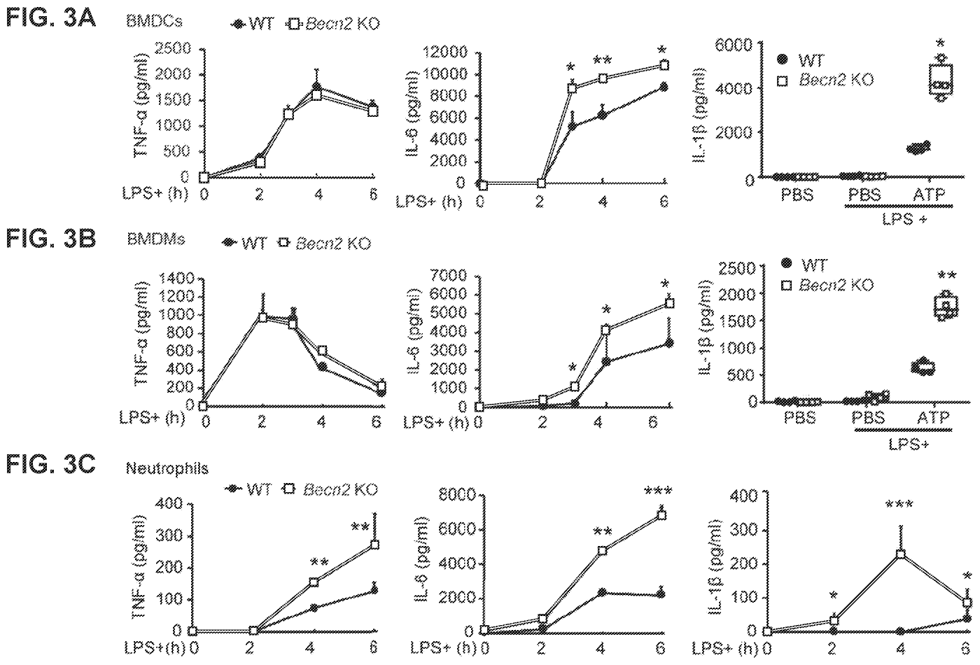

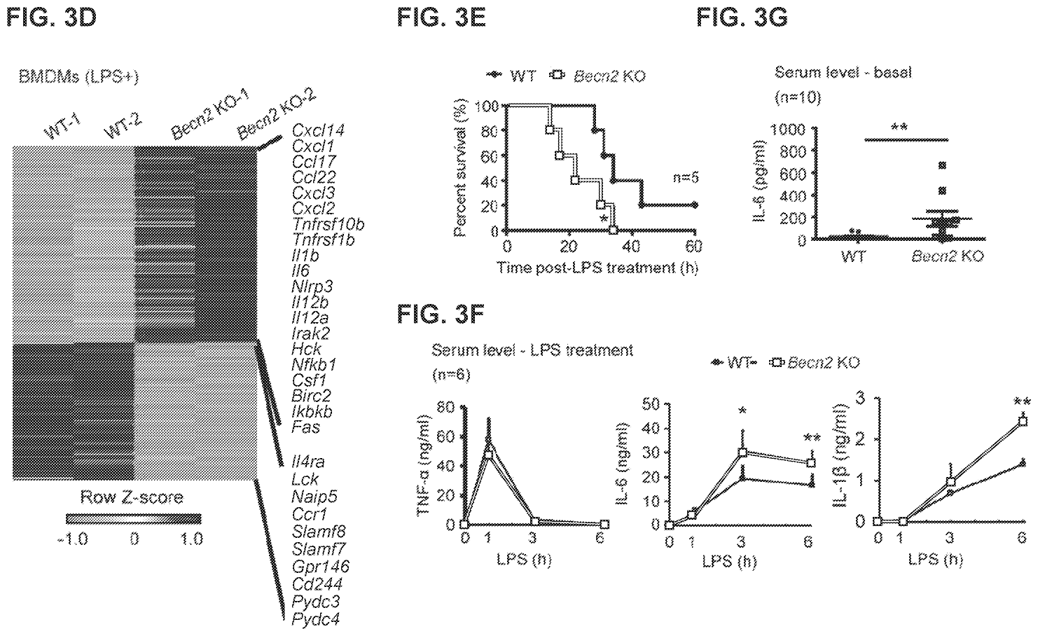

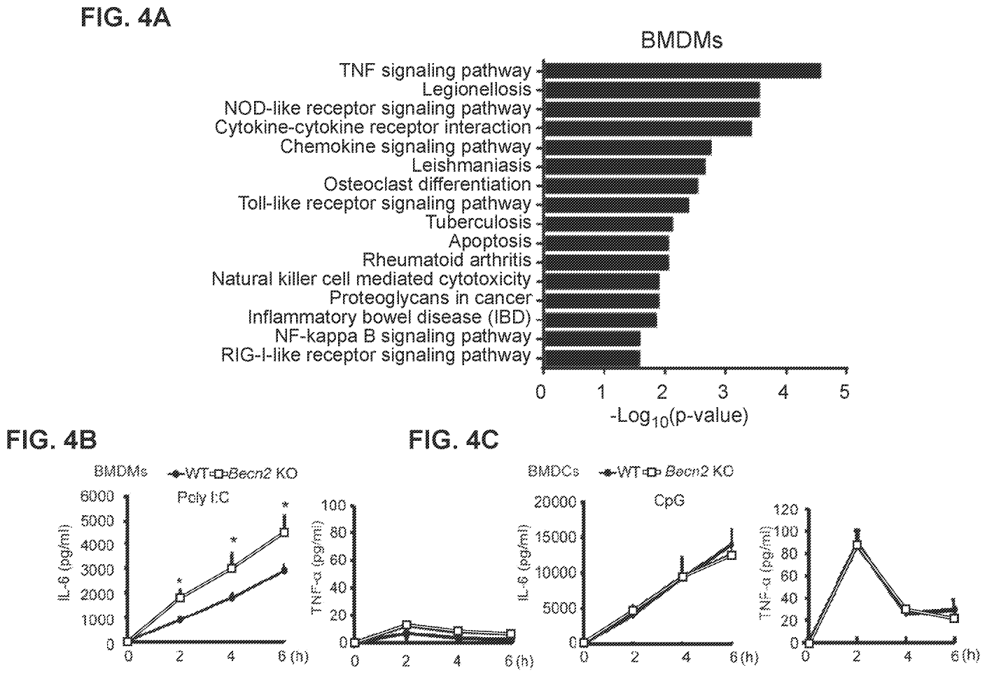

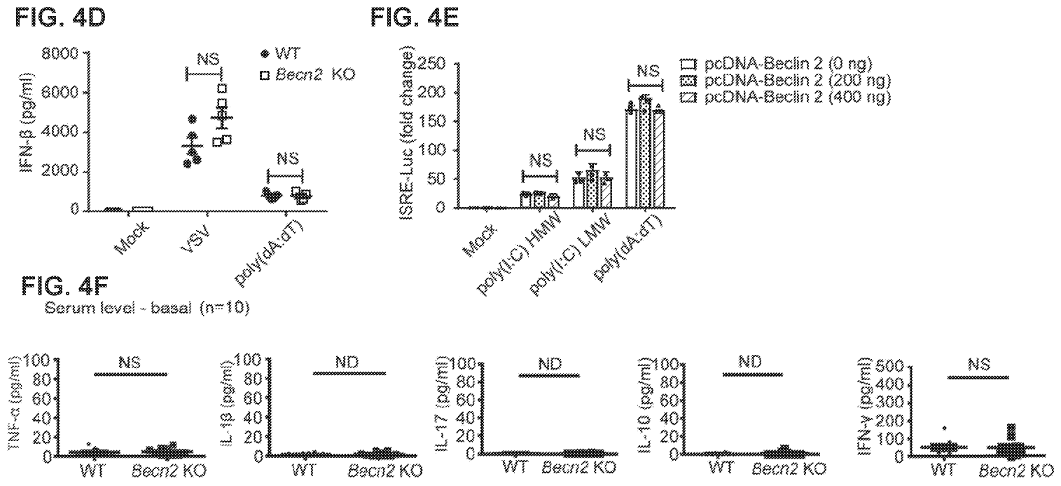

Beclin 2 deficiency increases proinflammatory cytokine production through ERK and NF-κB signaling. Next determined was the levels of proinflammatory cytokines in WT and Becn2 KO immune cells after Toll-like receptor (TLR) ligand stimulation. It was found that Becn2-deficient bone-marrow-derived dendritic cells (BMDCs) and bone-marrow-derived macrophages (BMDMs) produced more IL-6, but not tumor necrosis factor (TNF)-α, than WT cells after lipopolysaccharide (LPS, a TLR4 ligand) treatment (FIGS. 3A and 3B). LPS-primed DCs and macrophages from Becn2-deficient mice also produced more IL-1β than corresponding WT cells after ATP treatment (FIGS. 3A and 3B). Furthermore, Becn2-deficient neutrophils produced significantly more TNF-α, IL-6, and IL-1β than WT controls after LPS treatment (FIG. 3C). Consistent with the cytokine production, RNA-seq analyses showed increased expressions of IL-6 and IL-1β as well as increased expression of other cytokines and chemokines in LPS-treated Becn2 KO BMDMs (FIG. 3D). Ingenuity pathway analysis using RNA-seq data further revealed that the TNF signaling, NOD-like receptor signaling, cytokine-cytokine receptor interaction, and chemokine signaling pathways were markedly increased in Becn2 KO macrophages compared to WT cells (FIG. 4A). To determine the specificity of the innate immune response, proinflammatory cytokine expression was examined following stimulation with poly(I:C) (a ligand of TLR3) and CpG oligonucleotides (a ligand of TLR9). The experiment found that Becn2-deficient macrophages produced more IL-6, but not TNF-α, than WT macrophages after poly(I:C) treatment (FIG. 4B). However, no appreciable difference in IL-6 or TNF-α production was observed between Becn2-deficient and WT DCs after stimulation with CpG oligonucleotides (FIG. 4C). These results indicate that Beclin 2 deficiency negatively regulates IL-6, IL-1β or TNF-α production in a cell type- and TLR ligand-specific manner. Since TRIF is the adapter for both TLR3/TLR4 and type I interferon signaling, the role of Beclin 2 in type I interferon signaling pathway was examined by detecting the IFN-β production in BMDMs after VSV infection or poly(dA:dT) treatment. No appreciable difference in IFN-β production was observed between WT and Becn2 KO macrophages (FIG. 4D). Consistently, no significant change in ISRE activity was detected in poly(I:C) or poly(dA:dT) stimulated 293T cells transfected with ISRE-luciferase reporter and increasing amounts of Beclin 2 expression plasmids (FIG. 4E), indicating that Beclin 2 does not play a critical role in type I interferon signaling.

To substantiate these findings under physiological conditions, the sensitivity of Becn2 KO mice to LPS-induced septic shock was determined. After intraperitoneal (i.p.) injection of LPS at 30 mg/kg body weight, Becn2 KO mice exhibited a significantly shortened survival and rapidly died within 34 h, while 40% of WT counterparts survived over 40 h (FIG. 3E). Consistently, increased serum levels of IL-6 and IL-1β were observed in Becn2 KO mice compared with WT mice after LPS treatment (FIG. 3F). Notably, the serum level of IL-6, but not other cytokines tested, was significantly elevated in Becn2 KO mice than that in WT mice even prior to LPS treatment (FIG. 3G and FIG. 4F), indicating higher spontaneous production (basal level) of IL-6 in Becn2 KO mice.

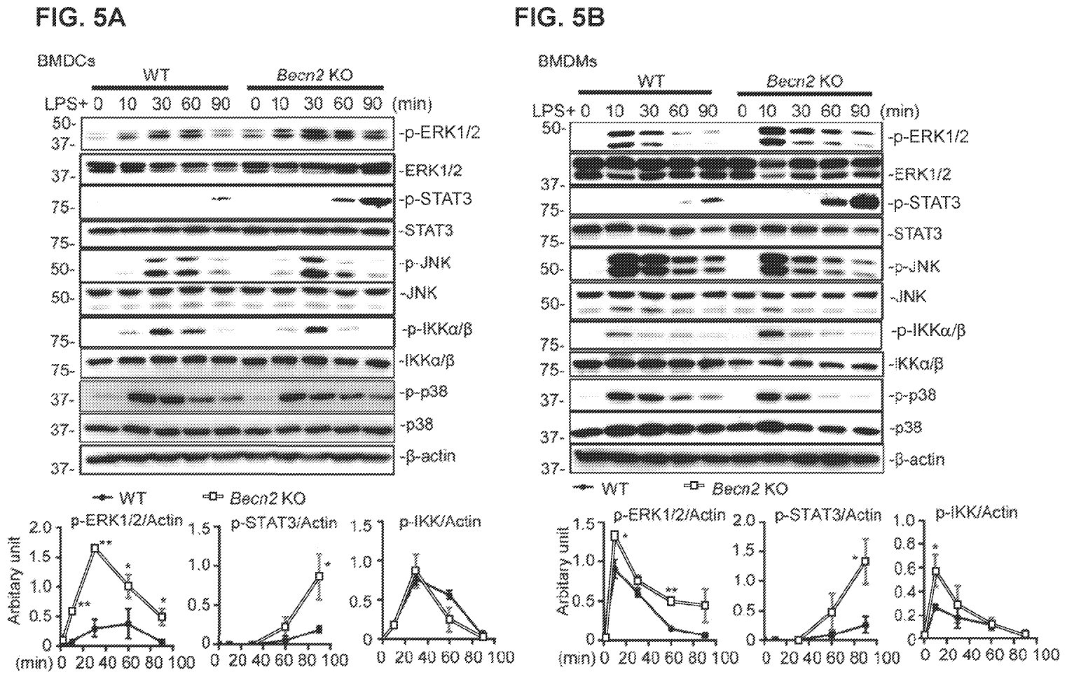

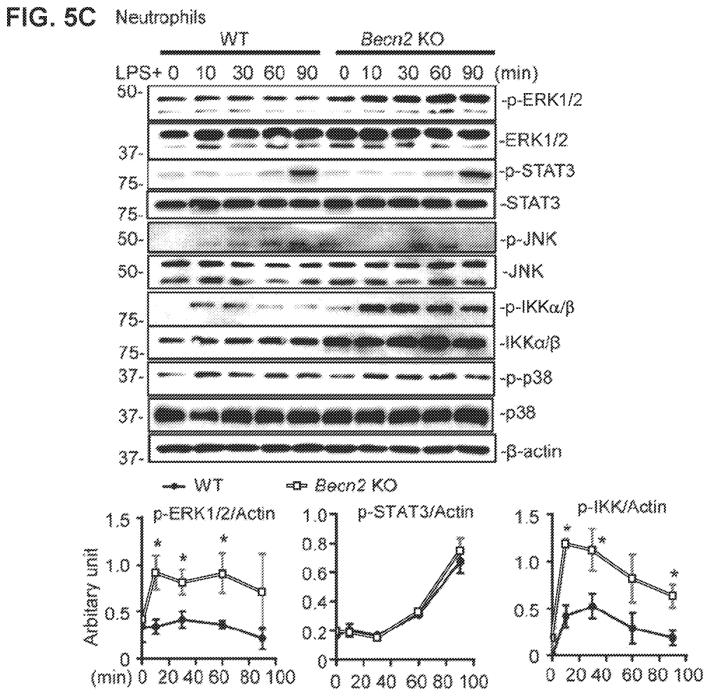

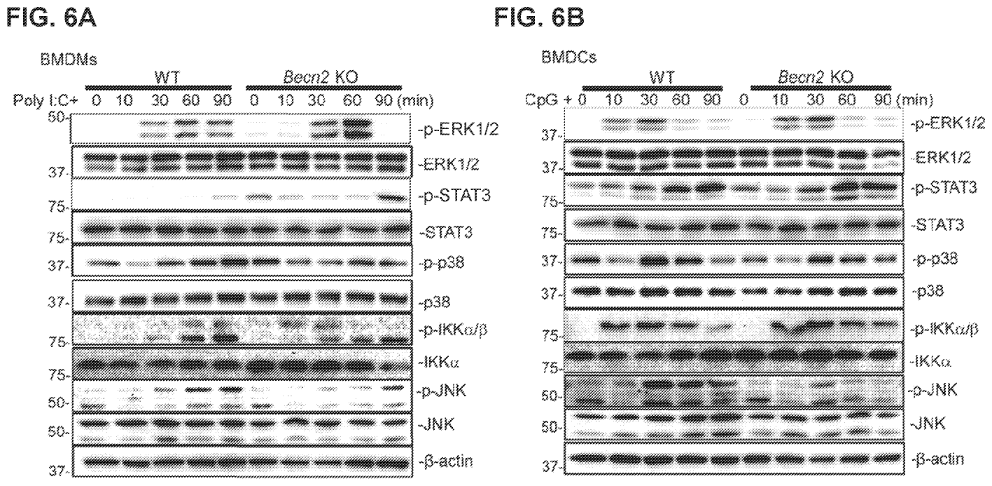

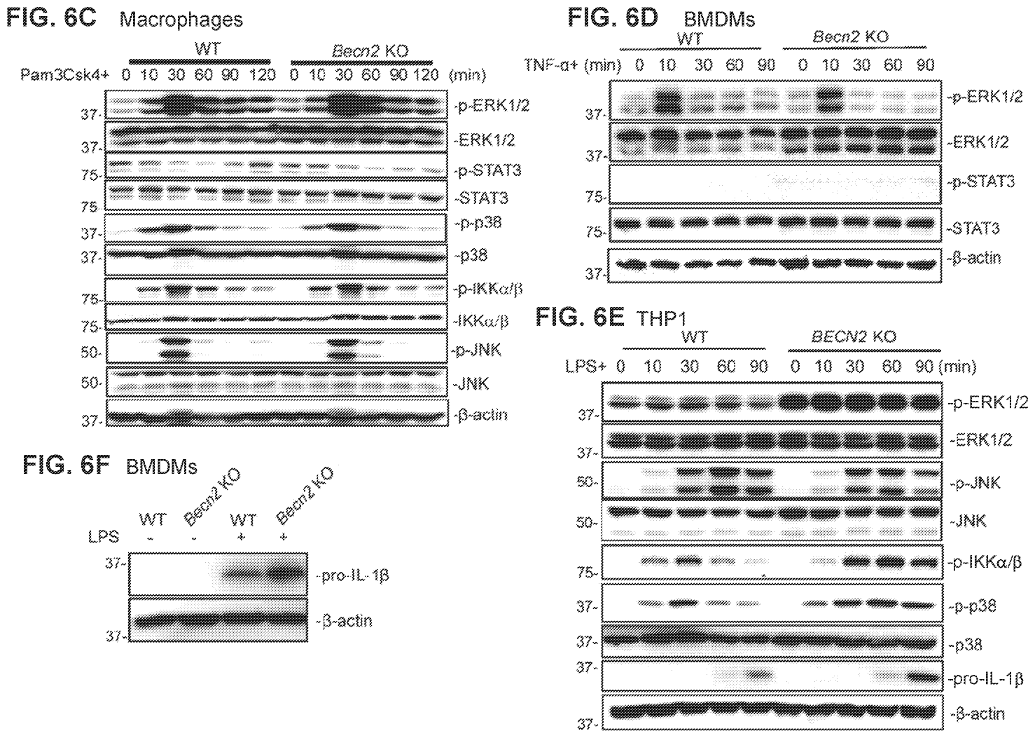

To understand the molecular mechanisms responsible for the elevated levels of proinflammatory cytokines in Becn2-deficient mice, whether the NF-κB and mitogen-activated protein kinase (MAPK) signaling pathways were affected by Beclin 2 deficiency was next tested. Western blot analysis using different immune cells revealed that the phosphorylation of ERK1/2 was enhanced in Becn2-deficient DCs (FIG. 5A), macrophages (FIG. 5B and FIG. 6A), and peritoneal neutrophils (FIG. 5C) compared with WT control cells after LPS or poly(I:C) treatment. The phosphorylation of STAT3 (signal transducer and activator of transcription 3) was also markedly enhanced in Becn2-deficient DCs and macrophages, but not in neutrophils, after LPS or poly(I:C) treatment (FIGS. 5A-5C and FIG. 6A). Notably, a moderate but significant increase in IKK phosphorylation was observed in Becn2-deficient macrophages and neutrophils upon LPS stimulation (FIGS. 5B and 5C). However, no appreciable difference in ERK1/2 or STAT3 phosphorylation was observed between Becn2 KO and WT DCs or macrophages upon CpG oligonucleotides, Pam3CSK4, or TNF-α treatment (FIGS. 6B-6D), consistent with the cytokine production in FIG. 4C. Furthermore, the signaling pathways were tested in BECN2 KO human monocytes (THP1) upon LPS stimulation and found that BECN2 KO THP1 cells generated by the CRISPR/Cas9 system had increased ERK1/2 and IKK signaling activities compared with WT controls (FIG. 6E). In addition, the IL-1β precursor (pro-IL-1β) levels in BECN2 KO THP1 cells and BMDMs were both increased, compared to that in WT cells after LPS treatment (FIGS. 6E and 6F).

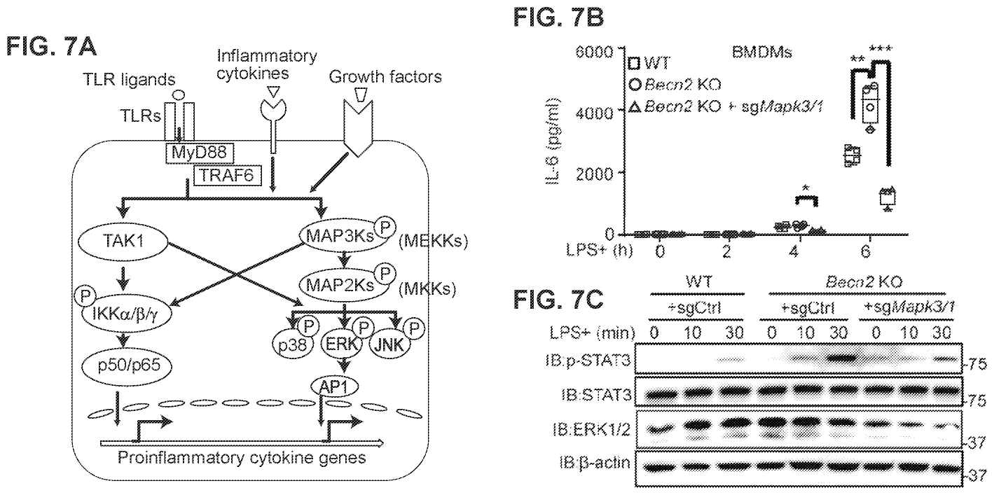

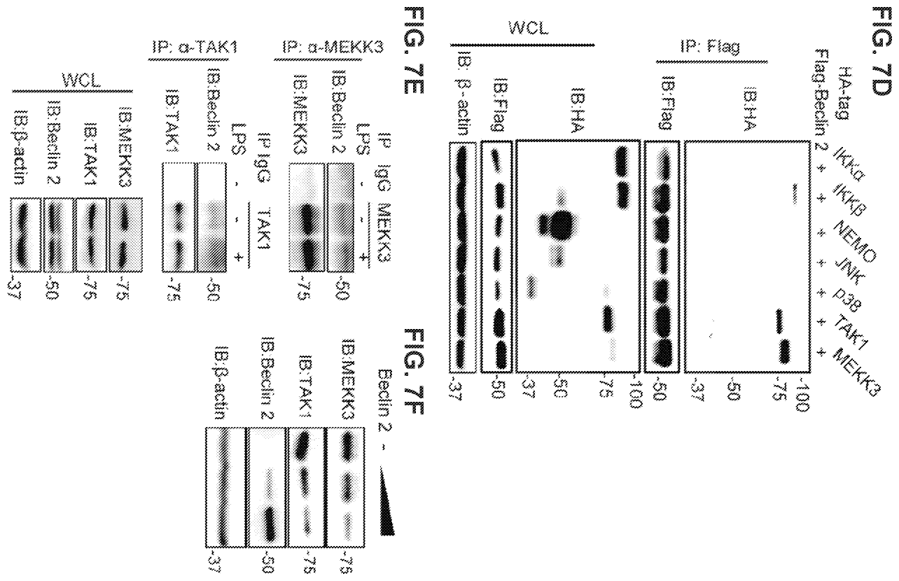

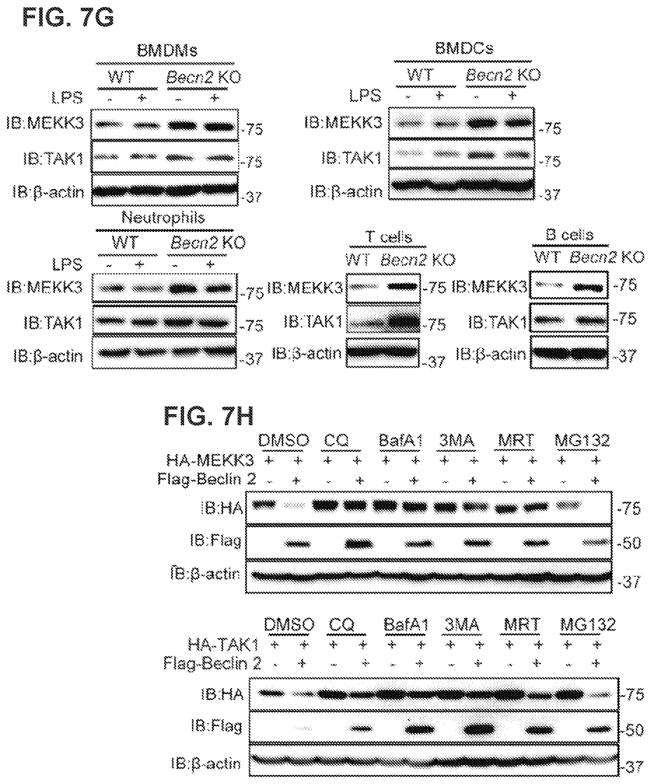

Beclin 2 inhibits ERK1/2 signaling by targeting MEKK3 and TAK1 for autophagic degradation. The NF-κB and p38 signaling pathways have been known to control proinflammatory cytokine production (FIG. 7A), but the role of ERK1/2 signaling in IL-6 expression is not clear. For this reason, Mapk3/1 (the genes encoding ERK1/2) was knocked out in Becn2-deficient macrophages using the CRISPR/Cas9 system. Co-transduction of Mapk3/1-specific sgRNAs and Cas9 into Becn2-deficient macrophages markedly reduced IL-6 production compared with the transduction of control sgRNA (FIG. 7B). Consistently, the phosphorylation of STAT3 in Becn2-deficient macrophages transduced with Mapk3/1-specific sgRNA was also reduced to a level similar to that in WT cells (FIG. 7C). These results indicate a predominant role of ERK signaling in IL-6 production and STAT3 signaling in Becn2-deficient macrophages.

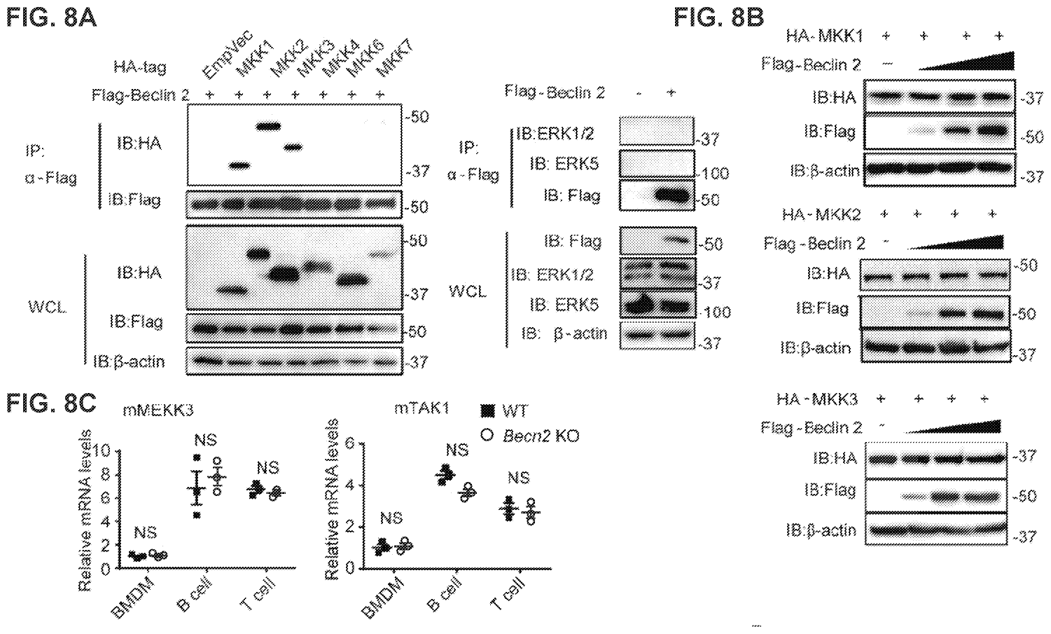

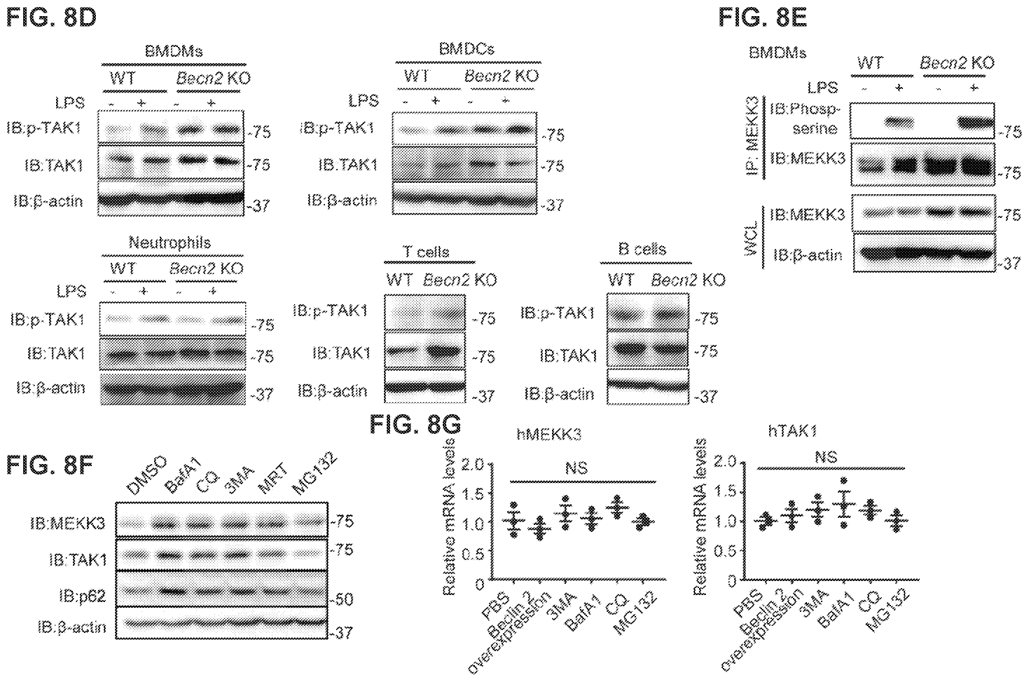

The next experiment sought to determine how Beclin 2 negatively regulates ERK1/2 signaling. Because signaling cascades of MAPK pathways, such as the ERK1/2 signaling pathway, are known to be controlled via a three-tiered process from MAP3Ks to MAP2Ks and then MAPKs (FIG. 7A), co-immunoprecipitation and western blot analyses were performed to identify the interacting proteins of Beclin 2. The data showed that Beclin 2 strongly interacted with MEKK3 (Map3k3), but weakly interacted with IKKβ and TAK1 (Map3k7) (FIG. 7D). The endogenous interactions of Beclin 2 with TAK1 and MEKK3 were also detected, but such interactions were not affected by LPS stimulation (FIG. 7E). Furthermore, Beclin 2 strongly interacted with MKK1, MKK2, and MKK3 but not with ERK1, ERK2, ERK5, JNK or p38 (FIG. 8A), indicating that Beclin 2 regulates ERK1/2 signaling through the control of its upstream kinases. To further determine how Beclin 2 regulates TAK1- and MEKK3-mediated signaling, whether Beclin 2 can target TAK1 and MEKK3 for degradation was tested. 293T cells were transfected with increasing amounts of Beclin 2 expression vector and found that the endogenous protein levels of both TAK1 and MEKK3 were markedly reduced with increasing Beclin 2 expression (FIG. 7F). By contrast, we did not observe appreciable changes in HA-tagged MKK1, MKK2, or MKK3 protein levels with increasing Beclin 2 expression (FIG. 8B). Consistently, the protein levels of MEKK3 and TAK1 were markedly increased in Becn2-deficient immune cells, such as DCs, macrophages, neutrophils, and T cells compared with WT controls (FIG. 7G), while the mRNA levels of both MEKK3 and TAK1 had no appreciable differences between WT and Becn2 KO cells (FIG. 8C). The levels of p-TAK1 and p-MEKK3 in immune cells were next determined to test the possibility that loss of Beclin 2 can induce more TAK1 or MEKK3 phosphorylation for enhanced ERK signaling. Although increased p-TAK1 levels (in Beclin 2 KO BMDMs, BMDCs, and T cells) and p-MEKK3 (in BMDMs) were evident in Becn2 KO cells compared to WT cells, the total TAK1 and MEKK3 levels were also increased in these cell types (FIGS. 8D and 8E). By contrast, no appreciable difference in the phosphorylation of TAK1 was observed between WT and KO cells in the cell types that express comparable TAK1 levels, including neutrophils and B cells (FIG. 8D), indicating that Beclin 2 mainly regulates the protein level of TAK1 but not its phosphorylation.

To further determine how Beclin 2 mediates the degradation of TAK1 and MEKK3 degradation, the autophagy inhibitors 3-methyladenine (3MA), bafilomycin A (BafA), chloroquine (CQ), or MRT68921 (ULK1/2 dual kinases inhibitor) were used to inhibit autophagy-dependent degradation, and a proteasome inhibitor (MG132) was used to inhibit the proteasomal degradation pathway. It was found that the Beclin 2-mediated degradation of TAK1 and MEKK3 was significantly inhibited by the autophagy inhibitors, but not by MG132 (FIG. 7H). Consistently, the endogenous TAK1 and MEKK3 were also increased in autophagy/lysosome inhibitor-treated 293T cells at the protein level, but with little changes at the mRNA level (FIGS. 8F and 8G), indicating that the autophagic pathway is required for the degradation of TAK1 and MEKK3 proteins by Beclin 2.

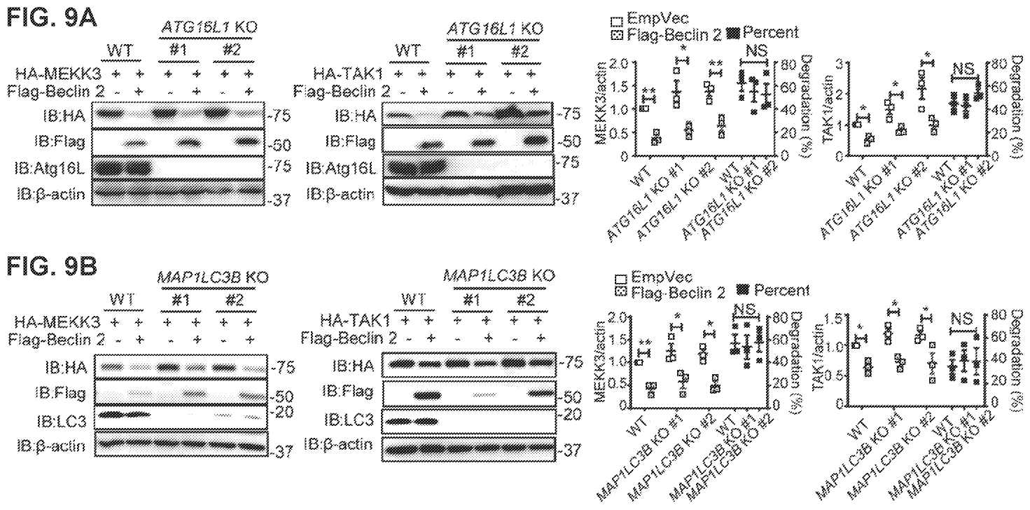

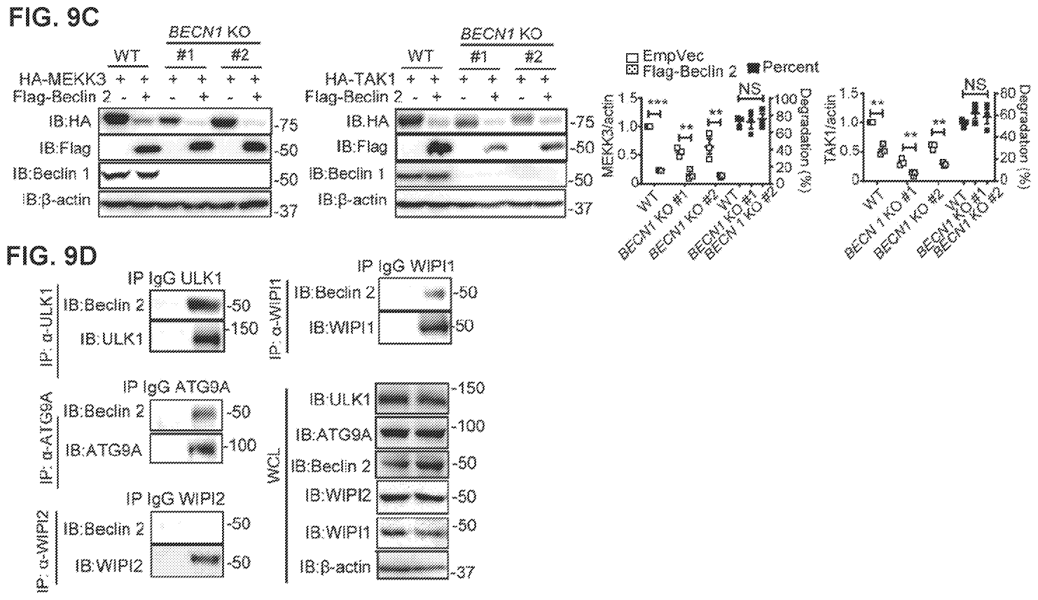

Beclin 2 mediates the degradation of TAK1 and MEKK3 through an ATG9A-dependent but ATG16L/LC3B/Beclin 1-independent autophagic pathway. Beclin 2 is involved in autophagy and interacts with known binding partners of Beclin 1 in the class III phosphoinositide 3-kinase (PI3K) complex, including ATG14, VPS34, and AMBRA1, but how Beclin 2 mediates degradation of target proteins through autophagic pathway remains unknown. Macroautophagy requires the hierarchically ordered activities of ATG proteins recruited at the phagophore assembly site (PAS) to form a double-membrane autophagosome. However, recent studies indicate that autophagy can occur in alternative forms that do not require the hierarchical actions of all ATG proteins to form autophagosomes, but rather a set of ATG proteins are recruited to a pre-existing double-membrane structure for autophagosome formation. To further decipher the molecular mechanisms of Beclin 2-mediated autophagic degradation of TAK1 and MEKK3, whether Beclin 2-mediated degradation can be blocked in ATG protein-deficient cells was first examined. A IG16L and MAP1LC3B (two genes that are essential for autophagosome membrane elongation in macroautophagy) were knocked out in 293T cells, and it was found that Beclin 2-mediated TAK1 and MEKK3 degradation can not be blocked in the cells deficient in either ATG16L or LC3B (FIGS. 9A and 9B). Ablation of BECN1 (a gene essential for nucleation in macroautophagy) also failed to block the degradation of TAK1 and MEKK3 (FIG. 9C). Quantitative analysis of TAK1 and MEKK3 protein levels and degradation efficiency in WT and KO cells also indicated that Beclin 2-mediated TAK1 and MEKK3 degradation was not compromised in ATG16I/MAP1LC3B/BECN1-deficient cells, compared to WT cells (FIG. 9A-9C), indicating that ATG16L, LC3B and Beclin 1 are not essentially required for Beclin 2-mediated degradation of TAK1 and MEKK3.

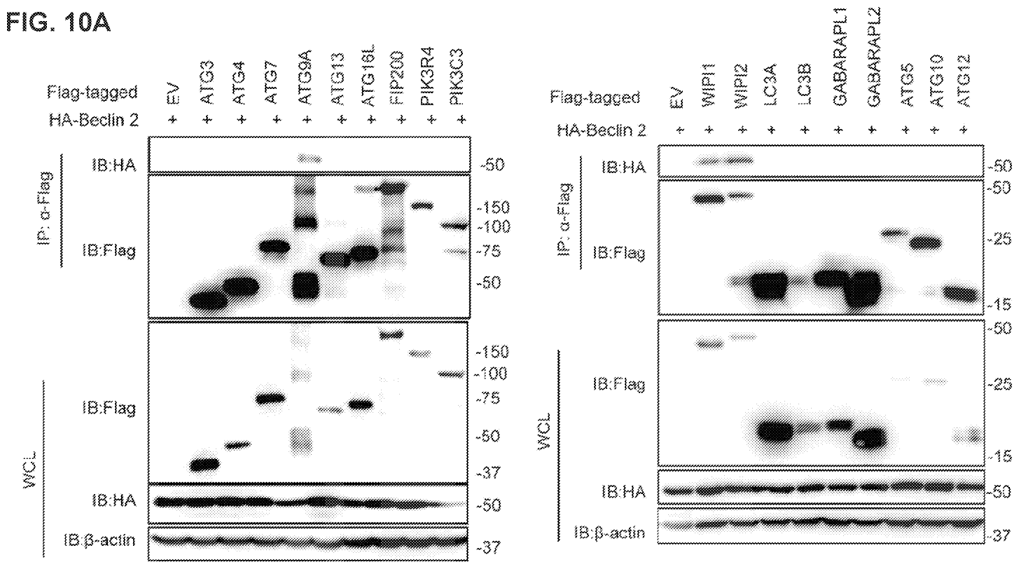

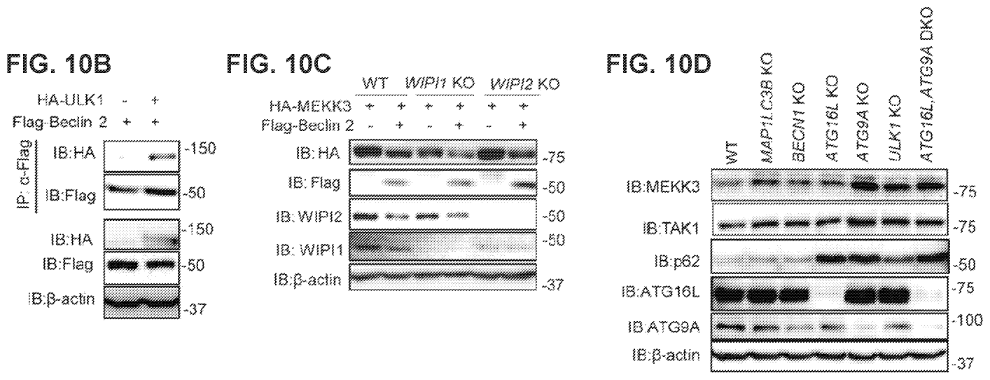

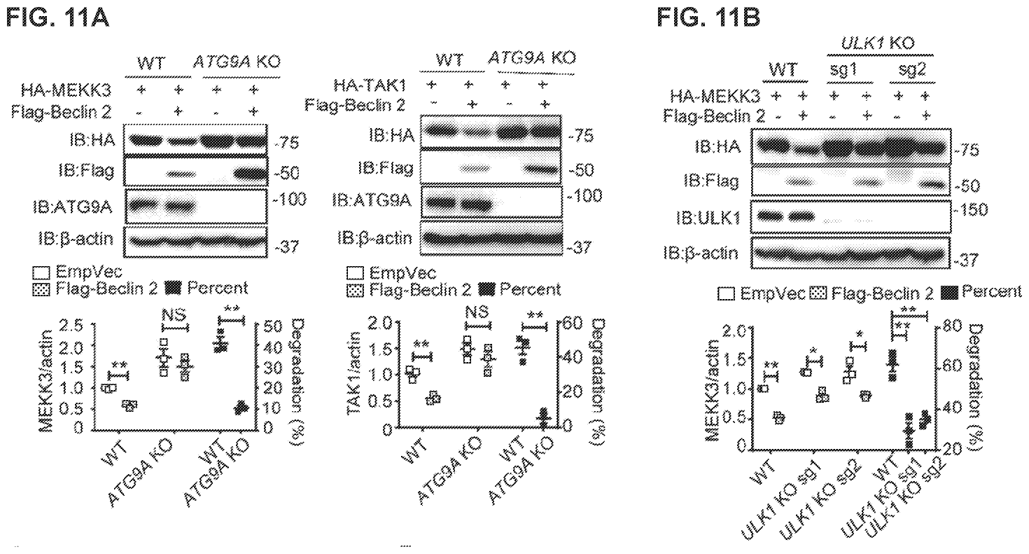

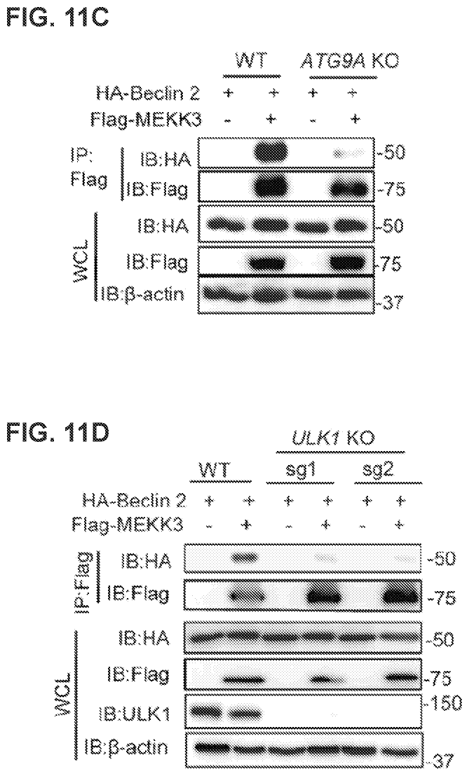

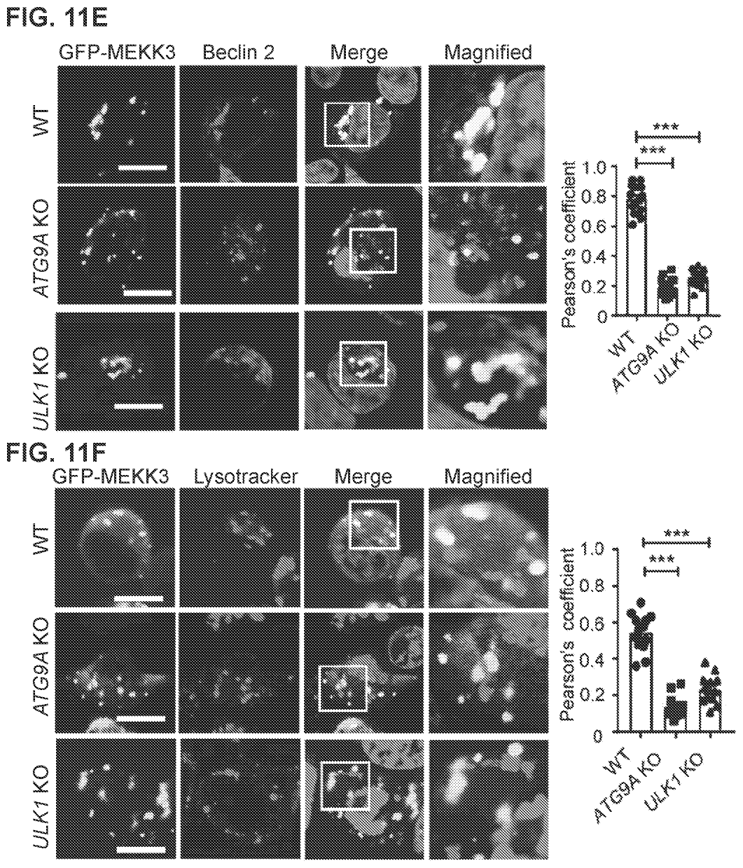

What was next determined was the key autophagic proteins that are essential for Beclin 2-mediated degradation of TAK1 and MEKK3. Co-immunoprecipitation of Beclin 2 and its potential binding partners involved in autophagy machinery revealed that Beclin 2 interacted with WIPI1, WIPI2, ATG9A, and ULK1 (FIGS. 10A and 10B). It was also confirmed the endogenous interactions of Beclin 2 with ULK1, ATG9A, WIPI1, but not with WIPI2 (FIG. 9D). ATG9A KO, ULK1 KO and WIPI1/2 KO cells were next generated using CRISPR/Cas9 technology and it was found that ATG9A ablation completely abolished Beclin 2-mediated TAK1 and MEKK3 degradation, while knockout (KO) of ULK1 partially blocked the degradation (FIGS. 11A and 11B). However, WIPI1 or WIPI2 deficiency had little or no effect on Beclin 2-mediated MEKK3 degradation (FIG. 10C). The endogenous protein levels of MEKK3 and TAK1 in different ATG KO cells were further compared, as well as in ATG9A:ATG16L DKO cells. Although the protein levels of MEKK3 and TAK1 in MAP1LC3B KO, ATG16L1 KO, and BECN1 KO cells were slightly increased (FIG. 10D), much higher levels of MEKK3 and TAK1 were observed in ATG9A KO and ULK1 KO cells. Furthermore, these two protein levels in ATG9A:ATG16L DKO cells were similar to those in ATG9A single KO cells, but much higher than those in ATG16L single KO cells (FIG. 10D). These data further demonstrate that ATG9A and ULK1, but not ATG16L/LC3/Beclin 1, play a dominant role in the degradation of MEKK3 and TAK1.

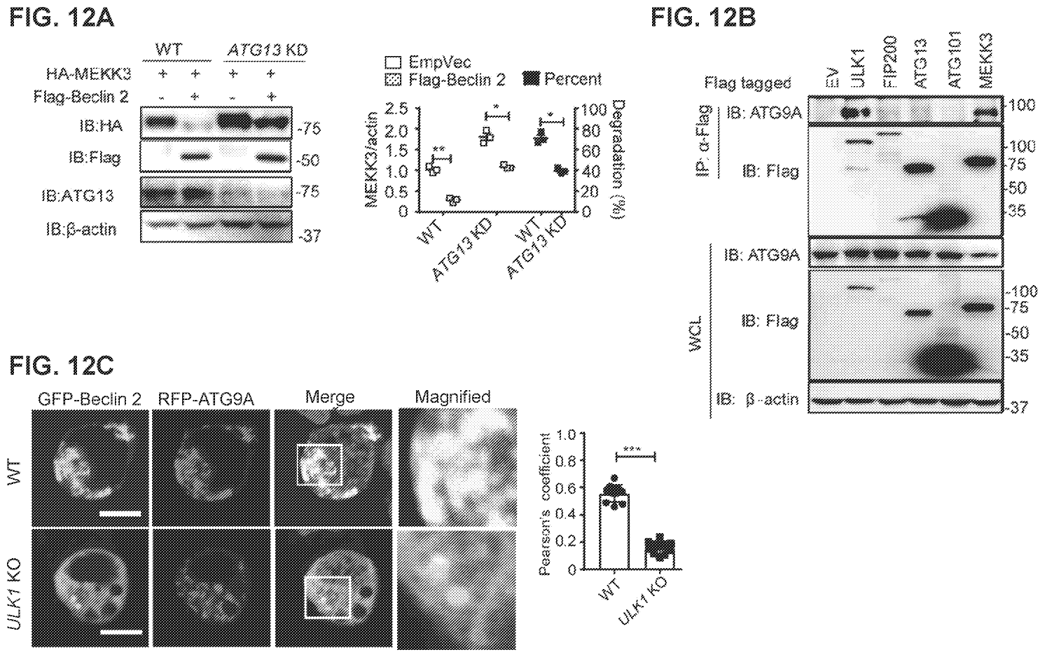

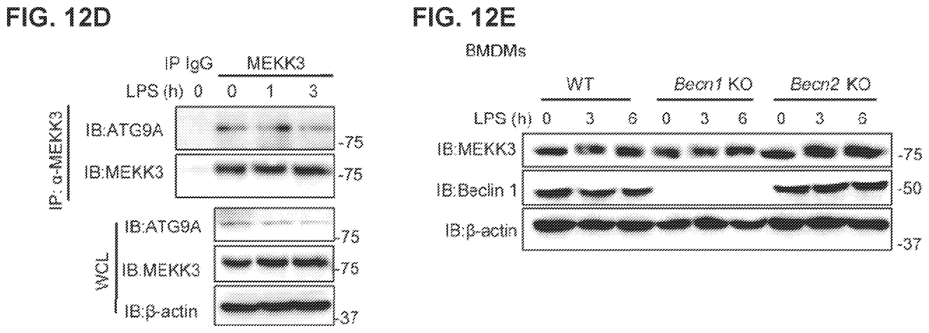

Beclin 2 promotes the fusion of MEKK3-associated ATG9A+ vesicles with phagophores by interacting with STX5 and STX6 for MEKK3 degradation. ATG9A is the only transmembrane ATG protein and the ATG9A-associated vesicle is essential for the membrane assembling in autophagosome formation. The recruitment of ATG9A-vesicle to phagophores requires the activation of the ULK1 complex, including ULK1 and ATG13. Indeed, it was shown herein that Beclin 2-mediated MEKK3 degradation was markedly impaired in AG13 shRNA-knockdown cells compared to WT cells (FIG. 12A). However, further experiments showed that ATG9A interacted with ULK1 and MEKK3 but not with ATG13 or FIP200 (FIG. 12B). To further understand the role of ATG9A and ULK1 in Beclin 2-mediated MEKK3 degradation, whether ATG9A and ULK1 were required for the interaction between Beclin 2 and MEKK3 was tested. Co-immunoprecipitation and immunoblot analysis revealed that Beclin 2 almost completely lost the ability to interact with MEKK3 in ATG9A KO and ULK1 KO cells (FIGS. 11C and 11D). Beclin 2 also failed to be recruited to ATG9A+ vesicles in ULK1 KO cells (FIG. 12C). These results indicate that ATG9A-ULK1 is the key to bridge the interaction between Beclin 2 and MEKK3. To further demonstrate their intracellular interactions, confocal microscopic analysis was performed, which found that the co-localization of GFP-MEKK3 and Beclin 2 was significantly impaired in ATG9A KO and ULK1 KO cells, compared to WT cells (FIG. 11E). Furthermore, GFP-MEKK3 was translocated into lysosome in Flag-Beclin 2 overexpressed WT cells, but such translocation was significantly compromised in ATG9A- or ULK1-deficient cells as shown by Pearson's coefficient analysis (FIG. 11F). These data demonstrate the critical role of ULK1 and ATG9A in Beclin 2-mediated degradation of TAK1 and MEKK3 through the engagement of ATG9A as a bridge for Beclin 2-ATG9A-MEKK3 complex formation. To confirm the ATG9A engagement in MEKK3 degradation in primary myeloid cells, and to determine if the degradation requires LPS stimulation, the endogenous interaction between MEKK3 and ATG9A in BMDMs was determined and the experiment found that MEKK3 can bind to ATG9A regardless of LPS stimulation (FIG. 12D). Furthermore, no appreciable change in MEKK3 level was detected before or after LPS stimulation in WT, Becn1 KO or Becn2 KO BMDMs (FIG. 12E), indicating that Beclin 2-mediated ATG9A-dependent autophagic degradation of MEKK3 is independent of LPS-induced TLR4 signaling.

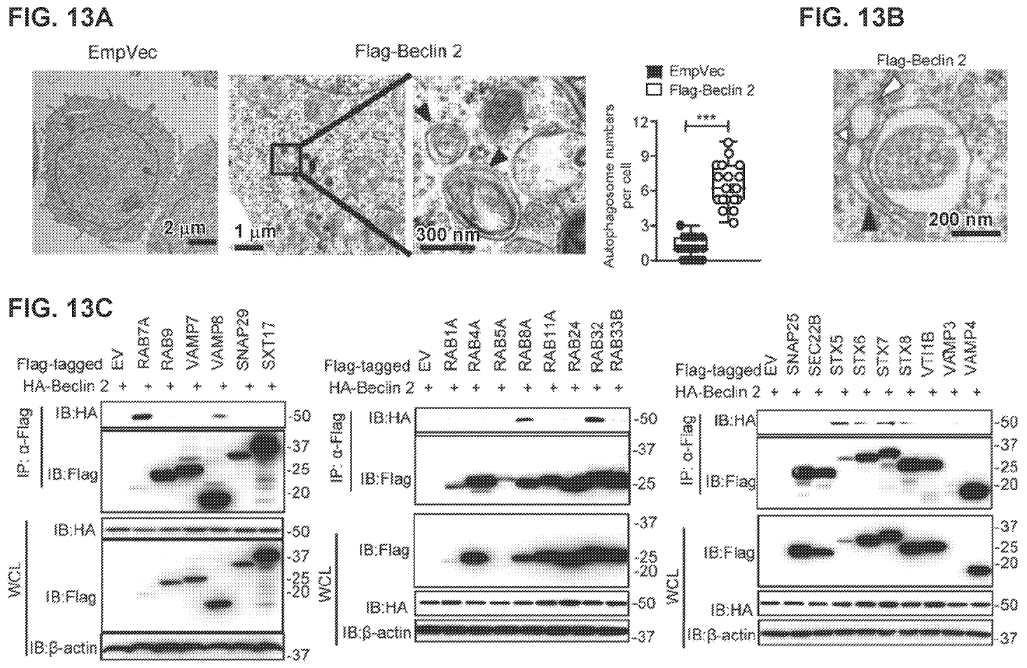

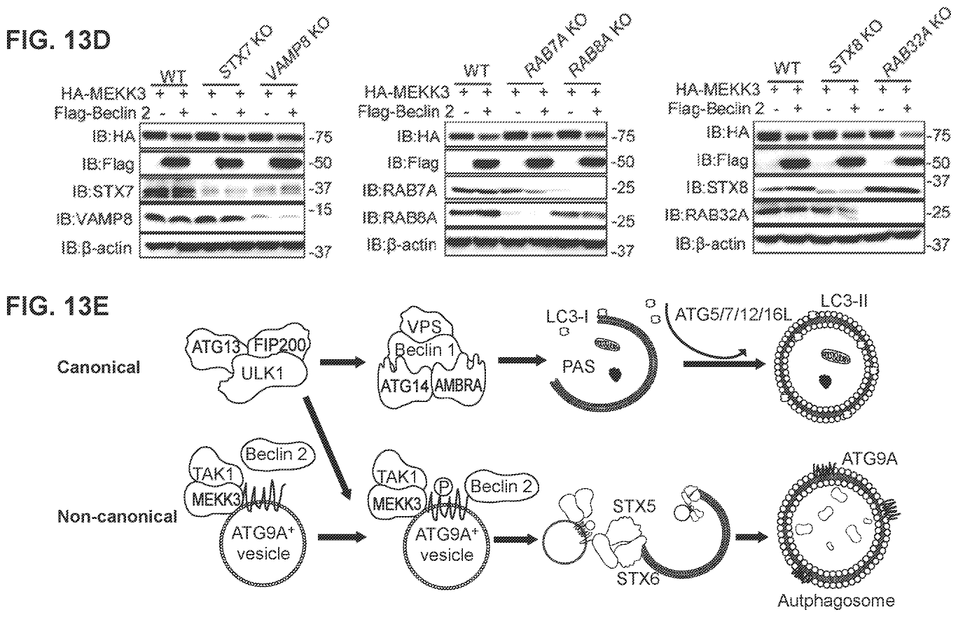

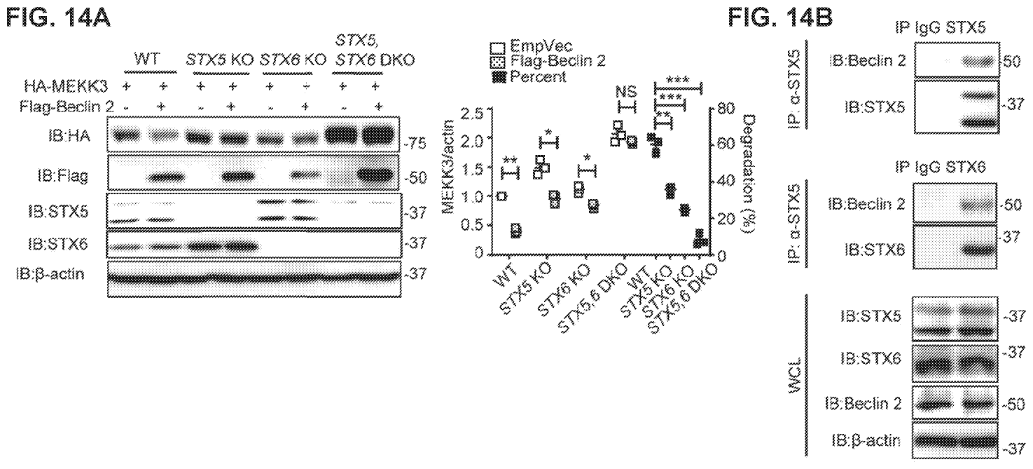

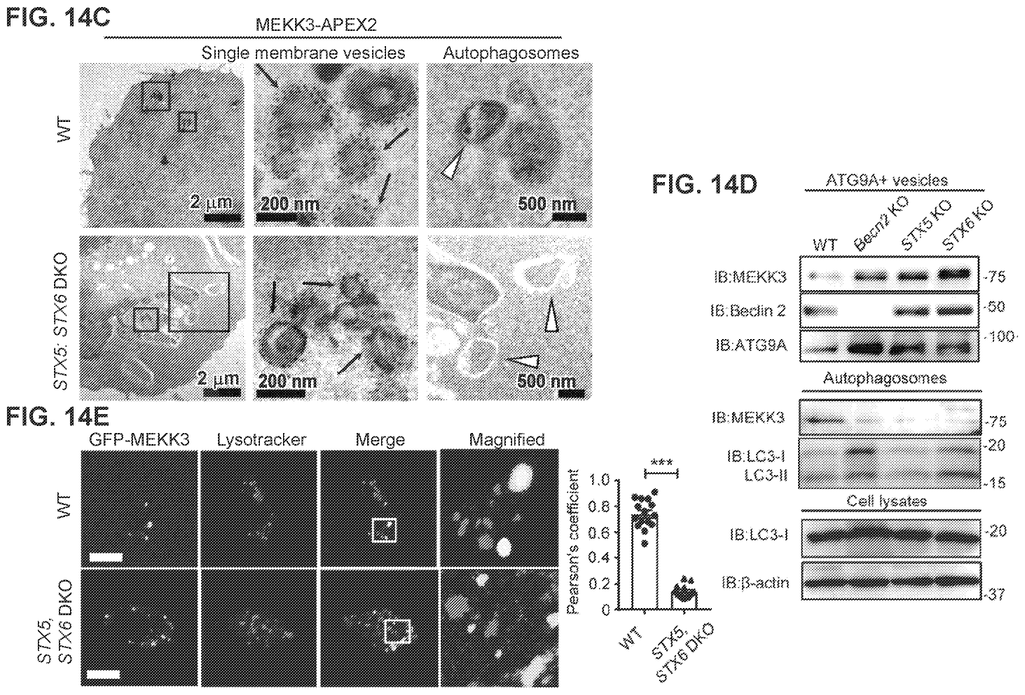

To further dissect the molecular mechanisms by which Beclin 2 mediates MEKK3 degradation through ATG9A⁺ vesicles, it was reasoned that Beclin 2 can promote the membrane fusion of ATG9A-vesicles with phagophore to form autophagosome. Indeed, ectopic expression of Beclin 2 in 293T cells increased the vesicle fusion with phagophores for autophagosomes formation, as shown in the transmission electron microscopy (TEM) images (FIGS. 13A and 13B). Therefore, the next experiment sought to determine the interaction between Beclin 2 and a series of RAB GTPases and SNARE proteins for their involvement in membrane fusion. Co-immunoprecipitation and western blot analyses show that Beclin 2 can interact with RAB7A, RAB8A, RAB32, VAMP8, syntaxin 5 (STX5), STX6, STX7, and STX8 (FIG. 13C). KO cells of each Beclin 2-interacting partner were next generated using CRISPR/Cas9 technology to test whether specific gene KO can partially or completely block the MEKK3 degradation. It was found that ablation of either STX5 or STX6 can partially block the Beclin 2-mediated MEKK3 degradation (FIG. 14A), while KO of other genes had little or no effect (FIG. 13D). Importantly, Beclin 2-mediated MEKK3 degradation can be completely blocked in STX5:STX6 double KO (DKO) cells (FIG. 14A). To further investigate the physiological role of STX5 and STX6 in Beclin 2-mediated MEKK3 degradation, the endogenous interactions of Beclin 2 with STX5 and STX6 were demonstrated (FIG. 14B). The next experiment examined the translocation of MEKK3 in WT and STX5:STX6 DKO cells using TEM analysis (by APEX2-enabled staining) and it was found that MEKK3 can be associated with single-membrane vesicles or transported to autophagosomes (double-membrane structures) in WT cells. In contrast, MEKK3 was barely detected in autophagosomes in STX5:STX6 DKO cells, but accumulated with single-membrane vesicles (FIG. 14C). In line with the EM observation, membrane fractionation was performed to enrich autophagosomes and immune-isolated Flag-tagged ATG9A⁺ vesicles, respectively, from cells of different genotypes. Significant amounts of MEKK3 was found in ATG9A-vesicles among all genotypes (FIG. 14D). Moreover, the increased amounts of MEKK3 can be detected in ATG9A⁺ vesicle fractions from BECN2 KO, STX5 KO, and STX6 KO cells compared with WT counterparts (FIG. 14D). However, the enrichment of MEKK3 in autophagosomes can only be detected in WT cells but not in BECN2 KO, STX5 KO or STX6 KO cells, indicating the translocation of MEKK3 from ATG9A⁺ vesicles to autophagosomes is markedly blocked in these KO cells (FIG. 14D). Consistently, the translocation of MEKK3 into the lysosomes was also diminished in STX5:STX6 DKO 293T cells even with Beclin 2-overexpression (FIG. 14E). Taken together, these results show that the interaction between Beclin 2 and STX5/STX6 promotes the membrane fusion of ATG9A⁺ vesicles with phagophores to form autophagosomes for MEKK3 degradation (FIG. 13E).

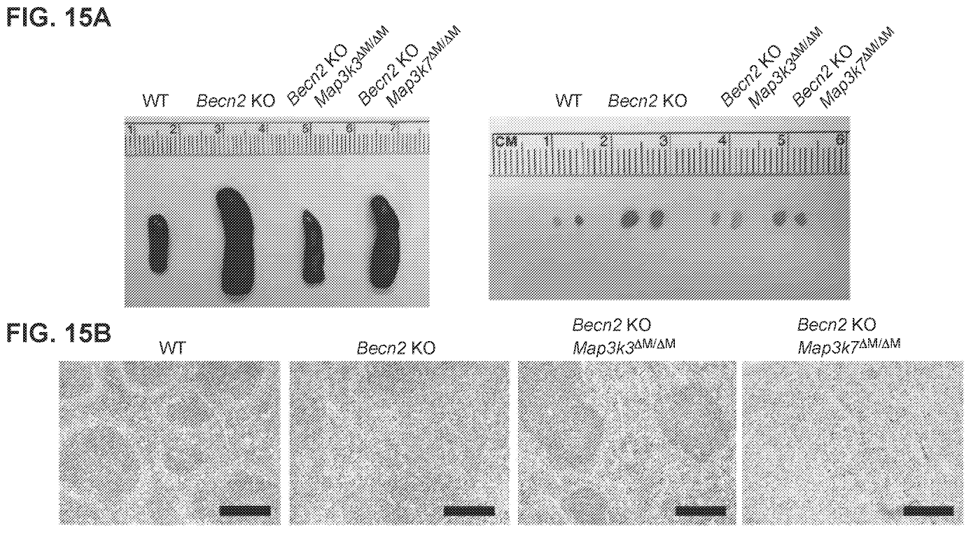

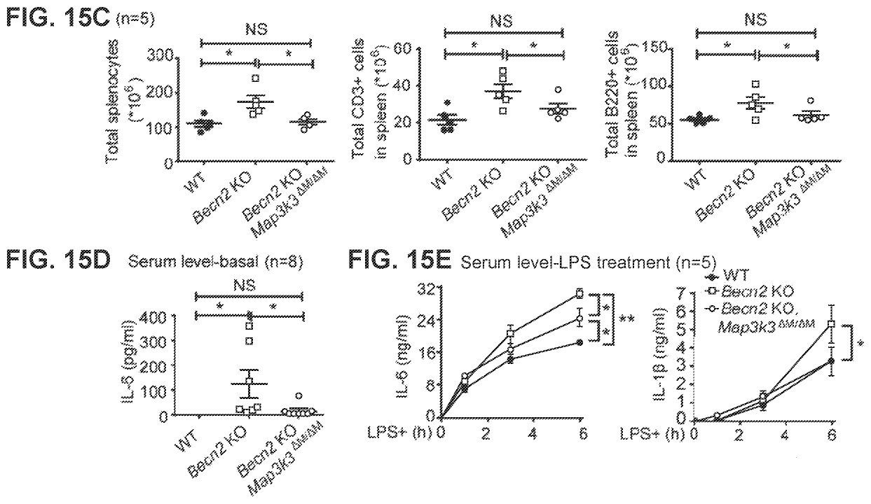

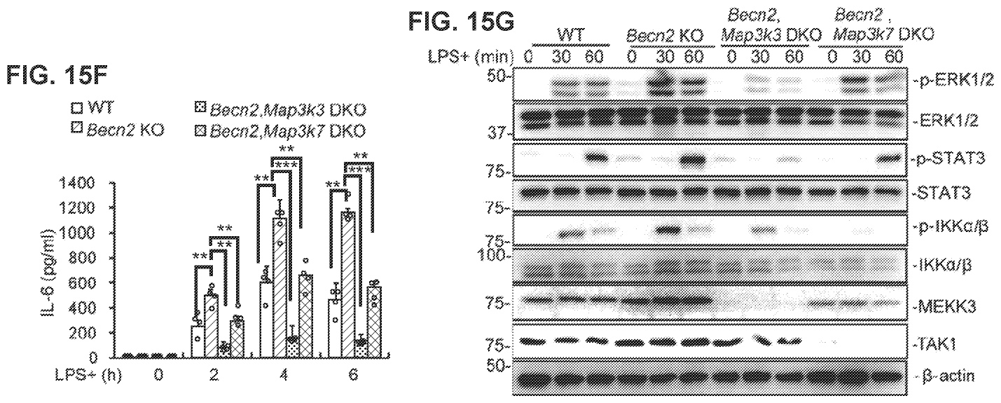

Ablation of Map3k3 rescues phenotypes observed in Becn2 KO mice. To further explore the physiological function of Beclin 2-mediated MEKK3 and TAK1 degradation, whether specific ablation of MEKK3 or TAK1 can rescue the phenotypes observed in Becn2-deficient mice was examined. Becn2 KO mice were crossed with myeloid-specific TAK1-deleted (Map3k7^{ΔM/ΔM}) mice or myeloid-specific MEKK3-deleted (Map3k3^{ΔM/ΔM}) mice, and found that myeloid-specific ablation of MEKK3 (Map3k3^{ΔM/ΔM}:Becn2 KO) completely rescued the phenotypes (splenomegaly and lymphadenopathy) observed in Becn2-deficient mice (FIG. 15A), whereas TAK1 deficiency (Map3k7^{ΔM/ΔM}:Becn2 KO) only partially rescued the phenotypes. Consistently, histological examination revealed that Map3k3^{ΔM/ΔM}:Becn2 KO mice restored the disorganized spleen architectures observed in Becn2-deficient mice to a level similar to WT mice, while Map3k7^{ΔM/ΔM}:Becn2 KO mice only partially restored the germinal center and nodular architecture (FIG. 15B). Furthermore, the total numbers of splenocytes, B220⁺ and CD3⁺ lymphocytes, as well as the serum cytokine production in Map3k3^{ΔM/ΔM}:Becn2 KO mice under the untreated (basal) and LPS-induced conditions, were either partially or completely restored to the levels comparable with WT mice (FIGS. 15C-15E). These results were further supported by a marked reduction of IL-6 in macrophages isolated from Map3k3^{ΔM/ΔM}:Becn2 KO and Map3k7^{ΔM/ΔM}:Becn2 KO mice, compared to Becn2 KO cells (FIG. 15F). Consistent with the cytokine production, ablation of Map3k3 or Map3k7 in Becn2 KO macrophages markedly reduced the phosphorylation of ERK1/2 and IKKα/β to the levels similar to or even lower than those in WT macrophages (FIG. 15G). These results indicate that myeloid-specific TAK1 and MEKK3 are critical in mediating IL-6 production, but MEKK3 plays a predominant role in the development of splenomegaly and lymphadenopathy in Becn2 KO mice.

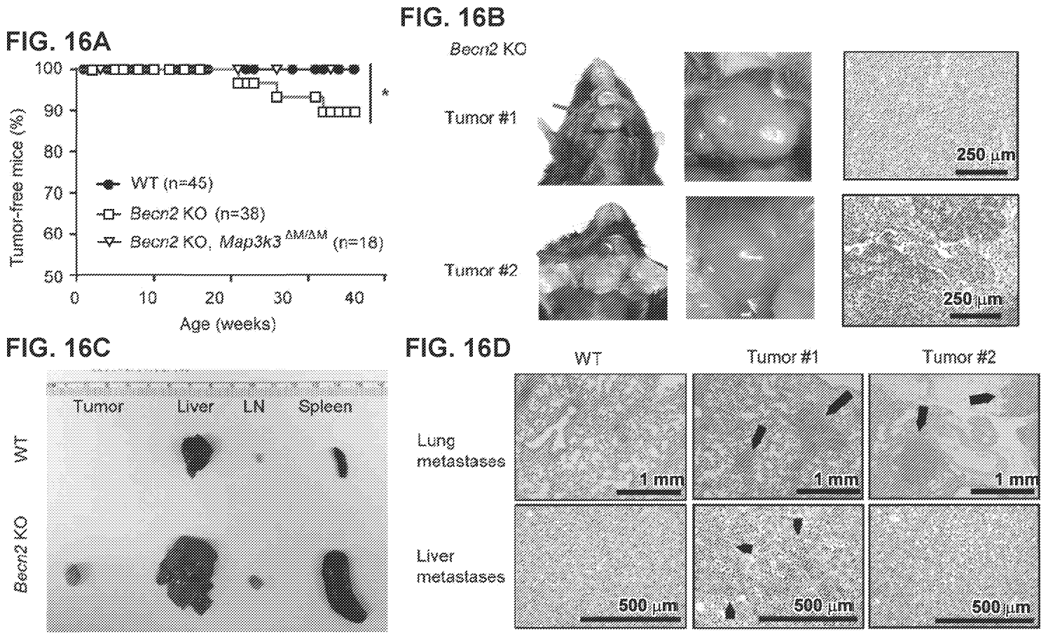

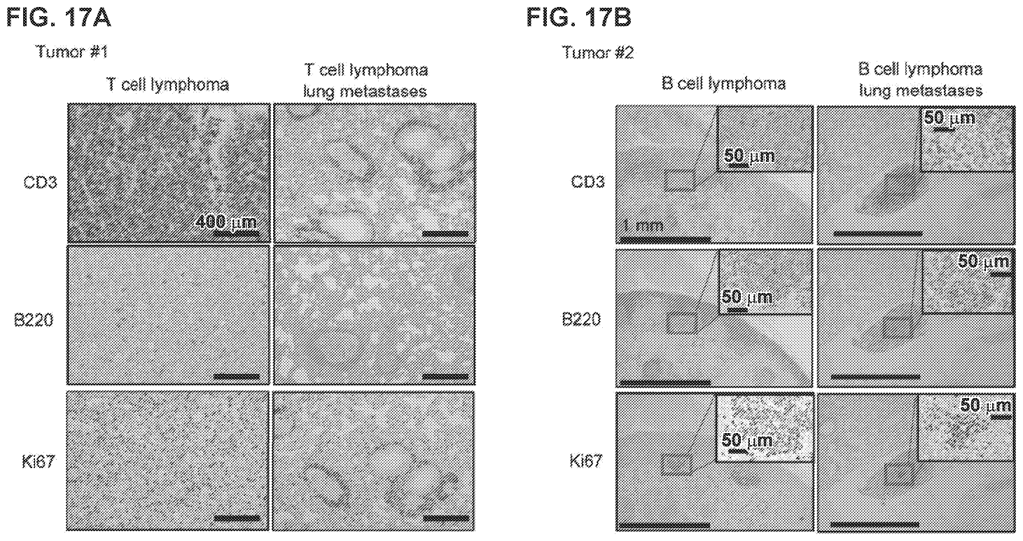

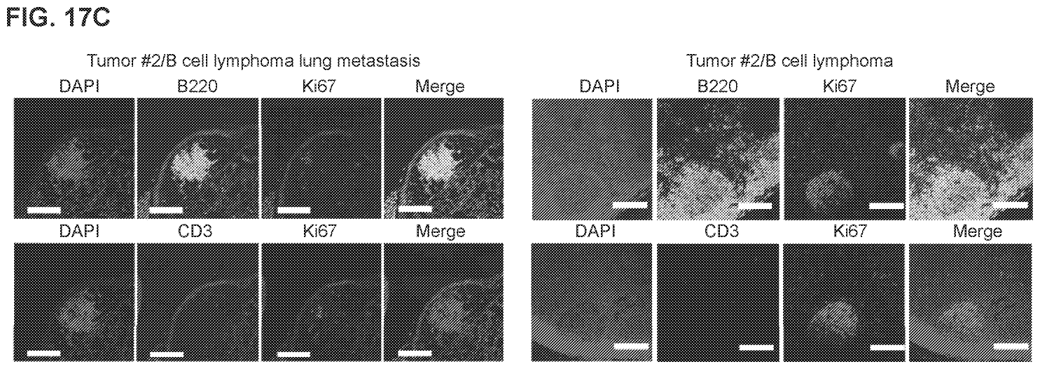

Increased incidence of metastatic lymphoma development in Becn2 KO mice. Based on these findings that Becn2 KO mice produce large amounts of proinflammatory cytokines such as IL-6, it was reasoned that these KO mice can have a higher risk of developing cancer. Indeed, tumor development was observed in homozygous Becn2 KO mice at the age of approximately 20-32 weeks (FIG. 16A). Among 38 Becn2 KO mice (from 6 to 36 weeks old), 5 (13.2%) developed spontaneous tumors, compared with no tumor development in WT mice. To determine whether myeloid-specific ablation of Map3k3 can also rescue the spontaneous tumor development in Becn2 KO mice, 18 Becn2 KO:Map3k3^{ΔM/ΔM} mice were checked at the matched ages and it was found that none of these mice developed tumor (FIG. 16A). These results indicate that excess MEKK3-mediated signaling in Becn2 KO mice plays a critical role in spontaneous tumor development. The tumors found in Becn2 KO mice were near the neck and collarbone region (FIG. 16B) and had different pathological architectures. The sizes of the livers, inguinal lymph nodes and spleens from tumor-bearing Becn2 KO mice were dramatically increased compared with those from WT mice (FIG. 16C). Based on the H&E staining of tissues from tumor-bearing Becn2 KO mice, it was also detected lung metastases in mice harboring tumor #1 and tumor #2, and liver metastases in the mouse harboring tumor #1 (FIG. 16D). The immunostaining was further performed on primary tumors and metastatic tissues to determine the tumor types in Becn2 KO mice. Immunohistochemical staining of CD3, B220, and Ki67 showed that the primary tumor and lung metastases from mouse #1 can be characterized as T cell lymphoma (FIG. 17A), while the staining of tumor #2 showed a mixed population of T cells and B cells (FIG. 17B). To further define the lymphoma type of tumor #2, confocal microscopy was used to identify the co-localization of Ki67 with either CD3 or B220, which identified it as B cell lymphoma based on the co-localization of Ki67 and B220 (FIG. 17C). Among 5 lymphomas developed in Becn2 KO mice, one was T cell lymphoma and four were B cell lymphomas. These results indicate that Beclin 2 deficiency promotes lymphoma development with metastasis in the lung and liver.

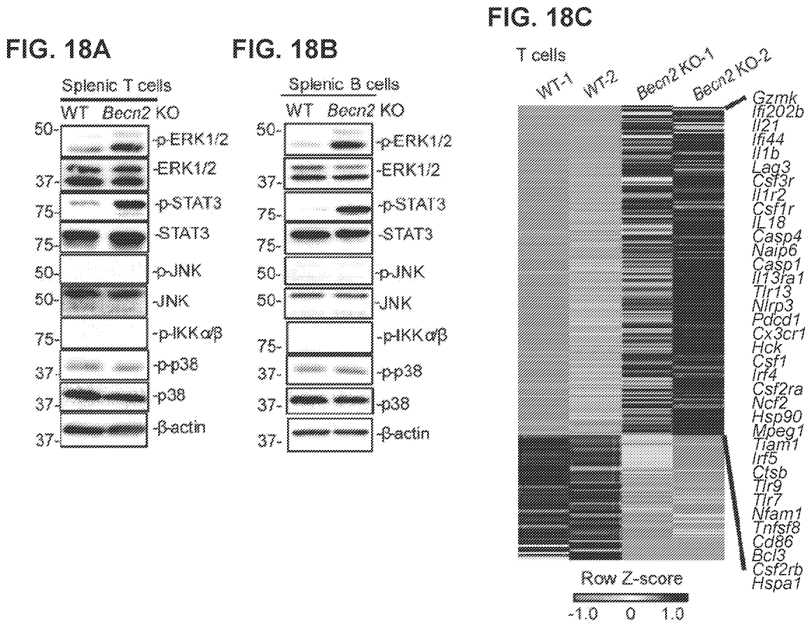

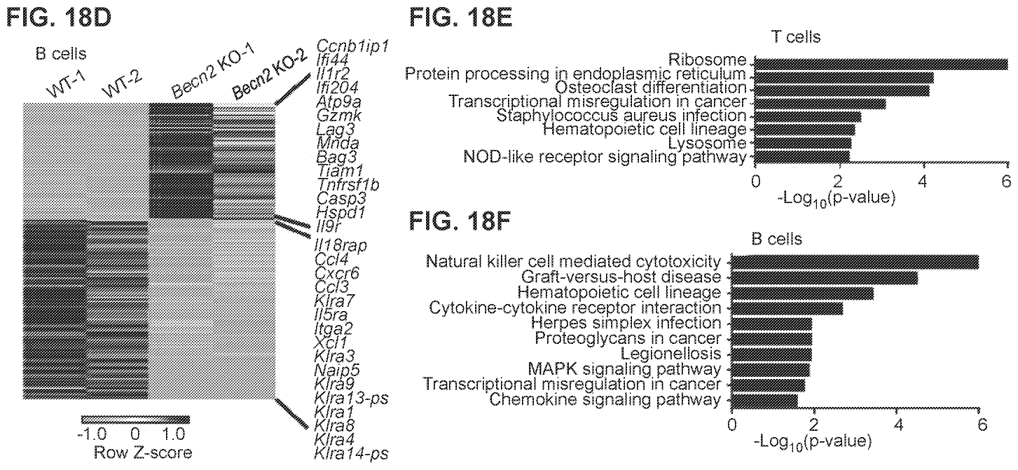

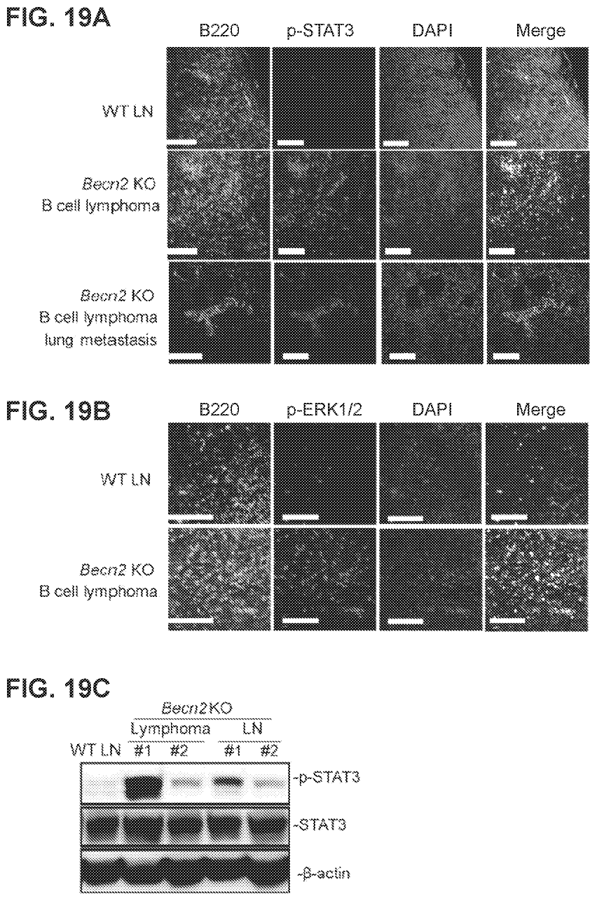

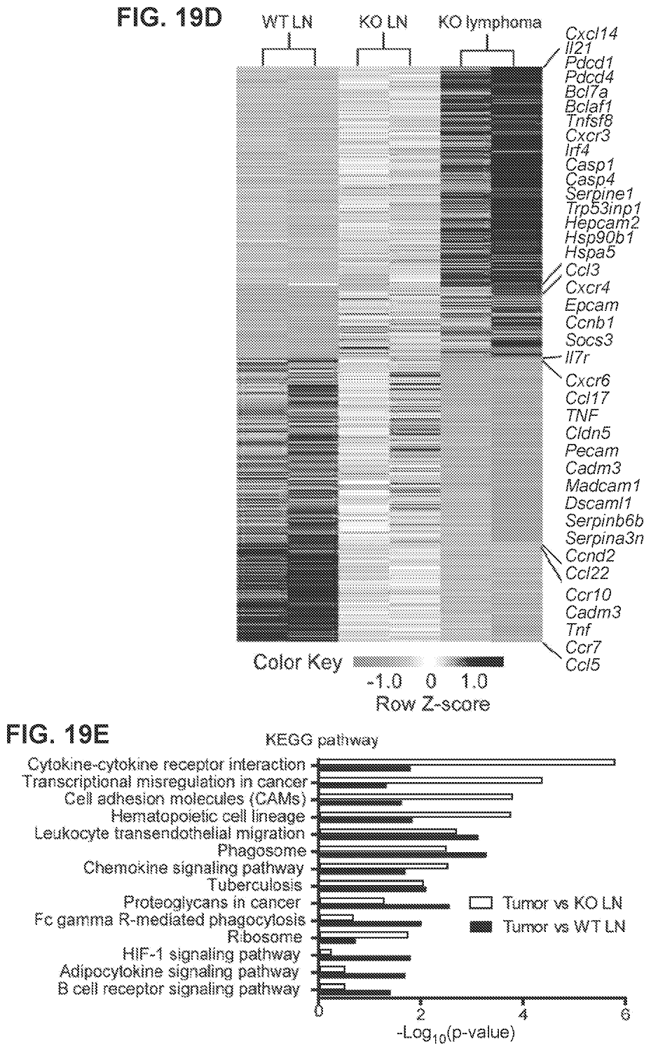

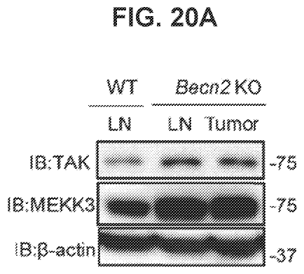

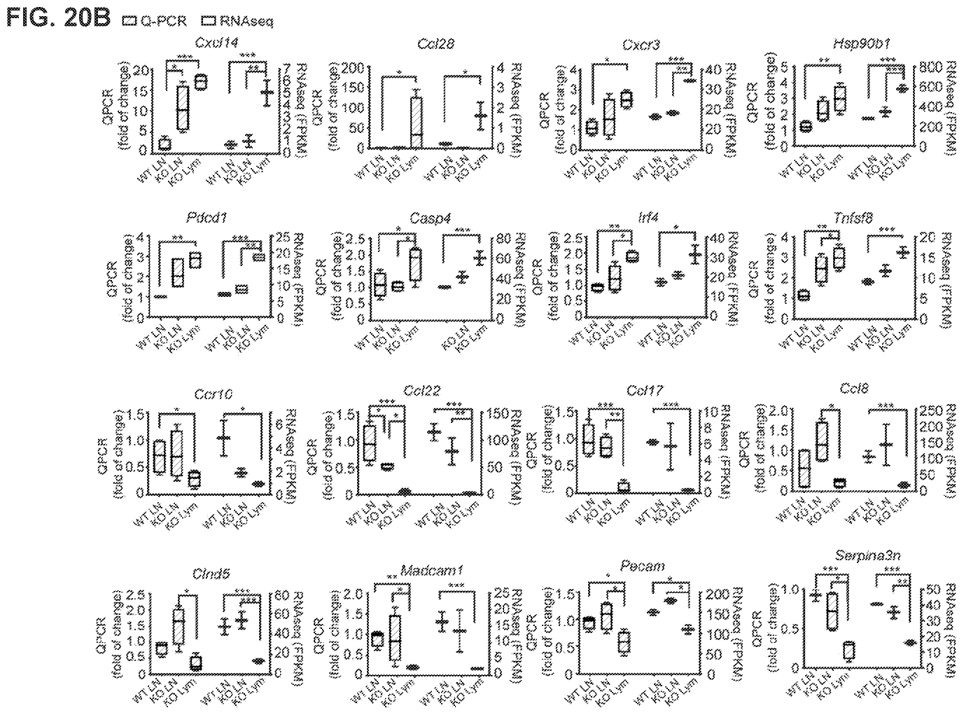

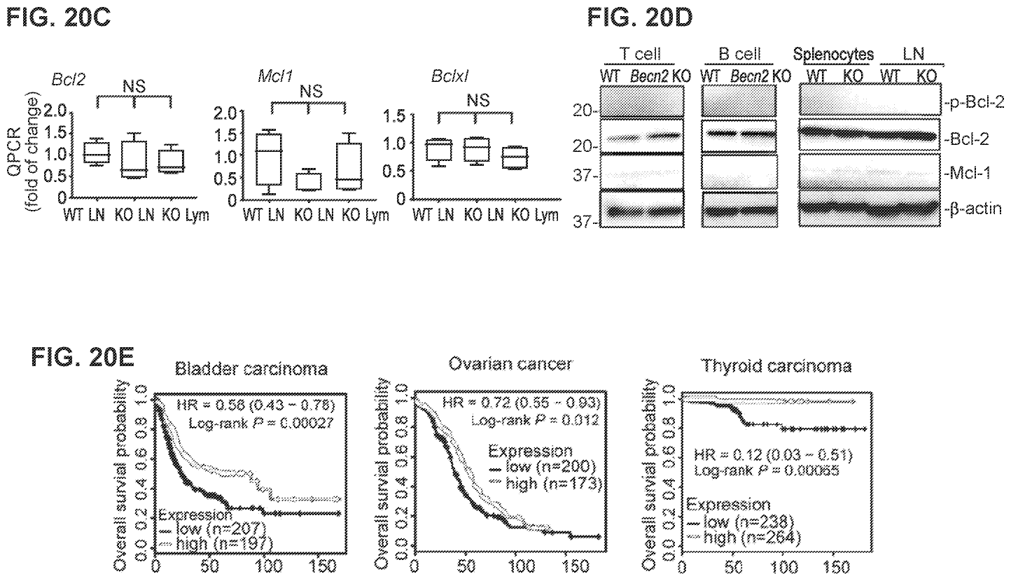

Enhanced STAT3 activation and cytokine/chemokine expression in lymphomas of Becn2 KO mice. Since the loss of Beclin 2 leads to increased MEKK3 protein levels and ERK signaling for IL-6 production, it was reasoned that the elevated IL-6 production and persistent STAT3 activation in T and B lymphocytes in Becn2 KO mice plays a critical role in the tumorigenesis. Therefore, whether IL-6 directly affected T or B cells for the promotion of lymphoma development in Becn2 KO mice was first determined. The NF-κB and MAPK signaling in splenic T cells and B cells was checked from Becn2-deficient mice that were affected by elevated basal IL-6, and it was found that both ERK1/2 and STAT3 signaling were persistently activated in Becn2-deficient T cells and B cells (FIGS. 18A and 18B). Furthermore, RNA-seq analysis showed that many genes involved in immune responses to infectious diseases, transcriptional misregulation in cancer, NOD-like receptor and cytokine-cytokine receptor signaling, and MAPK signaling were dysregulated in Becn2 KO T cells and B cells compared to WT control cells (FIGS. 18C-18F). Next, STAT3 and ERK status was examined by immunofluorescence staining in tumors or normal lymph nodes and it was found dramatic increases in both phosphorylated-STAT3 and -ERK levels in lymphomas from Becn2 KO mice compared with lymph nodes from WT mice (FIGS. 19A and 19B). Immunoblot analysis further demonstrated the increased STAT3 phosphorylation in Becn2 KO lymphomas and lymph nodes compared with WT control samples (FIG. 19C). Notably, MEKK3 and TAK1 expression in lymphomas and lymph nodes from Becn2 KO mice were consistently increased (FIG. 20A). These results indicate that STAT3 signaling is constitutively activated in Becn2-deficient lymphocytes by elevated IL-6 production to promote lymphoma development.

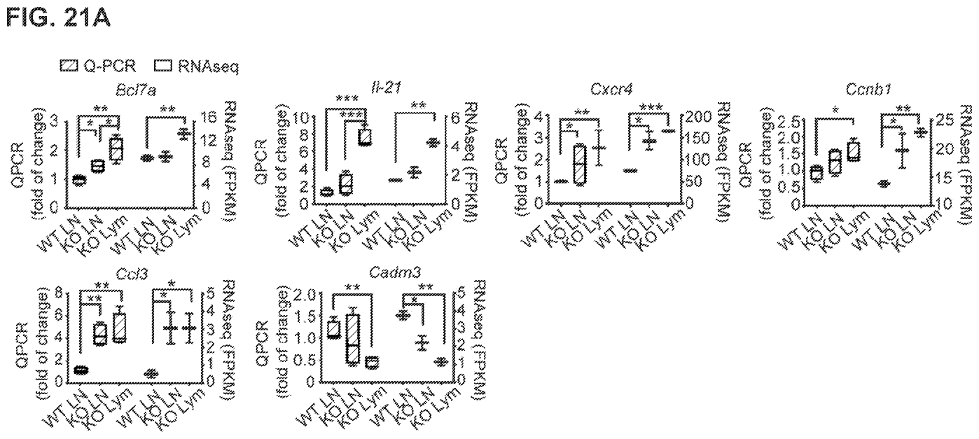

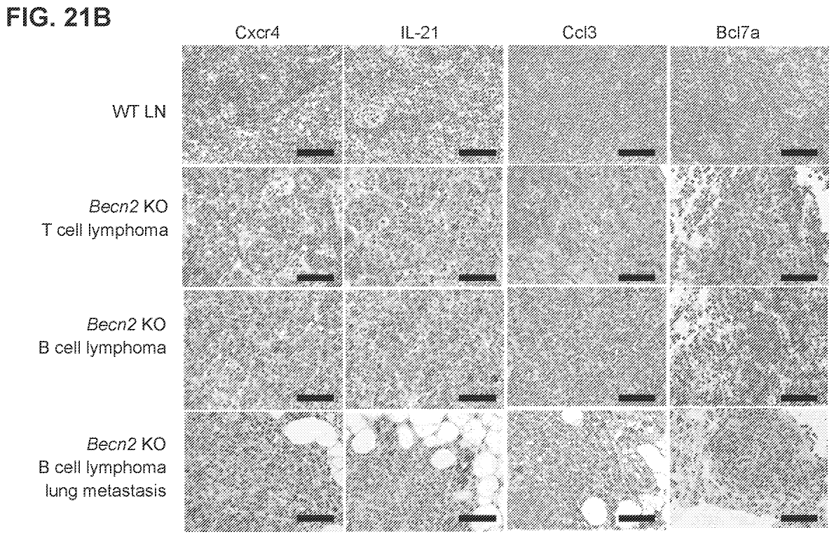

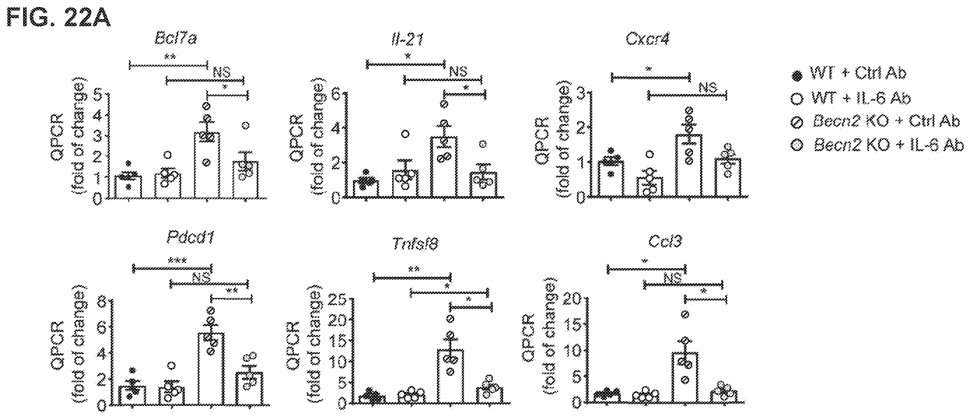

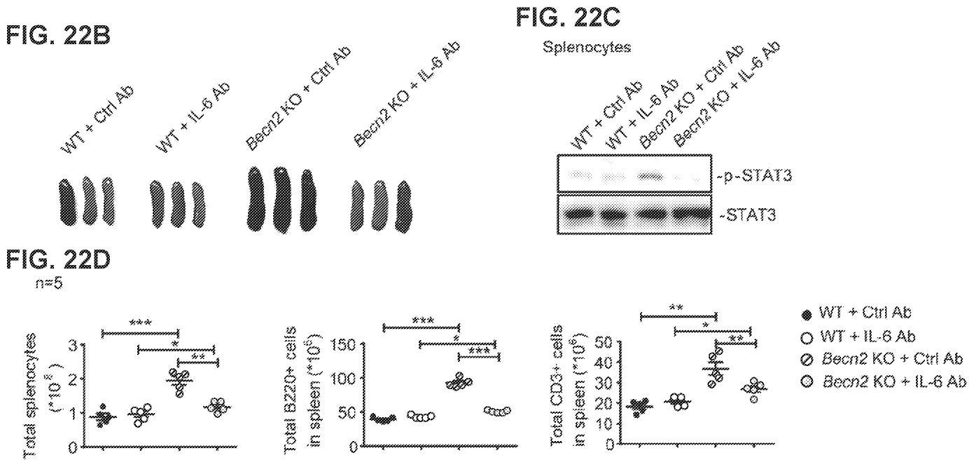

Gene expression profiles were compared among WT lymph nodes, Becn2 KO lymph nodes, and Becn2-deficient lymphomas using RNA-seq analysis. It was found that many genes involved in inflammation, cell proliferation, and tumor metastasis were upregulated in Becn2 KO lymph nodes and/or lymphomas compared with WT lymph nodes (FIG. 19D). In particular, the cytokine-cytokine receptor interaction, transcriptional dysregulation in cancer, cell adhesion, and cell lineage pathways were significantly altered in Becn2 KO lymphomas compared with either Becn2 KO or WT lymph nodes (FIG. 19E). Furthermore, the upregulation of essential tumorigenic proinflammatory cytokines, chemokines, and oncogenes, as well as the downregulation of cell-cell adhesion molecules, were confirmed by real-time PCR (FIG. 21A and FIG. 20B) and immunohistochemical staining (FIG. 21B). In particular, Cxcr4, IL-21, Ccl3, and Bcl-7a were robustly expressed in tumor tissues (FIGS. 21A and 21B). There was no appreciable difference in Bcl-2, p-Bcl-2, Bcl-xl or Mcl-1 expression between WT and KO lymph nodes (FIGS. 20C and 20D), although these proteins have been reported to bind to Beclin 1 and have been implicated to promote tumorigenesis in patients with low Beclin 1 expression. These results indicate that the persistent activation of STAT3 signaling, as well as the upregulation of Cxcr4, Ccl3, IL-21 and Bcl-7a expression, can promote lymphoma development in Becn2-deficient mice. To further determine the role of IL-6 in the spontaneous lymphoma development in Becn2 KO mice, IL-6 neutralizing antibody was used to treat the mice for 4 consecutive weeks, and it was found that the levels of these upregulated key genes that identified in Becn2 KO lymphoma samples were significantly reduced to levels similar to WT counterparts, including Bcl7a, 11-21, Pdcd1, Cxcr4, Tnfsf8, and Ccl3 (FIG. 22A). Moreover, IL-6 neutralizing antibody treatment also significantly rescued the splenomegaly observed in Becn2-deficient mice (FIG. 22B). The level of p-STAT3 in Becn2 KO splenocytes was restored to a level similar to WT cells (FIG. 22C). Consistently, the total numbers of splenocytes, B220⁺ and CD3⁺ lymphocytes were significantly reduced, compared with those in control antibody-treated Becn2 KO mice (FIG. 22D). These results indicate that the elevated IL-6 production is closely associated with the persistent STAT3 activation and the development of splenomegaly and spontaneous lymphoma in Becn2 KO mice. The Kaplan-Meier plot analysis using human cancer databases revealed that Beclin 2 expression levels were associated with the overall survival rates for patients with cancer. In particular, for patients with bladder carcinoma and thyroid carcinoma, the higher levels of Beclin 2 expression were significantly associated with prolonged overall survival compared to those with low Beclin 2 expression (P<0.001) (FIG. 20E). Furthermore, higher expression levels of Beclin 2 were also associated with an extended overall survival for ovarian cancer patients (P<0.05) (FIG. 20E), but to a less extent. These data further indicate that Beclin 2 has a tumor-suppressive function through innate immune regulation, STAT3 signaling, and chemokine expression.

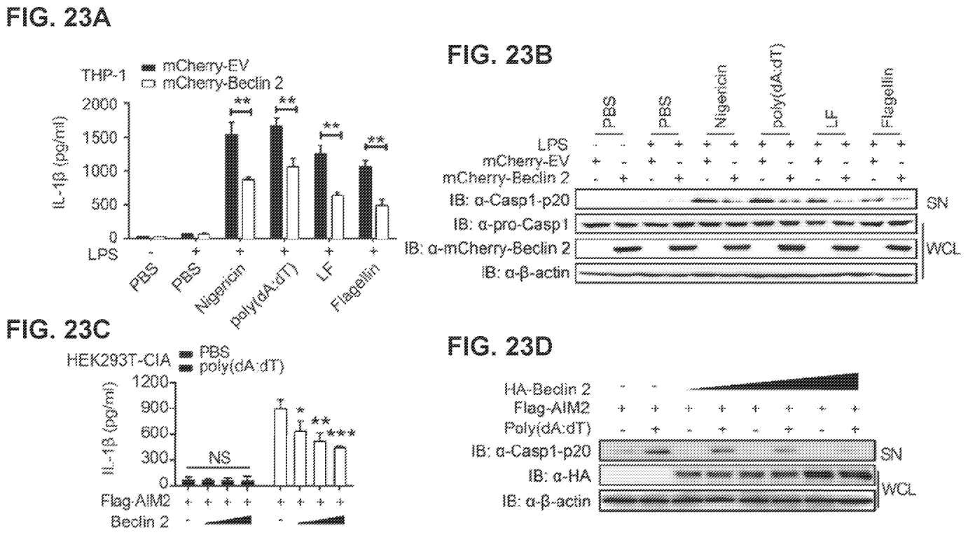

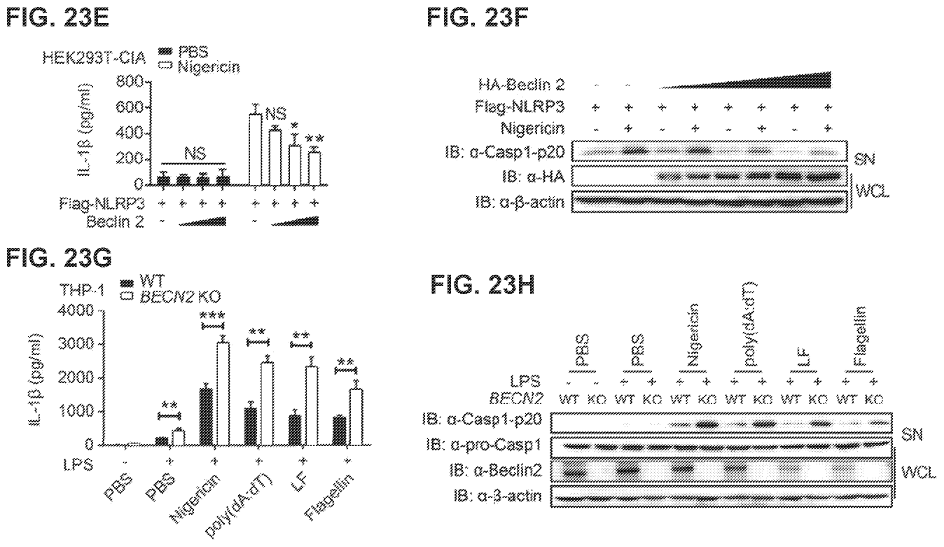

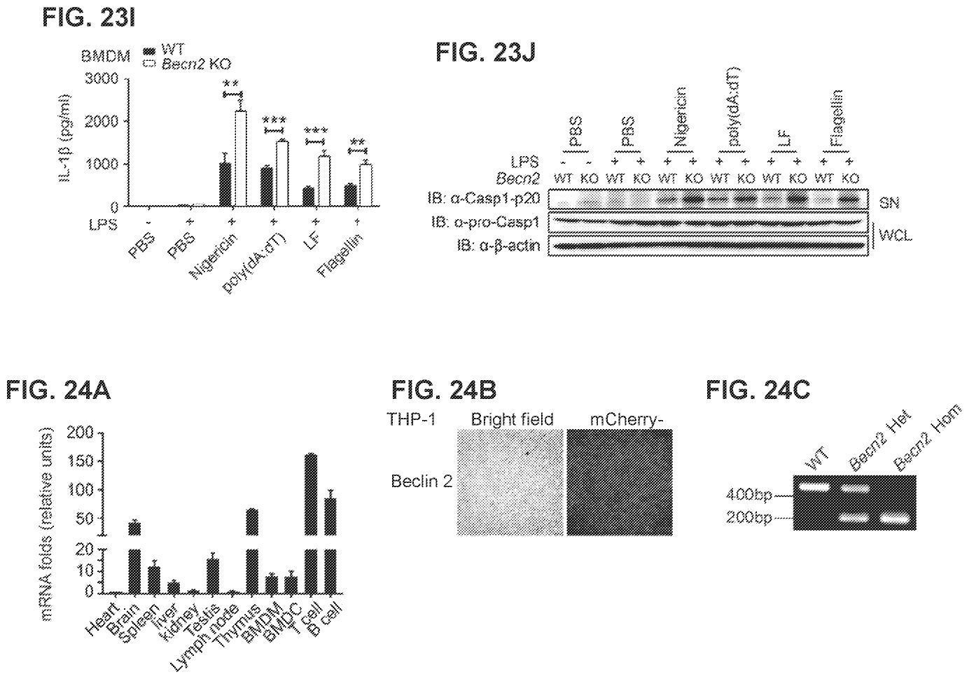

Beclin 2 deficiency enhances inflammasome activation. To determine the role and involvement of Beclin 2 in the inflammasome pathway, the mRNA level of Becn2 in different organs and cell types from WT mice. Becn2 was highly expressed in brain, spleen, testis, bone marrow-derived macrophages (BMDMs) and bone marrow-derived dendritic cells (BMDCs), especially in the thymus, T cells and B cells (FIG. 24A), indicating a function in innate and adaptive immune systems. To test the functional relevance of Beclin 2 in inflammasome activation, mCherry-tagged Beclin 2 was stably expressed in THP-1 cells by retrovirus transduction (FIG. 24B), and the it was found that the IL-1β production and cleaved caspase 1 levels were markedly reduced in mCherry-Beclin 2 transduced THP-1 cells compared to mCherry-empty vector (EV) transduced control cells, in response to the stimulation from multiple ligands including nigericin (NLRP3 ligand), poly(dA:dT) (AIM2 ligand), anthrax lethal factor (LF, NLRP1 ligand), and flagellin (NLRC4 ligand) (FIGS. 23A and 23B). Similar results were observed in Beclin 2-expressing HEK293T-CIA cells (293T cells stably expressing caspase 1, pro-IL-1β, and ASC). After transfecting plasmids encoding AIM2 or NLRP3 and stimulating the cells with poly(dA:dT) or nigericin, respectively, the IL-1β production and cleaved caspase 1 levels in the supernatant were markedly reduced in the cells co-transfected with increasing amounts of Beclin 2 expression plasmid DNAs in a dose-dependent manner (FIGS. 23C-23F). Consistently, knockout (KO) of BECN2 in THP-1 cells significantly enhanced the IL-1β production and caspase 1 cleavage under the treatment of nigericin, poly(dA:dT), LF, or flagellin (FIGS. 23G and 23H). To further evaluate the inhibitory function of Beclin 2 in controlling inflammasome activation under physiological conditions, we used the homozygous Becn2 KO mice with validation of the allele deletion by polymerase chain reaction (PCR) (FIG. 24C). The LPS-primed BMDMs isolated from Becn2 KO mice had significantly higher levels of IL-1β production and cleaved caspase 1 than those from WT mice after ligand stimulation (FIGS. 231 and 23J). Together, these data show that the loss of Beclin 2 markedly enhances the inflammasome activities upon ligand stimulations, particularly for NLRP3, AIM2, NLRP1 and NLRC4 sensors.

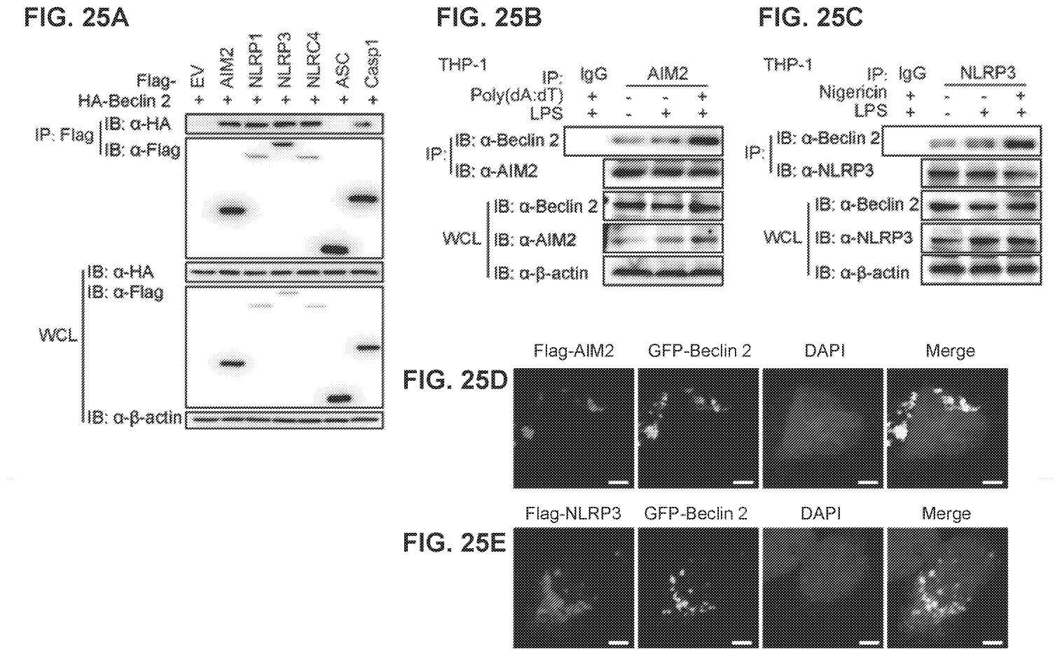

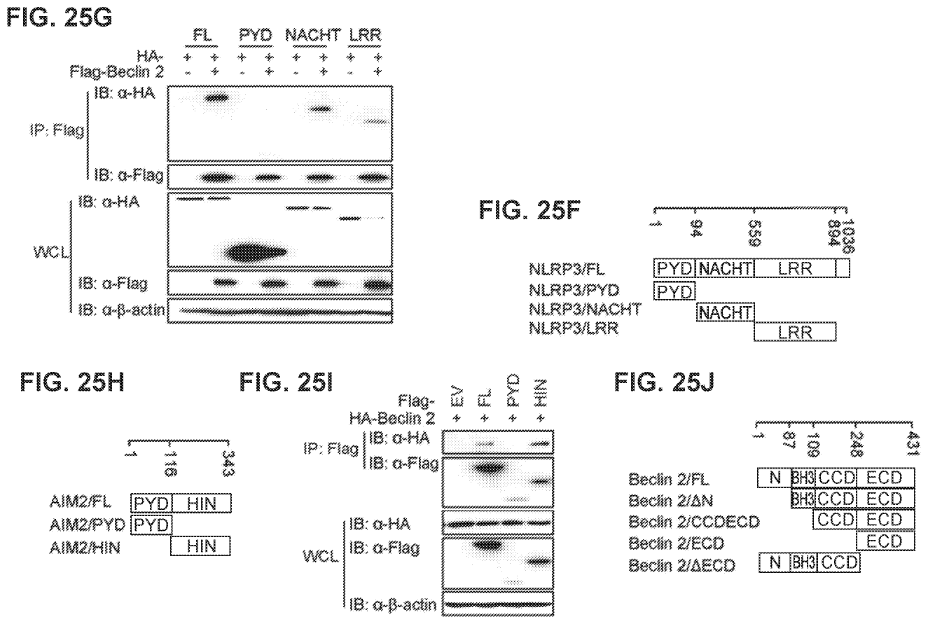

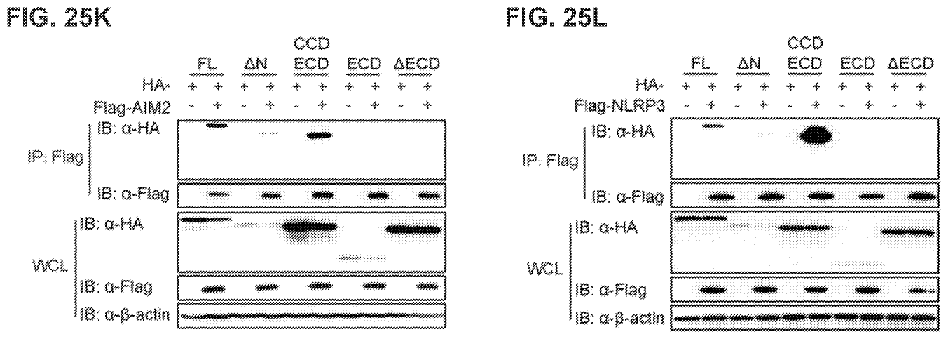

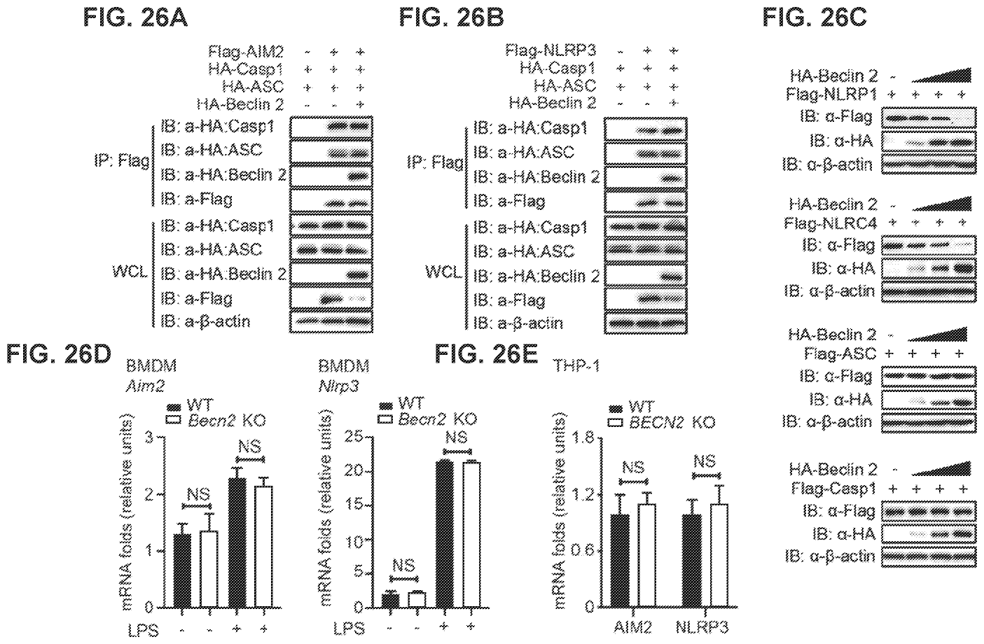

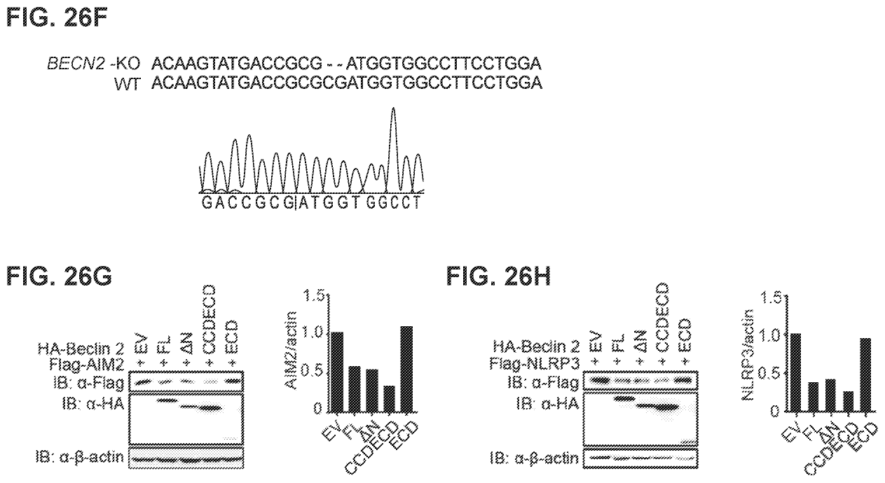

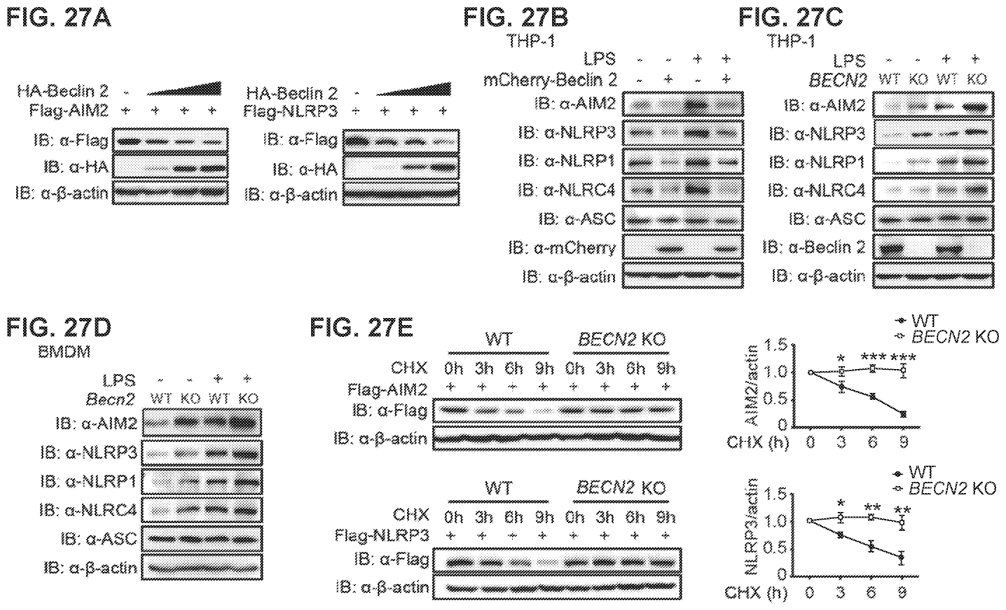

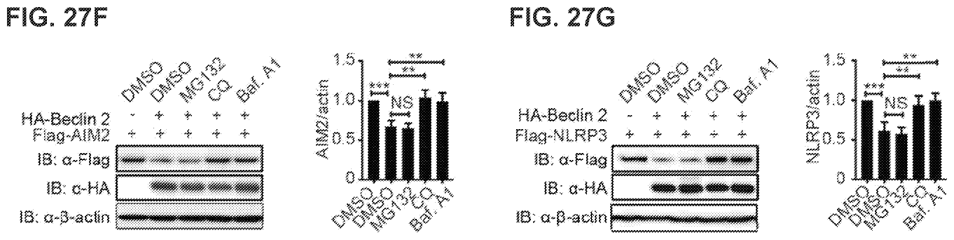

Beclin 2 interacts with inflammasome sensors through CCD-ECD domains. Next, how Beclin 2 negatively regulates inflammasome activation was determined. Co-immunoprecipitation and western blot analyses were performed to determine if Beclin 2 can directly interact with inflammasome components by co-transfection of HEK293T cells with expression plasmids for ASC, caspase 1, NLRP3, AIM2, NLRP1, or NLRC4 together with Beclin 2. Beclin 2 can interact with NLRP3, AIM2, NLRP1, NLRC4 and caspase 1, but not with ASC (FIG. 25A). Immunoprecipitation of endogenous AIM2 or NLRP3 further revealed that the endogenous Beclin 2 can bind to endogenous AIM2 or NLRP3 even prior to stimulation, and such interaction was increased after poly(dA:dT) or nigericin treatment, respectively (FIGS. 25B and 25C). Consistently, confocal microscopic analysis revealed the colocalization of GFP-Beclin 2 with AIM2, or with NLRP3, in the cytosol of Hela cells (FIGS. 25D and 25E). To further determine which domains of AIM2 and NLRP3 are responsible for their interactions with Beclin 2, a series of truncated constructs (NLRP3-PYD, NLRP3-NACHT, NLRP3-LRR, AIM2-PYD, and AIM2-HIN domains) were generated and it was found that the HIN domain of AIM2, as well as the NACTH and LRR domains of NLRP3, were responsible for their specific interactions with Beclin 2 (FIGS. 25F-25I). To identify which domain of Beclin 2 is required for its interaction with AIM2 and NLRP3, we generated four truncated Beclin 2 constructs, including N-terminal deletion fragment (Beclin 2/AN), CCD and ECD domains (Beclin 2/CCDECD), ECD domain alone, and ECD domain deletion fragment (Beclin 2/AECD) (FIG. 25J). The co-immunoprecipitation of these truncations with AIM2 or NLRP3 revealed that the Beclin 2 CCD-ECD domains, but not other domains, interacted with AIM2 and NLRP3 (FIGS. 25K and 25L). To determine if the interaction between Beclin 2 and inflammasome sensors affects the assembling of inflammasome complexes, HEK293T cells were co-transfected with HA-ASC and HA-caspase 1 along with Flag-AIM2 or Flag-NLRP3 in the control or Beclin 2-overexpressing 293T cells. It was shown that the assembly of inflammasome components was not affected by Beclin 2 ectopic expression, as determined by co-immunoprecipitation assay (FIGS. 26A and 26B), indicating that Beclin 2 inhibits inflammasome activation by interacting with Aim 2 or NLRP3 without disruption of inflammasome assembly.

Beclin 2 degrades inflammasome sensors through a lysosomal pathway. To understand how Beclin 2 inhibits inflammasome activation, it was reasoned that Beclin 2 targeted inflammasome sensors for degradation, since Beclin 2 has been reported to degrade G-protein coupled receptors through a lysosomal degradation pathway. HEK293T cells were transfected with key inflammasome components along with increasing amounts of Beclin 2 plasmids, and it was found that the protein levels of inflammasome sensors (AIM2, NLRP3, NLRP1, and NLRC4), but not ASC or caspase 1, were markedly reduced with increasing Beclin 2 expression (FIG. 25A and FIG. 26C). Consistently, the endogenous inflammasome sensors, but not ASC, were also markedly decreased in mCherry-Beclin 2-expressing THP-1 cells, compared with mCherry-EV control (FIG. 25B). Elevated levels of inflammasome sensors were observed in BECN2-deficient THP-1 cells and macrophages from Becn2 KO mice before and after LPS-stimulation, compared with WT controls (FIGS. 25C and 25D). By contrast, no appreciable differences were found at the mRNA levels of AIM2 and NLRP3 between WT and Becn2 KO BMDMs or THP-1 cells (FIGS. 26D and 26E). In addition, a cycloheximide (CHX, protein synthesis inhibitor) “chase” assay was performed in WT and BECN2 KO HEK293T cells (KO of BECN2 in 293T cell clone was identified by sequencing of gDNA in FIG. 26F), and it was found the deficiency of Beclin 2 in HEK293T cells prolonged the half-life of AIM2 and NLRP3, indicating that Beclin 2 accelerates the degradation of AIM2 and NLRP3 (FIG. 25E). To determine the functional domain of Beclin 2 in mediating the degradation of inflammasome sensors, Flag-AIM2 or Flag-NLRP3 were co-transfected along with different HA-Beclin 2 domains, respectively. The immunoblotting analysis showed that the Beclin 2 CCD-ECD domain, as the interactive domain with AIM2 and NLRP3, was also responsible for their degradation function (FIGS. 26G and 26H).

Since there are two major (proteasomal and lysosomal) pathways for protein degradation, the next experiment sought to identify which pathway is required for Beclin 2-mediated degradation of inflammasome sensors. Cells were treated with lysosomal inhibitors such as bafilomycin A (BafA) and chloroquine (CQ) or proteasome inhibitor (MG132), and it was found that the degradation of AIM2 and NLRP3 was completely blocked by the lysosomal inhibitors (BafA and CQ), but not by the proteasomal inhibitor MG132 (FIGS. 25F and 25G). Taken together, these results indicate that Beclin 2 mediates the degradation of inflammasome sensors through a lysosomal pathway.

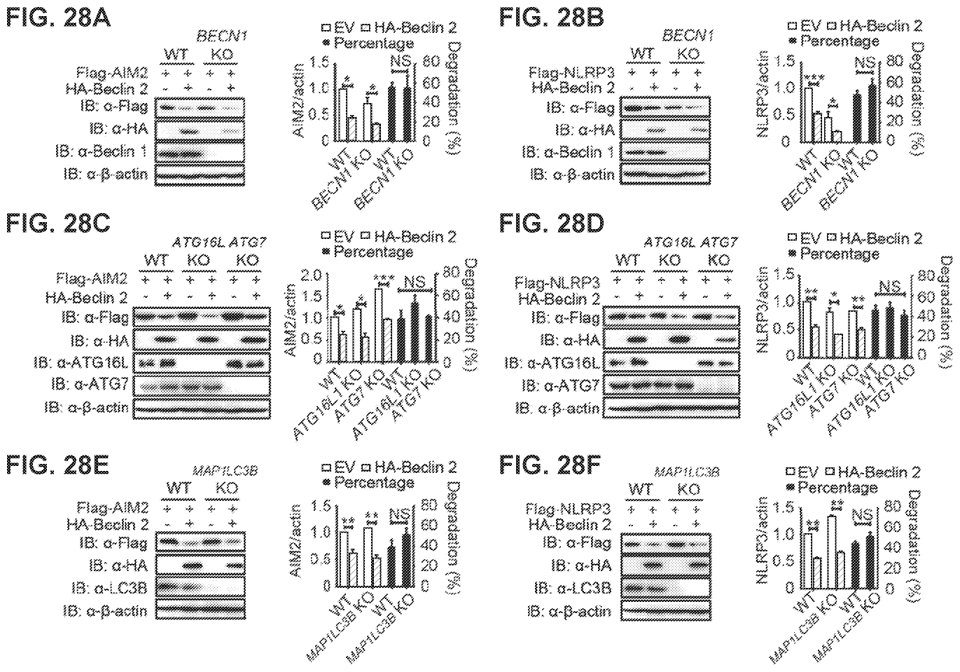

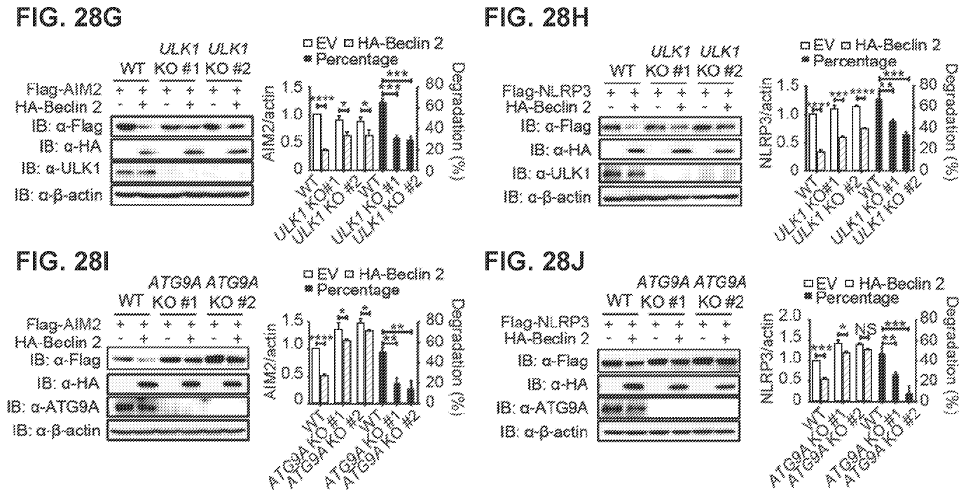

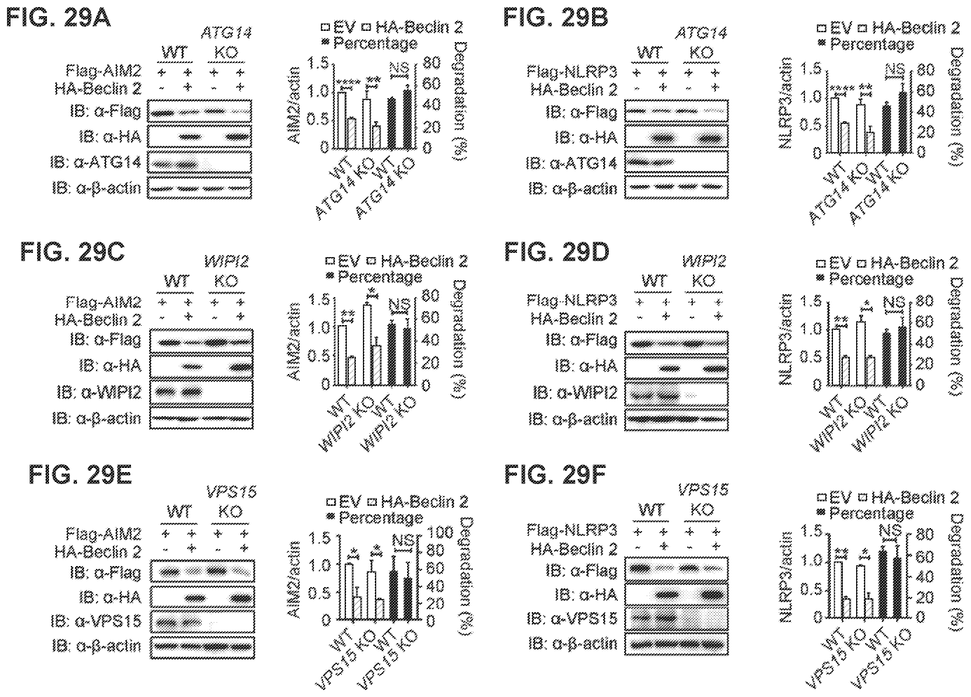

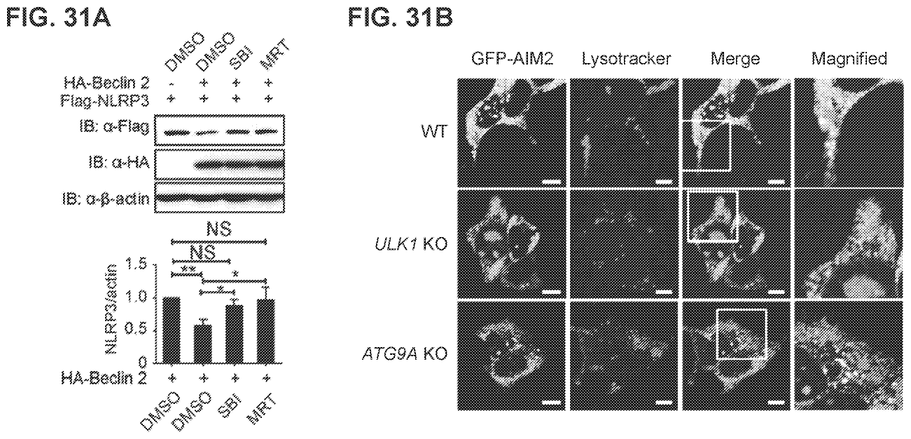

Beclin 2 degrades inflammasome sensors through the ULK/ATG9A-dependent but ATG16L/LC3/Beclin 1/WIPI2-independent lysosomal pathway. Beclin 2 is an autophagy protein and interacts with known binding partners of Beclin 1 including ATG14, VPS34 and AMBRAL. Whether Beclin 2 can target inflammasome components for autophagic degradation was tested. It has been known that classical macroautophagy is controlled by a set of evolutionarily conserved autophagy gene-related proteins (ATG proteins) in a process that includes: (i) initiation of the phagophore formation through mTOR; (ii) nucleation through class III phosphatidylinositol 3-kinase complex (PI3K complex); (iii) elongation to form the autophagosomes through Atg12/ATG7/Atg5/Atg16 and LC3/Atg8 controlled ubiquitin-like conjugation systems; and (iv) maturation and degradation via fusion with lysosomes. To further determine whether Beclin 2 mediates the degradation of inflammasome sensors through autophagy, a series of central components in each complex involved in macroautophagy were knocked out. Surprisingly, it was found that Beclin 2-mediated degradation was not affected in the cells deficient in key ATGs of PI3K complex or ubiquitin-like conjugation systems, such as Beclin 1, ATG14, VPS15, ATG7, ATG16L, WIPI2, or LC3B (FIGS. 28A-28F, and FIGS. 29A-29F). By contrast, the deletion of the ATGs in the initiation step including ULK1 and ATG9A can markedly inhibit the Beclin 2-mediated sensor degradation (FIGS. 28G-28J). Consistently, Beclin 2-mediated degradation can be blocked by the ULK1 complex inhibitors SBI-0206965 and MRT 68921 (FIG. 31A). These results indicate that Beclin 2-mediated degradation of inflammasome sensor proteins requires a ULK1- and ATG9A-dependent, but classical autophagy-independent pathway.

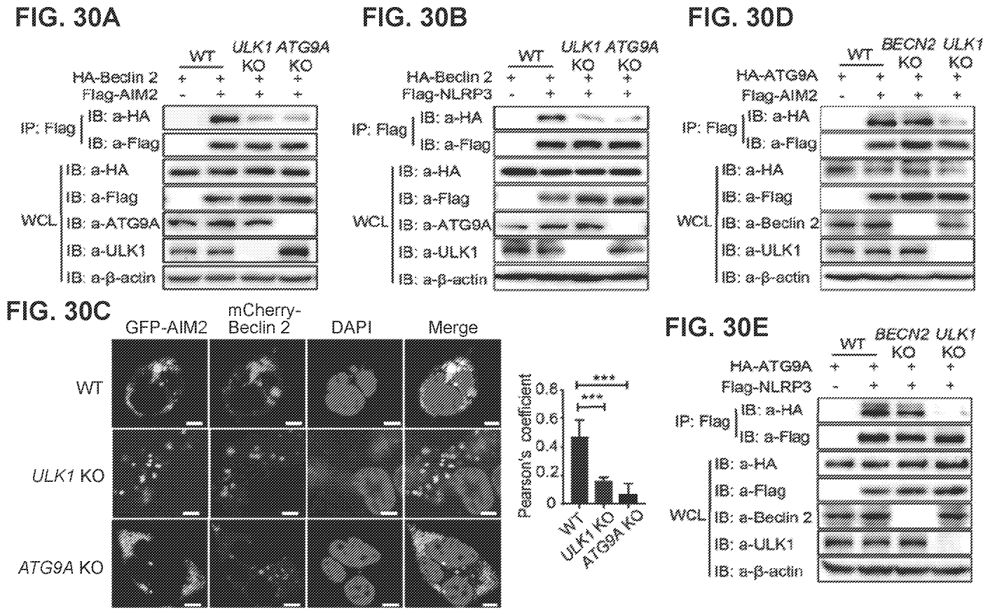

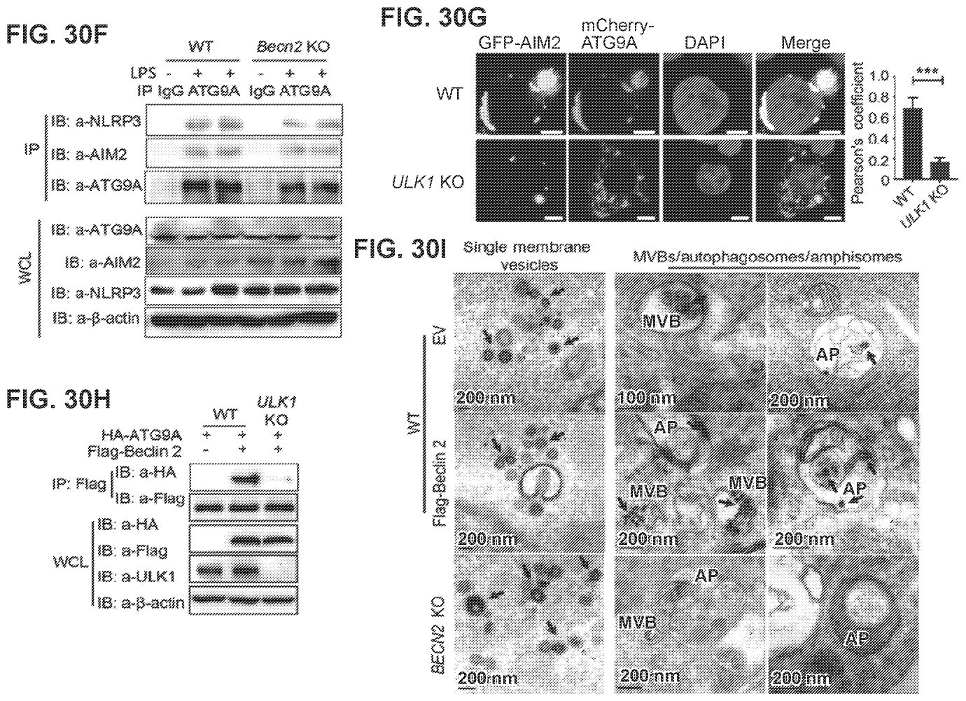

To further explore the molecular mechanism by which Beclin 2-mediated the degradation of inflammasome sensors, whether the interactions between Beclin 2 and inflammasome sensors can be compromised in ULK1- or ATG9A-deficient cells was examined. Indeed, it was found that interactions between Beclin 2 and AIM2 or NLRP3 in ULK1- and ATG9A-deficient cells were markedly reduced, compared to WT cells (FIGS. 30A and 30B). The confocal microscopic analyses further revealed that mCherry-Beclin 2 failed to be co-localized with GFP-AIM2 in ULK1 or ATG9A KO HEK293T cells (FIG. 30C). Consistently, the transportation of AIM2 to lysosome was largely impaired in ULK1 KO and ATG9A KO cells, compared to WT cells (FIG. 31B).

To elucidate the role of ULK1 and ATG9A in the degradation of inflammasome sensors, the interaction between ATG9A and inflammasome sensors was examined in WT, BECN2KO, and ULK1 KO cells. Co-immunoprecipitation analysis showed that AIM2 or NLRP3 can interact with ATG9A-vesicles, and such interactions required ULK1 but not Beclin 2 (FIGS. 30D and 30E). Consistently, the endogenous interactions of ATG9A with NLRP3 and AIM2 were also detected in BMDMs, but such interactions were not affected by LPS stimulation or Beclin 2 deficiency (FIG. 30F). Confocal microscopic images consistently showed that the colocalization between AIM2 and ATG9A was significantly compromised in ULK1 KO cells (FIG. 30G). Likewise, the interaction between Beclin 2 and ATG9A was also ULK1-dependent (FIG. 30H). These results show that ATG9A functions as a central bridge to bring Beclin 2 and the inflammasome sensors together, while ULK1 is required for initiating the formation of inflammasome-ATG9A-Beclin 2 complex. To further understand the process of ATG9A-Beclin 2-mediated inflammasome sensor degradation, the translocation of AIM2 was examined by transmission electron microscopy (TEM) in WT, Beclin 2-overexpressed WT cells, and BECN2 KO 293T cells through APEX2-enabled staining. It was found that AIM2 was frequently associated with single-membrane vesicles, multivesicular bodies (MVBs), and autophagosomes or amphisomes (intermediate organelles formed by fusion of autophagosomes and endosomes/MVBs) in WT cells (FIG. 30I). The presence of APEX2-AIM2 in autophasosomes/amphisomes was significantly increased after Beclin 2 overexpression. However, AIM2 was barely detected in autophasosomes in BECN2 KO cells, but mainly associated with single-membrane vesicles (FIG. 30I). These observations indicate that Beclin 2 can facilitate the AIM2-associated vesicles transporting to autophagosomes for degradation through ATG9A⁺ endosomes/MVBs fusion with phagophores.

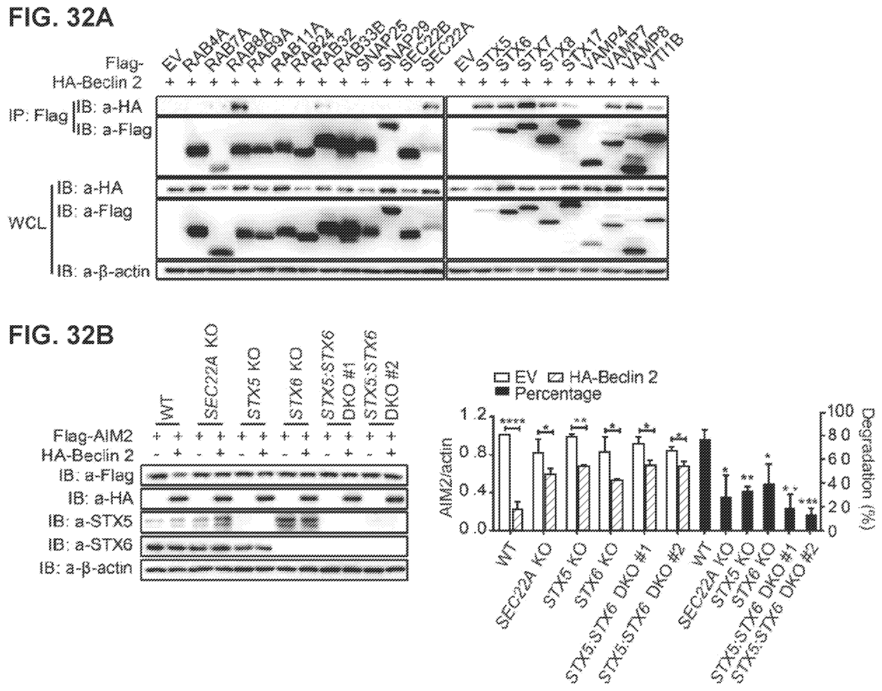

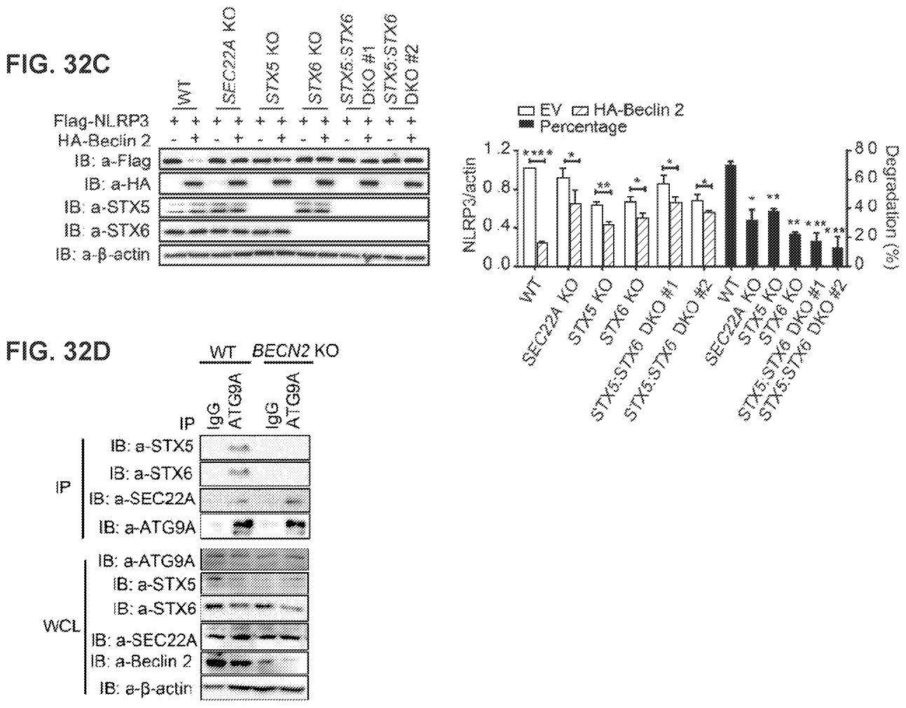

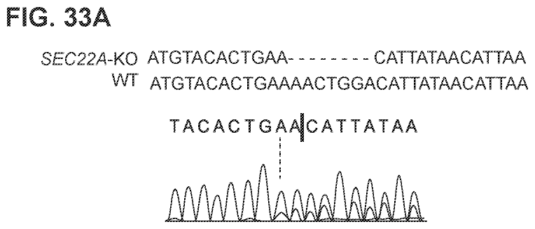

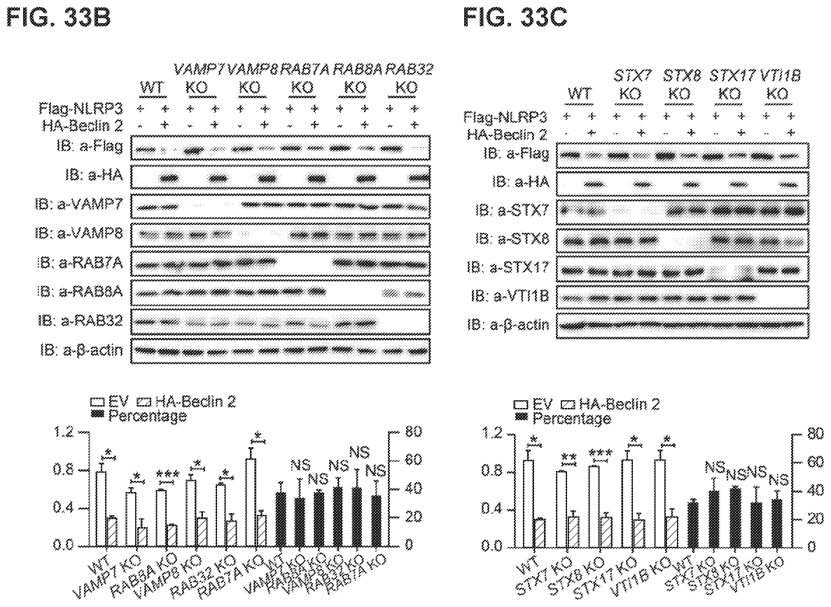

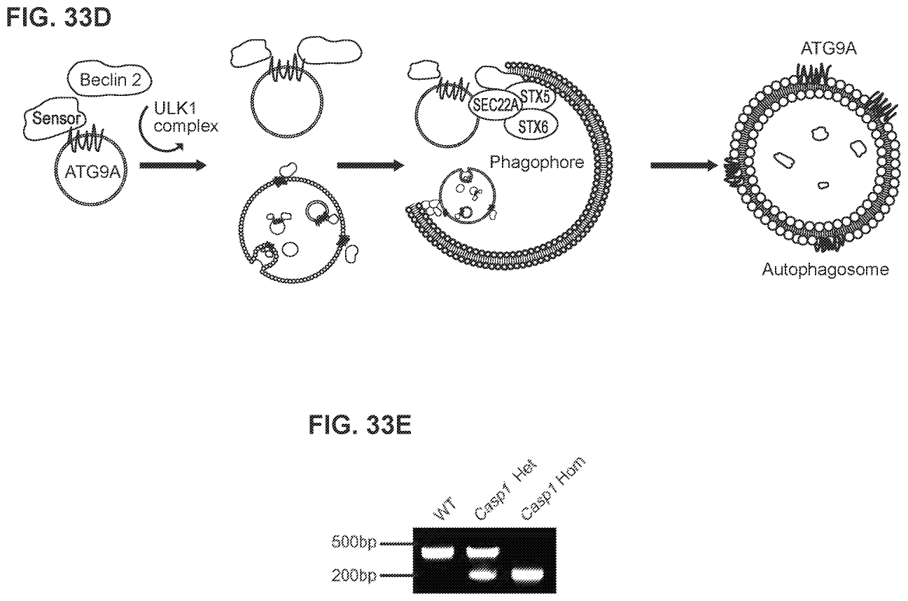

SEC22A-STX5-STX6 SNAREs are required for the degradation of inflammasome sensors. ATG9A is a multispanning transmembrane ATG protein that cycles between the trans-Golgi network (TGN) and endosomes/lysosomes. The ATG9A⁺-vesicle recruitment to pre-autophagosomal structure (PAS) is also an important resource for the membrane assembling during autophagosome formation. Based on the above findings that AIM2 was frequently associated with single membrane vesicles (presumably to be ATG9A-vesicles) and can be markedly transported to autophagosomes after Beclin 2 overexpression, it was reasoned that Beclin 2 promotes the degradation of inflammasome sensors through multiple steps of vesicle membrane fusions. RABs and soluble NSF attachment protein receptors (SNAREs) machinery have been reported to play a central role in membrane docking/fusion process To test this possibility, the interactions between Beclin 2 and some members of SNAREs and RAB GTPases were examined. Co-immunoprecipitation experiments showed that Beclin 2 can strongly interact with RAB8A, SEC22A, syntaxin 5 (STX5), STX6, STX7, STX8, VAMP7, and VAMP8, and weakly interact with RAB7A, RAB32, VTI1B, and STX17 (FIG. 32A). The next experiment investigated whether the deficiency of these Beclin 2-binding proteins can completely or partially block the inflammasome sensor degradation. For this reason, a series of KO cell lines were generated, and it was found that specific ablation of STX5, STX6, or SEC22A in HEK293T cells using CRISPR/Cas9 technology can partially block the Beclin 2-mediated AIM2 or NLRP3 degradation (FIG. 33A, FIGS. 32B and 32C). By contrast, KO of other RAB or SNARE genes had little or no effect on NLRP3 degradation (FIGS. 33B and 33C). To further determine their effects, the cell line with double KO (DKO) of STX5 and STX6 were generated and it was shown that it had much stronger inhibitory effects on Beclin 2-mediated inflammasome degradation than those in single KO cells (FIGS. 32B and 32C). To determine the role of Beclin 2 in STX5/STX6/SEC22A-mediated the ATG9A-vesicle membrane fusion, we also examined the endogenous interaction between ATG9A and STX5/STX6/SEC22A in WT and BECN2 KO THP-1 cells. The endogenous interactions of ATG9A with STX5 and STX6 were detected in WT THP-1 cells, but diminished by the ablation of BECN2. By contrast, the interaction of ATG9A and SEC22A remain unchanged regardless of Beclin 2 expression (FIG. 32D). These data show the critical role of SEC22A-STX5-STX6 SNAREs in the degradation of inflammasome sensor proteins in a Beclin 2-ATG9A-dependent lysosomal pathway through vesicle membrane fusion processes (FIG. 33D).

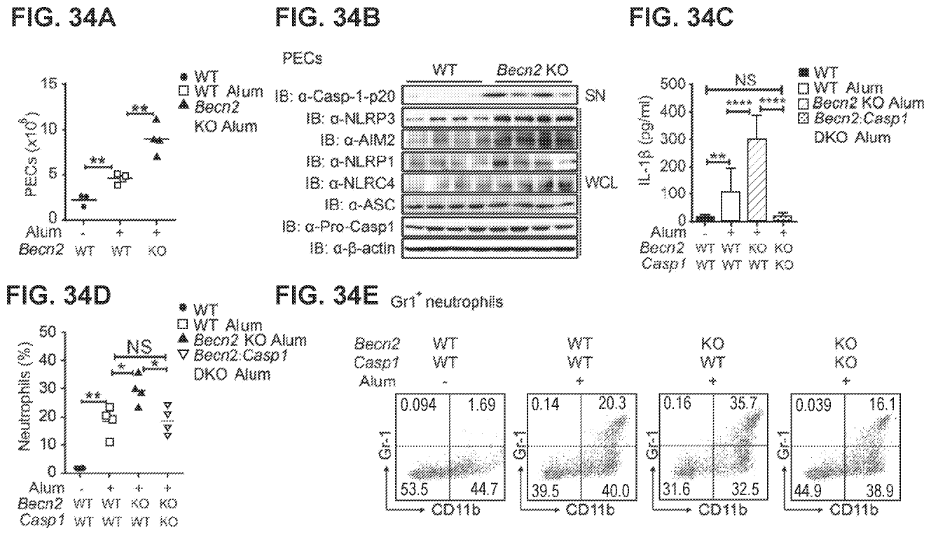

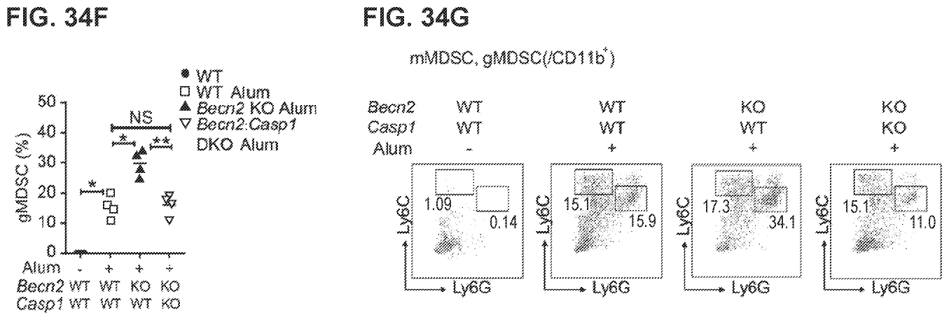

Loss of Beclin 2 exacerbates alum-induced peritonitis. To further substantiate these findings in vivo, the phenotypes of Becn2 deficient mice in alum-induced peritonitis model were investigated. Mouse peritonitis was induced by an intraperitoneal injection of alum at a dose of 700 μg/mouse. Peritoneal exudate cells (PECs) were collected by peritoneal lavage and found a significant increase in the total number of PECs in Becn2 KO mice (FIG. 34A). The protein levels of NLRP3, AIM2, NLRP1, NLRC4, as well as the cleavage of caspase 1 were all dramatically increased in PECs from Becn2 KO mice, as compared with WT controls (FIG. 34B). Nevertheless, no comparable difference was observed in the protein levels of pro-caspase 1 and ASC (FIG. 34B). Consistently, the IL-1β levels were significantly increased in the lavage fluid from Becn2 KO mice after alum challenge (FIG. 34C). Furthermore, the neutrophils (CD11b⁺Gr1⁺) and granulocytic myeloid-derived suppressor cells (gMDSC, CD11b⁺Ly6C^{int}Ly6G^{high}) subsets were both significantly elevated in the peritoneal fluid from alum-treated Becn2-deficient mice, whereas the percentage of monocytic MDSC (mMDSC, CD11b⁺Ly6C^{high}Ly6G^{low}) did not show an appreciable difference (FIGS. 34D-34G). To further determine whether the deletion of Casp1, the downstream effector for all inflammasomes, can rescue the enhanced peritonitis in alum-challenged Becn2-deficient mice, Casp1 KO mice were crossed with Becn2 KO mice to generate Becn2:Casp1 DKO strain, and the successful deletion of Casp1 was validated by PCR analysis (FIG. 33E). The increased IL-1β production and percentages of neutrophils and gMDSC in peritoneal lavage fluid from Becn2-deficient mice were restored in DKO mice to the levels similar to those in WT control (FIGS. 34C-34G). Taken together, these data indicate that loss of Beclin 2 exacerbates alum-induced peritonitis by promoting the inflammasome activation and the recruitment of excessive inflammatory immune cells.

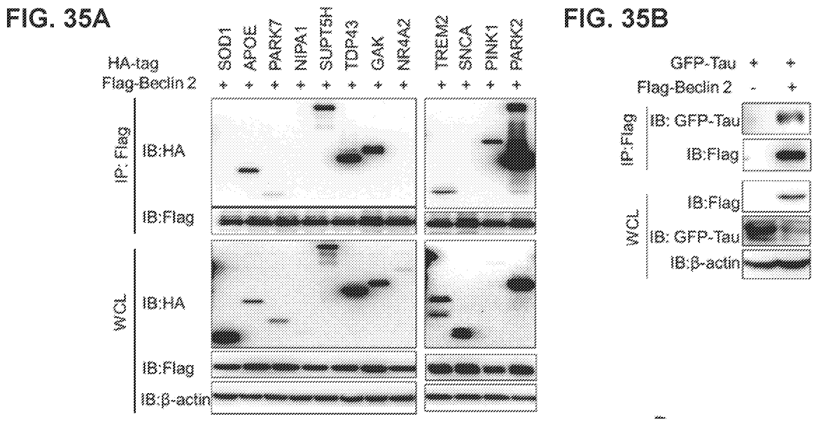

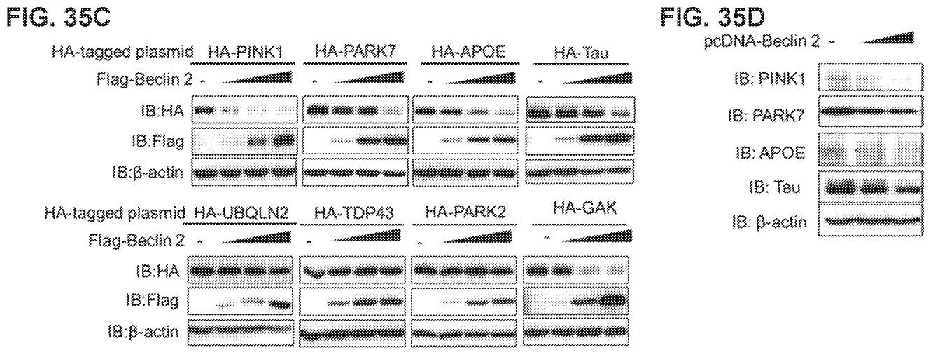

Beclin 2 negatively regulates neurodegenerative diseases. Mutations or loss of autophagy genes can also cause neurodegenerative diseases such as Alzheimer's disease, Parkinson's disease, Huntington's disease, amyotrophic lateral sclerosis (ALS), and static encephalopathy of childhood with neurodegeneration in adulthood (SENDA). Whether Beclin 2 can interact with key molecules involved in neurodegenerative diseases to mediate their degradation was determined. Co-immunoprecipitation of Beclin 2 and neurodegenerative related proteins revealed that Beclin 2 can interacted with APOE, SUPT5H TDP43, PINK1, PARK2, PARK7, TREM2, and TAU (FIGS. 35A and 35B). Furthermore, Beclin2 can induce the degradation of APOE, PINK1, PARK7, GAK, and TAU in a dose-dependent manner (FIGS. 35C and 35D). The degradation of endogenous proteins was confirmed when Beclin 2 were overexpressed in in 293T cells, indicating the role of Beclin 2 in the regulation of neurodegenerative diseases. These findings have identified a previously unrecognized role of Beclin 2 in the regulation of innate immune signaling, tumor development and neurodegenerative diseases. Therefore, Beclin 2 can serve as a target for the prevention and treatment of inflammatory diseases, cancer, and neurodegenerative disease. The applications are exampled as below:

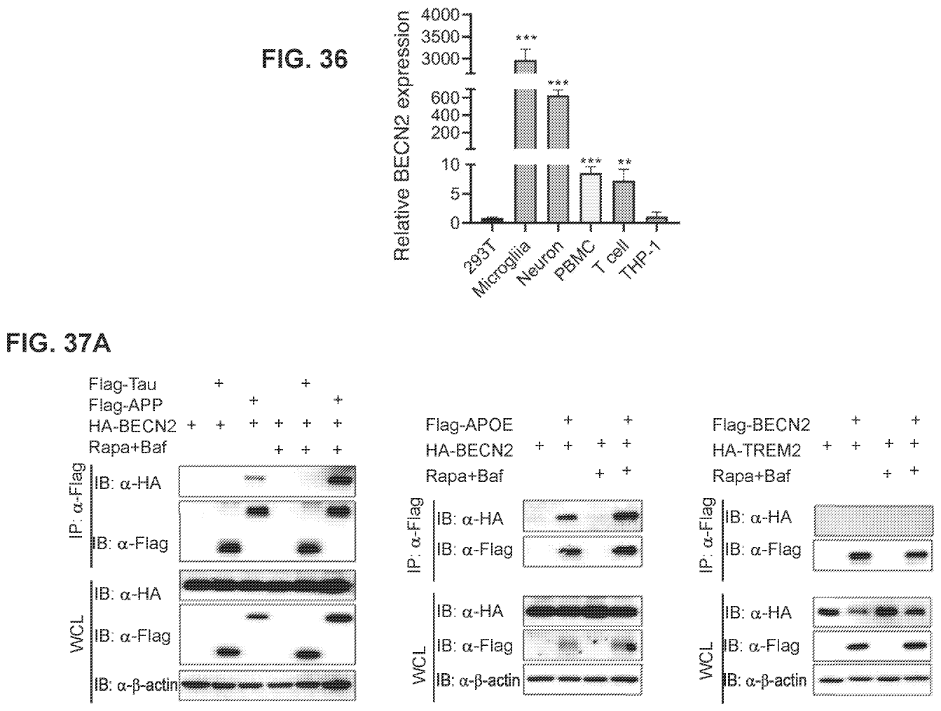

BECLIN2 expression in microglia and neuron cells. BECN2 gene is highly expressed in iPSC-derived neurons and microglia, compared with PBMCs and T cells (positive controls) and 293T and THP-1 cells (as a base level control) (FIG. 36).

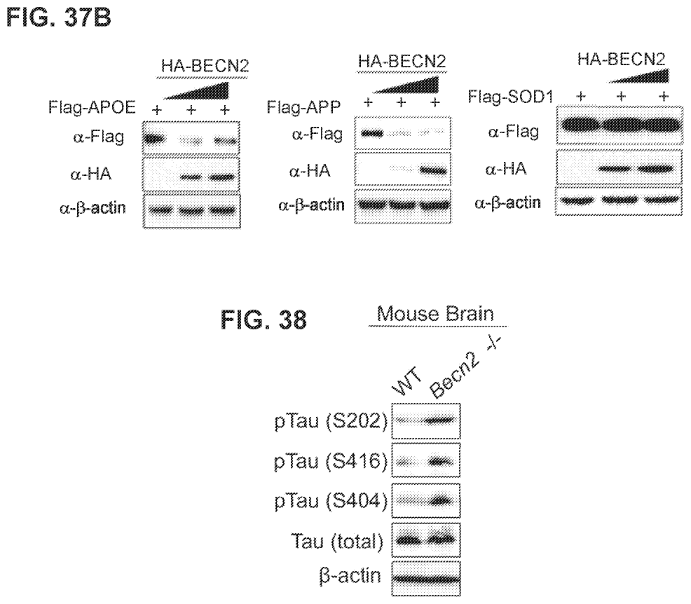

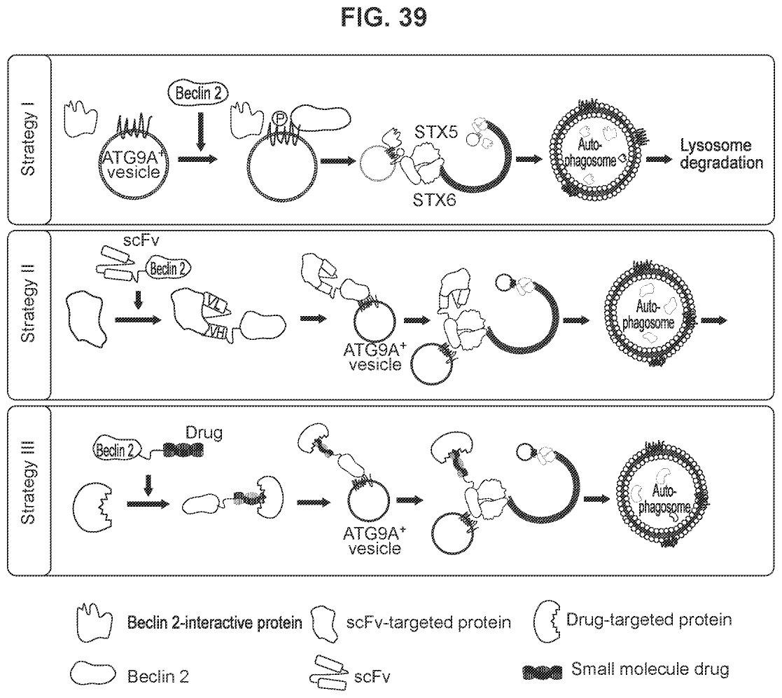

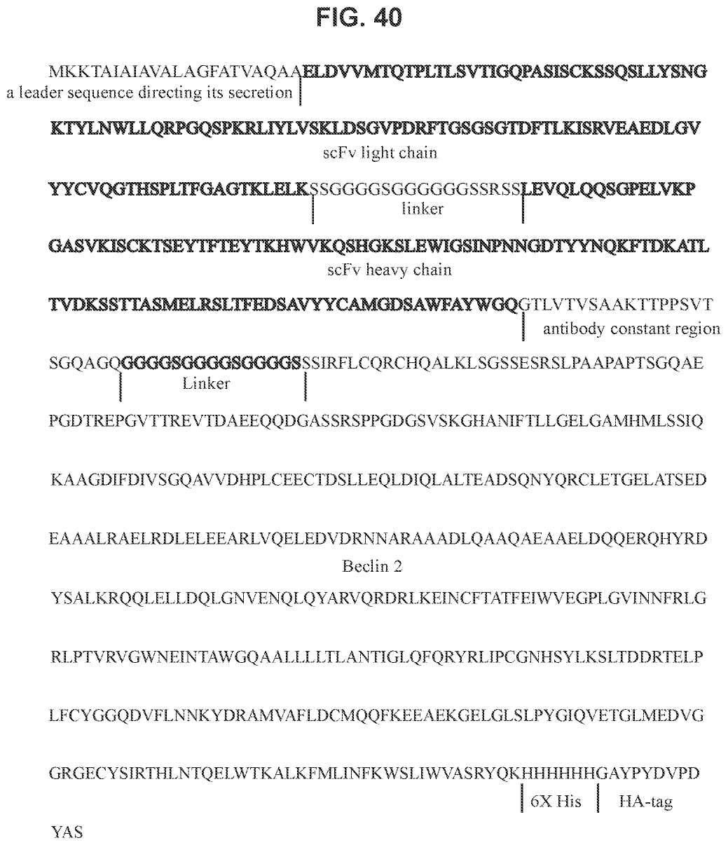

Interaction of BECN2 with Key pathogenic proteins in Alzheimer's disease and cause them for degradation. To determine whether BECN2 could directly interact with key pathogenic proteins, experiments were performed in 293T cells expressing BECN2 along with genes coding for APP, Tau, APOE4 or TREM2. Co-immunoprecipitation and western blotting analyses revealed that BECN2 interacted with APP and APOE4, but not with Tau or TREM2 (FIG. 37, A). Furthermore, BECN2 also targeted APP and APOE4 for lysosomal degradation (FIG. 37, B). SOD1 served as a negative control for degradation. These results strongly support that BECN2 targets APP and APOE for degradation.

BECN2 deficiency increases Tau phosphorylation in Becn2 KO mice. Becn2 KO mice enhanced phosphorylated Tau, compared with those in WT mice, while the total Tau was similar between WT and Becn2 KO mice (FIG. 38), suggesting a critical role of BECN2 in regulating Tau phosphorylation in Becn2 KO mice.

Specific manipulation of the levels of Beclin 2 in myeloid-lineage cells for the treatment of inflammatory diseases or cancer (FIG. 36, strategy I). The approaches for Beclin 2 manipulation include but not limit to recombinant DNA-, siRNA-, or sgRNA-Crisper-technology or small molecules induction for specific overexpression/knockdown/knockout of Beclin 2 in myeloid-lineage cells. These approaches can be applied to control the levels of MEKK3, TAK1, or inflammasome sensors for the treatment of inflammatory diseases or cancer.

Specific manipulation of the levels of Beclin 2 in brain can impede the development of neurodegenerative diseases (FIG. 36, strategy I). Using but not limit to recombinant DNA-, siRNA-, or sgRNA-Crisper-technology or small molecules induction for specific overexpression/knockdown/knockout of Beclin 2 to control the levels of a series of critical molecules in neurodegenerative diseases including APOE, SUPT5H TDP43, GAK, PINK1, PARK2, PARK7, TREM2, and TAU. These approaches can be applied in the prevention and treatment of neurodegenerative diseases such as Alzheimer's disease, Parkinson's disease, Huntington's disease, amyotrophic lateral sclerosis (ALS), and static encephalopathy of childhood with neurodegeneration in adulthood (SENDA).

Generation of a Beclin 2 fusion protein with target molecule-binding single-chain fragment variable (scFv) can lead the degradation of targeted molecules through Beclin 2-mediated non-conventional autophagy (FIG. 36, strategy II). In this application, either full-length Beclin 2 or the ATG9A-binding domain of Beclin 2 can be utilized for the generation of Beclin 2-scFv for sorting and degrading the Beclin 2-interactive molecules. One example of this application is to control the degree of inflammation and to impede the excessive protein aggregates-caused neurodegenerative diseases through Beclin 2-mediated non-conventional autophagy. The complete coding sequencing for a fusion protein of scFv against human TAU protein and Beclin 2 (SEQ ID NO: 6) is enclosed.

Generation of Beclin 2-small molecule drug complex to induce target protein ubiquitination and degradation through proteasome pathway (FIG. 36, strategy III). In this application, Beclin 2 protein can be chemically linked to a small molecule drug through a PEG-based linker. Beclin 2 functions to interact with the target protein, while small molecules were designed to function as a ligand for endogenous protein. The connection of target endogenous protein with Beclin 2 through small molecules can lead the protein degradation through Beclin 2-mediated ATG9A-dependent non-conventional autophagy pathway.

Tables 1 and 2 below describe the most relevant key proteins and diseases targeted by altering Beclin 2 regulation are provided below.

TABLE 1
Upregulation of Beclin 2 and subsequent
NF-κB and MAPK signaling

| Signaling | Upstream | Intermediate | Downstream | |
|---|---|---|---|---|
| Pathway | proteins | proteins | proteins | Disease |
| NF-κB | ↓ TAK1 (via | P38 | ↓ IL-6 | ↓ Cancer |
| | degradation) | | | (Lymphoma |
| | | | | animal model) |
| MAPK | ↓ MEKK3 (via | ERK | | |
| | degradation) | | | |
| | | JNK | | |

TABLE 2
Upregulation of Beclin 2 and subsequent Inflammasome activation

| Inflammasome | Downstream | |
|---|---|---|
| Sensor | proteins | Disease |
| ↓ NLRP3 (via | ↓ IL-1β | ↓ Neurodegenerative |
| reduced | | Diseases (Alzheimer's and |
| activation) | | Parkinson's disease) |
| ↓ AIM2 (via | | |
| reduced | | |
| activation) | | |
| NLRC4 | | |

This application shows that homozygous deletion of Becn2 led to splenomegaly, PG-2T lymphadenopathy, and enhanced inflammatory responses. Elevated IL-6 production was evident in Becn2-deficient macrophages, DCs, and neutrophils upon stimulation, while the TNF-α level was increased in Becn2-deficient neutrophils but not in other immune cells tested. IL-1β was also highly produced by Becn2-deficient immune cells compared with WT control cells, due to elevated expression of pro-IL-1β after LPS treatment. In contrast, enhanced IL-1β production, but not elevated pro-IL-1β levels, was found in heterozygous Becn 1 KO macrophages compared to WT macrophages. Consistent with these findings, Becn2-deficient mice were more sensitive to LPS-induced septic shock, indicating that Beclin 2 functions as a negative regulator of the innate immune signaling pathways to control the expression of proinflammatory cytokines such as IL-6 and IL-1β.

Although NF-κB signaling is known to drive TNF-α and IL-6 expression, Becn2-deficient DCs showed increased IL-6 production, despite comparable IKKα/β activity between WT and Becn2 KO cells after LPS treatment. Further experiments showed that the ablation of ERK1/2 (Mapk3/1) by CRISPR/Cas9 system in Becn2-deficient cells restored IL-6 production to a level similar to that in WT cells, indicating that elevated ERK1/2 signaling is responsible for the increase in IL-6 production. These results are consistent with the previous finding that ERK1/2 signaling antagonists inhibited IL-6 production. Interestingly, it was found that ERK signaling only yield differences between WT and Becn2 KO cells after LPS and poly I:C stimulation, but not CpG or Pam3CSK4 stimulation. TRIF is the adapter for TLR3/TLR4 signaling and type I interferon signaling, however, no appreciable difference in type I interferon signaling was found between WT and Becn2 KO cells, indicating that Beclin 2 negatively regulates the ERK signaling mainly through targeting the downstream kinases, such as MEKK3, but not through TRIF signaling molecule. The downstream signaling of TLRs and their cross-regulation with MAPK (JNK, ERK, p38) pathways are not fully understood, our data show that the activation of MEKK3 is differentially regulated by TLR2-, TLR3-, TLR4- and TLR9-ligand stimulation. This finding is supported by a previous report, showing that MEKK3 interacts with TRAF6 for signaling transduction in response to IL-1 and LPS, but not to CpG, for IL-6 cytokine production. Therefore, loss of Beclin 2 results in elevated MEKK3 levels, which can enhance ERK signaling for IL-6 production.

To further understand the mechanisms by which Beclin 2 deficiency increases ERK1/2 and IKKα/β signaling, compelling evidence was provided to show that Beclin 2 targets TAK1 and MEKK3 for autophagic degradation independent of ATG16-LC3 conjugation machinery and Beclin 1. By contrast, ablation of ULK1 or ATG9A can significantly block the MEKK3 degradation. Beclin 2-mediated TAK1 and MEKK3 degradation requires ULK1-initiated interaction between Beclin 2 and TAK1/MEKK3 that bridged by ATG9A-vesicles. It was further demonstrated that the ULK1 complex (ULK1 and ATG13) was required for Beclin 2-mediated transportation of TAK1I/MEKK3-associated ATG9A-vesicles to autophagosomes/lysosomes for degradation, which is supported by the previous finding that ULK1 and ATG13 were required for the recruitment of ATG9A-vesicles to PAS for autophagosome formation. During the ATG16L/LC3B/Beclin 1-independent degradation process of MEKK3, whether other autophagy protein can function redundantly or differently for executing this degradation remain unclear. For example, MEKK3 degradation is partially blocked in ULK1 KO cells, whether this is due to the ULK2 compensation for the function of ULK1 remains unknown. The LC3/GABARAP family proteins share a high sequence similarity, yet can act differently in autophagosome biogenesis. Deficiency in the LC3 subfamily leads to the generation of smaller autophagosomes, whereas deficiency of GABARAP subfamily leads to the biogenesis of larger autophagosomes. Further studies are warranted to determine whether and how other LC3/GABARAP family proteins compensate for the loss of LC3B to participate in the biogenesis of autophagosomes during LC3B-independent autophagic degradation of MEKK3. Although LPS has been reported to induce autophagy through TRIF-dependent TLR4 signaling, these results show that neither the interaction between ATG9A and MEKK3 nor the MEKK3 protein level was affected by LPS stimulation, indicating that LPS-induced TLR-signaling is not be required in the Beclin 2-mediated autophagic degradation of MEKK3.

SNARE or RAB GTPase family proteins have been reported to play a critical role in membrane fusion in autophagy. STX5 and STX6 are two t-SNAREs (target-SNAP receptor) serving for specific vesicle docking and fusion. STX5 and STX6 have been reported to drive the fusion of autophagosomes with lysosomes and regulate the fusion between endosomes and autophagosomes, respectively. These data show that Beclin 2 can interact with STX5 and STX6, both of which are required for the Beclin 2-mediated MEKK3-associated ATG9A-vesicles fusion to phagophores for autophagic degradation. Although a previous report shows that cells deficient in STX5 have a compromised cathepsin B activity, here no significant changes were detected in lysosomal degradation or Cathepsin B activity among WT, STX5 or STX6 KO cells (data not shown). Overall, three lines of evidence were shown to support the molecular mechanisms of Beclin 2-mediated MEKK3 degradation through an ATG9-dependent but ATG16L/LC3/Beclin1-independent autophagic pathway (FIG. 13E): 1) both Beclin 2 and ATG9A interact with MEKK3 and ULK1, but not with other ULK1 complex subunits (ATG13, FIP200 or ATG101); ULK1 KO or AG13 KD markedly but not completely blocks MEKK3 degradation; 2) The interaction between Beclin 2 and MEKK3 requires ULK complex initiation and ATG9A engagement. ATG9A KO completely blocks Beclin 2-mediated MEKK3 degradation; 3) Beclin 2 interacts with STX5/6 to promote the fusion of MEKK3-associated ATG9A-vesicles with phagophores for MEKK3 degradation. Loss of STX5/6 blocks the Beclin 2-mediated MEKK3 degradation due to the failure of this fusion.

Consistent with these in vitro observations, deletion of Map3k3 completely rescued the proinflammatory phenotypes (such as splenomegaly and lymphadenopathy) observed in Becn2-deficient mice, while ablation of Map3k7 partially rescued these phenotypes. The critical role of MEKK3 in NF-κB activation has been reported through direct phosphorylation of IKKα/β upon induction with TNF-α. However, it was previously shown that ablation of Map3k7 (TAK1) in myeloid lineage enhanced the NF-κB and p38 MAP kinase activation in neutrophils, and induced splenomegaly and lymphadenopathy in mice, while specific deletion of Map3k3 (MEKK3) partially reduced the splenomegaly observed in Map3k7^{ΔM/ΔM} mice. Therefore, the findings that myeloid-specific ablation of Map3k3, but not Map3k7, rescued the phenotypes in Becn2 KO mice, were consistent with the previous study. Collectively, it was demonstrated that MEKK3 played a dominant role in the control of ERK1/2 signaling, proinflammatory cytokine production, splenomegaly and lymphadenopathy in Becn2 KO mice.

Heterozygous deletion of Becn 1 in mice increases the incidence of spontaneous tumors, indicating that Becn 1 is a haploinsufficient tumor-suppressor gene. Heterozygous deletion of Becn2 leads to defective autophagy, obesity and insulin resistance. However, it is unclear whether the ablation of Becn2 results in tumor development. This application provides compelling evidence that homozygous KO of Becn2 increases the incidence of spontaneous B and T cell lymphomas, which is consistent with the hyper-proliferation of T and B cells in peripheral lymphoid tissues, the elevated proinflammatory cytokines, and the enhanced ERK and STAT3 signaling in Becn2-deficient mice. Beclin 1 has been shown to interact with anti-apoptotic Bcl-2 family members via its BH3 domain and restrain tumorigenesis through Mcl-1 destabilization. By contrast, it was shown herein that loss of Beclin 2 neither increased the expression of Bcl-2 or Mcl-1 nor increased the phosphorylation of Bcl-2. Instead, the persistent activation of STAT3 in immune cells and tumor tissues, in concert with the elevated IL-6 production, plays a critical role in inflammatory signaling and tumor development in Becn2-deficient mice. Elevated STAT3 and ERK1/2 signaling pathways have been reported to play important roles in initiating a pre-metastatic tumor niche and promoting tumor development and metastasis. The RNA-seq analysis further supports the notion that the lymphoma development in Becn2-deficient mice is associated with persistent activation of STAT3 signaling and increased expression of pro-tumorigenic cytokines, chemokines, and oncogenes. By contrast, cell-cell junction and adhesion molecules are downregulated in Becn2-deficient mice, thereby facilitating tumor invasion and metastasis. By using IL-6 neutralizing antibody to treat Becn2 KO mice, the levels of the upregulated key genes identified in Becn2 KO lymphoma, the levels of p-STAT3, and the total numbers of splenocytes and lymphocytes were all significantly reduced compared to those in Becn2 KO mice treated with a control antibody, indicating the important role of IL-6 production in the promotion of lymphoma development in Becn2 KO mice. Based on these findings, a working model illustrates how Beclin 2 controls the stability of MEKK3 and TAK1 through an ATG9-dependent but ATG16L/LC3/Beclin1-independent autophagic pathway, thus regulating ERK and IKK signaling-mediated IL-6 production, which in turn activates the STAT3 signaling pathway. The activation of ERK and IL-6-STAT3 signaling further promotes tumor development and metastasis in Becn2 KO mice. Our results have identified an important role of Beclin 2 in innate immune signaling and tumor development, thus providing therapeutic targets for the prevention and treatment of cancer.

In this application, the critical role of Beclin 2 in the negative regulation of inflammasome activation was identified. Genetic deletion of BECN2 enhanced the activities of inflammasomes in response to NLRP3, AIM2, NLRC4, and NLRP1 ligand stimulations, while overexpression of Beclin 2 suppressed the inflammasome activation. Previous reports show that autophagy protein can function in the removal of inflammasome components through selective autophagy by p62 recognition of K63 (Lys 63)-linked polyubiquitinated ASC. Additionally, Beclin 1, a homolog of Beclin 2, is involved in the removal of damaged mitochondria through autophagy, and the loss of Beclin 1 enhanced the inflammasome activity due to excessive mtDNA. Interestingly, a unique pathway was identified for Beclin 2 in the inhibition of inflammasome activation through ATG16L/LC3/Beclin 1-independent lysosomal degradation pathway, which differs from the reported conventional pathway of ATGs in the regulation of inflammasome activation.

Beclin 2 interacts with inflammasome sensors through its CCD-ECD domain, which is also responsible for the degradation of these sensors, indicating that the interaction between Beclin 2 and inflammasome sensors is a critical event for their degradation. By knocking out of a series of key autophagy genes using CRISPR/Cas9 technology, it was shown that Beclin 2-mediated degradation of inflammasome sensors is independent of Beclin 1/ATG14/VPS15-mediated nucleation or ATG16L/ATG7/LC3-controlled ubiquitin-like conjugation systems, but rather relies on ULK1 and ATG9A, as the interaction between Beclin 2 and inflammasome sensors can be disrupted in ATG9A- and ULK1-deficient cells. Given that ULK1 is a serine/threonine kinase and stimulates autophagy via the phosphorylation of ATG9A, the phosphorylated ATG9A can serve as an interactive target for both Beclin 2 and inflammasome sensors. Consistently, neither inflammasome sensors nor Beclin 2 can interact with ATG9A in ULK1 KO cells, indicating the requirement of ULK1 for initiating the assembly of inflammasome sensors-ATG9A-Beclin 2 complex as an upstream protein. On the contrary, inflammasome sensors and ATG9A remain associated in BECN2 KO cells, indicating that their interaction is independent of Beclin 2, and ATG9A functions as a bridge to bring Beclin 2 with AIM2 or NLRP3 together.

ATG9A is the only transmembrane ATG protein that mainly localizes to the trans-Golgi network (TGN) and endosomes, and serves as an important membrane resource for autophagosome initiation. However, it was demonstrated in this application that ATG9A-Beclin 2-dependent inflammasome sensor degradation is independent of Beclin 1/ATG16L/ATG7/LC3-mediated classical autophagic pathway. Emerging roles of ATG proteins have been shown in the autophagy-independent vesicular trafficking processes such as endocytosis, phagocytosis and vesicular secretion. A previous report shows that ULK1-FIP200 and ATG9A are required for ferritin turnover through alternative lysosomal pathway but lacks of involvement of ATG8 lipidation system. The TEM results herein indicate that AIM2 can be frequently delivered to autophagosomes/amphisomes after Beclin 2 overexpression, while such translocation was impeded by the ablation of Beclin 2. These observations indicate that Beclin 2 might facilitate the autophagic degradation of AIM2 through the fusion of AIM2-associated ATG9A⁺-endosomes/MVBs with phagophores, but independent of the Beclin 1/ATG16L/ATG7/LC3-mediated classical autophagic pathway. Three key SNAREs (STX5, STX6 and SEC22A) are critically required for the degradation of inflammasome sensors through interaction with Beclin 2. SNARE family proteins mainly function to drive membrane fusion, in which one part of the SNAREs embeds in the vesicle membrane (v-SNAREs) and the other part in the target site (t-SNAREs). Matched pairs of v- and t-SNAREs interact and pull the opposing membranes into a closer association for fusion. Previous reports show that STX5 and STX6 function as t-SNAREs to drive the fusion of autophagosomes and lysosomes, as well as the fusion between endosomes and autophagosomes, respectively. It was demonstrated herein that the interaction between ATG9A and STX5/STX6 were Beclin 2-dependent, indicating the important role of Beclin 2 in mediating the STX5- and STX6-dependent degradation of inflammasome sensor through membrane fusion. However, the detailed mechanisms by which STX5, STX6 and SEC22A are involved in the membrane fusion between Beclin2⁺ATG9A⁺-endosomes/MVBs with phagophores or autophagosomes for inflammasome sensor degradation remain to be defined.

Inflammasome activation must be tightly regulated, otherwise, uncontrolled inflammasome activation can lead to autoinflammatory syndromes, metabolic diseases, and neurodegenerative diseases. The findings in this application illustrate the in vivo physiological relevance of Beclin 2 in regulating inflammasome-mediated inflammation via alum-induced peritonitis model. This application showed the elevated IL-1β in peritoneal lavage fluid, the increased inflammatory neutrophils and granulocytic myeloid-derived suppressor cells (gMDSC, CD11b⁺Ly6C^{int}Ly6G^{high}), as well as enhanced cleavage of caspase 1 in Becn2 KO PECs, compared with WT controls. The ablation of Casp1 in Becn2 KO mice can restore the alum-induced peritonitis to a level similar to WT mice. Overall, this application has identified a previously unrecognized role of Beclin 2 in the negative regulation of inflammasome activation by targeting inflammasome sensors for degradation. Mechanistically, Beclin 2 interacts with inflammasome sensors to direct them to lysosomes for degradation in a ULK1- and ATG9A-dependent manner. In particular, SEC22A, STX5, and STX6 play essential roles in Beclin 2-mediated lysosomal degradation of inflammasome sensors. Therefore, these findings have identified a previously unrecognized role of Beclin 2 in the negative regulation of several key inflammasome sensor proteins and provided molecular insights into the mechanisms by which Beclin 2 mediates inflammasome sensors for degradation in a ULK1- and ATG9A-dependent lysosomal pathway. This application further indicates that Beclin 2 and its interacting proteins can serve as therapeutic targets for the prevention and treatment of inflammation-associated diseases.

### Methods and Materials.

Reagents and antibodies. Monoclonal anti-HA peroxidase antibody (H6533), and anti-WIPI1 antibody (W2394) were from Sigma. Anti-CD3 antibody (ab5960), VeriBlot for IP detection reagent (HRP) (endogenous IP 2^{nd} antibody, Ab131366), anti-phosphoserine antibody (ab9332), and anti-CD45R antibody (ab64100) were from Abcam. Anti-Mcl-1 antibody (sc-819) was from Santa Cruz. Anti-CXCR4 (PA3-305), anti-ATG9A antibody (for endogenous IP, PA5-85515), anti-STX7 antibody (PA5-76333), anti-STX8 antibody (PA5-48080), anti-RAB7A antibody (PA5-78238), anti-RAB8A antibody (PA5-79906), anti-RAB32A antibody (PA5-68304), anti-VAMP8 antibody (PA5-35300), and anti-Bcl7a (PA5-27123) were from Thermo Fisher. Anti-mouse CD3e-PerCP Cy5.5 (145-2C11, 45-0031-80), anti-mouse F4/80-PE (BM8, 12-4801-82), anti-mouse CD3e-pacific blue (eBio500A2, 48-0033-80), anti-mouse CD3e-PE (145-2C11, 12-0031-81), and anti-mouse Gr-1-FITC (RB6-8C5, 11-5931-81) were obtained from Invitrogen. Anti-IL-21 (06-1074) was from EMD Millipore. Anti-Ki-67 antibody (12202S), anti-SQSTM1/p62 antibody (5114S), anti-Bcl-2 antibody (3498S), anti-p-STAT3 antibody (Tyr705) (9145L), anti-IL-1β antibody (12242S), anti-MEKK3 antibody (5727S), anti-Stat3 antibody (9139S), anti-Erk5 antibody (3372S), anti-p-IKKα/β antibody (2697S), anti-p-p44/42 MAPK antibody (Erk/2) (9101S), anti-p44/42 MAPK antibody (9102S), anti-p-p38 MAPK antibody (9211S), anti-p38 MAPK antibody (9212S), anti-p-JNK antibody (9251S), anti-JNK antibody (9252S), anti-phospho-Stat3 (Tyr75) (9145L), anti-Beclin 1 (D40C5) antibody (3495S) were obtained from Cell Signaling Technology. InVivoMAb anti-mouse IL-6 (Clone MP5-20F3) was obtained from Bio X cell. Information regarding reagents, commercially available kits, and plasmids used in this study are listed in Table 3.

TABLE 3

| REAGENT or RESOURCE | SOURCE | IDENTIFIER |
|---|---|---|
| Antibodies | | |
| Anti-Beclin 2 antibody | Novus | Cat#: NB110- |
| | Biologicals | 60984 |
| Anti-cleaved | Cell Signaling | Cat#: 673145 |
| caspase 1 (Asp296) | Technology | |
| Anti-caspase 1 (D7F10) | Cell Signaling | Cat#: 3866S |
| rabbit | Technology | |
| Anti-Atg5 antibody | Cell Signaling | Cat#: 12944S |
| Anti-Atg7 antibody | Cell Signaling | Cat#: 8558S |
| Anti-Atg16L antibody | Cell Signaling | Cat#: 8089S |
| Anti-Atg14 (D1A1N) | Cell Signaling | Cat#: 96752S |
| antibody | | |
| Anti-ULK1 (D8H5) antibody | Cell Signaling | Cat#: 8054S |
| Anti-WIPI2 antibody | Cell Signaling | Cat#: 8567 |
| Anti-WIPI1 antibody | Cell Signaling | Cat#: 12124S |
| Anti-STX5 antibody | Cell Signaling | Cat#: 14151S |
| Anti-STX6 (C34B2) antibody | Cell Signaling | Cat#: 2869S |
| Anti-WIPI2 antibody | Cell Signaling | Cat#: 8567 |
| Anti-WIPI1 antibody | Cell Signaling | Cat#: 12124S |
| Anti-AIM2 antibody | Cell Signaling | Cat#: #13095 |
| (mouse specific) | | |
| Anti-NALP1/NLRP1 | Lifespan | Cat#: |
| antibody (aa323-372) | Bioscience | LS-B5845-50 |
| IHC-plus ™ | | |
| anti-NLRP3/NALP3, | AdipoGen | Cat#: AG-20B- |
| mAb (Cryo-2) | | 0014-C100 |
| Anti-LC3 antibody | MBL | Cat#: PM036 |
| Anti-Atg 16L antibody | MBL | Cat#: PM040 |
| Anti-Ipaf (NLRC4) antibody | EMD Millipore | Cat#: 06-1125 |
| Anti-Beclin-2 antibody | EMD Millipore | Cat#: MABC266 |
| Anti-NALP1 antibody (B-2) | Santa Cruz | Cat#: sc-166368 |
| ASC Antibody (B-3) | Santa Cruz | Cat#: sc-514414 |
| Anti-Beclin 1 (H-300) | Santa Cruz | Cat#: sc-11427 |
| Anti-caspase 1 p10 (M-20) | Santa Cruz | Cat#: sc-514 |
| DsRed2 | Santa Cruz | Cat#: 101526 |
| Anti-PI3-kinase p100 | Santa Cruz | Cat#: sc-365404 |
| antibody | | |
| Anti-β-actin antibody | Santa Cruz | Cat#: sc-47778 |
| Goat anti-mouse IgG, | Thermo Fisher | Cat#: A11032 |
| Alexa Fluor 594 | | |
| Anti-AIM2 antibody | Thermo Fisher | Cat#: 14-6008-93 |
| Anti-FIP200 antibody | Thermo Fisher | Cat#: PA5-71583 |
| Anti-ATG9A antibody | Thermo Fisher | Cat#: PA1-16993 |
| Anti-FLAG M2-Affinity Gel | Sigma | Cat#: A2220 |
| Anti-HA-Peroxidase (HRP) | Sigma | Cat#: |
| | | H653312013819001 |
| Anti-FLAG M2- | Sigma | Cat#: A8592 |
| peroxidase (HRP) | | |
| Anti-FLAG ® M2 | Sigma | Cat#: M8823 |
| magnetic beads | | |
| Protein A/G agarose | Pierce | Cat#: 20421 |
| Anti-mouse CD11b- | Invitrogen | Cat#: RM2828 |
| pacific blue (M1/70) | | |
| Chemicals and | | |
| Recombinant Proteins | | |
| Lipopolysaccharides | Sigma | Cat#: L3024 |
| Bafilomycin A1 | Sigma | Cat#: B1793 |
| (autophagy inhibitor) | | |
| MG132 | Invivogen | Cat#: tlrl-mg132 |
| Poly(dA:dT)/LyoVe | Invivogen | Cat#: tlrl-patc |
| FLA-ST | Invivogen | Cat#: tlrl-stfla |
| Anthrax lethal factor (LF), | List Lab | Cat#: 172D |
| Recombinant from | | |
| B. anthracis | | |
| Recombinant mouse | Thermo Fisher | Cat#: BMS325 |
| GM-CSF | | |
| Recombinant mouse IL-4 | Thermo Fisher | Cat#: BMS338 |
| 3-Methyladenine (3-MA) | Sigma | M9281 |
| Bafilomycin A1 | Sigma | B1793 |
| (autophagy inhibitor) | | |
| Rapamycin (autophagy | Invivogen | tlrl-rap |
| inducer) | | |
| Pam3CSK4 | Invivogen | tlrl-pms |
| Critical Commercial Kits | | |
| Human Interferon β | PBL | Cat#: 41410 |
| ELISA Kit | | |
| Gateway Cloning System | Thermo Fisher | Cat#: S7020 |
| Direct-zol ™ RNA MiniPrep | ZYMO | Cat#: R2071 |
| Plus w/ TRI Reagent ® | Research | |
| SuperScript III Reverse | Thermo Fisher | Cat#: 18080093 |
| Transcriptase | | |
| Quick-DNA Miniprep Kit | ZYMO | Cat#3006 |
| | Research | |
| ProLong ® Gold Antifade | Life | P36941 |
| Mountant with DAPI | Technology | |
| DAB solution | Electron | #13060 |
| | Microscopy | |
| | Sciences | |
| Dynabeads ® Untouched ™ | Thermo Fisher | 11413D |
| Mouse T Cells Kit | Scientific | |
| MagniSort Mouse B cell | Thermo Fisher | 8804-6827-74 |
| Enrichment Kit | Scientific | |
| NEBuilder ® HiFi DNA | NEB | E2621 |
| Assembly Kit | | |
| pMXs-puro-RFP-ATG9A | Addgene | #60609 |
| p3xFLAG-CMV10-hFIP200 | Addgene | #24300 |
| pUC57-APEX2 | Addgene | #40306 |

Mouse breeding and experiments All animal experiments were performed in animal housing facilities under specific pathogen-free conditions at Houston Methodist Research Institute. All animal studies were performed according to the NIH guidelines for the use and care of live animals and approved by the Animal Care and Use Committee of the Houston Methodist Research Institute. Wild-type C57BL6 mice, Casp1 KO C57BL6 mice and lysozyme-Cre (Lyz2-Cre) mice were obtained from Jackson Laboratory, Becn2 KO mice were kindly provided by Dr. Beth Levine at University of Texas Southwestern Medical Center, Dallas. According to their publication, the heterozygous Becn2 KO mice were backcrossed for more than 10 generations to C57BL/6J mice (Jackson Laboratories). Both Becn2 heterozygous and homozygous KO mice were maintained for breeding. Casp1 heterozygous and homozygous KO mice were maintained for breeding. Becn2 KO mice were generated from both Becn2 heterozygous and homozygous KO breeding pairs, and Becn2:Casp1 DKO mice were generated from Becn2 heterozygous:Casp1 homozygous KO breeding pairs. WT mice were from Becn2 heterozygous KO breeding pairs. The primer sequences for Becn2 KO mice genotyping were listed in Table 4. All mice were 6-12 weeks of age for experimental use, with the exception of mice for spontaneous tumor development. Becn 2 KO mice were bred with Map3k7^{ΔM/ΔM} mice (Map3k^{flox/flox} is provided by M. D. Schneider, Baylor College of Medicine and bred with Lyz2-Cre to obtain Map3k7^{ΔM/ΔM}) or Map3k3^{ΔM/ΔM} mice to generate Map3k7^{ΔM/ΔM}:Becn2 KO and Map3k3^{ΔM/ΔM}:Becn2 KO, respectively. Becn1^{flox/flox} mice (from Knockout Mouse Project [KOMP] Repository) were bred with Lyz2-Cre mice (Jax Lab) to obtain Becn1^{ΔM/ΔM} mice. For the LPS-induced endotoxic shock model, mice were i.p. injected with LPS (30 mg/kg body weight) and monitored for survival. Meanwhile, blood samples were collected to examine the proinflammatory cytokine levels. For macrophage depletion, clodronate-containing liposome was applied at 150 μL per mouse 16 h prior to LPS injection. For induction of peritonitis, WT, Becn2-deficient, and Casp1-deficient mice (females, 6 weeks' old) were i.p. injected with 700 μg alum/mouse. To analyze the IL-1β levels and PECs in the peritoneal cavity, peritoneal cavities were lavaged with cold PBS at 12 h post i.p. injection of alum.

TABLE 4
Oligo sequences for gene knockout, genotyping,
and RT-PCR
Oligo sequences for gene knockout, genotyping,
and RT-PCR

Primer for RT-PCR human (BECN2), forward
ACTACAGTGCCTTGAAGCGG (SEQ ID NO: 10)
Primer for RT-PCR human (BECN2), reverse
CTCAAACGTGGCGGTGAAAC (SEQ ID NO: 11)
Primer for RT-PCR mouse MEKK3, forward1
ATAAGGACACAGGTCACCCAA (SEQ ID NO: 12)
Primer for RT-PCR mouse MEKK3, reverse1
TGCTCCACATCTTCGTATCTCA (SEQ ID NO: 13)
Primer for RT-PCR mouse MEKK3, forward2
AGGTTCGGATCAAGCCTTCC (SEQ ID NO: 14)
Primer for RT-PCR mouse MEKK3, reverse2
TGTCGCTCAGGTACATATCCC (SEQ ID NO: 15)
Primer for RT-PCR mouse TAK1, forward1
CGGATGAGCCGTTACAGTATC (SEQ ID NO: 16)
Primer for RT-PCR mouse TAK1, reverse1
ACTCCAAGCGTTTAATAGTGTCG (SEQ ID NO: 17)
Primer for RT-PCR mouse TAK1, forward2
TGCCTTACTACACTGCTGCTC (SEQ ID NO: 18)
Primer for RT-PCR mouse TAK1, reverse2
AAGCTGTACCAAAATCGCAGA (SEQ ID NO: 19)
Primer for RT-PCR human MEKK3, forward1
GGCGAATTATAGCGTTCAGCC (SEQ ID NO: 20)
Primer for RT-PCR human MEKK3, reverse1
GGGACAACAGCAATATCCTAAGG (SEQ ID NO: 21)
Primer for RT-PCR human MEKK3, forward2
CAGGTGCGGATCAAGGCTT (SEQ ID NO: 22)
Primer for RT-PCR human MEKK3, reverse2
CCGCTCAGGAACATAGCCAG (SEQ ID NO: 23)
Primer for RT-PCR human TAK1, forward1
CCGGTGAGATGATCGAAGCC (SEQ ID NO: 24)
Primer for RT-PCR human TAK1, reverse1
GCCGAAGCTCTACAATAAACGC (SEQ ID NO: 25)
Primer for RT-PCR human TAK1, forward2
AAACCACCAAACTTACTGCTGG (SEQ ID NO: 26)
Primer for RT-PCR human TAK1, reverse2
CGCGTTATCACTTCCCAAAGAA (SEQ ID NO: 27)
Becn2 KO mouse genotyping, KO forward
GTGAGTCGTATTAATTTCGATAAGCCAG
(SEQ ID NO: 28)
Becn2 KO mouse genotyping, WT forward
CCCGGCTTAGACTTTTTTCTAAAGATG
(SEQ ID NO: 29)
Becn2 KO mouse genotyping, reverse
GAGGTAAGCAGAGTAAAAGTGCAGAG
(SEQ ID NO: 30)
sgRNA for human BECN2 knockout
AAGAGCAGCGGCGGATCTCC (SEQ ID NO: 31)
sgRNA for mouse ERK1 knockout
CCACGTGCGCAAGACCAGAG (SEQ ID NO: 32)
sgRNA for mouse ERK2 knockout
GGTGCAGAACGTTAGCTGAA (SEQ ID NO: 33)
sgRNA for human ATG16L1 knockout
CAATTTAGTCCCGGACATGA (SEQ ID NO: 34)
sgRNA for human BECN1 knockout
ATTTATTGAAACTCCTCGCC (SEQ ID NO: 35)
sgRNA for human LC3B (MAP1LC3B) knockout
TTCAAGCAGCGCCGCACCTT (SEQ ID NO: 36)
sgRNA for human ATG9A knockout
TCTGGAAACGGAGGATGCGG (SEQ ID NO: 37)
sgRNA#1 for human ULK1 knockout
TCTGCGGTTTCAGGTCGCGG (SEQ ID NO: 38)
sgRNA#2 for human ULK1 knockout
ATGATGGCGGCCACACTCTG (SEQ ID NO: 39)
sgRNA#1 for human STX5 knockout
TTAGACCCGTAGCGTTTCCG (SEQ ID NO: 40)
sgRNA#2 for human STX5 knockout
GCTTGGCAAATGTGTTGCTA (SEQ ID NO: 41)
sgRNA#1 for human STX6 knockout
TTGCCGAGTACTTGTAATGA (SEQ ID NO: 42)
sgRNA#2 for human STX6 knockout
CAAGTACTCGGCAAGTTGTC (SEQ ID NO: 43)
sgRNA for human VAMP8 knockout
TTATGACCCAGAATGTGGAG (SEQ ID NO: 44)
sgRNA for human RAB7A knockout
ACGGTTCCAGTCTCTCGGTG (SEQ ID NO: 45)
sgRNA for human RAB8A knockout
GTTGTCGAAGGACTTCTCGT (SEQ ID NO: 46)
sgRNA for human RAB32 knockout
GTCCCAGTTGAGGACCTTGA (SEQ ID NO: 47)
sgRNA for human STX7 knockout
GGATGTTAGAAGAGATCCTC (SEQ ID NO: 48)
sgRNA for human STX7 knockout
GAGTTACAAGATCATCCAAG (SEQ ID NO: 49)

Cell culture. HEK293T (CRL-3216), Hela cells (CRM-CCL-2) and THP-1 cells were purchased from ATCC. HEK293T and Hela cells were grown in DMEM supplemented with 10% fetal bovine serum and 0.5% penicillin/streptomycin·THP-1 cells were grown in RPMI-1640 medium supplemented with 10% FBS, 0.5% penicillin/streptomycin, and 0.05 mM β-mercaptoethanol. BMDCs were prepared as previously described. Bone marrow was obtained from mouse femurs and tibias. Bone marrow progenitor cells were cultured in complete RPMI-1640 medium containing mouse granulocyte/macrophage colony-stimulating factor (GM-CSF, 20 ng/ml), mouse IL-4 (10 ng/ml) and β-Me (55 μM) for BMDC generation. Bone marrow progenitor cells were cultured in L929-cell conditioned medium for 5-6 days to obtain BMDMs. Peritoneal neutrophils were obtained from the peritoneal cavity by i.p. injection of 3 ml 4% (v/v) thioglycollate for 3 h, followed by peritoneal lavage with cold RPMI media/2% FBS. Cells were stained with anti-Gr-1-PE antibody (eBioscience) and purified using PE-positive selection magnetic beads (Stem Cell Technologies). For inflammasome activation studies, BMDCs or BMDMs were primed for 3 h with LPS (100 ng/ml), followed by treatment with ATP (5 mM) for 1 h. T cells were isolated from spleen and lymph nodes of 6- to 8-week-old mice and purified by untouched T cell selection kit (Thermo Fisher). Splenic B cells were isolated from spleen and lymph nodes of 6- to 8-week-old mice by MagniSort Mouse B cell Enrichment Kit (Thermo Fisher). In another example of inflammasome activation studies, BMDMs, HEK293T-CIA and THP-1 cells were primed for 3 h with LPS (Sigma-Aldrich) (100 ng/ml), then stimulated with nigericin (Invivogen) (1 μM, 6 h), poly(dA:dT) (poly(dA:dT)/LyoVec, tlrl-patn, Invivogen) (1 μg/ml, 6 h), anthrax lethal factor (LF) (recombinant from B. anthracis, 172D, List Lab) (1 μg/ml, 4-6 h), or flagellin (FLA-ST, tlrl-stfla, Invivogen) (20 μg/ml, 6-8 h). For cycloheximide chase assays, cells were treated with cycloheximide (Cell Signaling Technology) at 100 mg/ml for different time points starting from 24 h post-transfection.

Flow cytometry Cell suspensions were obtained from mouse tissues and stained for 20 min at 4° C. in PBS containing 1% FCS and 10 mM EDTA with the indicated antibodies for cell surface staining. Flow cytometric analysis was performed with a BD FACSCalibur or BD FACSAria system (Becton Dickson). The acquired data were analyzed with FlowJo software. For characterizing mouse peritoneal exudates cells (PECs), mouse PECs were obtained from peritoneal lavage fluid and stained with indicated antibodies for cell surface staining for 20 min at 4° C. in PBS containing 1% FCS and 10 mM EDTA. CD11b and Gr-1(Ly6G/Ly6C) were used to label neutrophils. Monocytic myeloid-derived suppressor cells (mMDSC) was characterized as CD11b⁺Ly6C^{high}Ly6G^{low}, and granulocytic MDSC (gMDSC) was characterized as CD11b⁺Ly6C^{int}Ly6G^{high}. Flow cytometric analysis was performed with a BD FACSCalibur or BD FACSAria system (Becton Dickson). The acquired data were analyzed with FlowJo software.

CYTOF Spleen tissues isolated from WT and Becn2 KO mice were mashed through 70-μm Nylon cell strainers and treated with RBC lysis buffer. Cells were washed twice with RPMI-1640 medium supplemented with 10% FBS, then stimulated with phorbol 12-myristate 13-acetate (PMA) for 4 h to activate the T cells. The single cell suspension was stained with metal-tag viability dye for 5 min and wash with cell staining buffer (Fluidigm), followed by staining of surface markers and intracellular markers separately. Cells were then stained with Cell ID Intercalator Ir (Fluidigm) at 4° C. overnight. The next day, cells were washed and prepared for acquisition with Helios (Fluidigm). Cytobank (cytobank.org) was used for data analysis and generation of viSNE maps. Briefly, ungated live cell populations were analyzed for an equal number of events per sample after normalization, followed by gating on CD45⁺ cell population. Lymphocyte populations were clustered in each viSNE map by CD4, CD8α, CD3ε, CD19, B220, IFN-γ, IL-4, IL-17A, Foxp3, CD10, CD23, CD21, and GL7 markers to display the lymphocyte subpopulations with different colors. The ratio of different cell populations within CD45⁺ cells was quantified by Cytobank and plotted with Prism. The viSNE plots are shown as two-dimensional scatter plots with the x- and y-axes identified by tSNE1 and tSNE2.

Immunoprecipitation and immunoblot analyses. Cells were lysed using RIPA buffer (150 mM NaCl, 1.0% IGEPAL CA-630, 0.5% sodium deoxycholate, 0.1% SDS, 50 mM Tris, pH 8.0 protease inhibitor cocktail [Roche]) on ice for 15 min. Immunoblotting was performed by loading the samples on SDS-PAGE gels, conducting electrophoresis, transferring the samples to PVDF membranes (Bio-Rad), and then incubating the membranes with the indicated antibodies. For all immunoblots, the Luminata Western HRP Chemiluminescence Substrates (Millipore) and ChemiDoc XRS+ System with Image Lab (Bio-rad) was used for protein detection.

To determine the interactions between endogenous Beclin 2 and the binding partners, 5 million cells were lysed using RIPA buffer and incubate with 5 uL primary antibody against each partner protein respectively along with 30 uL Protein A/G beads, the immunoprecipitates were eluted with 2×SDS loading buffer. The secondary antibody (Veriblot for IP detection reagent HRP) that only recognized native IgG but not denatured IgG was applied for immunoblotting of proteins in immunoprecipitates. For immunoprecipitation of Flag-tagged proteins, cell lysates were incubated with anti-Flag beads (Sigma) at 4° C. overnight. The beads were washed four times with RIPA lysis buffer, and immunoprecipitates were eluted with 2×SDS loading buffer. Immunoblotting was performed by loading the samples on SDS-PAGE gels, conducting electrophoresis, transferring the samples to PVDF membranes (Bio-Rad), and then incubating the membranes with the indicated antibodies. For all immunoblots, the LumiGLO Chemiluminescent Substrate System from KPL (Gaithersburg, MD) was used for protein detection.

Whole cell lysates obtained 24 h post-transfection or via ligand stimulation were obtained using RIPA lysis buffer and shook on ice for 15 min. The HEK293T cell lysates were immunoprecipitated with anti-Flag conjugated beads (BioLegend) at 4° C. overnight. The lysates of THP-1 cells or BMDMs were immunoprecipitated with 2 μg/ml anti-AIM2, anti-NLRP3 antibody, or anti-ATG9A antibody along with protein A/G-agarose beads at 4° C. overnight. The beads were washed five times with RIPA lysis buffer, and immunoprecipitates were eluted with 2×SDS loading buffer and subjected to SDS-PAGE gel electrophoresis, followed by immunoblotting. The secondary antibody (Veriblot for IP detection, HRP-conjugated) that only recognized native IgG but not denatured IgG was applied for immunoblotting of endogenous proteins in immunoprecipitates. For immunoblotting of the cleaved caspase 1 in cell culture supernatants, supernatants (400 μl) were precipitated by add 400 μl methanol and 100 μl chloroform, then vortexed and centrifuged for 15 min at 14,000 g. The upper phase was discarded and another 400 μl methanol was added to the interphase. This mixture was centrifuged for 15 min at 14,000 g and the protein pellet was dried at room temperature, resuspended with 2×SDS loading buffer and boiled for 5 min at 95° C.

ELISA. Bone marrow macrophages, HEK293T-CIA cells or THP-1 cells were seeded in 24-well plates and cultured overnight. After priming with 200 ng/ml LPS for 3 h and stimulating with nigericin (1 uM, 6 h), poly(dA:dT) (1 μg/ml, 6 h), anthrax lethal factor (LF) (1 μg/ml, 4-6 h) or flagellin (20 μg/ml, 6-8 h), the supernatants were collected and measured for IL-1β concentrations using IL-1β ELISA kits (human interferon beta ELISA kit 41410, PBL, eBioscience or anti-mouse IL-1β ELISA kit: E05277-1531; 1:500 dilution, E03232-1632; 1:1000 dilution, eBioscience) according to the manufacturer's protocols. Capture and detection antibodies for mouse TNF-α, IL-6, IL-1β, IL-10, IL-17, and IFN-γ (eBioscience) were used for the measurement of cytokines in cell supernatants and mouse sera according to the manufacturer's protocols.

Plasmids and cloning A complete open reading frame of human ATG proteins, RABs, and SNAREs, unless otherwise specified, were obtained from entry clone library (Human ORFeome library from Thermo Fisher or Baylor Ultimate ORF LITE) and subsequently subcloned into pcDNA3.1 or pEGFP-C2 vectors using PCR-based Gateway technology (Life Technologies). Plasmids encoding Flag-FIP200, RFP-ATG9A, and pUC57-APEX2 were obtained from Addgene (see Table 3). Plasmids encoding MEKK3-APEX2 was cloned into pcDNA-3.1 vectors by homologous recombination using NEBuilder® HiFi DNA Assembly Cloning Kit. Plasmids encoding human Beclin 2, AIM2, NLRP3, NLRC4, NLRP1, ASC, Caspase 1, ATG9A, RAB4A, RAB7A, RAB8A, RAB9A, RABl1A, RAB24, RAB32, RAB33B, SNAP25, SNAP29, SEC22B, SEC22A, STX5, STX6, STX7, STX8, STX17, VAMP4, VAMP7, VAMP8, and VTI1B were generated from Human ORFeome library and/or Baylor Ultimate ORF LITE using the gateway cloning system (S7020, ThermoFisher), or cloned using HEK293T cDNA (see primer sequence in Table 5). Vectors including pcDNA-ccdB-FLAG-B (Gateway), pcDNA-ccdB-HA-B (Gateway), pcDNA-ccdB-eGFP-B (Gateway) or pcDNA-mCherry2-C1 vectors were transcribed under the control of the CMV promoter. Truncation of NLRP3 (PYD 1-94, NACHT 95-559, LRR 560-894), AIM2 (PYD 1-116, HIN 117-343), Beclin 2 (N 1-87, ΔN 88-431, CCDECD 110-431, ΔECD 1-248) were generated by PCR using primers listed in Table 5 and subcloned into final constructs containing affinity tag. The plasmid encoding pUC57-APEX2 was obtained from Addgene. AIM2-APEX2 encoding sequence was cloned into pcDNA-3.1 vectors by homologous recombination using NEBuilder® HiFi DNA Assembly Cloning Kit.

TABLE 5
Sequences of primers used for constructing
gene expression plasmids, related to Methods.

| Gene | Primer Sequence |
|---|---|
| Beclin 2 | F: GGGGACAAGTTTGTACAAAAAAGCAGGCTTCATG |
| | TCTTCCATCCGCTT (SEQ ID NO: 50) |
| | R: GGGGACCACTTTGTACAAGAAAGCTGGGTCCTAC |
| | TTTTGATACCTTG (SEQ ID NO: 51) |
| AIM2 | F: GGGGACAAGTTTGTACAAAAAAGCAGGCTTCATG |
| | GAGAGTAAATACAAG (SEQ ID NO: 52) |
| | R: GGGGACCACTTTGTACAAGAAAGCTGGGTCCTAT |
| | GTTTTTTTTTTGGC (SEQ ID NO: 53) |
| NLRP3 | F: GGGGACAAGTTTGTACAAAAAAGCAGGCTTCATG |
| | AAGATGGCAAGCAC (SEQ ID NO: 54) |
| | R: GGGGACCACTTTGTACAAGAAAGCTGGGTCCTAC |
| | CAAGAAGGCTCAA (SEQ ID NO: 55) |
| NLRP1 | F: GGGGACAAGTTTGTACAAAAAAGCAGGCTTCATG |
| | GCTGGCGGAGCCTG (SEQ ID NO: 56) |
| | R: GGGGACCACTTTGTACAAGAAAGCTGGGTCCTAG |
| | TATCTCCTGGCGTC (SEQ ID NO: 57) |
| NLRC4 | F: GGGGACAAGTTTGTACAAAAAAGCAGGCTTCATG |
| | AATTTCATAAAGG (SEQ ID NO: 58) |
| | R: GGGGACCACTTTGTACAAGAAAGCTGGGTCCTAT |
| | TAAGCAGTTACTAGTT (SEQ ID NO: 59) |
| ATG9A | F: GGGGACAAGTTTGTACAAAAAAGCAGGCTTCATG |
| | GCGCAGTTTGAC (SEQ ID NO: 60) |
| | R: GGGGACCACTTTGTACAAGAAAGCTGGGTCCTAT |
| | ACCTTGTGCACC (SEQ ID NO: 61) |
| ULK1 | F: GGGGACAAGTTTGTACAAAAAAGCAGGCTTCATG |
| | GAGCCCGGCCG (SEQ ID NO: 62) |
| | R: GGGGACCACTTTGTACAAGAAAGCTGGGTCCTAT |
| | CAGGCACAGATGCCAG (SEQ ID NO: 63) |
| Beclin | F: GGGGACAAGTTTGTACAAAAAAGCAGGCTTCGGC |
| 2/ΔN | GCCATGCACATGC (SEQ ID NO: 64) |
| | R: GGGGACCACTTTGTACAAGAAAGCTGGGTCCTAC |
| | TTTTGATACCTTGAGGC (SEQ ID NO: 65) |
| Beclin 2/ | F: GGGGACAAGTTTGTACAAAAAAGCAGGCTTCCAA |
| CCDECD | GCAGTTGTGGACC (SEQ ID NO: 66) |
| | R: GGGGACCACTTTGTACAAGAAAGCTGGGTCCTAC |
| | TTTTGATACCTTGAGGC (SEQ ID NO: 67) |
| Beclin | F: GGGGACAAGTTTGTACAAAAAAGCAGGCTTCATC |
| 2/ECD | AACTGTTTCAC (SEQ ID NO: 68) |
| | R: GGGGACCACTTTGTACAAGAAAGCTGGGTCCTAC |
| | TTTTGATACCTTGAGGC (SEQ ID NO: 69) |
| Beclin | F: GGGGACAAGTTTGTACAAAAAAGCAGGCTTCATG |
| 2/ΔECD | TCTTCCATCCGCTTCCT (SEQ ID NO: 70) |
| | R: GGGGACCACTTTGTACAAGAAAGCTGGGTCCTAT |
| | TCCTTCAGCCGGTCCC (SEQ ID NO: 71) |
| AIM2/PY | F: GGGGACAAGTTTGTACAAAAAAGCAGGCTTCATG |
| D | GAGAGTAAATACAAGG (SEQ ID NO: 72) |
| | R: GGGGACCACTTTGTACAAGAAAGCTGGGTCCTAA |
| | TTTCTGATGGCTGCAG (SEQ ID NO: 73) |
| AIM2/HI | F: GGGGACAAGTTTGTACAAAAAAGCAGGCTTCGAT |
| N | GTCGCAAAGCAACG (SEQ ID NO: 74) |
| | R: GGGGACCACTTTGTACAAGAAAGCTGGGTCCTAT |
| | GTTTTTTTTTTGGC(SEQ ID NO: 75) |
| mCherry- | F: ATTGCTAGCCACCATGTCTTCCATCCGCTTCC |
| Beclin 2 | (SEQ ID NO: 76) |
| | R: TATCTCGAGCTTTTGATACCTTGAGGCAAC |
| | (SEQ ID NO: 77) |
| mCherry- | F: GGGAGACCCAAGCTGGCTAGCCACC ATGGCGCA |
| ATG9A | GTTTGACAC (SEQ ID NO: 78) |
| | R: CCCTTGCTCACCATCTCGAGTACCTTGTGCACCT |
| | GAGGGGGT (SEQ ID NO: 79) |
| mCherry- | F: AGCTGTACAAGTAGGGCTCCATGTCCTGCCGGGA |
| STX5 | TCGGACC (SEQ ID NO: 80) |
| | R: CACTGTGCTGGATATCTGCATCAAGCAAGGAAGA |
| | CCACAAA (SEQ ID NO: 81) |
| mCherry- | F: AGCTGTACAAGTAGGGCTCCATGTCCATGGAGGA |
| STX6 | CCCCTTC (SEQ ID NO: 82) |
| | R: CACTGTGCTGGATATCTGCATCACAGCACTAAGA |
| | AGAGGAT (SEQ ID NO: 83) |
| mCherry- | F: AGCTGTACAAGTAGGGCTCCATGTCTATGATTTT |
| SEC22A | ATCTGCC (SEQ ID NO: 84) |
| | R: CACTGTGCTGGATATCTGCATCAGACATCATAAT |
| | CGGGAGC (SEQ ID NO: 85) |
| mCherry- | F: AGCTGTACAAGTAGGGCTCCATGGAGAGTAAATA |
| AIM2 | CAAGGA (SEQ ID NO: 86) |
| | R: CACTGTGCTGGATATCTGCACTATGTTTTTTTTT |
| | TGGCCT (SEQ ID NO: 87) |

Gene knockout by CRISPR technology in cells Gene knockout with CRISPR technology in THP1 cells and 293T cells were performed using the pLenti-CRISPR-Cas9 v1 or v2 vectors (Addgene) containing gene-specific sgRNAs, followed by selection with Zeocin at 400 μg/ml for 5 days. Transduction of BMDMs using pLenti-CRISPR-Cas9 system was started at day 2 after isolating progenitor cells from bone marrow. Lentiviruses produced by 293T cells were concentrated by centrifuging at 20,000 g for 2 h and resuspended in L929-cell conditioned medium for transduction. 16 h after transduction, the medium was replaced by the fresh L929-cell conditioned medium (for differentiation) and cells were recovered for 12 h, followed by selection with zeocin at 400 μg/ml for 4 days. On day 7, zeocin selection was withdrawal and LPS stimulation was started on day 7.5. The KO efficiency was confirmed by immunoblot analysis. The sgRNAs used for gene KO are listed in Table 4.

Human BECN2, BECN1, ATG16L, ATG7, MAP1LC3B, ATG14, WIPI2, VPS15, STX5, STX6, SEC22A, ULK1, and ATG9A sgRNAs were designed using an online CRISPR design tool (crispr.mit.edu) by inputting targeted exon sequence. VAMP7, VAMP8, RAB7A, RAB8A, RAB32, STX7, STX8, STX17, VTILB sgRNAs were from Library (LentiArray human CRISPR library, Thermofisher). Designed sgRNAs were cloned into the BsmB1 site of pLenti-Crispr-Cas9 v2 vectors (Addgene) containing Cas9-P2Apuromycin and were verified by sequencing analysis. The sgRNA-containing plasmids were transfected into HEK293T cells with psPAX2 (12260, Addgene) and pMD2.G (12259, Addgene) plasmids. After two days, the virus-containing medium was subjected to ultracentrifugation (20,000 g at 4° C. for 2 h) and frozen at −80° C. HEK293T cells were transduced with control sgRNA- or gene-targeting sgRNA-containing lentiCRISPR viruses. Transduced cells were selected in the presence of puromycin (Invivogen) at 2 μg/ml for 3 days. The KO efficiency was confirmed by immunoblot analysis or genomic DNA sequencing. A list of sgRNA sequences for KO is presented in Table 6.

TABLE 6
Design of the single-guide RNA sequence
(sgRNA) for gene knockout by the Cas9-
expression vector pLentiCRISPR-v2,
related to Methods.

| Gene | sgRNA guide-sequence |
|---|---|
| hBECN2 target 1 | AAGAGCAGCGGCGGATCTCC |
| | (SEQ ID NO: 88) |
| hBECN2 target 2 | TAACAAGTATGACCGCGCGA |
| | (SEQ ID NO: 89) |
| hMAP1LC3B target | TTCAAGCAGCGCCGCACCTT |
| | (SEQ ID NO: 90) |
| hATG16L target | CAATTTAGTCCCGGACATGA |
| | (SEQ ID NO: 91) |
| hBECN1 target | ATTTATTGAAACTCCTCGCC |
| | (SEQ ID NO: 92) |
| hATG9A target 1 | TCTGGAAACGGAGGATGCGG |
| | (SEQ ID NO: 93) |
| hATG9A target 2 | CCTCGGCGACGTGCACCAAC |
| | (SEQ ID NO: 94) |
| hULK1 target 1 | TCTGCGGTTTCAGGTCGCGG |
| | (SEQ ID NO: 95) |
| hULK1 target 2 | ATGATGGCGGCCACACTCTG |
| | (SEQ ID NO: 96) |
| hWIPI2 target 1 | CACAAATCTGGAGTTCGTCA |
| | (SEQ ID NO: 97) |
| hWIPI2 target 2 | GAAGACCTGCACCTCTCCGA |
| | (SEQ ID NO: 98) |
| hATG7 target | TCCGTGACCGTACCATGCAG |
| | (SEQ ID NO: 99) |
| hATG14 target 1 | TGTGCAACACTACCC GCCGG |
| | (SEQ ID NO: 100) |
| hATG14 target 2 | GGTGGACTCCGTGGACGATG |
| | (SEQ ID NO: 101) |
| hVPS15 target | ATCGTTAGACTAGCCTATGC |
| | (SEQ ID NO: 102) |
| hSTX5 target | TTAGACCCGTAGCGTTTCCG |
| | (SEQ ID NO: 103) |
| hSTX6 target | TTGCCGAGTACTTGTAATGA |
| | (SEQ ID NO: 104) |
| hSEC22A target | GATAGATGTACACTGAAAAC |
| | (SEQ ID NO: 105) |
| hVAMP7 target | AACTCGCTATTCATGGCATA |
| | (SEQ ID NO: 106) |
| hVAMP8 target | TTATGACCCAGAATGTGGAG |
| | (SEQ ID NO: 107) |
| hRAB7A target | ACGGTTCCAGTCTCTCGGTG |
| | (SEQ ID NO: 108) |
| hRAB8A target | GTTGTCGAAGGACTTCTCGT |
| | (SEQ ID NO: 109) |
| hRAB32 target | GTCCCAGTTGAGGACCTTGA |
| | (SEQ ID NO: 110) |
| hSTX7 target | GGATGTTAGAAGAGATCCTC |
| | (SEQ ID NO: 111) |
| hSTX8 target | GAGTTACAAGATCATCCAAG |
| | (SEQ ID NO: 112) |
| hSTX17 target | CAATTTCTCGGATATTGGAT |
| | (SEQ ID NO: 113) |
| hVTI1B target | CCTTGCTAAACTCCATCGGG |
| | (SEQ ID NO: 114) |

Immunohistochemistry and immunofluorescence. For immunohistochemistry, tissues were fixed overnight at room temperature in freshly prepared 4% paraformaldehyde and then embedded in paraffin. Formalin-fixed, paraffin-embedded tissues were sectioned into slices at a thickness of 5 μm then mounted onto glass slides. All sections used for immunohistochemistry were deparaffinized and hydrated using a graded ethanol series and deionized water. The tissues were incubated overnight at 4° C. with primary antibodies, followed by labeling with HRP-conjugated or fluorescent probe-conjugated secondary antibodies (Alexa Fluor 488 anti-rat IgG or Alexa Fluor 555 anti-rabbit IgG). For immunofluorescence, tissues were mounted using ProLong® Gold Antifade Mountant with DAPI (P36941, Life Technology). Immunofluorescence using 293T cells was performed by transfecting cells with GFP-tagged MEKK3, GFP-Beclin 2, RFP-ATG9A, and/or Flag-Beclin 2 plasmids. To determine the transportation of GFP-MEKK3 into lysosome, cells were incubated with Lysotracker (Life Technology) and Hoechst 33342 at 24 h post-transfection. To determine the co-localization of GFP-MEKK3 and Flag-Beclin 2, cells were fixed by 4% paraformaldehyde and then incubated with anti-Flag antibody, followed by Alexa Fluor 633-conjugated anti-mouse IgG labeling. Microscopy was performed using a confocal microscope (Olympus, FV1000).

Hela or HEK293T cells can also be fixed for 15 min with 4% paraformaldehyde 24 h post-transfection and then permeabilized in methyl alcohol for 10 min at −20° C. After washing three times with PBS, fixed cells were blocked in 10% normal goat serum for 1 h, incubated with primary antibody overnight, and incubated with goat anti-mouse IgG, Alexa Fluor 594 secondary antibodies (A11032, ThermoFisher). Nuclei were stained with DAPI (ab104139, Abcam or 33342, 1:1,000 dilution, Hoechst) at 24 h post-transfection. Lysosome was stained with lysotracker-blue (1662594; 1:1,000 dilution, 40 min, 37° C., Life Technology) 24 h post-transfection, then washed twice with fresh medium. Microscopy was performed using a confocal microscope (Olympus, FV3000).

RNA extraction and real-time PCR. Total RNA was extracted from cells or homogenized tissues using TRIzol reagent (Invitrogen) or Direct-Zol™ RNA MiniPrep Plus w/TRI Reagent® (ZYMO Research, R2071) following the manufacturer's protocol. cDNA was prepared using SuperScript IV Reverse Transcriptase (Thermo Fisher), and quantitative RT-PCR was performed using SYBR™ Green PCR Master Mix (Thermo Fisher) on a QuantStudio 6 Flex Real-time PCR System (Applied Biosystems). All the data were normalized to GAPDH expression. Primer sequences for RT-PCR analysis of gene expression for human AIM2 (F: TCAAGCTGAAATGAGTCCTGC (SEQ ID NO: 115); R: CTTGGGTCTCAAACGTGAAGG (SEQ ID NO: 116)), mouse Aim2 (F: GTCACCAGTTCCTCAGTTGTG (SEQ ID NO: 117): R: CACCTCCATTGTCCCTGTTTAT (SEQ ID NO: 118)), human NLRP3 (F: CGTGAGTCCCATTAAGATGGAGT (SEQ ID NO: 119); R: CCCGACAGTGGATATAGAACAGA (SEQ ID NO: 120)), and mouse Nlrp3 (F: ATCAACAGGCGAGACCTCTG (SEQ ID NO: 121); R: GTCCTCCTGGCATACCATAGA (SEQ ID NO: 122)) are included as above. Additional RT-PCR primer sequences for each specific gene were listed in Table 4.

RNA sequencing and analysis. Total RNA was prepared from approximately 10 million cells or 20 μg of tissues by using TRIzol or the Direct-zol RNA MiniPrep Kit (Zymo Research). Each sample group contained two biological replicates. An RNA-seq library was prepared using Novogene's protocol. Briefly, mRNA was enriched using oligo(dT) beads and then fragmented randomly in fragmentation buffer. Next, cDNA was synthesized from these fragments using random hexamers and reverse transcriptase. After first-strand synthesis, a custom second-strand synthesis buffer (Illumina) was added along with dNTPs, RNase H and Escherichia coli polymerase I to generate the second strand via nick translation. The final cDNA library was sequenced using the Illumina HiSeq platform at Novogene. To compare gene expression levels under different conditions, a diagram of the distribution of fragments per kilobase of transcript sequence per million base pairs sequenced (FPKM) and a violin plot are used. DESeq, an R package based on a negative binomial distribution that models the number of reads from RNA-seq experiments, was applied for the analysis of differentially expressed genes (DEGs). The threshold for DEGs was set as padj<0.05 when using biological replicates in the experiments.

Enrichment of ATG9A⁺ vesicles and autophagosomes. 80% confluent WT, STX5 KO, and SIX6 KO 293T cells were transfected with Flag-ATG9A and HA-Beclin 2, while Becn2 KO 293T cells were transfected with Flag-ATG9A alone. Eight 15-cm dishes for each genotype were used for subsequent procedures. Half of the cells (4 dishes) were left untreated for immuno-isolation of Flag-ATG9A⁺ vesicles, the other half were treated with CQ (10 PM, 4 h) to inhibit the fusion of autophagosomes with lysosomes to enrich autophagosomes prior to membrane fractionation. Membrane fractionation to enrich autophagosomes were performed as previously described. To isolate ATG9A-associated vesicles, cells were rinsed once in cold PBS at 24 h post-transfection, then scraped, spun down and resuspended in 2.7× of fractionation buffer (140 mM KCl, 1 mM EGTA, 5 mM MgCl₂, 50 mM Sucrose, 20 mM HEPES, pH 7.4, supplemented with protease inhibitor). Cells were mechanically broken by spraying 4-5 times through a 23G needle attached to a 1 ml syringe, then spun down at 2000 g for 10 min, yielding a post-nuclear supernatant (PNS). Anti-Flag (100 μl, packed volume) was added to a 1.5 ml PNS aliquots and mixed by rotation at 4° C. overnight. Beads with the associated membranes were washed with 1 ml immunoisolation buffer three times and membranes bound to the beads were eluted and lysed using RIPA buffer, followed by immunoblotting analysis. Immunoblotting of R-actin in whole cell lysates served as an input control. Autophagosome inputs were adjusted by similar LC3 amount for WT, Becn2 KO, STX5 KO, and STX6 KO samples.

Transmission electron microscopy (TEM) and APEX2-enabled staining WT 293T cells with or without transfection of Flag-Beclin 2 were fixed in 2.5% (vol/vol) glutaraldehyde. The ultrathin sections (70-100 nm) of cell pellets were stained with lead citrate and uranyl acetate. The samples were viewed under a JEOL JEM-1400 TEM. For EM imaging of MEKK3-APEX2, WT, Becn2 KO, STX5 KO or STX6 KO cells were transfected and processed for staining following methods described previously. Briefly, cells were fixed with Karnovsky's fixative, incubated in 3,3′-Diaminobenzidine (DAB) solution, post-fixed in osmium tetroxide, stained with uranyl acetate, dehydrated in a series of graded ethanol and embedded in epoxy resin. Sections of 100 nm thickness were cut using a Leica EM UC7 ultramicrotome. Electron micrographs were collected using a JEOL JEM-1230 TEM equipped with a Gatan CCD camera.

For EM imaging of AIM2-APEX2, WT and BECN2 KO 293T cells were transfected with AIM2-APEX2 encoding plasmid alone or along with Flag-Beclin 2. At 24 h post-transfection, cells were fixed in 2.5% (vol/vol) glutaraldehyde and processed for staining. Briefly, cells were fixed with Karnovsky's fixative, incubated in 3,3′-Diaminobenzidine (DAB) solution, followed by post-fixation in osmium tetroxide and stained with uranyl acetate. After dehydrated in a series of graded ethanol and embedded in epoxy resin, samples were sectioned at 100 nm thickness using a Leica EM UC7 ultramicrotome. Electron micrographs were obtained under a JEOL JEM-1230 TEM equipped with a Gatan CCD camera.

Quantification and statistical analysis. Statistical analyses were performed using GraphPad Prism v6.0 and Excel, with a minimum of three biological independent samples for significance. Log-rank test was used for mouse survival or tumor incidence analysis. A one-way ANOVA or unpaired two-tailed Student's t-test was applied for other comparisons. P values of less than 0.05 were considered statistically significant.

Study Approval. Animal experiments described in this application were approved and carried out following the protocol (AUP-0115-0005 and AUP-0618-0036) provided by the IACUC at Houston Methodist Research Institute. IACUC uses the NIH Guide for the Care and Use of Laboratory Animals, which is based on the US Government Principles for Utilization and Care of Vertebrate Animals Used in testing, research, and training.

RNA-seq Data. All sequencing data that support the findings of this application have been deposited in NCBI's GEO (accession code GSE111539) for RNA-seq.

SEQUENCES

```
Beclin-2 protein (amino acid sequence)
SEQ ID NO: 1
MSSIRFLCQRCHQALKLSGSSESRSLPAAPAPTSGQAEPGDTREPGVTTREVTDAEEQQD
GASSRSPPGDGSVSKGHANIFTLLGELGAMHMLSSIQKAAGDIFDIVSGQAVVDHPLCEE
CTDSLLEQLDIQLALTEADSQNYQRCLETGELATSEDEAAALRAELRDLELEEARLVQE
LEDVDRNNARAAADLQAAQAEAAELDQQERQHYRDYSALKRQQLELLDQLGNVENQ
LQYARVQRDRLKEINCFTATFEIWVEGPLGVINNFRLGRLPTVRVGWNEINTAWGQAA
LLLLTLANTIGLQFQRYRLIPCGNHSYLKSLTDDRTELPLFCYGGQDVFLNNKYDRAMV
AFLDCMQQFKEEAEKGELGLSLPYGIQVETGLMEDVGGRGECYSIRTHLNTQELWTKA
LKFMLINFKWSLIWVASRYQK
Beclin-2 gene (nucleic acid sequence)
SEQ ID NO: 2
atgtcttccatccgcttcctgtgccagcgctgccaccaggccctgaagctgagcggctcctcggagtctaggagcctccctgcagccccg
gcgcccacctctgggcaggctgagcccggagacacccgggagcccggcgtcaccaccagggaggtgacagacgctgaggagcaaca
ggacggtgcctctagcagatcccctccaggcgatggcagtgtgtccaagggccatgccaacatcttcaccctgctgggggagcttggcgc
catgcacatgctcagtagcatccagaaggcagctggtgacatttttgacatagtctctggccaagcagttgtggaccatcccctgtgtgaaga
atgcaccgacagtcttttagagcagctggacatccagctcgctctcacagaagctgacagtcagaactaccaacgctgcctggagaccgg
ggagctggcgaccagcgaggacgaggcggcggcgctgcgggcggagctgcgggacctggagctggaggaggccaggctggtgca
ggagctggaggatgtggacaggaacaatgcaagagcagcggcggatctccaggcagcccaggcagaggctgcggagctggaccagc
aggagaggcagcactacagggactacagtgccttgaagcggcagcagctggaactgcttgatcagctggggaacgtggagaaccagct
gcagtatgccagggtccagagggaccggctgaaggaaatcaactgtttcaccgccacgtttgagatctgggggagggccccttgggcgt
catcaataacttcaggttgggccgcctccccactgtccgtgtgggctggaatgagattaacactgcctggggacaggcggccttgctgctcc
ttaccctggccaatacaattggactgcagtttcagaggtatcgactcatcccctgcggaaaccattcgtatctgaagtctttaacagatgaccg
cactgagctgccgttgttctgttatggggggcaggatgttttcctcaataacaagtatgaccgcgcgatggtggccttcctggactgcatgca
gcagttcaaggaagaggctgagaagggtgagctgggcctctctctgccctacgggatccaggtggagacaggcctgatggaggacgttg
gcggccgaggggaatgctattccatcagaacccatctgaacacgcaggagctgtggacaaaggcactcaagttcatgcttataaatttcaa
gtggagtctcatctgggttgcctcaaggtatcaaaagtag
ATG9A-binding domain of Beclin 2 protein (amino acid sequence)
SEQ ID NO: 3
MQAVVDHPLCEECTDSLLEQLDIQLALTEADSQNYQRCLETGELATSEDEAAALRAELR
DLELEEARLVQELEDVDRNNARAAADLQAAQAEAAELDQQERQHYRDYSALKRQQLE
LLDQLGNVENQLQYARVQRDRLKEINCFTATFEIWVEGPLGVINNFRLGRLPTVRVGW
NEINTAWGQAALLLLTLANTIGLQFQRYRLIPCGNHSYLKSLTDDRTELPLFCYGGQDV
FLNNKYDRAMVAFLDCMQQFKEEAEKGELGLSLPYGIQVETGLMEDVGGRGECYSIRT
HLNTQELWTKALKFMLINFKWSLIWVASRYQK
ATG9A-binding domain of Beclin 2 gene (nucleic acid sequence)
SEQ ID NO: 4
atgcaagcagttgtggaccatcccctgtgtgaagaatgcaccgacagtcttttagagcagctggacatccagctcgctctcacagaagctga
cagtcagaactaccaacgctgcctggagaccggggagctggcgaccagcgaggacgaggcggcggcgctgcgggcggagctgcgg
gacctggagctggaggaggccaggctggtgcaggagctggaggatgtggacaggaacaatgcaagagcagcggcggatctccaggc
agcccaggcagaggctgcggagctggaccagcaggagaggcagcactacagggactacagtgccttgaagcggcagcagctggaact
gcttgatcagctggggaacgtggagaaccagctgcagtatgccagggtccagagggaccggctgaaggaaatcaactgtttcaccgcca
cgtttgagatctgggtggagggccccttgggcgtcatcaataacttcaggttgggccgcctccccactgtccgtgtgggctggaatgagatt
aacactgcctggggacaggcggccttgctgctccttaccctggccaatacaattggactgcagtttcagaggtatcgactcatcccctgcgg
aaaccattcgtatctgaagtctttaacagatgaccgcactgagctgccgttgttctgttatggggggcaggatgttttcctcaataacaagtatg
accgcgcgatggtggccttcctggactgcatgcagcagttcaaggaagaggctgagaagggtgagctgggcctctctctgccctacggga
tccaggtggagacaggcctgatggaggacgttggcggccgaggggaatgctattccatcagaacccatctgaacacgcaggagctgtgg
acaaaggcactcaagttcatgcttataaatttcaagtggagtctcatctgggttgcctcaaggtatcaaaagtag
Complete coding sequence for scFv-Tau-Beclin 2-His-HA (amino acid
sequence and mapping)
SEQ ID NO: 5

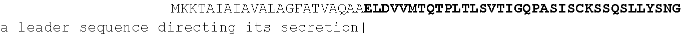

KTYLNWLLQRPGQSPKRLIYLVSKLDSGVPDRFTGSGSGTDFTLKISRVEAEDLGV
                                              scFv light chain

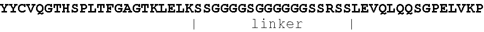

GASVKISCKTSEYTFTEYTKHWVKQSHGKSLEWIGSINPNNGDTYYNQKFTDKATL
                                              scFv heavy chain

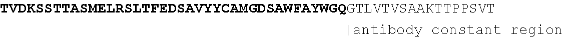

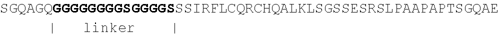


PGDTREPGVTTREVTDAEEQQDGASSRSPPGDGSVSKGHANIFTLLGELGAMHMLSSIQ
KAAGDIFDIVSGQAVVDHPLCEECTDSLLEQLDIQLALTEADSQNYQRCLETGELATSED
EAAALRAELRDLELEEARLVQELEDVDRNNARAAADLQAAQAEAAELDQQERQHYRD
                                                    Beclin 2
YSALKRQQLELLDQLGNVENQLQYARVQRDRLKEINCFTATFEIWVEGPLGVINNFRLG
RLPTVRVGWNEINTAWGQAALLLLTLANTIGLQFQRYRLIPCGNHSYLKSLTDDRTELP
LFCYGGQDVFLNNKYDRAMVAFLDCMQQFKEEAEKGELGLSLPYGIQVETGLMEDVG

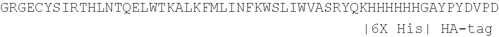

YAS
Complete coding sequence for scFv-Tau-Beclin 2-His-HA (nucleic acid
sequence)
SEQ ID NO: 6
ATGAAGAAAACCGCCATTGCCATTGCCGTGGCTCTGGCCGGATTTGCCACAGTTGCT
CAGGCCGCCGAACTGGACGTTGTGATGACACAGACCCCTCTGACACTGAGCGTGAC
CATTGGACAGCCTGCCAGCATCAGCTGCAAGAGCAGTCAGAGCCTGCTGTACTCCA
ACGGCAAGACCTACCTGAACTGGCTGCTGCAGAGGCCTGGCCAGAGTCCTAAGAGA
CTGATCTACCTGGTGTCCAAGCTGGACAGCGGCGTGCCCGATAGATTCACAGGCTCT
GGCAGCGGCACCGACTTCACCCTGAAGATCTCTAGAGTGGAAGCCGAGGACCTGGG
CGTGTACTATTGTGTGCAGGGCACACACAGCCCTCTGACCTTTGGAGCCGGCACAAA
GCTGGAACTGAAGTCTAGCGGAGGCGGCGGATCTGGTGGTGGTGGCGGAGGATCTA
GCAGATCCAGTCTGGAAGTCCAGCTGCAGCAGAGCGGACCTGAGCTTGTGAAACCT
GGCGCCTCCGTGAAGATTTCCTGCAAGACCAGCGAGTACACCTTCACCGAGTACAC
AAAGCACTGGGTCAAGCAGTCCCACGGCAAGAGCCTGGAATGGATCGGCAGCATCA
ACCCCAACAACGGCGACACCTACTACAACCAGAAGTTCACCGACAAGGCCACACTG
ACCGTGGACAAGAGCAGCACCACAGCCAGCATGGAACTGCGGAGCCTGACCTTCGA
AGATAGCGCCGTGTACTACTGCGCCATGGGCGACTCTGCTTGGTTTGCCTATTGGGG
CCAGGGCACCCTGGTTACAGTGTCTGCCGCCAAGACAACCCCTCCTAGCGTTACATC
TGGACAGGCCGGACAAGGTGGCGGTGGTTCTGGCGGCGGAGGTAGTGGCGGAGGC
GGATCTTCTTCCATCCGCTTCCTGTGCCAGCGCTGCCACCAGGCCCTGAAGCTGAGC
GGCTCCTCGGAGTCTAGGAGCCTCCCTGCAGCCCCGGCGCCCACCTCTGGGCAGGCT
GAGCCCGGAGACACCCGGGAGCCCGGCGTCACCACCAGGGAGGTGACAGACGCTG
AGGAGCAACAGGACGGTGCCTCTAGCAGATCCCCTCCAGGCGATGGCAGTGTGTCC
AAGGGCCATGCCAACATCTTCACCCTGCTGGGGGAGCTTGGCGCCATGCACATGCTC
AGTAGCATCCAGAAGGCAGCTGGTGACATTTTTGACATAGTCTCTGGCCAAGCAGTT
GTGGACCATCCCCTGTGTGAAGAATGCACCGACAGTCTTTTAGAGCAGCTGGACATC
CAGCTCGCTCTCACAGAAGCTGACAGTCAGAACTACCAACGCTGCCTGGAGACCGG
GGAGCTGGCGACCAGCGAGGACGAGGCGGCGGCGCTGCGGGCGGAGCTGCGGGAC
CTGGAGCTGGAGGAGGCCAGGCTGGTGCAGGAGCTGGAGGATGTGGACAGGAACA
ATGCAAGAGCAGCGGCGGATCTCCAGGCAGCCCAGGCAGAGGCTGCGGAGCTGGA
CCAGCAGGAGAGGCAGCACTACAGGGACTACAGTGCCTTGAAGCGGCAGCAGCTG
GAACTGCTTGATCAGCTGGGGAACGTGGAGAACCAGCTGCAGTATGCCAGGGTCCA
GAGGGACCGGCTGAAGGAAATCAACTGTTTCACCGCCACGTTTGAGATCTGGGTGG
AGGGCCCCTTGGGCGTCATCAATAACTTCAGGTTGGGCCGCCTCCCCACTGTCCGTG
TGGGCTGGAATGAGATTAACACTGCCTGGGGACAGGCGGCCTTGCTGCTCCTTACCC
TGGCCAATACAATTGGACTGCAGTTTCAGAGGTATCGACTCATCCCCTGCGGAAACC
ATTCGTATCTGAAGTCTTTAACAGATGACCGCACTGAGCTGCCGTTGTTCTGTTATG
GGGGGCAGGATGTTTTCCTCAATAACAAGTATGACCGCGCGATGGTGGCCTTCCTGG
ACTGCATGCAGCAGTTCAAGGAAGAGGCTGAGAAGGGTGAGCTGGGCCTCTCTCTG
CCCTACGGGATCCAGGTGGAGACAGGCCTGATGGAGGACGTTGGCGGCCGAGGGG
AATGCTATTCCATCAGAACCCATCTGAACACGCAGGAGCTGTGGACAAAGGCACTC
AAGTTCATGCTTATAAATTTCAAGTGGAGTCTCATCTGGGTTGCCTCAAGGTATCAA
AAGCATCATCATCATCATCATGGCGCGTATCCGTATGATGTGCCGGATTATGCGAGC
TAG
light chain sequence of scFv-Tau-Beclin 2-His-HA
SEQ ID NO: 7
ELDVVMTQTPLTLSVTIGQPASISCKSSQSLLYSNGKTYLNWLLQRPGQSPKRLIYLVSK
LDSGVPDRFTGSGSGTDFTLKISRVEAEDLGVYYCVQGTHSPLTFGAGTKLELK,
heavy chain sequence of scFv-Tau-Beclin 2-His-HA
SEQ ID NO: 8
LEVQLQQSGPELVKPGASVKISCKTSEYTFTEYTKHWVKQSHGKSLEWIGSINPNNGDT
YYNQKFTDKATLTVDKSSTTASMELRSLTFEDSAVYYCAMGDSAWFAYWGQ,
(light and heavy chain sequences of scFv-Tau-Beclin 2-His-HA)
SEQ ID NO: 9
ELDVVMTQTPLTLSVTIGQPASISCKSSQSLLYSNGKTYLNWLLQRPGQSPKRLIYLVSK
LDSGVPDRFTGSGSGTDFTLKISRVEAEDLGVYYCVQGTHSPLTFGAGTKLELKSSGGG
GSGGGGGGSSRSSLEVQLQQSGPELVKPGASVKISCKTSEYTFTEYTKHWVKQSHGKSL
EWIGSINPNNGDTYYNQKFTDKATLTVDKSSTTASMELRSLTFEDSAVYYCAMGDSAW
FAYWGQ
```

1. A recombinant protein or polypeptide comprising i) a modified or unmodified Beclin 2 polypeptide, protein, or a fragment thereof and ii) a targeting moiety.

2. The recombinant protein or polypeptide of claim 1, wherein the modified or unmodified Beclin 2 polypeptide, protein, or fragment thereof is at least 70% identical to SEQ ID NO: 1.

3. The recombinant protein or polypeptide of claim 1, wherein the modified or unmodified Beclin 2 polypeptide, protein, or fragment thereof comprises an ATG9A-binding domain.

4. The recombinant protein or polypeptide of claim 3, wherein the ATG9A-binding domain comprises a polypeptide sequence at least 70% identical to SEQ ID NO: 3.

5. (canceled)

6. The recombinant protein or polypeptide of claim 1, wherein the targeting moiety specifically binds to a peptide, protein, and/or pathogenic molecule related to a neurodegenerative disease, the peptide, protein, and/or pathogenic molecule comprising TAU, β-amyloid, APOE, SUPT5H TDP43, GAK, PINK1, PARK2, PARK7, or TREM2.

7. (canceled)

8. The recombinant protein or polypeptide of claim 1, wherein the targeting moiety specifically binds to a peptide, protein, and/or pathogenic molecule related to a cancer and/or metastasis, and/or the targeting moiety specifically binds to TAK1, MEKK3, TAK1 MEKK3, a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LiCAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COLIA1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIPIL1, FLI1, FLT3, FLT4, FMS, FNBP1, FOXOIA, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, or ZNFN1A1.

9. The recombinant protein or polypeptide of claim 1, wherein the targeting moiety comprises an antibody or a functional fragment thereof, or a small molecule.

10. The recombinant protein or polypeptide of claim 9, wherein;
the antibody fragment is selected from the group consisting of a Fab antibody, a single-chain variable fragment (scFv) antibody, and a V_{H}H antibody;
the antibody or antibody fragment specifically binds to TAU;
the antibody or antibody fragment comprises a light chain variable region comprising a polypeptide sequence at least 80% identical to SEQ ID NO: 7 and/or a heavy chain variable region comprising a polypeptide sequence at least 80% identical to SEQ ID NO: 8; and/or
the antibody or antibody fragment comprises a polypeptide sequence at least 80% identical to SEQ ID NO: 9.

11-16. (canceled)

17. A recombinant polynucleotide encoding the recombinant protein or polypeptide of claim 1 or an engineered Beclin 2 protein or polypeptide, the recombinant polynucleotide comprising a polynucleotide sequence at least 80% identical to SEQ ID NO: 6.

18. A vector comprising the recombinant polynucleotide of claim 17.

19. (canceled)

20. A method of treating, inhibiting, reducing, decreasing, ameliorating, and/or preventing a cancer or metastasis in a subject in need thereof, the method comprising administering to the subject a therapeutically effective amount of the recombinant protein or polypeptide of claim 1.

21. The method of claim 20, wherein the subject is a cancer patient, and the cancer comprises metastatic lymphoma, lung cancer, T cell lymphoma, or B cell lymphoma.

22. The method of claim 20, wherein the recombinant protein or polypeptide increases a level of Beclin-2 polypeptide in a cancer cell, decreases a level of TAK1, and/or MEKK3 in a cancer cell and/or decreases cancer cell proliferation.

23. (canceled)

24. A method of treating, inhibiting, reducing, decreasing, ameliorating, and/or preventing a neurodegenerative disease in a subject in need thereof and/or increasing a level of Beclin-2 polypeptide in a neural cell, the method comprising administering to the subject a therapeutically effective amount of the recombinant protein or polypeptide of claim 1.

25. The method of claim 24, wherein the subject is a neurodegenerative disease patient, and the neurodegenerative disease comprises Alzheimer's disease (AD), Parkinson's disease (PD), Huntington's Disease, Amyotrophic Lateral Sclerosis (ALS), or Multiple Sclerosis (MS).

26. The method of claim 24, wherein the recombinant protein or polypeptide decreases a level of an inflammatory cytokine comprisiing IL-1b or IL6; decreases a level of AIM2, NLRP3, NLRP1, NLRC4, in a cell; and/or decreases a level of inflammation or a neurogenerative desease-related molecule in a cell.

27-29. (canceled)

30. The method of claim 26, wherein the disease-related molecule comprises TAU, β-amyloid, APOE, SUPT5H TDP43, GAK, PINK1, PARK2, PARK7, or TREM2 protein and/or the cell, in which the level of inflammation or a neurogenerative disease-related molecule decreases, is a neural or immune cell.

31-47. (canceled)

48. A method of treating, inhibiting, reducing, decreasing, ameliorating, and/or preventing a cancer or metastasis in a subject in need thereof, the method comprising administering to the subject a therapeutically effective amount of a) an engineered Beclin 2 protein or polypeptide and/or b) a recombinant protein or polypeptide comprising a modified or unmodified Beclin 2 protein, polypeptide, or fragment thereof.

49. The method of claim 48, wherein the recombinant protein or polypeptide further comprises a targeting moiety operatively linked to the Beclin 2 polypeptide or a fragment thereof.

50. The method of claim 48, wherein:
the Beclin 2 protein or polypeptide is at least 70% identical to SEQ ID NO: 1;
the Beclin 2 polypeptide comprises an ATG9A-binding domain; and/or
the ATG9A-binding domain comprises a polypeptide sequence at least 70% identical to SEQ ID NO: 3.

51-53. (canceled)

54. The method of claim 49, wherein the targeting moiety specifically binds to a peptide, protein, and/or pathogenic molecule related to a cancer; and/or the targeting moiety specifically binds to NLRP3, NLRP1, NLRC4, AIM2, TAK1 MEKK3, a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin B1, lectin-reactive AFP, Fos-related antigen 1, ADRB3, thyroglobulin, AKAP-4, OY-TES1, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen-1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGFII, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LiCAM, MAGE proteins, MAGE-A1, MLANA, MSLN, MUC1, MUC2, MUC3, MUC4, MUC5, MUC16, MUM1, ANKRD30A, NY-ESO1 (CTAGIB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides. ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14^{ARF}, CDKN2A-p16^{INK4A}, CDX2, CEBPA, CEP1, CHEK2, CHIC2, CHN1, CLTC, c-met, c-myc, COLIA1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIPIL1, FLI1, FLT3, FLT4, FMS, FNBP1, FOXOIA, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKARIA, PRO1073, PSIP2, PTCH, PTEN, PTPN11, RABSEP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, or ZNFN1A1.

55. The method of claim 49, wherein:
the targeting moiety comprises an antibody or functional fragment thereof; and/or
the antibody fragment is selected from the group consisting of a Fab antibody, a single-chain variabile fragment (scFv) antibody, and a V_{H}H antibody.

56-57. (canceled)

58. The method of claim 48, wherein the cancer comprises metastatic lymphoma, lung cancer, T cell lymphoma, or B cell lymphoma.

59. The method of claim 48, wherein the recombinant protein or polypeptide decreases a level of TAK1 and/or MEKK3 in a cancer or immune cell and/or decreases cancer cell proliferation.

60. (canceled)

61. A method of treating, inhibiting, reducing, decreasing, ameliorating, and/or preventing a neurodegenerative disease in a subject in need thereof, the method comprising administering to the subject a therapeutically effective amount of a) an engineered Beclin-2 protein or polypeptide and/or b) a recombinant protein or polypeptide comprising a modified or unmodified Beclin 2 protein, polypeptide, or fragment thereof.

62. The method of claim 61, wherein the recombinant protein or polypeptide further comprises a targeting moiety operatively linked to the Beclin 2 polypeptide or fragment thereof.

63. The method off claim 61, wherein:
the modified or unmodified Beclin 2 protein or polypeptide is at least 70% identical to SEQ ID NO: 1;
the Beclin 2 polypeptide comprises an ATG9A-binding domain; and/or
the ATG9A-binding domain comprises a polypeptide sequence at least 70% identical to SEQ ID NO: 3.

64-65. (canceled)

66. The method of claim 62, wherein the targeting moiety specifically binds to a peptide, protein, and/or pathogenic molecule related to a neurodegenerative disease and/or the targeting moiety specifically binds to TAU, β-amyloid, APOE, SUPT5H TDP43, GAK, PINK1, PARK2, PARK7, or TEM2 protein.

67. (canceled)

68. The method of claim 62, wherein:
the targeting moiety comprises an antibody or functional fragment thereof, or a small molecule;
the antibody fragment is selected from the group consisting of a Fab antibody, a single-chain variable fragment (scFv) antibody, and a V_{H}H antibody;
the antibody or antibody fragment specifically binds to TAU;
the antibody or antibody fragment comprises a light chain variable region comprising a polypeptide sequence at least 80% identical to SEQ ID NO: 7 and/or a heavy chain variable region comprising a polypeptide sequence at least 80% identical to SEQ ID NO: 8; and/or
the antibody or antibody fragment comprises a polypeptide sequence at least 80% identical to SEQ ID NO: 9.

69-73. (canceled)

74. The method of claim 61, wherein the neurodegenerative disease comprises Alzheimer's disease (AD), Parkinson's disease (PD), Huntington's Disease, Amyotrophic Lateral Sclerosis (ALS), or Multiple Sclerosis (MS).

75. The method of claim 62, wherein:
the recombinant protein or polypeptide decreases a level of an inflammatory cytokine;
the inflammatory cytokine comprises IL-1b or IL-6;
the recombinant protein or polypeptide decreases a level of AIM2, NLRP3, NLRP1, and/or NLRC4 in an immune cell;
the recombinant protein or polypeptide decreases a level of a neurogenerative disease-related molecule in a neural cell; and/or
the neurogenerative disease-related molecule comprises TAU, β-amyloid, APOE, SUPT5H TDP43, GAK, PINK1, PARK2, PARK7, or TREM2 protein.

76-79. (canceled)
